(19) United States
(12) Patent Application Publication
(10) Pub. No.: US 2025/0263412 A1
(43) Pub. Date: Aug. 21, 2025

(54) NON-HYDROXAMATE HDAC6 INHIBITORS AND RELATED METHODS OF USE

(71) Applicant: The Regents of the University of Michigan, Ann Arbor, MI (US)

(72) Inventors: Mathivanan Packiarajan, Ann Arbor, MI (US); Pil Lee, Ann Arbor, MI (US); Andrew White, Ann Arbor, MI (US); Isin Cakir, Ann Arbor, MI (US); Roger Cone, Ann Arbor, MI (US)

(21) Appl. No.: 18/282,424

(22) PCT Filed: Mar. 15, 2022
(86) PCT No.: PCT/US2022/020364

§ 371 Date: Sep. 15, 2023

Related U.S. Application Data
(60) Provisional application No. 63/161,129, filed on Mar. 15, 2021.

(51) Int. Cl. C07D 471/08; A61K 31/444; A61K 31/4995; A61K 31/5377; C07D 471/10; C07D 487/08; C07D 487/10

(52) U.S. Cl. CPC C07D 471/08; A61K 31/444; A61K 31/4995; A61K 31/5377; C07D 471/10; C07D 487/08; C07D 487/10

(57) ABSTRACT

This invention is in the field of medicinal chemistry. In particular, the invention relates to a new class of small-molecules having a heteroaryl substituted oxadiazole structure which function as non-hydroxamate histone deacetylase 6 (HDAC6) inhibitors, and their use as therapeutics for the treatment of metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

This application claims priority to U.S. provisional patent application Ser. No. 63/161,129, filed Mar. 15, 2021, which is incorporated herein by reference in its entirety.

### FIELD OF THE INVENTION

This invention is in the field of medicinal chemistry. In particular, the invention relates to a new class of small-molecules having a heteroaryl substituted oxadiazole structure which function as non-hydroxamate histone deacetylase 6 (HDAC6) inhibitors, and their use as therapeutics for the treatment of metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

### BACKGROUND OF THE INVENTION

Obesity and associated disorders including type II Diabetes, cardiovascular diseases and cancer have reached epidemic rates in the world¹. A hallmark of diet-induced obesity is hyperleptinemia. Leptin is a 16 kDa hormone produced mainly by the adipose tissue in proportion to the size of the fat depots, and acts through its receptors (LepRb) expressed predominantly in the central nervous system (CNS) including the hypothalamus, brain stem and midbrain². Leptin administration to leptin deficient mice (ob/ob) reduces food intake and increases energy expenditure, resulting in profound weight loss. In diet-induced obesity, the circulating leptin levels rise proportionally to adiposity, and may reach levels 10-40 fold higher than the lean state³. Despite this hyperleptinemic state, obese rodents and humans maintain their increased adiposity, and show a blunted response to exogenous leptin administration, which has been characterized as leptin resistance⁴.

Defective protein homeostasis (proteostasis) has emerged as a contributing factor to the metabolic syndrome⁵⁻⁸. Impairments in proteostatic processes such as autophagy^{9,10}, the heat shock response¹¹⁻¹⁴, ubiquitin-proteasome pathway^{8,15,16}, and integrated stress responses^{17,18} have been implicated in the pathophysiology of obesity and Diabetes. A central component of these proteostatic mechanisms is histone deacetylase 6 (HDAC6), a microtubule-associated member of the HDAC family that is predominantly localized to the cytoplasm. In addition to its deacetylase activity, and E-3 ligase activity¹⁹, HDAC6 has non-enzymatic functions largely due to its C-terminal ubiquitin-binding domain (UBD)^{20,21}, making it a unique HDAC that can interact with proteins normally targeted to degradation through the proteasome. Cellular processes regulated by HDAC6 include aggresome and stress granule formation^{22,23}, autophagy ²⁴, heat shock response, and recycling of dysfunctional mitochondria through mitophagy^{25,26}

Deregulation of HDAC6 activity has been associated to a variety of diseases including cancer, neurodegenerative diseases and pathological autoimmune response (see, Seidel C, et al., Epigenomics. 2015; 7 (1): 103-18).

Accordingly, effective HDAC6 inhibitors are needed for the treatment of such conditions related to HDAC6 activity.

The present invention addresses this need.

### SUMMARY OF THE INVENTION

Obesity and overweight affect more than one third of the world population, and are significant risk factors for type II Diabetes, cardiovascular diseases and cancer. Common obesity is usually accompanied by hyperleptinemia and leptin resistance. Experiments conducted during the course of developing embodiments for the present invention designed, synthesized and biologically evaluated compounds having a heteroaryl substituted oxadiazole structure which function as HDAC6 inhibitors.

Accordingly, this invention relates to a new class of small-molecules having a heteroaryl substituted oxadiazole structure which function as HDAC6 inhibitors, and their use as therapeutics for the treatment of metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

Certain a heteroaryl substituted oxadiazole (or similar) compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, compounds encompassed within the following formula (Formula I) are provided:

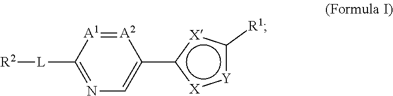

including pharmaceutically acceptable salts, solvates, and/or prodrugs thereof.

In some embodiments, the compound of Formula I is selected from Formulas (Iaa), (Iaaa₁), (Iaaa₂), (Iaaa₃), (Iaaa₄), or (Iaaa₅):

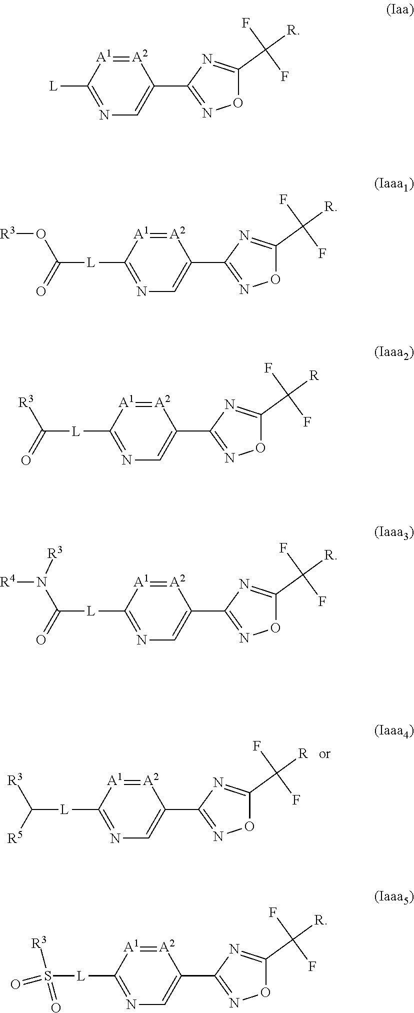

In some embodiments, the compound of Formula I is selected from Formulas (Iba), (Ibaa₁), (Ibaa₂), (Ibaa₃), (Ibaa₄), or (Ibaa₅):

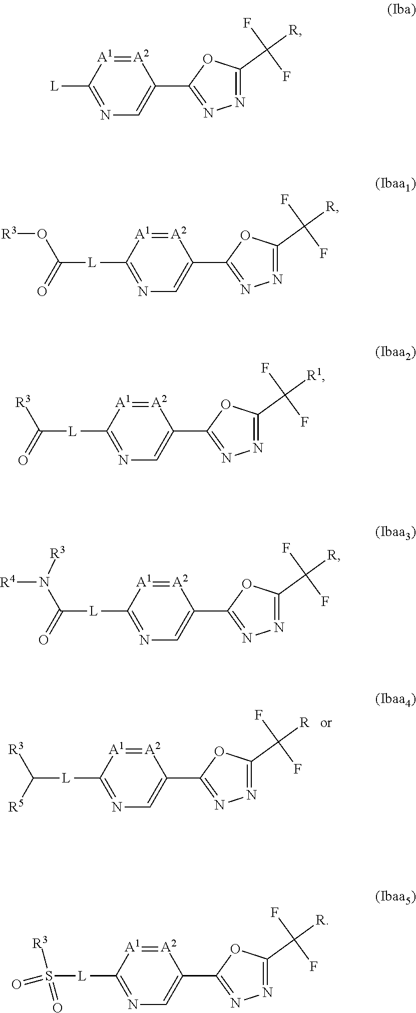

In some embodiments, the compound of Formula I is selected from (Ia), (Ib), (Ic) and (Id):

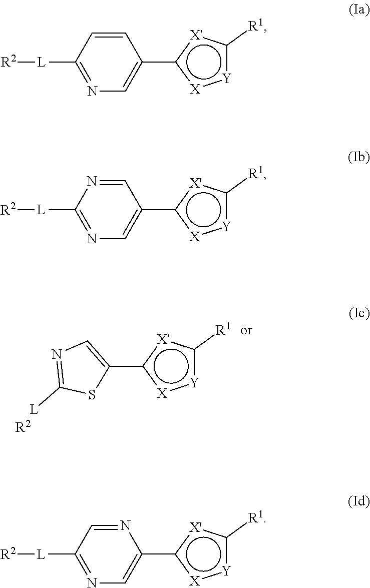

Formula I (and any formulas encompassed within Formula I) is not limited to a particular chemical moiety for A¹, A², L, X, X′, Y, R, R¹, R², R³, and R⁴. In some embodiments, the particular chemical moieties for A¹, A², L, X, X′, Y, R, R¹, R², R³, and R⁴ independently include any chemical moiety that permits the resulting compound to inhibit HDAC6 activity.

Such embodiments are not limited to a particular chemical moiety for X, X′ and Y. In some embodiments, X and X′ are N when Y is O, or X and Y are N when X′ is O.

Such embodiments are not limited to a particular chemical moiety for A¹ and A². In some embodiments, A¹ and A² are independently CH or N, or A¹ and A² are independently CH, or A¹ and A² taken together as S.

Such embodiments are not limited to a particular chemical moiety for R. In some embodiments, R is fluorine or hydrogen.

Such embodiments are not limited to a particle chemical moiety for R¹. In some embodiments, R¹ is CF₃ or CF₂H.

In some embodiments,

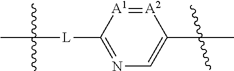

is selected from the group consisting of:

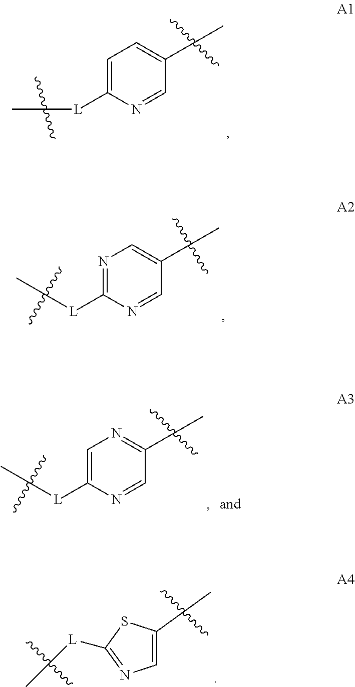

Such embodiments are not limited to a particular chemical moiety for L. In some embodiments, L is a bicyclic or spirocyclic diamine.

In some embodiments, L is a class of bicyclic diamines, where the bicyclic diamines are 2,5-diazabicyclo[2.2.1]heptane, or 3,6-diazabicyclo[3.1.1]heptane, 3,6-diazabicyclo[3.1.0]hexane, 3,7-diazabicyclo[4.1.0]heptane, octahydropyrrolo[3,4-c]pyrrole, octahydro-1H-pyrrolo[3,4-c]pyridine, 3,8-diazabicyclo[3.2.1]octane, 3,6-diazabicyclo[3.2.1]octane, 3,9-diazabicyclo[3.3.1]nonane, 2,5-diazabicyclo[2.2.2]octane, or 2,5-diazabicyclo[4.2.0]octane,

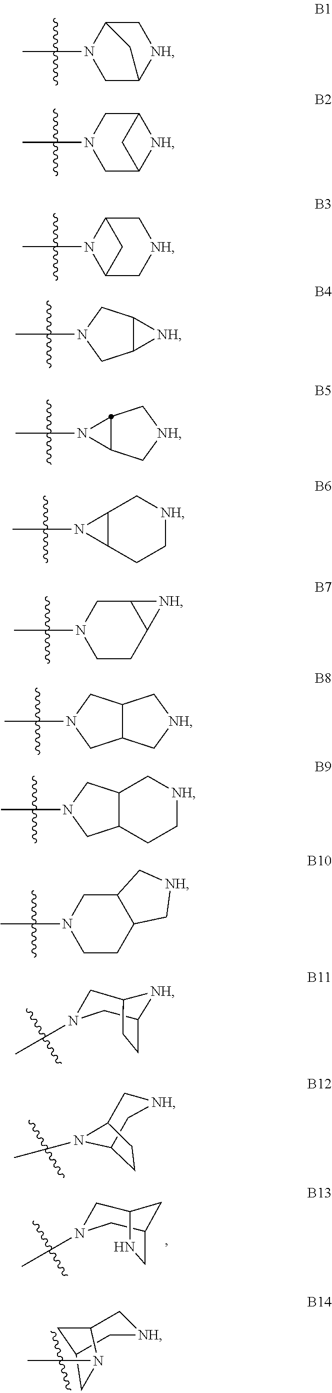

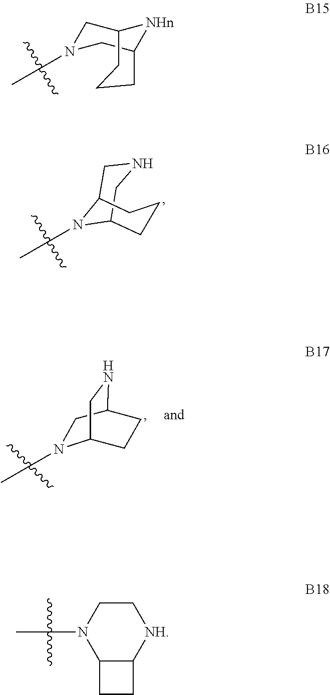

In some embodiments, L is a class of spirocyclic diamines, where the spirocyclic diamines are 2,6-diazaspiro[3.3]heptane, 2,6-diazaspiro[3.4]octane, 2,7-diazaspiro[4.4]nonane, 2,7-diazaspiro[3.5]nonane, 2,7-diazaspiro[3.5]nonane, 2,8-diazaspiro[4.5]decane, 2,9-diazaspiro[5.5]undecane, 3,9-diazaspiro[5.5]undecane and 4,7-diazaspiro[2.5]octane,

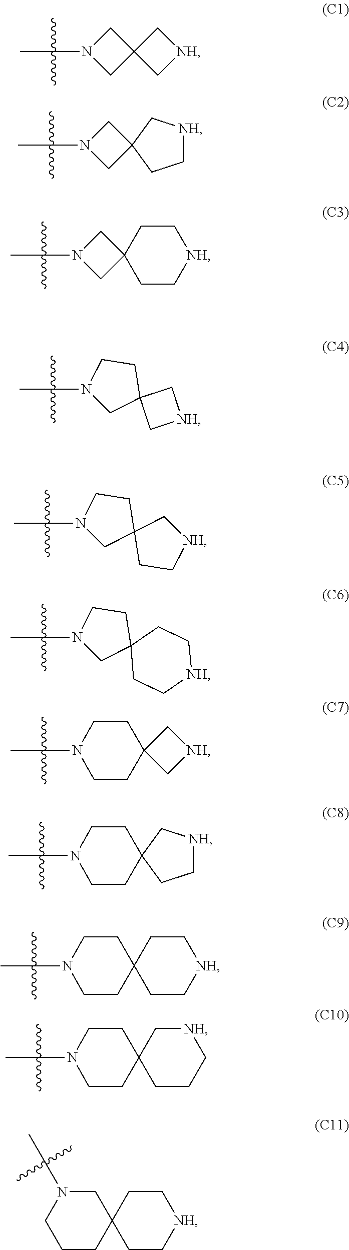

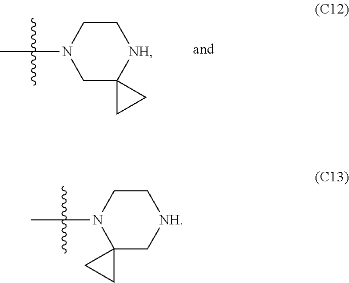 [SMILES: *N1CCNC2(CC2)C1 ; *N1CCNCC12CC2]

Such embodiments are not limited to a particular chemical moiety for R². In some embodiments, R² is hydrogen, —COR³, —CO₂R³, —(CH₂)ₙNR³R⁴, —CONR³R⁴, —(CH₂)ₙR³R⁴, and —SO₂R³, wherein n=1 or 2 optionally substituted with a CH₃ group.

Such embodiments are not limited to a particular chemical moiety for R³. In some embodiments, R³ is hydrogen, C₁-C₆alkyl or C₁-C₆branchedalkylalkyl or C₁-C₆cycloalkyl, aryl, cycloalkylalkyl, heterocyclyl, heteroaryl, arylalkyl, arylaryl, or aryl, each of which is optionally mono-, di-, or tri-substituted independently with C₁-C₆alkyl, halogen, cycloalkyl, and aryl.

Such embodiments are not limited to a particular chemical moiety for R⁴. In some embodiments, R⁴ is C₁-C₆alkyl, cycloalkyl, cycloalkylalkyl, heterocyclyl, heteroaryl, arylalkyl, or aryl, each of which is optionally mono-, di-, or tri-substituted independently with C₁-C₆alkyl, halogen, cycloalkyl, and aryl.

In some embodiments, R³ and R⁴ are each independently aryl, heteroaryl, alkyl, cycloalkyl, ketocycloalkyl, heterocyclyl, or —N(R⁴)R⁵, each of which is optionally mono- or di-substituted independently with C₁-C₆alkyl, cycloalkyl, alkoxy, aryl, cyano, halogen, —N(R⁴) R⁵, or —N(R⁵)R⁶.

In some embodiments, R³ and R⁴ are each independently aryl, heteroaryl, alkyl, cycloalkyl, ketocycloalkyl, heterocyclyl, or —N(R⁴)R⁵, each of which is optionally mono- or di-substituted independently with C₁-C₆alkyl, cycloalkyl, alkoxy, aryl, cyano, halogen or —N(R⁵)R⁶.

Such embodiments are not limited to a particular chemical moiety for R⁵. In some embodiments, R⁵ is hydrogen or CH₃.

Such embodiments are not limited to a particular chemical moiety for R⁶. In some embodiments, R⁶ is hydrogen, C₁-C₃alkyl, CF₃, CHF₂, or C₃-C₆cycloalkyl.

In some embodiments, R⁵ and R⁶ taken together with the N to which they attach form H, alkyl, heteroaryl, cycloalkyl, or spirocyclic, which is optionally mono-, di-, or tri-substituted independently with C₁-C₆alkyl, C₁-C₆alkoxy, aryl, cycloalkyl, aryl, ═O, or halogen. In some embodiments, cycloalkyl is a cyclopropyl, cyclobutyl, cyclopentyl, or cyclohexyl, which is optionally substituted independently with C₁-C₆alkyl, C₁-C₆alkoxy, aryl, heteroaryl, cycloalkyl, aryl, halogenoalkyl. In some embodiments, the heterocyclyl is pipreazinyl, piperidinyl, pyrrolidinyl, tetrahydrofuranyl, tetrahydropyranyl, oxetanyl, tetrahydrothiopyranyl, or tetrahydrothiofuranyl which is optionally substituted independently with C₁-C₆alkyl, C₁-C₆alkoxy, aryl, Heteroaryl, cycloalkyl, aryl, ═O, halogenoalkyl, or alkylsulfonamido.

In a particular embodiment, compounds encompassed within Formula II are provided:

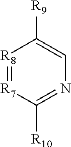

(Formula II); including pharmaceutically acceptable salts, solvates, and/or prodrugs thereof.

Formula II is not limited to a particular chemical moiety for R7, R8, R9 and R10. In some embodiments, the particular chemical moieties for R7, R8, R9 and R10 independently include any chemical moiety that permits the resulting compound to inhibit HDAC6 activity.

Such embodiments are not limited to a specific chemical moiety for R7. In some embodiments, R7 is Carbon or Nitrogen.

Such embodiments are not limited to a specific chemical moiety for R8. In some embodiments, R8 is Carbon or Nitrogen.

In some embodiments, R7 and R8 together is Sulfur such that the resulting structure is

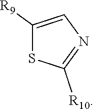

Such embodiments are not limited to a specific chemical moiety for R9. In some embodiments, R9 is selected from

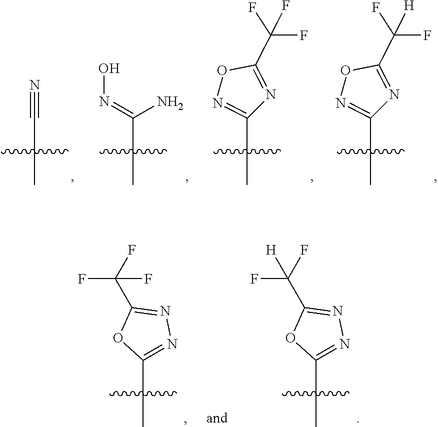

Such embodiments are not limited to a specific chemical moiety for R10. In some embodiments, R10 is selected from hydrogen, halogen (e.g., Chlorine),

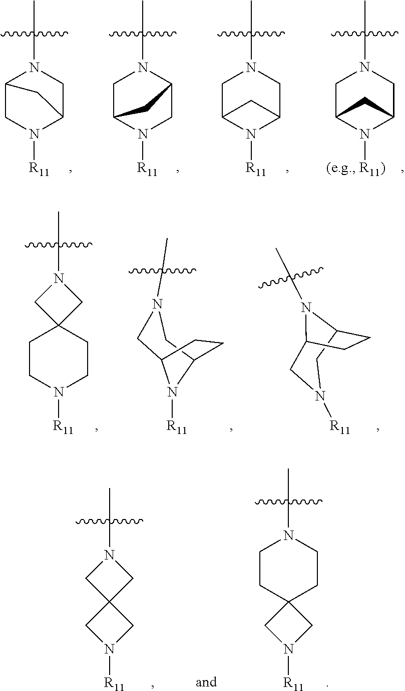

Such embodiments are not limited to a specific chemical moiety for R11. In some embodiments, R11 is selected from hydrogen,

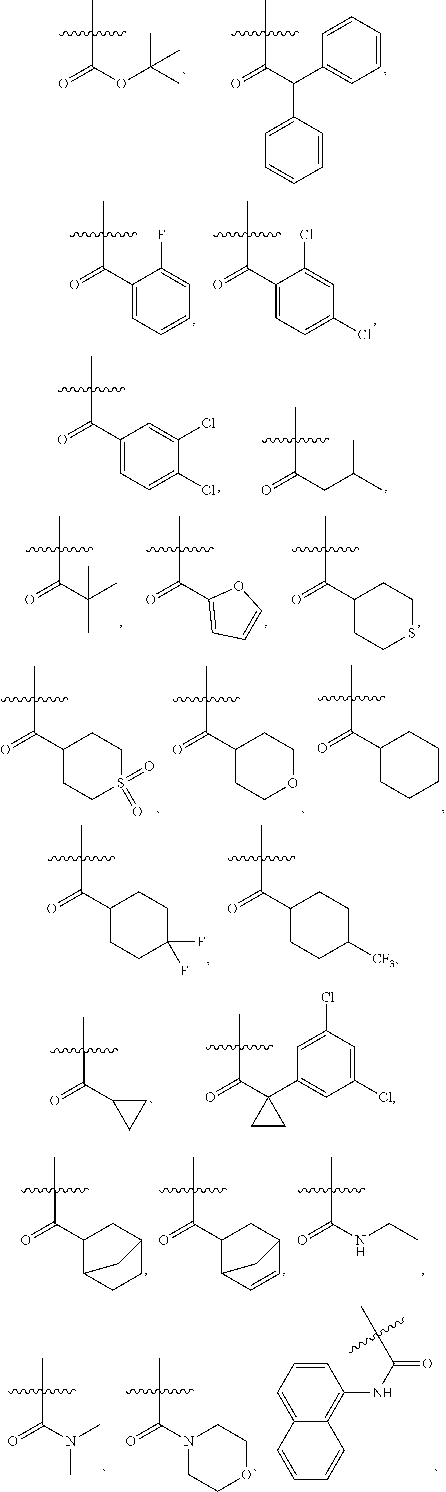

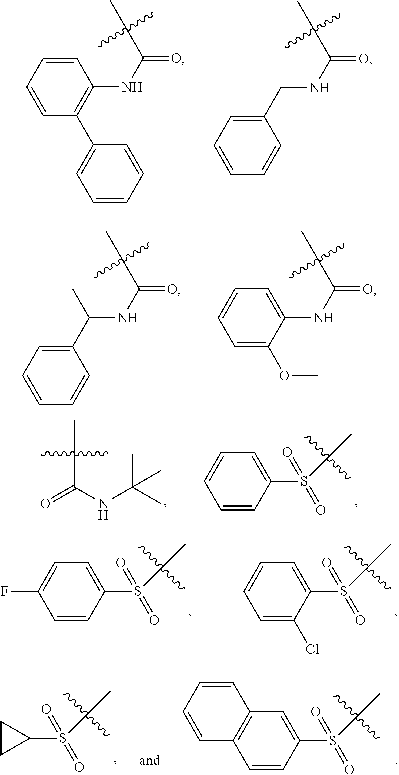

In certain embodiments, the compound encompassed within Formulas I or II is selected from any of the compound recited in Example B (e.g., Tables I, II, III, and IV).

The invention further provides processes for preparing any of the compounds of the present invention.

The invention also provides the use of compounds to not only inhibit HDAC6 activity but also signaling pathways dependent upon HDAC6 activity. The invention also relates to the use of compounds for sensitizing cells to additional agent(s), such as agents known to be effective in the treatment of metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

The compounds of the invention are useful for the treatment, amelioration, or prevention of disorders associated with HDAC6 activity (e.g., metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

The compounds of the invention are useful for increasing leptin sensitivity in tissue (e.g., adipose tissue) in patients suffering from or at risk of suffering from disorders associated with HDAC6 activity (e.g., metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents, e.g., agents useful in treating disorders associated with HDAC6 activity (e.g., metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

The present disclosure further provides bifunctional compounds that function to recruit endogenous proteins to an E3 Ubiquitin Ligase for degradation, and methods of using the same. In particular, the present disclosure provides bifunctional or proteolysis targeting chimeric (PROTAC) compounds, which find utility as modulators of targeted ubiquitination of a variety of polypeptides and other proteins, which are then degraded and/or otherwise inhibited. An exemplary advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as any type of disorders characterized with HDAC6 activity (e.g., metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

In an additional aspect, the disclosure provides bifunctional or PROTAC compounds, which comprise an E3 Ubiquitin Ligase binding moiety (e.g., a ligand for an E3 Ubquitin Ligase or “ULM” group), and a moiety that binds a target protein (e.g., a protein/polypeptide targeting ligand or “PTM” group) (e.g., an HDAC6 inhibitor) such that the target protein/polypeptide is placed in proximity to the ubiquitin ligase to effect degradation (and inhibition) of that protein (e.g., inhibit HDAC6 activity). In certain embodiments, the PTM is any of the compounds as described herein showing inhibitory activity against HDAC6 activity. In some embodiments, the ULM is a VHL, cereblon, mouse double minute 2 (MDM2), and/or inhibitor of apoptosis protein (IAP) E3 ligase binding moiety. For example, the structure of the bifunctional compound can be depicted as PTM-ULM.

The respective positions of the PTM and ULM moieties, as well as their number as illustrated herein, is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as PTM-L-ULM, where PTM is a protein/polypeptide targeting moiety (e.g., any of the compounds as described herein showing inhibitory activity against HDAC6), L is a linker, and ULM is a VHL, cereblon, MDM2, or IAP E3 ligase binding moiety binding moiety.

Such embodiments are not limited to a specific type of linker. In some embodiments, the linker group is optionally substituted (poly) ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical. In some embodiments, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

The ULM group and PTM group may be covalently linked to the linker group through any group which is appropriate and stable to the chemistry of the linker. In exemplary aspects of the present invention, the linker is independently covalently bonded to the ULM group and the PTM group in certain embodiments through an amide, ester, thioester, keto group, carbamate (urethane), carbon or ether, each of which groups may be inserted anywhere on the ULM group and PTM group to provide maximum binding of the ULM group on the ubiquitin ligase and the PTM group on the target protein to be degraded. In certain aspects where the PTM group is a ULM group, the target protein for degradation may be the ubiquitin ligase itself. In certain exemplary aspects, the linker may be linked to an optionally substituted alkyl, alkylene, alkene or alkyne group, an aryl group or a heterocyclic group on the ULM and/or PTM groups.

In certain embodiments, the compounds as described herein comprise multiple ULMs, multiple PTMs, multiple chemical linkers, or any combinations thereof. In some embodiments, the present invention provides a method of ubiquitinating/degrading AR receptor activity and/or AR expression in a cell comprising administering a bifunctional compound as described herein comprising an ULM and a PTM, in certain embodiments linked through a linker moiety, as otherwise described herein, wherein the ULM is coupled to the PTM and wherein the ULM recognizes a ubiquitin pathway protein and the PTM recognizes the target protein such that degradation of the target protein occurs when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

### BRIEF DESCRIPTION OF THE DRAWINGS

FIG. 1-F: A non-hydroxamate HDAC6-specific inhibitor reverses diet-induced obesity. a, Chemical structures of Tubastatin A, Trichostatin A (TSA) (top) and Compound-4 (Example-1) (bottom). 1b. The X-ray crystal structure of human HDAC6 CD2 domain in complex with Trichostatin A (TSA) shown in green (pdb code: 5edu). Docking of Compound-4 (Example-1) in the inhibitor binding site of HDAC6 is shown in magenta. The A chain of the 5edu.pdb was used for docking. The oxygen in the oxadiazole ring in Compound-4 is binding to Zinc as the hydroxamate in Trichostatin A. The trifluoro methyl group fits in the binding pocket well and also making hydrogen bond to His610. The pyridine ring is sandwiched between Phe680 at the top and Phe620 at the bottom making x-x interactions with them. The docking experiment was carried out using the GOLD docking software with the OPLS-AA force field implemented in the MOE software. c, Dose response curves of HDAC6 inhibition for the HDAC6-specific inhibitors CAY10603, tubastatin, and SE-7552. d, Immunoblots from lysates from N1 cells treated with the indicated compounds for 24 hr. e, f, Body weight change of SE-7552 (50 mg/kg, i.p.) treated wild-type DIO mice (n=12-13) (e) or db/db mice (n=6) (f).

FIG. 2A-F: Tubastatin A Reverses Diet-Induced Obesity. a, Effect of daily intraperitoneal (i.p.) Tubastatin (TubA, 25 mg/kg, n=6) or vehicle (Veh, n=6) administration on wild-type DIO mice. b, Body weight of TubA-treated WT (n=17) and HDAC6 KO (n=14) DIO mice. c, d, Growth curves of wild-type mice on regular diet (c) (chow) or HFD (d) treated by daily i.p. vehicle or tubastatin (25 mg/kg) (n=9 mice per group). e, Lean and fat mass of DIO mice determined by nuclear magnetic resonance (NMR) before and 32 days after TubA or Veh treatment (n=6). f, Hematoxylin and eosin (H&E) staining of liver sections of DIO mice after one month of vehicle or TubA treatment. *P<0.05, **P<0.01, ***P<0.001 as analyzed by one-way or two-way analysis of variance (ANOVA) or mixed-effect analysis with Tukey's post-hoc test for multiple comparison, or two-tailed Student's t-test. Data are represented as mean±s.e.m in all Figures.

FIG. 3A-F: CCG359470, a novel non-hydroxamate HDAC6 inhibitor, reverses diet-induced obesity. Mice were fed high fat diet to induce obesity. Subsequently, obese mice were treated by daily intraperitoneal injections of vehicle or CCG359470 (50 mg/kg). a, Body weight change and b, percent body weight change of the mice. c, Body composition and d, 6 hr fasting blood glucose of the mice after two-week treatment. e, Food intake of the animals during the Veh and CCG359470 treatment period. f, Percent weight loss of DIO wild-type mice treated with indicated doses of CCG359470 compared to the vehicle-treated mice.

FIG. 4: DIO wild-type mice were treated with the indicated compounds daily. The body weight of the mice were monitored daily. The change in the body weights of the mice after 4-day treatment is presented as a percentage of their initial body weights. Compounds were dissolved in the vehicle solution (20% DMSO-D6, 50% PEG-400, 30% PBS) and administered to diet-induced obese (DIO) wild-type C57BL/6J mice by intraperitoneal injection at 25 mg/kg dose within one hour before dark cycle, once a day. Injection volume was 50 uL. Change in the body weight of the mice after 4 day injection compared to their initial body weights is plotted.

### DETAILED DESCRIPTION OF THE INVENTION

Experiments conducted during the course of developing embodiments for the present invention designed, synthesized and biologically evaluated compounds having a heteroaryl substituted oxadiazole structure which function as HDAC6 inhibitors.

Accordingly, this invention relates to a new class of small-molecules having a heteroaryl substituted oxadiazole structure which function as HDAC6 inhibitors, and their use as therapeutics for the treatment of metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

In a particular embodiment, compounds encompassed within the following formula (Formula I) are provided:

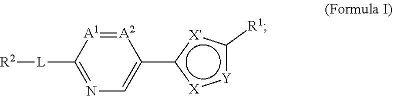

including pharmaceutically acceptable salts, solvates, and/or prodrugs thereof.

In some embodiments, the compound of Formula I is selected from Formulas (Iaa), (Iaaa₁), (Iaaa₂), (Iaaa₃), (Iaaa₄), or (Iaaa₅):

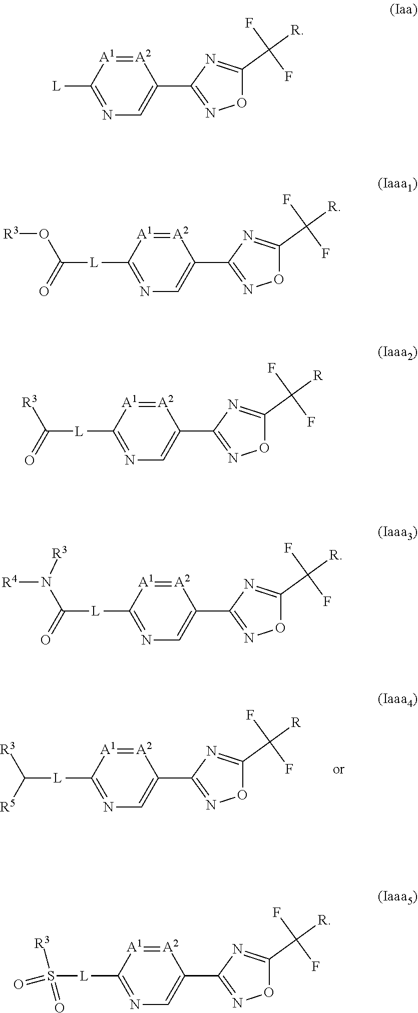

In some embodiments, the compound of Formula I is selected from Formulas (Iba), (Ibaa₁), (Ibaa₂), (Ibaa₃), (Ibaa₄), or (Ibaa₅):

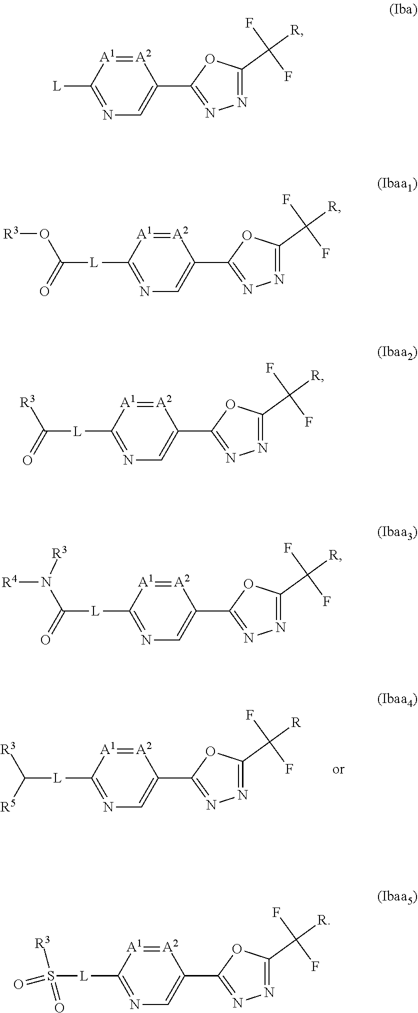

In some embodiments, the compound of Formula I is selected from (Ia), (Ib), (Ic) and (Id):

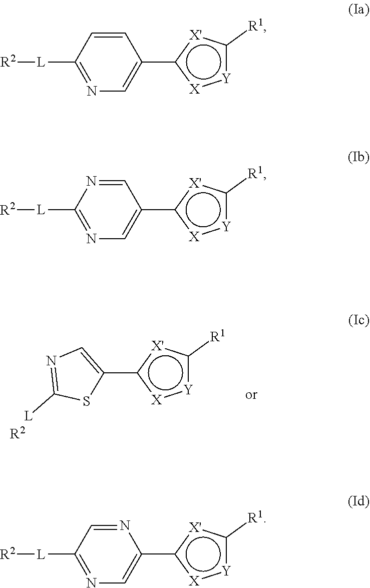

Formula I (and any formulas encompassed within Formula I) is not limited to a particular chemical moiety for A¹, A², L, X, X′, Y, R, R¹, R², R³, and R⁴. In some embodiments, the particular chemical moieties for A¹, A², L, X, X′, Y, R, R¹, R², R³, and R⁴ independently include any chemical moiety that permits the resulting compound to inhibit HDAC6 activity.

Such embodiments are not limited to a particular chemical moiety for X, X′ and Y. In some embodiments, X and X′ are N when Y is O, or X and Y are N when X′ is O.

Such embodiments are not limited to a particular chemical moiety for A¹ and A². In some embodiments, A¹ and A² are independently CH or N, or A¹ and A² are independently CH, or A¹ and A² taken together as S.

Such embodiments are not limited to a particular chemical moiety for R. In some embodiments, R is fluorine or hydrogen.

Such embodiments are not limited to a particle chemical moiety for R¹. In some embodiments, R¹ is CF₃ or CF₂H.

In some embodiments,

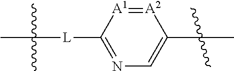

is selected from the group consisting of:

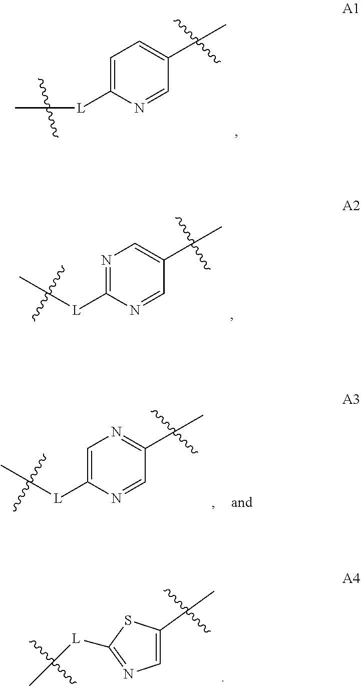

Such embodiments are not limited to a particular chemical moiety for L. In some embodiments, L is a bicyclic or spirocyclic diamine.

In some embodiments, L is a class of bicyclic diamines, where the bicyclic diamines are 2,5-diazabicyclo[2.2.1]heptane, or 3,6-diazabicyclo[3.1.1]heptane, 3,6-diazabicyclo[3.1.0]hexane, 3,7-diazabicyclo[4.1.0]heptane, octahydropyrrolo[3,4-c]pyrrole, octahydro-1H-pyrrolo[3,4-c]pyridine, 3,8-diazabicyclo[3.2.1]octane, 3,6-diazabicyclo[3.2.1]octane, 3,9-diazabicyclo[3.3.1]nonane, 2,5-diazabicyclo[2.2.2]octane, or 2,5-diazabicyclo[4.2.0]octane,

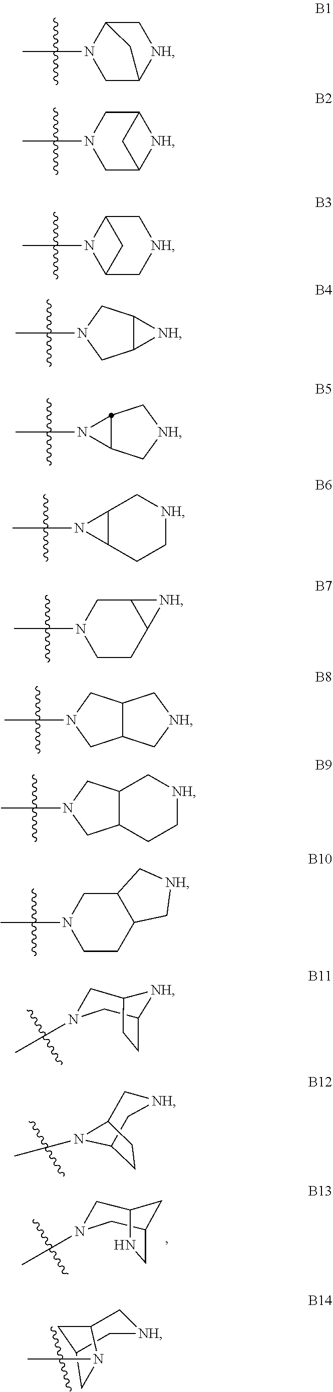

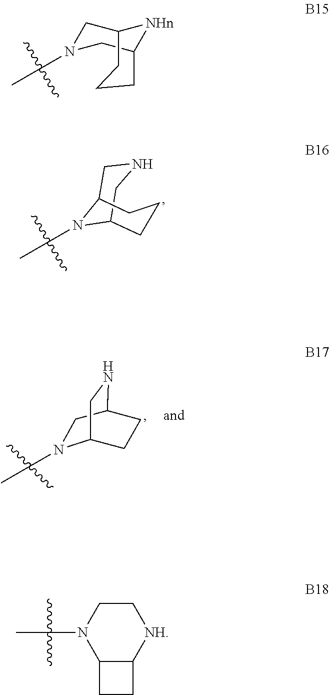

In some embodiments, L is a class of spirocyclic diamines, where the spirocyclic diamines are 2,6-diazaspiro[3.3]heptane, 2,6-diazaspiro[3.4]octane, 2,7-diazaspiro[4.4]nonane, 2,7-diazaspiro[3.5]nonane, 2,7-diazaspiro[3.5]nonane, 2,8-diazaspiro[4.5]decane, 2,9-diazaspiro[5.5]undecane, 3,9-diazaspiro[5.5]undecane and 4,7-diazaspiro[2.5]octane,

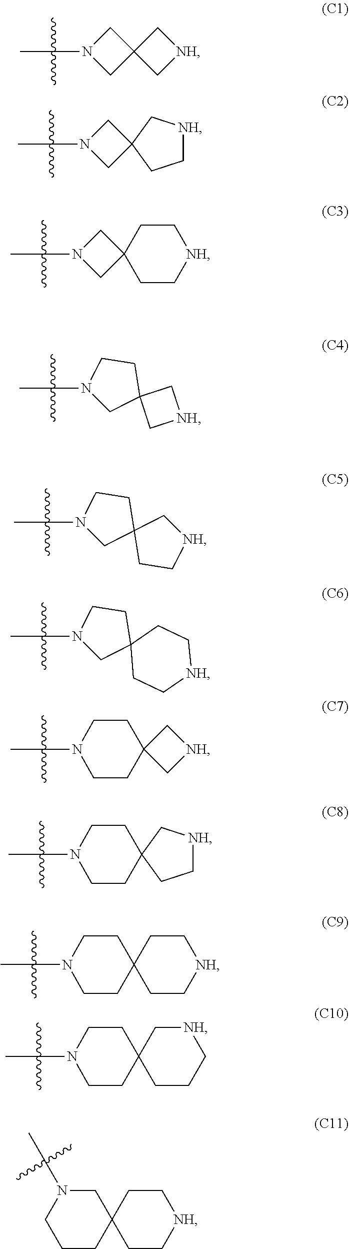

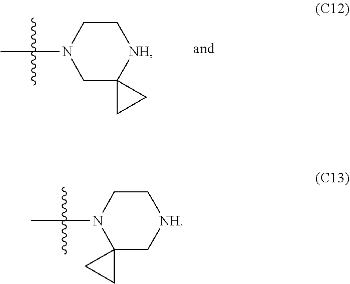 [SMILES: *N1CCNC2(CC2)C1 ; *N1CCNCC12CC2]

Such embodiments are not limited to a particular chemical moiety for R². In some embodiments, R² is hydrogen, —COR³, —CO₂R³, —(CH₂)ₙNR³R⁴, —CONR³R⁴, —(CH₂)ₙR³R⁴, and —SO₂R³, wherein n=1 or 2 optionally substituted with a CH₃ group.

Such embodiments are not limited to a particular chemical moiety for R³. In some embodiments, R³ is hydrogen, C₁-C₆alkyl or C₁-C₆branchedalkylalkyl or C₁-C₆cycloalkyl, aryl, cycloalkylalkyl, heterocyclyl, heteroaryl, arylalkyl, arylaryl, or aryl, each of which is optionally mono-, di-, or tri-substituted independently with C₁-C₆alkyl, halogen, cycloalkyl, and aryl.

Such embodiments are not limited to a particular chemical moiety for R⁴. In some embodiments, R⁴ is C₁-C₆alkyl, cycloalkyl, cycloalkylalkyl, heterocyclyl, heteroaryl, arylalkyl, or aryl, each of which is optionally mono-, di-, or tri-substituted independently with C₁-C₆alkyl, halogen, cycloalkyl, and aryl.

In some embodiments, R³ and R⁴ are each independently aryl, heteroaryl, alkyl, cycloalkyl, ketocycloalkyl, heterocyclyl, or —N(R⁴)R⁵, each of which is optionally mono- or di-substituted independently with C₁-C₆alkyl, cycloalkyl, alkoxy, aryl, cyano, halogen, —N(R⁴)R⁵, or —N(R⁵)R⁶

In some embodiments, R³ and R⁴ are each independently aryl, heteroaryl, alkyl, cycloalkyl, ketocycloalkyl, heterocyclyl, or —N(R⁴)R⁵, each of which is optionally mono- or di-substituted independently with C₁-C₆alkyl, cycloalkyl, alkoxy, aryl, cyano, halogen or —N(R⁵)R⁶.

Such embodiments are not limited to a particular chemical moiety for R⁵. In some embodiments, R⁵ is hydrogen or CH₃.

Such embodiments are not limited to a particular chemical moiety for R⁶. In some embodiments, R⁶ is hydrogen, C₁-C₃alkyl, CF₃, CHF₂, or C₃-C₆cycloalkyl.

In some embodiments, R⁵ and R⁶ taken together with the N to which they attach form H, alkyl, heteroaryl, cycloalkyl, or spirocyclic, which is optionally mono-, di-, or tri-substituted independently with C₁-C₆alkyl, C₁-C₆alkoxy, aryl, cycloalkyl, aryl, —O, or halogen. In some embodiments, cycloalkyl is a cyclopropyl, cyclobutyl, cyclopentyl, or cyclohexyl, which is optionally substituted independently with C₁-C₆alkyl, C₁-C₆alkoxy, aryl, heteroaryl, cycloalkyl, aryl, halogenoalkyl. In some embodiments, the heterocyclyl is pipreazinyl, piperidinyl, pyrrolidinyl, tetrahydrofuranyl, tetrahydropyranyl, oxetanyl, tetrahydrothiopyranyl, or tetrahydrothiofuranyl which is optionally substituted independently with C₁-C₆alkyl, C₁-C₆alkoxy, aryl, Heteroaryl, cycloalkyl, aryl, ═O, halogenoalkyl, or alkylsulfonamido.

In a particular embodiment, compounds encompassed within Formula II are provided:

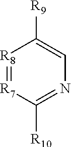

(Formula II); including pharmaceutically acceptable salts, solvates, and/or prodrugs thereof.

Formula II is not limited to a particular chemical moiety for R7, R8, R9 and R10. In some embodiments, the particular chemical moieties for R7, R8, R9 and R10 independently include any chemical moiety that permits the resulting compound to inhibit HDAC6 activity.

Such embodiments are not limited to a specific chemical moiety for R7. In some embodiments, R7 is Carbon or Nitrogen.

Such embodiments are not limited to a specific chemical moiety for R8. In some embodiments, R8 is Carbon or Nitrogen.

In some embodiments, R7 and R8 together is Sulfur such that the resulting structure is

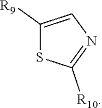

Such embodiments are not limited to a specific chemical moiety for R9. In some embodiments, R9 is selected from

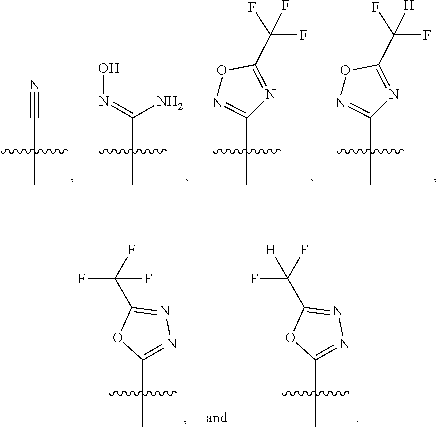

Such embodiments are not limited to a specific chemical moiety for R10. In some embodiments, R10 is selected from hydrogen, halogen (e.g., Chlorine),

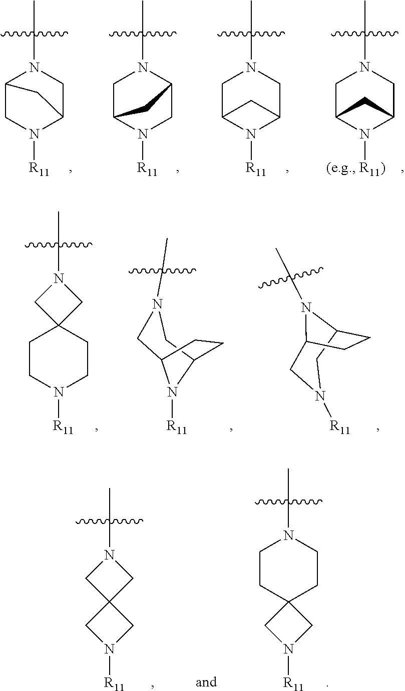

Such embodiments are not limited to a specific chemical moiety for R11. In some embodiments, R11 is selected from hydrogen,

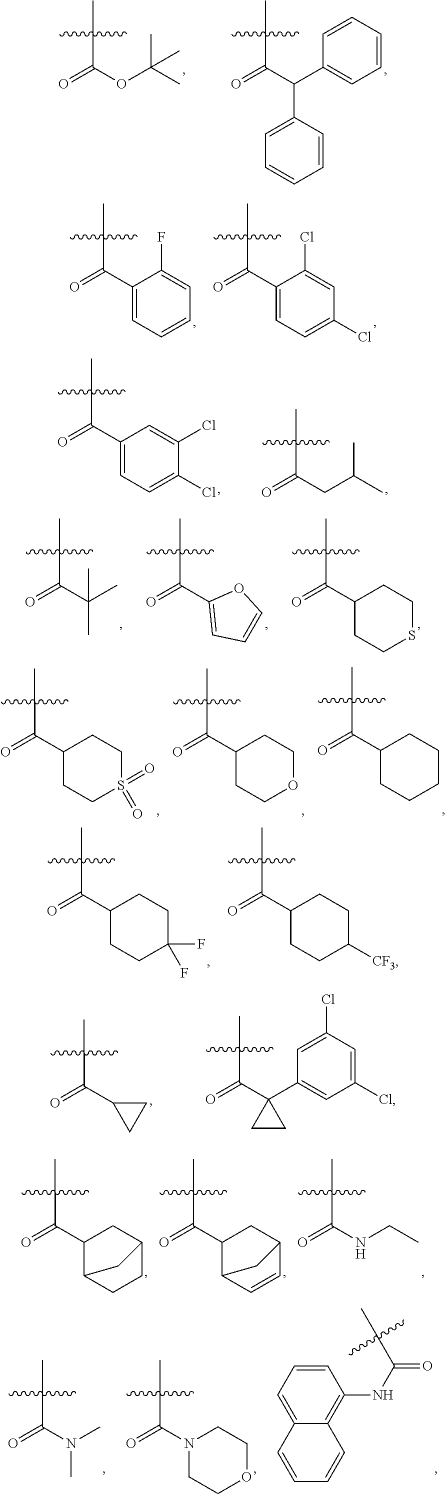

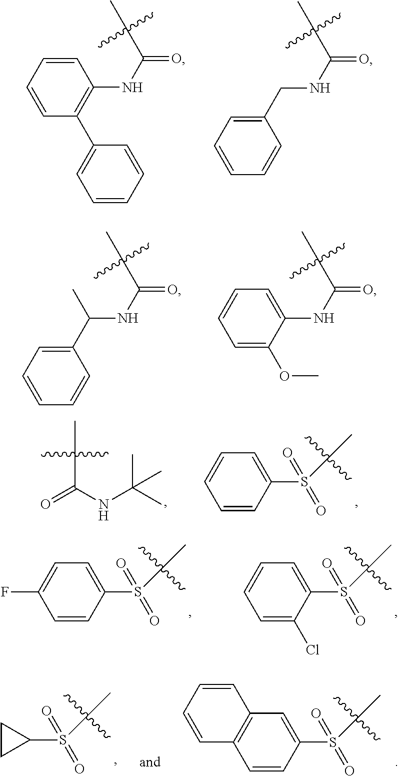

In certain embodiments, the compound encompassed within Formulas I or II is selected from any of the compound recited in Example B (e.g., Tables I, II, III, and IV).

The invention further provides processes for preparing any of the compounds of the present invention.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

Some embodiments of the present invention provide methods for administering an effective amount of a compound of the invention and at least one additional therapeutic agent (including, but not limited to, any agent useful in treating metabolic disorders (e.g., obesity, Diabetes), neurological disorders (e.g., Alzheimer's disease, Parkinson disease, Huntington disease), cancer (e.g., multiple myeloma, biliary tract cancer, non-small cell lung cancer, chronic lymphocytic leukemia) and other conditions related to HDAC6 activity (e.g., Rett syndrome (RTT), inherited retinal disorders (IRDS), idiopathic pulmonary fibrosis (IPF), and Charcot-Marie-Tooth disease (CMT)).

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example A

The compounds of the present invention may be prepared, without limitation, according to one of the general methods outlined below. For example, Schemes 1-12 that follow are intended as an illustration of some embodiments of the invention and no limitation of the present invention is implied because of them.

The following defines acronyms as used herein unless specified otherwise in a particular instance.
ACN=Acetonitrile
ABq=AB quartet
t-BOC=Tertiary butoxy carbonate
br.,=broad
CDI=N,N′-Carbonyldiimidazole,
1,2-DCE=1,2-Dichloroethane
DCM=Dichloromethane or Methylene chloride
ddd=doublet of doublet doublet
ddt=doublet of doublet triplet
DFAA=Difluoroacetic anhdride
DIEA or DIEPA=Diisopropyl ethyl amine
DMAP=Dimethyl aminopyridine
DMF=Dimethyl formamide
DMSO-D6=Dimethyl sulfoxide
DSC=Disuccinoyl carbonate
EtOAc=Ethyl acetate
EA=Ethyl acetate
EDC-HCl=N-Ethyl-N′-(3-dimethylaminopropyl) carbodiimide hydrochloride
Et₃N=Triethyl amine
EtOH=Ethanol
Et₂O=Diethyl ether or Ether
HBTU=2-(1H-Benzotriazol-1-yl)-1,1,3,3-Tetramethyluronium hexafluorophosphate,
HCl=Hydrochloric acid
Hex=Hexanes
HOBT=1-Hydroxybenzotriazole
HCl=Hydrochloric acid
1M=1 Molar
4N=4 Normal
NH₂OH=Hydroxylamine
NH₂OH. HCl=Hydroxylamine Hydrochloride
K₂CO₃=Potassium carbonate
LiOH=Lithium hydroxide
MeOH=Methanol
NaH=Sodium hydride
NaBH (OAc) 3=Sodium triacetoxyborohydride
NaBH4=Sodium borohydride
NBS=N-Bromosuccinimide
NMP=N-Methyl Pyrrolidinone
Na₂SO₄=Sodium sulfate
PyBOP=Benzotriazol-1-yl-oxytripyrrolidinophosphonium hexafluorophosphate,
RT=room temperature or retention time, as the case may be.
RT=Retention time
RT=room temperature
TEA=Triethylamine
TFA=Trifluoroacetic acid
TFA salt=Trifluoroacetate salt
TFAA=Trifluoroacetic anhdride
THF=Tetrahydrofuran
tt=triplet of triplet
p-TsOH=p-Toluene sulfonic acid
ZnCl₂=Zinc Chloride
ZnBr₂=Zinc Bromide

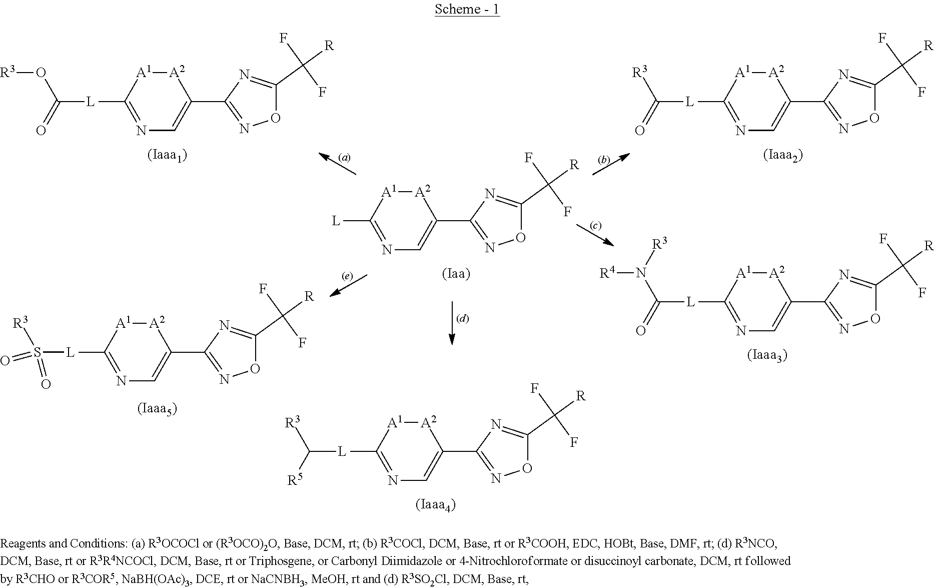

Heteroaryl Oxadiazoles of formula (Iaaa₁-Iaaa₅) can be prepared can be prepared via the process outlined in Scheme 1 using customary coupling procedures from starting compounds of formula (Iaa) where R¹-R⁵ are as previously defined herein. Compounds of formula (Iaaa₁) can be synthesized by treatment of compounds of formula (Iaa) with R³OCOCl or (R³OCO)₂O in the presence of base in DCM or THF at room temperature. Similarly, the compounds of the invention compounds of formula (Iaaa₂) can be synthesized by treatment of compounds of formula (Iaa) with R³COCl in the presence of base or treatment of (Iaa) with R³COOH using customary coupling procedures such as EDC/HOBt or HATU or HBTU in the presence of base (Carpenter, R. D. J. Comb. Chem. 2006, 8, 907; Dunetz, J. R. Org. Proc. Res. Dev. 2016, 20, 140). Compounds of formula (Iaaa₃) can be synthesized by treatment of compounds of formula (Iaa) with R³NCO in the presence of base (WU, J. H., WO 2016/049774) or R³R⁴NCOCl in the presence of base or with triphosgene (Majer, P.; Randad, R. S. J. Org. Chem. (1994), 59, 1937) or carbonyl diimidazole (CDI) or 4-nitrophenyl chloroformate or disuccinoyl carbonate (DSC) followed by treatment with R³R⁴NH. Compounds of formula (Iaaa₄) can be synthesized by treatment of compounds of formula (Iaa) with R³CHO or R³COR⁵ NaBH(OAc)₃ in DCE (Abdel-Magid, A. F. et. al., J. Org. Chem. 1996, 61, 3849) or with NaCNBH₃ in MeOH. Compounds of formula (Iaaa₅) can be are synthesized by coupling of the amines with R³SO₂Cl.

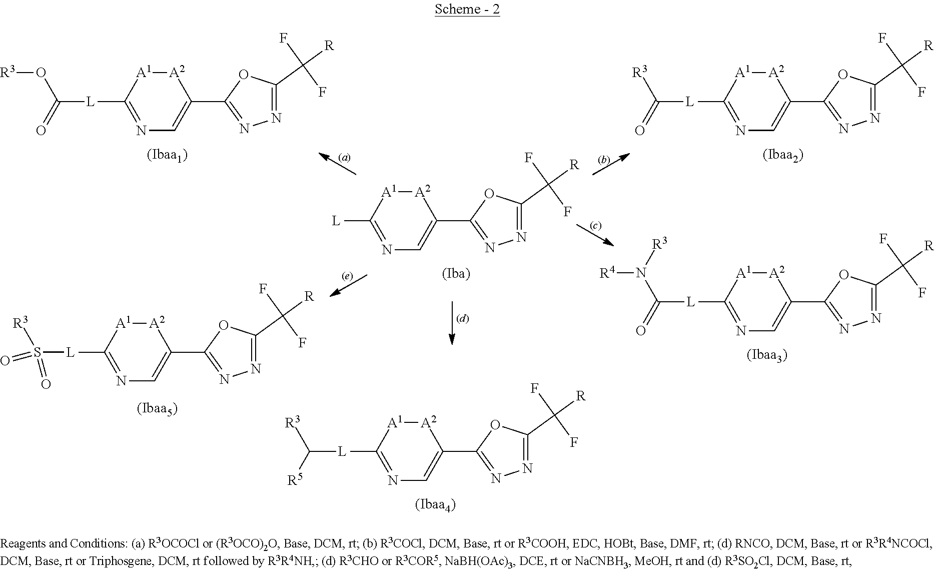

Heteroaryl Oxadiazoles of formula (Ibaa₁-Ibaa₅) can be prepared can be prepared via the process outlined in Scheme 2 using customary coupling procedures from starting compound (Iba) where R¹-R⁵ are as previously defined herein. Compounds of formula (Ibaa₁) can be synthesized by treatment of compounds of formula (Iba) with R³OCOCl or (R³OCO)₂O in the presence of base in DCM or THF. Similarly, the compounds of the invention compounds of formula (Ibaa₂) can be synthesized by treatment of compounds of formula (Iba) with R³COCl in the presence of base or treatment of (Iba) with R³COOH using customary coupling procedures such as EDC/HOBt or HATU or HBTU in the presence of base (Carpenter, R. D. J. Comb. Chem. 2006, 8, 907; Dunetz, J. R. Org. Proc. Res. Dev. 2016, 20, 140). Compounds of formula (Ibaa₃) can be synthesized by treatment of compounds of formula (Iba) with R³NCO in the presence of base (WU, J. H., WO 2016/049774) or R³R⁴NCOCl in the presence of base, or with triphosgene (Majer, P.; Randad, R. S. J. Org. Chem. (1994), 59, 1937) or carbonyl diimidazole (CDI) or 4-nitrophenylchloroformate or disuccinoyl carbonate (DSC) followed by treatment with R³R⁴NH. Compounds of formula (Ibaa₄) can be synthesized by treatment of compounds of formula (Ibaa) with R³CHO or R³COR⁵ with NaBH(OAc)₃ in DCE (Abdel-Magid, A. F. et. al., J. Org. Chem. 1996, 61, 3849) or with NaCNBH₃ in MeOH. Compounds of formula (Ibaa₅) can be are synthesized by coupling of the amines with R³SO₂Cl.

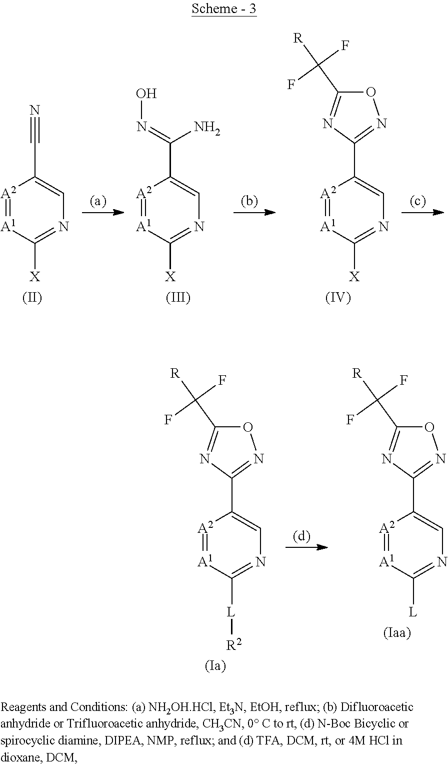

Heteroaryl Oxadiazoles of formula (Iaa) can be prepared can be prepared via the process outlined in Scheme 3. Treatment of compounds of formula (II) with hydroxylamine in the presence of base in ethanol under refluxing condition to give a compound of formula (III), which upon treatment with either trifluoroacetic or difluoroacetic anhydride to form the oxadiazoles of formula (IV) (Clarke, K. J. Chem. Soc., 1954, 4251; Bora, R. A. and Farooqui, M. J. Heterocycl. Chem. 2007, 44, 645; Ziga J. and Marija S. D., Cur.org. Chem, (2008), 12, 850). Base catalyzed or metal catalyzed amination of amines of formula R²-L to afford the compounds of formula (Ia) which upon deprotection under acidic condition with either TFA or HCl to afford the compounds of formula (Iaa).

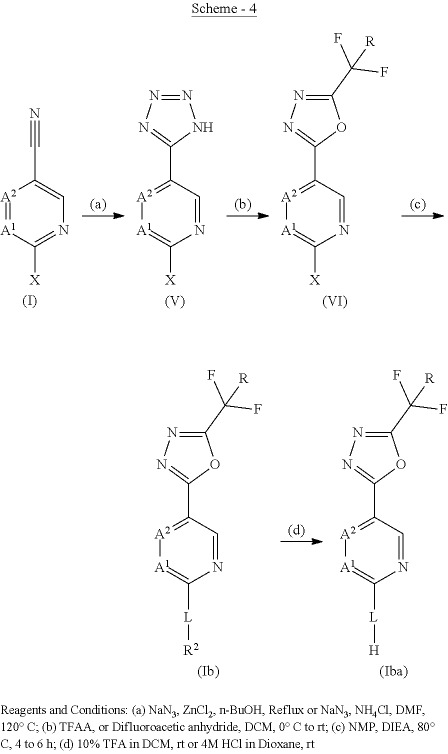

Heteroaryl Oxadiazoles of formula (Iba) can be prepared can be prepared via the process outlined in Scheme 4. Treatment of compounds of formula (II) with sodium azide in the presence of ZnCl₂ or ZnBr₂ under refluxing condition in n-BuOH or n-BuOH/H₂O (Nicolas et. al. WO 2010/108268) or heating with sodium azide at 120° C. with NH₄Cl in DMF or H₂O (Zachary P. Demko and K. Barry Sharpless The Journal of Organic Chemistry, (2001), 66, 24, 7945) give a compound of formula (V), which upon treatment with either trifluoroacetic or difluoroacetic anhydride to form the oxadiazoles of formula (VI) Clarke, K. J. Chem. Soc., 1954, 4251; Bora, R. A. and Farooqui, M. J. Heterocycl. Chem. 2007, 44, 645; Ziga J. and Marija S. D., Cur. Org. Chem, (2008), 12, 850). Base catalyzed or palladium catalyzed amination of formula R²-L to afford the compounds of formula (Ib) which upon deprotection under acidic condition with either TFA or HCl to afford the compounds of formula (Iba).

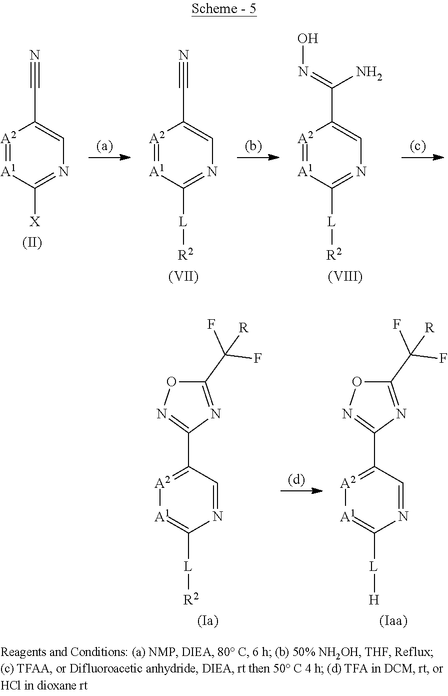

Heteroaryl Oxadiazoles of formula (Iaa) can also be prepared can be prepared via the process outlined in Scheme 5. Treatment of compound of formula (II) with base catalyzed amination or palladium catalyzed amination of formula R²-L of amines of formula R²-L to afford the compounds of formula (VII). Base catalyzed addition of NH₂OH. HCl to compounds of the formula (VII) in the presence of base in ethanol under refluxing condition or refluxing with 50% aqueous NH₂OH in THF to give a compounds of formula (VIII), which upon treatment with either trifluoroacetic or difluoroacetic anhydride to form the oxadiazoles of formula (Ia). Deprotection of the compounds of formula (Ia) under acidic condition with either TFA or HCl to afford the compounds of formula (Iaa).

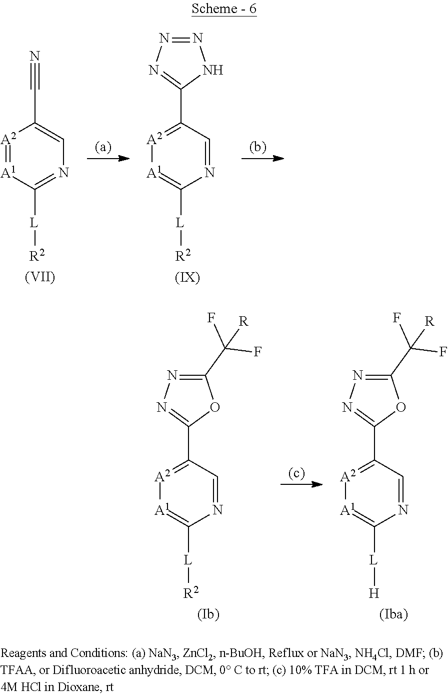

Heteroaryl Oxadiazoles of formula (Iba) can be prepared can be prepared via the process outlined in Scheme 6. Treatment of compounds of formula (VII) with sodium azide in the presence of ZnCl₂ or ZnBr₂ under refluxing condition in n-BuOH or n-BuOH/H₂O (Nicolas et. al. WO 2010/108268) or heating with sodium azide at 120° C. with NH₄Cl in DMF or H₂O (Zachary P. Demko and K. Barry Sharpless The Journal of Organic Chemistry, (2001), 66, 24, 7945) give a compound of formula (IX), which upon treatment with either trifluoroacetic or difluoroacetic anhydride to form the oxadiazoles of formula (VI) Clarke, K. J. Chem. Soc., 1954, 4251; Bora, R. A. and Farooqui, M. J. Heterocycl. Chem. 2007, 44, 645; Ziga J. and Marija S. D., Cur.org. Chem, (2008), 12, 850). Deprotection of the compounds of formula (Ia) under acidic condition with either TFA or HCl to afford the compounds of formula (Iba).

### Example B.

Unless specifically stated otherwise, the experimental procedures were performed under the following conditions. All operations were carried out at room temperature (about 18° C. to about 25° C.) under nitrogen atmosphere. Evaporation of solvent was carried out using a rotary evaporator under reduced pressure. The course of the reaction was followed by thin layer chromatography (TLC) or liquid chromatography (LC), and reaction times are given for illustration only. Silica gel chromatography was carried out on a CombiFlash® system (Teledyne Isco, Inc., Lincoln, NE, USA) with pre-packed silica gel cartridge or performed on Merck silica gel 60 (230-400 mesh) (Merck KGaA, Darmstadt, Germany). The structure and purity of all final products was assured by at least one of the following analytical methods: nuclear magnetic resonance (NMR) and LC-MS.

### a) NMR

NMR data was acquired at the Biochemical NMR Core Laboratory, College of Pharmacy, University of Michigan, on an Agilent 400 MHz MR spectrometer with a 5 mm ONE probe and operated by host software VNMRJ 3.4 Data was analyzed using MestreNova 14.1.0 (Mestrelab Research S.L.). Chemical shift (8) is given in parts per million (ppm) relative to tetramethyl silane (TMS) as an internal standard. Coupling constants (J) are expressed in hertz (Hz), and conventional abbreviations used for signal shape are: s=singlet; d=doublet; t=triplet; m=multiplet; br=broad; etc.

### b) ESI-MS

Unless specifically stated otherwise, the electrospray ioniozation-mass spectrometric (ESI-MS) procedures were performed using electrospray ionization (ESI) operating in positive mode via a Waters LCT time-of-flight (TOF) mass spectrometer (Waters Corp., Milford, MA, USA). The solvent carrier used is a gradient of 100% to 85% of solvent (A) Water with 0.1% Trifluoroacetic acid and 0% to 15% of Solvent (B) Acetonitile with 0.1% Trifluoroacetic acid MeOH (with 0.1% TFA). From mass spectra obtained, (M+H)⁺ and in some cases (M+H-56)⁺ are reported. From chromatographic spectra obtained, retention times in minutes (RT) are reported.

### c) Q-TOF LC/MS

Unless specifically stated otherwise, the Quadrupole Time of Flight-mass spectrometric (Q-TOF LC/MS) procedures were performed using Quadrupole Time of Flight-mass spectrometer (Q-TOF) operating in positive mode via a Agilent Technologies, 6520 Accurate Mass Q-TOF LC/MS (Agilent Technologies, Santa Clara, CA, USA). The solvent carrier used is a gradient of Water with 0.1% Formic acid and 95% Acetonitrile/0.5% Water with 0.1% Formic acid. From mass spectra obtained, (M+H)⁺ and in some cases (M+H−56)⁺ are reported.

### c) HPLC

Unless specifically stated otherwise, the liquid chromatographic analysis was performed using a Agilent LAB CDS Chemstation Edition for LC and LC/MS system. The chromatograms were analyzed using Agilent Open LAB Intelligent Reporting A.01.06.111 software. The samples were prepared using 1 mg/mL, and the sample injection volume is 5-10 μL. For elution, a gradient of a mobile phase with water:acetonitrile (90:10) to acetonitrile:water (90:10) over 13 minutes, with 0.1% TFA added to both water and acetonitrile (Method-1) using Agilent Eclipse C-18 column. The compounds are assayed at 250 nm UV wavelength and retention times (RT) are reported in minutes.

### 2) Intermediate of Formula s of the Invention

Unless specified otherwise, the reagents used in the preparation of compounds, including intermediates, of the present invention were purchased from Fisher Scientific, TCI Chemicals, Ambeed Inc., Enamine LLC, Matrix Inc., and Sigma-Aldrich Corporation.

### Intermediate 5: 3-(6-(2,5-diazabicyclo[2.2.1]heptan-2-yl)pyridin-3-yl)-5-(trifluoromethyl)-1,2,4-oxadiazole

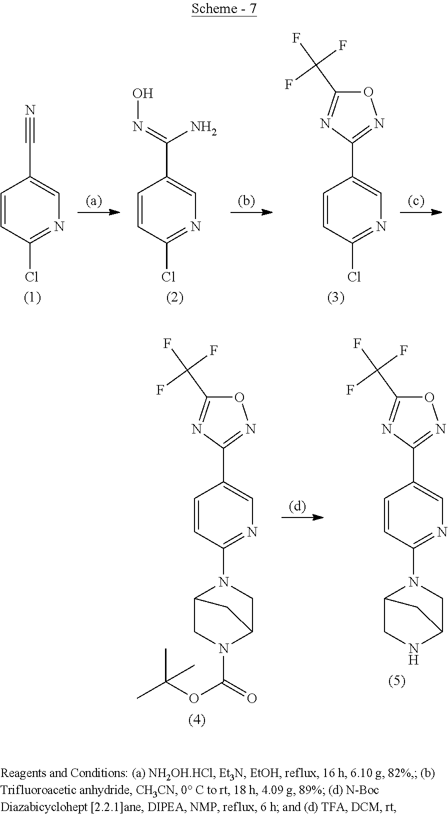

### Intermediate—5 was Prepared Via the Process of Scheme 7, Supra, as Follows

### Step 1: Intermediate of Formula—2:6-chloro-N′-hydroxynicotinimidamide (2)

To a stirred mixture of 6-chloronicotinonitrile (1, 3.00 g, 21.7 mmol) and hydroxylamine hydrochloride (3.31 g, 47.6 mmol) in ethanol (35.0 mL) was added triethylamine (6.64 mL, 4.82 g, 47.6 mmol) at room temperature. The resulting mixture was refluxed under nitrogen atmosphere for 16 h. After refluxing 16 h, the reaction was cool to room temperature and water (40 mL) was added with vigorous stirring. The product precipitated out and the solution was concentrated to half of its volume. Filtered the precipitate and washed it with water and dried at room temperature afforded the desired product as a white solid (2, 3.54 g, 95%). RT=1.106 Min. (HPLC Method-I). ¹H NMR (400 MHZ, DMSO-d₆) δ 9.93 (s, 1H), 8.66 (dd, J=2.5, 0.7 Hz, 1H), 8.05 (dd, J=8.4, 2.5 Hz, 1H), 7.52 (dd, J=8.4, 0.7 Hz, 1H), 6.03 (s, 2H).

### Step 2: Intermediate of Formula—3: 3-(6-chloropyridin-3-yl)-5-(trifluoromethyl)-1,2,4-oxadiazole (3)

To a stirred suspension of 6-chloro-N′-hydroxynicotinimidamide (2, 2.0 g, 11.70 mmol) in acetonitrile (25 mL) was added trifluoroacetic anhydride (8.22 mL, 12.2 g, 58.30 mmol) at 0° C. After the addition, the cold bath was removed and stirring was continued overnight at room temperature. The excess solvent was concentrated and the resulting crude product was extracted using dichloromethane. The organic portion was washed with aqueous citric acid solution, water, aqueous NaHCO₃ solution, water and brine. The organic portion was dried over anhydrous sodium sulfate and filtered followed by removal of solvent afforded the desired product as a white solid (3, 2.60 g, 84.5%). RT=6.84 min (HPLC Method-I). MS-ESI (m/z): Calculated [M+H]⁺=249.99; Observed [M+H]⁺=249.99.). ¹H NMR (400 MHZ, CDCl₃) δ 9.13 (dd, J=2.4, 0.7 Hz, 1H), 8.36 (dd, J=8.3, 2.4 Hz, 1H), 7.52 (dd, J=8.4, 0.8 Hz, 1H).

### Compound of Invention—1 (Example-1): Step 3: Intermediate of Formula-4: tert-butyl 5-(5-(5-(trifluoromethyl)-1,2,4-oxadiazol-3-yl)pyridin-2-yl)-2,5-diazabicyclo[2.2.1]heptane-2-carboxylate (4)

To a stirred mixture of 3-(6-chloropyridin-3-yl)-5-(trifluoromethyl)-1,2,4-oxadiazole (3, 1.00 g, 4.01 mmol) and tert-butyl 2,5-diazabicyclo[2.2.1]heptane-2-carboxylate (1.59 g, 8.01 mmol) in NMP (25.0 mL) was added DIEPA (1.61 mL, 1.190 g, 4.94 mmol) and the resulting solution was heated to 70° C. under nitrogen atmosphere. After heating 4 h, the reaction mixture was cool to room temperature and poured in to water (50 mL) with vigorous stirring. The product precipitated out, filtered, washed the precipitate with water and dried at room temperature in the air followed by high vacuum. The product was obtained as a pale brown color solid (4, 1.460 g, 67%). RT=6.06 min (HPLC Method-I). MS-ESI (m/z): Calculated [M+H]⁺=412.15; Observed [M+H−55]⁺=356.09. ¹H NMR (400 MHZ, CDCl₃) δ 8.85 (d, J=2.3 Hz, 1H), 8.07 (dd, J=8.7, 2.3 Hz, 1H), 6.40 (d, J=8.7 Hz, 1H), 5.00 (br., s, 1H), 4.645 (d, J=64 Hz, 1H), 3.56 (d, J=9.4 Hz, 1H), 3.51-3.33 (m, 3H), 1.98 (d, J=19.8 Hz, 2H), 1.42 (s, 9H).

### Compound of Invention—2 (Example-22): Step 4: Synthesis of 5-(5-(5-(trifluoromethyl)-1,2,4-oxadiazol-3-yl)pyridin-2-yl)-2,5-diazabicyclo[2.2.1]heptan-2-ium trifluoroacetate (5)

To a stirred solution of compound tert-butyl 5-(5-(5-(trifluoromethyl)-1,2,4-oxadiazol-3-yl)pyridin-2-yl)-2,5-diazabicyclo[2.2.1]heptane-2-carboxylate (1.05 g, 2.55 mmol) in DCM (20 m L) was added trifluoroacetic acid (2.0 mL) at room temperature under nitrogen atmosphere. After stirring 4 h at room temperature, the solvent and excess TFA were removed using rotovap. The resulting brown color gummy material was dissolved in methanol (1 mL) and triturated with hexanes. The product crashed out from the solution. Filtered and washed the product with hexane, followed by drying under high vacuum afforded the compound 5-(5-(5-(trifluoromethyl)-1,2,4-oxadiazol-3-yl)pyridin-2-yl)-2,5-diazabicyclo[2.2.1]heptan-2-ium trifluoroacetate (5, 1.33 g, 97%) as a pale brown color solid as its TFA salt RT=4.57 Min. (HPLC Method-I). MS-ESI (m/z): Calculated [M+H]⁺=313.11; Observed [M+H]⁺=313.15. ¹H NMR (400 MHZ, CDCl₃) δ 10.85 (s, 1H), 9.54 (s, 1H), 9.07 (s, 3H), 8.73 (d, J=2.3 Hz, 1H), 8.02 (dd, J=9.1, 2.3 Hz, 1H), 7.26 (s, 1H), 6.37 (d, J=8.8 Hz, 1H), 5.01 (s, 1H), 4.42 (s, 1H), 3.60 (AB q, J=52.0 and 12.0 Hz, 2H), 3.36 (s, 1H), 3.25 (s, 1H), 2.09 (AB q, J=32.0 and 12.0 Hz, 2H).

### Intermediate—8:5-(5-(5-(difluoromethyl)-1,2,4-oxadiazol-3-yl)pyridin-2-yl)-2,5-diazabicyclo[2.2.1]heptan-2-ium trifluoroacetate

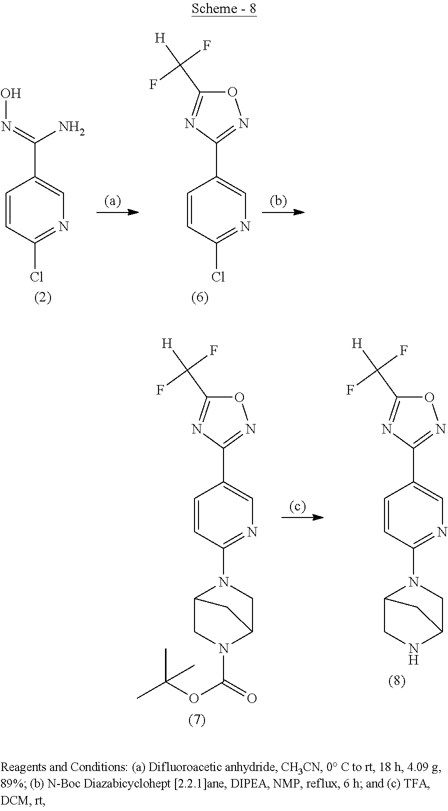

### Intermediate—8 was Prepared Via the Process of Scheme 8, Supra, as Follows

Step 1: Intermediate of Formula—6: The intermediate—6 was synthesized in a similar manner to the intermediate 3 as a white solid (6, 1.15 g, 85%). RT=6.20 Min. (HPLC Method-I). MS-ESI (m/z): Calculated [M+H]⁺=232.00; Observed [M+H]⁺=232.02. ¹H NMR (400 MHZ, CDCl₃) δ 9.13 (d, J=2.4 Hz, 1H), 8.35 (dd, J=8.3, 2.4 Hz, 1H), 7.51 (dd, J=8.3, 0.8 Hz, 1H), 6.89 (t, J=52.1 Hz, 1H).

### Compound of Invention—3 (Example-2): Step 2: Intermediate of Formula-7:3-(6-chloropyridin-3-yl)-5-(difluoromethyl)-1,2,4-oxadiazole

The intermediate—7 was synthesized in a similar manner to the intermediate 4.

The intermediate—7 was obtained as a pale brown color solid (7, 0.945 g, 91%). RT=5.43 Min. (HPLC Method-I). ¹H NMR (400 MHZ, CDCl₃) δ 8.84 (d, J=2.3 Hz, 1H), 8.07 (dd, J=8.8, 2.3 Hz, 1H), 6.84 (t, J=52.3 Hz, 1H), 6.40 (d, J=8.8 Hz, 1H), 4.64 (d, J=56.0 Hz, 1H), 4.57 (s, 1H), 3.56 (d, J=9.7 Hz, 1H), 3.51-3.32 (m, 3H), 1.97 (d, J=19.3 Hz, 2H), 1.45 (s, 9H).

### Compound of Invention—4 (Example-23): Step 3: Intermediate of Formula-8:5-(5-(5-(difluoromethyl)-1,2,4-oxadiazol-3-yl)pyridin-2-yl)-2,5-diazabicyclo[2.2.1]heptan-2-ium trifluoroacetate (8)

The intermediate 8 was synthesized in a similar manner to the intermediate—5. The intermediate 8 was obtained as a pale brown color solid as its TFA salt (8, 1.20 g, >99%). RT=4.57 Min. (HPLC Method-I). MS-ESI (m/z): Calculated [M+H]⁺=294.11; Observed [M+H−59]⁺=234.13 and 256.11. ¹H NMR (400 MHZ, CDCl₃) δ 10.85 (s, 1H), 9.54 (s, 1H), 9.07 (s, 3H), 8.73 (d, J=2.3 Hz, 1H), 8.02 (dd, J=9.1, 2.3 Hz, 1H), 7.26 (s, 1H), 6.37 (d, J=8.8 Hz, 1H), 5.01 (s, 1H), 4.42 (s, 1H), 3.66 (d, J=10.9 Hz, 1H), 3.53 (d, J=10.8 Hz, 1H), 3.36 (s, 1H), 3.25 (s, 1H), 2.18-1.98 (m, 2H), 1.13 (s, 5H).

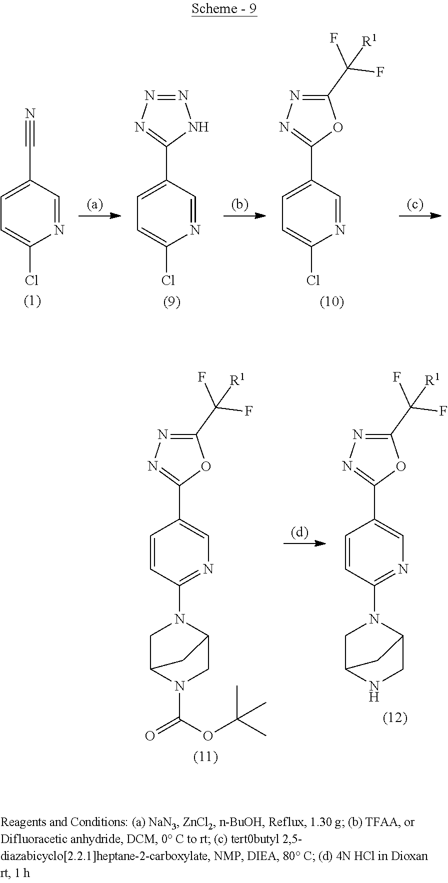

Intermediate—12 was Prepared Via the Process of Scheme 9, Supra, as Follows

### Step 1: Intermediate of Formula-9:2-chloro-5-(1H-tetrazol-5-yl)pyridine HCl Salt (9)

To a stirred solution of 6-chloronicotinonitrile (1, 1.5 0 g, 11.0 mmol) and sodium azide (1.2 equ. 0.840 g, 13.0 mmol) in n-butanol (25 mL) was added anhydrous ZnCl₂ (1.50 g, 11.0 mmol) under nitrogen atmosphere. The resulting mixture was heated to reflux overnight. After refluxing 16 h, the solvent was removed under high vacuum and the resulting mixture was treated with IN HCl (30 mL). The product is crashed out from the solution. Filtered and washed the white precipitate with water and dried at room temperature in the air for 24 h. The compound 2-chloro-5-(1H-tetrazol-5-yl)pyridine was obtained as a white solid (9, 1.70 g, 71%). MS-ESI (m/z): Calculated [M+H]⁺=182.02; Observed [M+H]⁺=182.01. ¹H NMR (400 MHZ, CDCl₃) δ 8.96 (d, J=2.4 Hz, 1H), 8.33 (dd, J=8.4, 2.5 Hz, 1H), 7.46 (d, J=8.4 Hz, 1H), 4.02 (br., s, 1H).

### Step 2: Intermediate of Formula-10a: 2-(6-chloropyridin-3-yl)-5-(difluoromethyl)-1,3,4-oxadiazole

To a stirred suspension of 2-chloro-5-(1H-tetrazol-5-yl)pyridine (1.0 g, 4.54 mmol) in DCM (25 mL) was cooled in an ice bath and added 2,2-difluoroacetic anhydride (2.82 mL, 3.95 g, 22.7 mmol). After the addition, the cold bath was removed, continued the resulting clear solution was stirred at room temperature overnight, then the excess solvent was removed using rotovap. The resulting crude product was extracted with dichloromethane and washed the organic portion with aqueous citric acid solution, water, aqueous NaHCO₃ solution, water and brine. The organic portion was dried over anhydrous sodium sulfate, filtered followed by removal of solvent afforded the titled compound as a white solid (10a, 1.11 g, 94%). RT=5.27 Min. (HPLC Method-I). MS-ESI (m/z): Calculated [M+H]⁺=232.008; Observed [M+H]⁺=232.008). ¹H NMR (400 MHZ, CDCl₃) δ 9.12 (dd, J=2.4, 0.7 Hz, 1H), 8.37 (ddd, J=8.4, 2.5, 0.5 Hz, 1H), 7.55 (dt, J=8.4, 0.6 Hz, 1H), 7.13-6.62 (m, 1H).

### Intermediate of Formula-10b: 2-(6-chloropyridin-3-yl)-5-(trifluoromethyl)-1,3,4-oxadiazole

The intermediate—10b: 2-(6-chloropyridin-3-yl)-5-(trifluoromethyl)-1,3,4-oxadiazole was synthesized in a similar manner to the intermediate 10a. Compound 2-(6-chloropyridin-3-yl)-5-(trifluoromethyl)-1,3,4-oxadiazole was obtained as a white solid (10b, 0.60 g, 99%). RT=5.97 Min. (HPLC Method-I). MS-ESI (m/z): Calculated [M+H]⁺=249.99; Observed [M+H]⁺=249.99.). ¹H NMR (400 MHZ, CDCl₃) δ 9.12 (dd, J=2.5, 0.7 Hz, 1H), 8.37 (dd, J=8.4, 2.4 Hz, 1H), 7.69-7.41 (m, 1H).

### Compound of Invention—5 (Example-5): Step 3: Intermediate of Formula-11a: tert-butyl 5-(5-(5-(difluoromethyl)-1,3,4-oxadiazol-2-yl)pyridin-2-yl)-2,5-diazabicyclo[2.2.1]heptane-2-carboxylate

To a stirred mixture of compound 2-(6-chloropyridin-3-yl)-5-(difluoromethyl)-1,3,4-oxadiazole (0.27 g, 1.20 mmol) and tert-butyl 2,5-diazabicyclo[2.2.1]heptane-2-carboxylate (0.490 g, 2.4 mmol) in NMP (5.0 mL) was added DIEPA (0.61 mL, 0.45 g, 3.5 mmol) and the resulting solution was heated to 70° C. under nitrogen atmosphere. After heating 4 h, the reaction mixture was cool to room temperature and quenched with water. The product was extracted with ethyl acetate and washed with water, citric acid solution, water and brine. Filtered and removal of solvent resulted the crude product. Purification by column chromatography on a silica gel column with a gradient of solvent 5% EA/Hex to 40% EA/Hex afforded the product as a colorless solid (11a, 0.430 g, 92%). RT=5.31 Min. (HPLC Method-I). MS-ESI (m/z): Calculated [M+H]⁺=394.16; Observed [M+H]⁺=394.16.). ¹H NMR (400 MHZ, CDCl₃) δ 8.79 (d, J=2.4 Hz, 1H), 8.04 (dd, J=8.9, 2.4 Hz, 1H), 6.87 (t, J=51.8 Hz, 1H), 6.38 (d, J=9.0 Hz, 1H), 4.63 (d, J=53.6 Hz, 1H), 3.54 (t, J=9.4 Hz, 1H), 3.43 (d, J=8.9 Hz, 2H), 3.38-3.31 (m, 1H), 2.34 (t, J=8.1 Hz, 1H), 2.12-1.86 (m, 2H), 1.44 (s, 9H).

### Compound of Invention—6 (Example-6): Intermediate of Formula-11b: tert-butyl 5-(5-(5-(trifluoromethyl)-1,3,4-oxadiazol-2-yl)pyridin-2-yl)-2,5-diazabicyclo[2.2.1]heptane-2-carboxylate

The intermediate—11b: tert-butyl 5-(5-(5-(trifluoromethyl)-1,3,4-oxadiazol-2-yl)pyridin-2-yl)-2,5-diazabicyclo[2.2.1]heptane-2-carboxylate was synthesized in a similar manner to the intermediate 11a. The compound tert-butyl 5-(5-(5-(trifluoromethyl)-1,3,4-oxadiazol-2-yl)pyridin-2-yl)-2,5-diazabicyclo[2.2.1]heptane-2-carboxylate was obtained as a colorless solid (11b, 0.170 g, 51%). RT=6.00 Min. (HPLC Method-I). MS-ESI (m/z): Calculated [M+H]⁺=412.15; Observed [M+H]⁺=412.158.). ¹H NMR (400 MHZ, CDCl₃) δ 8.76 (d, J=2.4 Hz, 1H), 8.01 (dd, J=8.9, 2.4 Hz, 1H), 6.37 (d, J=8.9 Hz, 1H), 4.61 (d, J=51.0 Hz, 1H), 3.52 (d, J=9.5 Hz, 1H), 3.46-3.36 (m, 2H), 3.34 (dd, J=8.7, 5.5 Hz, 1H), 2.32 (t, J=8.1 Hz, 1H), 2.07-1.83 (m, 2H), 1.42 (s, 9H).

### Compound of Invention—7 (Example-25): Step 4: Intermediate of Formula-12a: 2-(6-(2,5-diazabicyclo[2.2.1]heptan-2-yl)pyridin-3-yl)-5-(difluoromethyl)-1,3,4-oxadiazole

The intermediate 12a was synthesized in a similar manner to the intermediate—5. Compound 2-(6-(2,5-diazabicyclo[2.2.1]heptan-2-yl)pyridin-3-yl)-5-(difluoromethyl)-1,3,4-oxadiazole was obtained as a pale brown color solid (12a, 220 mg, 75%). RT=0.845 Min. (HPLC Method-I). MS-ESI (m/z): Calculated [M+H]⁺=294.11; Observed [M+H]⁺=234.13. ¹H NMR (400 MHZ, CD₃OD) δ 8.53-8.39 (m, 1H), 8.36-8.23 (m, 1H), 7.32 (s, 1H), 6.141 (t, J=56.0 Hz, 1H), 5.23 (s, 1H), 4.65 (s, 1H), 4.06-3.79 (m, 2H), 3.68-3.50 (m, 1H), 3.51-3.30 (m, 1H), 2.17 (d, J=12.1 Hz, 1H), 2.04-1.76 (m, 1H).

### Compound of Invention—8 (Example-24): Intermediate of Formula-12b: 2-(6-(2,5-diazabicyclo[2.2.1]heptan-2-yl)pyridin-3-yl)-5-(trifluoromethyl)-1,3,4-oxadiazole

The intermediate 12b was synthesized in a similar manner to the intermediate—5. The Compound 2-(6-(2,5-diazabicyclo[2.2.1]heptan-2-yl)pyridin-3-yl)-5-(trifluoromethyl)-1,3,4-oxadiazole was obtained as a tan color solid (12b, 70 mg, 83% HCl Salt) was obtained as a tan color solid. RT=0.858 Min. (HPLC Method-I). MS-ESI (m/z): Calculated [M+H]⁺=312.10; Observed [M+H]⁺=234.13. ¹H NMR (400 MHZ, CD₃OD) δ 8.74-8.57 (m, 1H), 8.56-8.21 (m, 1H), 7.39 (s, 1H), 5.40 (d, J=8.1 Hz, 1H), 4.85 (s, 1H), 4.08 (ddt, J=9.1, 6.2, 3.1 Hz, 2H), 3.79-3.55 (m, 2H), 2.52 (d, J=12.4 Hz, 1H), 2.37 (d, J=9.7 Hz, 1H).

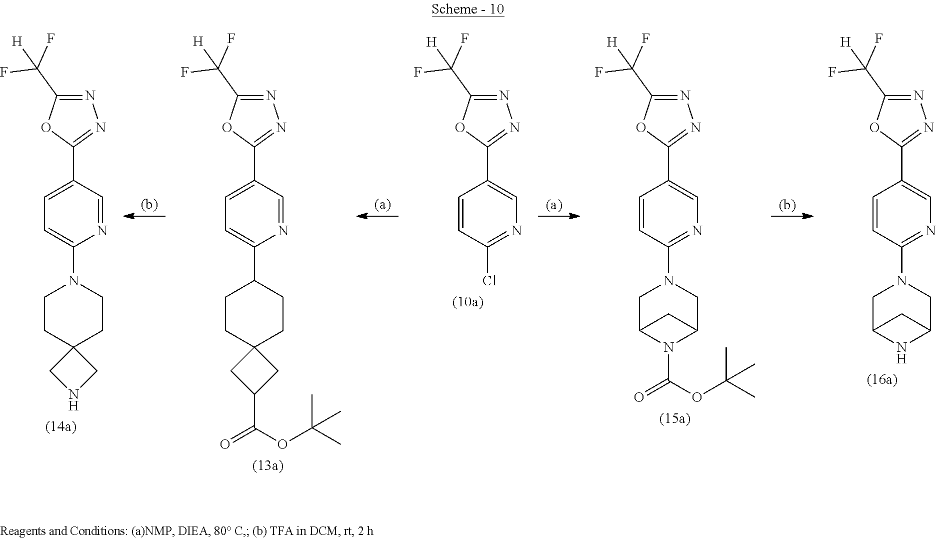

Intermediate-16a was prepared via the process of Scheme 10, supra, as follows

### Compound of Invention—9 (Example-8): Step 1: Intermediate of Formula-13a: tert-butyl 7-(5-(5-(difluoromethyl)-1,3,4-oxadiazol-2-yl)pyridin-2-yl)-2,7-diazaspiro[3.5]nonane-2-carboxylate

The intermediate 13a was synthesized in a similar manner to the intermediate—4 Compound tert-butyl 7-(5-(5-(difluoromethyl)-1,3,4-oxadiazol-2-yl)pyridin-2-yl)-2,7-diazaspiro[3.5]nonane-2-carboxylate was obtained as a brown color solid (13a, 0.230 g, 83%). RT=6.07 min, (HPLC Method-I). MS-ESI (m/z): Calculated [M+H]⁺=422.19; Observed [M+H]⁺=422.199. ¹H NMR (400 MHZ, CDCl₃) δ 8.83 (d, J=2.1 Hz, 1H), 8.07 (dt, J=9.0, 2.1 Hz, 1H), 7.03-6.74 (m, 1H), 6.72 (d, J=9.1 Hz, 1H), 3.72 (d, J=1.6 Hz, 4H), 3.67 (dd, J=8.1, 3.6 Hz, 4H), 1.84 (t, J=5.6 Hz, 4H), 1.46 (s, 9H).

### Compound of Invention—10 (Example-10): Intermediate of Formula-15a: tert-butyl 3-(5-(5-(difluoromethyl)-1,3,4-oxadiazol-2-yl)pyridin-2-yl)-3,6-diazabicyclo[3.1.1]heptane-6-carboxylate

The intermediate 15a was synthesized in a similar manner to the intermediate—4 Compound tert-butyl 3-(5-(5-(difluoromethyl)-1,3,4-oxadiazol-2-yl)pyridin-2-yl)-3,6-diazabicyclo[3.1.1]heptane-6-carboxylate was obtained as a colorless foam. (15a, 0.21 g, 82%). RT=5.76 min, (HPLC Method-I). MS-ESI (m/z): Calculated [M+H]⁺=394.16; Observed [M+H]⁺=394.16.). ¹H NMR (400 MHZ, CDCl₃) δ 8.90 (dd, J=2.4, 0.7 Hz, 1H), 8.13 (dd, J=9.0, 2.4 Hz, 1H), 6.89 (t, J=51.8 Hz, 1H), 6.62 (dd, J=9.0, 0.8 Hz, 1H), 4.31 (d, J=6.2 Hz, 2H), 4.26-4.08 (m, 2H), 3.55 (s, 2H), 2.70 (q, J=7.0 Hz, 1H), 1.51 (d, J=8.8 Hz, 1H), 1.35 (s, 9H).

### Compound of Invention—11 (Example-28): Step 2: Intermediate of Formula-14a: 7-(5-(5-(difluoromethyl)-1,3,4-oxadiazol-2-yl)pyridin-2-yl)-2,7-diazaspiro[3.5]nonan-2-ium trifluoroacetate

The intermediate 14a was synthesized in a similar manner to the intermediate—5. Compound 7-(5-(5-(difluoromethyl)-1,3,4-oxadiazol-2-yl)pyridin-2-yl)-2,7-diazaspiro[3.5]nonan-2-ium trifluoroacetate was obtained as its TFA salt (14a, 140 mg, >99%). RT=3.03 min, (HPLC Method-I). MS-ESI (m/z): Calculated [M+H]⁺=322.14; Observed [M+H]⁺=322.146. ¹H NMR (400 MHZ, CD₃OD) δ 9.43 (s, 1H), 8.71 (s, 1H), 8.15 (d, J=9.2 Hz, 1H), 6.97 (dd, J=30.6, 21.2 Hz, 2H), 3.90 (s, 4H), 3.79-3.36 (m, 4H), 2.49-1.75 (m, 4H).

### Compound of Invention—12 (Example-30): Intermediate of Formula-16a: 3-(5-(5-(difluoromethyl)-1,3,4-oxadiazol-2-yl)pyridin-2-yl)-3,6-diazabicyclo[3.1.1]heptan-6-ium trifluoroacetate

The intermediate 16a was synthesized in a similar manner to the intermediate—5. Compound 3-(5-(5-(difluoromethyl)-1,3,4-oxadiazol-2-yl)pyridin-2-yl)-3,6-diazabicyclo[3.1.1]heptan-6-ium trifluoroacetate was obtained as its TFA salt (16a, 190 mg, 98%). RT=3.23 min, (HPLC Method-I). MS-ESI (m/z): Calculated [M+H]⁺=294.11; Observed [M+H]⁺=294.116. ¹H NMR (400 MHZ, CD₃OD) δ 8.83 (d, J=2.3 Hz, 1H), 8.15 (dd, J=9.1, 2.3 Hz, 1H), 7.05-6.75 (m, 1H), 6.90 (t, J=52 Hz, 1H), 6.71 (d, J=9.0 Hz, 1H), 4.52 (d, J=6.4 Hz, 2H), 4.05 (s, 2H), 3.14 (dt, J=10.5, 6.5 Hz, 1H), 1.88 (d, J=10.5 Hz, 1H), 1.28-1.14 (m, 1H), 0.79 (dt, J=12.3, 7.4 Hz, 1H).

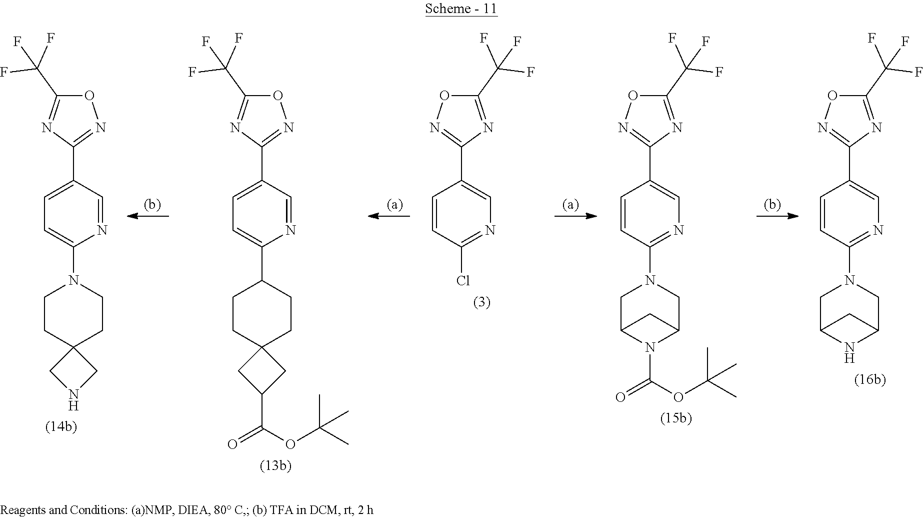

Intermediate—16b was prepared via the process of Scheme 11, supra, as follows

### Compound of Invention—13 (Example-7): Step 1: Intermediate of Formula-13b: tert-butyl 7-(5-(5-(trifluoromethyl)-1,2,4-oxadiazol-3-yl)pyridin-2-yl)-2,7-diazaspiro[3.5]nonane-2-carboxylate

The intermediate 13b was synthesized in a similar manner to the intermediate—4. Compound tert-butyl 7-(5-(5-(trifluoromethyl)-1,2,4-oxadiazol-3-yl)pyridin-2-yl)-2,7-diazaspiro[3.5]nonane-2-carboxylate was obtained as a brown color solid (13b, 0.170 g, 50%). RT=6.68 Min. (HPLC Method-I). MS-ESI (m/z): Calculated [M+H]⁺=440.18; Observed [M+H]⁺=440.189.). ¹H NMR (400 MHZ, CDCl₃) δ 8.86 (d, J=2.4 Hz, 1H), 8.07 (dd, J=9.1, 2.4 Hz, 1H), 6.76-6.61 (m, 1H), 3.71 (s, 4H), 3.66 (t, J=5.7 Hz, 4H), 1.92-1.73 (m, 4H), 1.45 (s, 9H).

### Compound of Invention—14 (Example-9): Intermediate of Formula-15b: tert-butyl 3-(5-(5-(trifluoromethyl)-1,2,4-oxadiazol-3-yl)pyridin-2-yl)-3,6-diazabicyclo[3.1.1]heptane-6-carboxylate

The intermediate 15b was synthesized in a similar manner to the intermediate—4. Compound tert-butyl 3-(5-(5-(trifluoromethyl)-1,2,4-oxadiazol-3-yl)pyridin-2-yl)-3,6-diazabicyclo[3.1.1]heptane-6-carboxylate was obtained as a colorless foam (15b, 80 mg, 32%). RT=6.45 Min. (HPLC Method-I). MS-ESI (m/z): Calculated [M+H]⁺=412.15; Observed [M+H]⁺=412.15 and 356.096.). ¹H NMR (400 MHZ, CDCl₃) δ 8.91 (d, J=2.3 Hz, 1H), 8.12 (dd, J=9.0, 2.4 Hz, 1H), 6.61 (d, J=9.0 Hz, 1H), 4.31 (d, J=6.2 Hz, 2H), 4.19 (s, 2H), 3.63-3.46 (m, 2H), 2.69 (h, J=7.7, 7.1 Hz, 1H), 1.50 (d, J=8.8 Hz, 1H), 1.35 (s, 9H).

### Compound of Invention—15 (Example-27): Step 2: Intermediate of Formula-14b: 7-(5-(5-(trifluoromethyl)-1,2,4-oxadiazol-3-yl)pyridin-2-yl)-2,7-diazaspiro[3.5]nonan-2-ium trifluoroacetate

The intermediate 14b was synthesized in a similar manner to the intermediate—5. Compound 7-(5-(5-(trifluoromethyl)-1,2,4-oxadiazol-3-yl)pyridin-2-yl)-2,7-diazaspiro[3.5]nonan-2-ium trifluoroacetate was obtained as its TFA salt as a brown color solid (14b, 150 mg, 89%). RT=4.38 Min. (HPLC Method-I). MS-ESI (m/z): Calculated [M+H]⁺=340.13; Observed [M+H]⁺=340.137. ¹H NMR (400 MHZ, CD₃OD) δ 8.71 (s, 1H), 8.28 (d, J=9.4 Hz, 1H), 7.16 (d, J=9.5 Hz, 1H), 3.93 (s, 4H), 3.71, s, 4H), 2.18-1.93 (m, 4H).

### Compound of Invention—16 (Example-29): Intermediate of Formula-16b: 3-(5-(5-(trifluoromethyl)-1,2,4-oxadiazol-3-yl)pyridin-2-yl)-3,6-diazabicyclo[3.1.1]heptan-6-ium trifluoroacetate

The intermediate 16b was synthesized in a similar manner to the intermediate—5. Compound 3-(5-(5-(trifluoromethyl)-1,2,4-oxadiazol-3-yl)pyridin-2-yl)-3,6-diazabicyclo[3.1.1]heptan-6-ium trifluoroacetate was obtained as its TFA salt (16b, 50 mg, 80%). RT=4.65 Min. (HPLC Method-I). MS-ESI (m/z): Calculated [M+H]⁺=312.10; Observed [M+H]⁺=312.106). ¹H NMR (400 MHZ, CDCl₃) δ 8.88 (d, J=2.3 Hz, 1H), 8.20 (dd, J=8.9, 2.3 Hz, 1H), 6.70 (d, J=9.0 Hz, 1H), 4.53 (d, J=6.1 Hz, 2H), 4.02 (d, J=25.5 Hz, 4H), 3.17 (dd, J=12.7, 6.4 Hz, 1H), 1.89 (d, J=10.3 Hz, 1H).

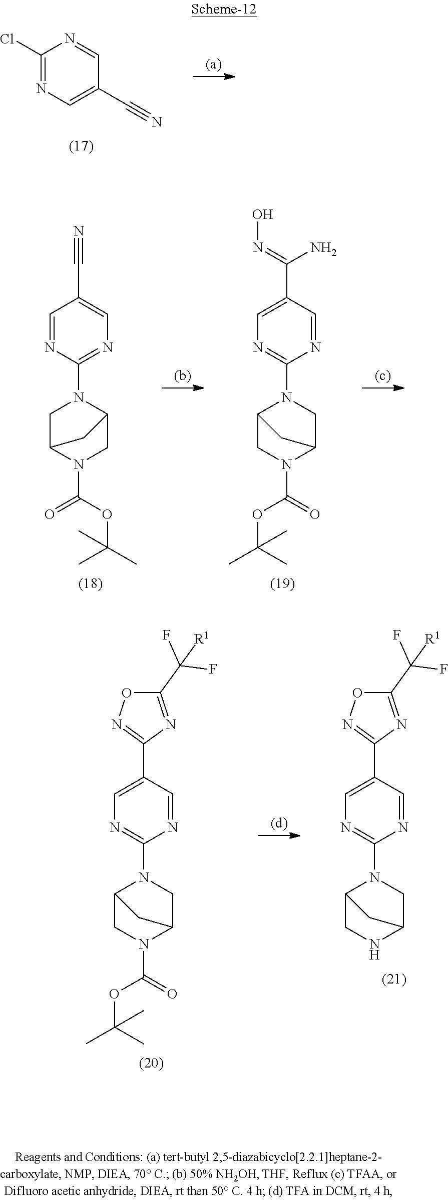

Intermediate—21 was prepared via the process of Scheme 12, supra, as

### Step 1: Intermediate of Formula-18: tert-butyl 5-(5-cyanopyrimidin-2-yl)-2,5-diazabicyclo[2.2.1]heptane-2-carboxylate

To a stirred mixture of compound2-chloropyrimidine-5-carbonitrile (17, 0.65 g, 4.70 mmol) and tert-butyl 2,5-diazabicyclo[2.2.1]heptane-2-carboxylate (1.40 g, 7.0 mmol) in NMP (15.0 mL) was added DIEPA (2.40 mL, 1.80 g, 14.0 mmol) and the resulting solution was heated to 70° C. under nitrogen atmosphere. After heating 4 h, and the dark brown color solution was poured dropwise in to a beaker containing water (50 mL) with vigorous stirring. The desired product precipitated out. Filtered and washed the tan color precipitate with water and dried at room temperature in the air overnight followed by high vacuum. The product tert-butyl 5-(5-cyanopyrimidin-2-yl)-2,5-diazabicyclo[2.2.1]heptane-2-carboxylate was obtained as a tan color solid (18, 1.30 g, 92%). RT=5.68 Min. (HPLC Method-I). MS-ESI (m/z): Calculated [M+H]⁺=302.10.13; Observed [M+H−100]⁺=202.109 and 246.0989 (m+−). ¹H NMR (400 MHZ, CDCl₃) δ 8.48 (dd, J=7.9, 3.0 Hz, 1H), 5.05 (s, 1H), 4.63 (d, J=64 Hz, 1H), 3.65 (t, J=11.2 Hz, 1H), 3.58 (t, J=2.7 Hz, 1H), 3.44 (q, J=11.3, 10.6 Hz, 2H), 1.95 (s, 2H), 1.43 (s, 9H).

### Step 2: Intermediate of Formula-16: tert-butyl 5-(5-(N′-hydroxycarbamimidoyl)pyrimidin-2-yl)-2,5-diazabicyclo[2.2.1]heptane-2-carboxylate

The intermediate 19 was synthesized in a similar manner to the intermediate 3. Compound tert-butyl 5-(5-(N′-hydroxycarbamimidoyl)pyrimidin-2-yl)-2,5-diazabicyclo[2.2.1]heptane-2-carboxylate as a white solid (19, 0.525 g, 79%). RT=4.31 Min. (HPLC Method-I). ¹H NMR (400 MHZ, DMSO-D6-d₆) δ 9.54 (s, 1H), 8.75 (s, 3H), 8.55 (s, 2H), 5.84 (s, 2H), 4.90 (d, J=24.3 Hz, 1H), 4.45 (d, J=22.2 Hz, 1H), 3.56 (d, J=21.5 Hz, 1H), 3.32 (m, 3H), 1.92 (s, 2H), 1.36 (d, J=19.2 Hz, 9H).

### Compound of Invention—17 (Example-4): Step 3: Intermediate of Formula-20a: tert-butyl 5-(5-(5-(trifluoromethyl)-1,2,4-oxadiazol-3-yl)pyrimidin-2-yl)-2,5-diazabicyclo[2.2.1]heptane-2-carboxylate

To a stirred suspension of compound (0.40 g, 1.20 mmol) in dry THF (10 mL) was added DIEA (0.652 mL, 0.46 g, 3.59 mmol) followed by addition of 2,2,2-trifluoroacetic anhydride (0.50 mL, 0.754 g, 3.59 mmol) at room temperature under nitrogen atmosphere. After stirring 15 at room temperature, the resulting solution is heated at 50° C. for 4 h. After heating 4 h at 50° C., the reaction mixture was cool to rt and quenched with water and the excess solvent was removed using rotovap. The crude product is redissolved in DCM and washed with aqueous citric acid solution, water and brine. Purification by column chromatography on a silica gel column using 30% EA/Hex followed by removal of solvent afforded the titled compound tert-butyl 5-(5-(5-(trifluoromethyl)-1,2,4-oxadiazol-3-yl)pyrimidin-2-yl)-2,5-diazabicyclo[2.2.1]heptane-2-carboxylate (20a, 90 mg, 18%) as a pale yellow color solid. RT=7.25 Min. (HPLC Method-I). MS-ESI (m/z): Calculated [M+H]⁺=413.15; Observed [M+H-56]⁺=357.09.). ¹H NMR (400 MHz, CDCl₃) δ 8.92 (q, J=3.2 Hz, 1H), 5.09 (s, 1H), 4.63 (d, J=60 Hz, 1H), 3.69-3.59 (m, 2H), 3.45 (d, J=8.5 Hz, 2H), 2.05-1.91 (m, 2H), 1.43 (d, J=16.7 Hz, 9H).

### Compound of Invention—18 (Example-3): Intermediate of Formula-20b: tert-butyl 5-(5-(5-(difluoromethyl)-1,2,4-oxadiazol-3-yl)pyrimidin-2-yl)-2,5-diazabicyclo[2.2.1]heptane-2-carboxylate

Compound 20b was synthesized in a similar manner to the intermediate 20a. Compound tert-butyl 5-(5-(5-(difluoromethyl)-1,2,4-oxadiazol-3-yl)pyrimidin-2-yl)-2,5-diazabicyclo[2.2.1]heptane-2-carboxylate (20b, 20 mg, 13%) was obtained as a pale yellow color solid. RT=6.60 Min. (HPLC Method-I).). ¹H NMR (400 MHZ, CDCl₃) δ 8.94 (d, J=3.4 Hz, 1H), 6.85 (t, J=52.1 Hz, 1H), 5.09 (s, 1H), 4.66 (d, J=64 Hz, 1H), 3.79-3.55 (m, 2H), 3.47 (d, J=11.1 Hz, 2H), 2.04-1.89 (m, 2H), 1.45 (s, 9H).

### Compound of Invention—19 (Example-24): Step 4: Intermediate of Formula-21a: 5-(5-(5-(trifluoromethyl)-1,2,4-oxadiazol-3-yl)pyrimidin-2-yl)-2,5-diazabicyclo[2.2.1]heptan-2-ium trifluoroacetate

Compound 21a was synthesized in a similar manner to the intermediate 20a. Compound 5-(5-(5-(trifluoromethyl)-1,2,4-oxadiazol-3-yl)pyrimidin-2-yl)-2,5-diazabicyclo[2.2.1]heptan-2-ium trifluoroacetate was obtained as its TFA salt as a brown color solid (21a, 0.050 g, >99%). RT=4.52 Min. (HPLC Method-I). ¹H NMR (400 MHZ, CDCl₃) δ 9.04 (s, 2H), 5.29 (d, J=5.3 Hz, 1H), 4.65 (br., s, 1H), 4.19-3.89 (m, 1H), 3.80 (d, J=20.5 Hz, 1H), 3.57 (br., s, 2H), 2.27 (s, 2H).

### Compounds of the Invention of formula (Iaaa and Iaaa1) and (Ibaa and Ibaa1)

### Compound of Invention—20 (Example-11): benzyl 5-(5-(5-(trifluoromethyl)-1,2,4-oxadiazol-3-yl)pyridin-2-yl)-2,5-diazabicyclo[2.2.1]heptane-2-carboxylate

To a stirred solution of compound 5-(5-(5-(trifluoromethyl)-1,2,4-oxadiazol-3-yl)pyridin-2-yl)-2,5-diazabicyclo[2.2.1]heptan-2-ium trifluoroacetate (0.052 g, 0.096 mmol) and DIPEA (0.086 mL, 0.062 g, 0.048 mmol) in DCM (3 mL) was added Cbz-Cl (0.02 g, 0.12 mmol) at room temperature. After stirring 2 h, the reaction was quenched with water and the product was extracted using DCM. Purification by a preparatory silica gel TLC using 40% EA/Hex followed by removal of solvent afforded the titled compound benzyl 5-(5-(5-(trifluoromethyl)-1,2,4-oxadiazol-3-yl)pyridin-2-yl)-2,5-diazabicyclo[2.2.1]heptane-2-carboxylatee (22, 20 mg, 43%) was obtained as a white solid. RT=6.18 Min. (HPLC Method-I). MS-ESI (m/z): Calculated [M+H]⁺=446.14; Observed [M+H]⁺=446.14. ¹H NMR (400 MHZ, CDCl₃) δ 8.84 (d, J=2.5 Hz, 1H), 8.07 (dd, J=8.9, 2.4 Hz, 1H), 7.37 (m, 3H), 7.32 (m, 2H), 6.39 (d, J=8.9 Hz, 1H), 5.28-4.99 (m, 3H), 4.74 (d, J=33.8 Hz, 1H), 3.70-3.36 (m, 4H), 2.00 (d, J=8.2 Hz, 2H).

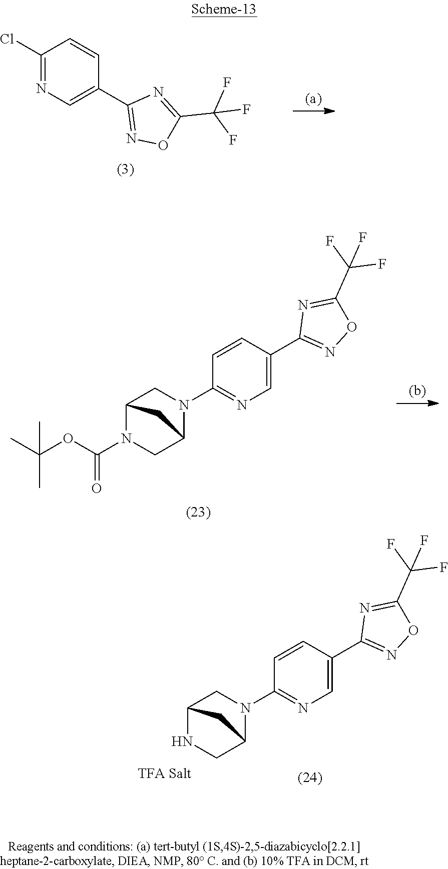 [SMILES: FC(F)(F)c1nc(-c2ccc(Cl)nc2)no1 ; CC(C)(C)OC(=O)N1C[C@@H]2C[C@H]1CN2c1ccc(-c2noc(C(F)(F)F)n2)cn1 ; FC(F)(F)c1nc(-c2ccc(N3C[C@@H]4C[C@H]3CN4)nc2)no1]

Intermediate—24 was prepared via the process of Scheme 13, supra, as

### Compound of Invention—21 (Example-17): tert-butyl (1S,4S)-5-(5-(S-(trifluoromethyl)-1,2,4-oxadiazol-3-yl)pyridin-2-yl)-2,5-diazabicyclo[2.2.1]heptane-2-carboxylate

To a stirred mixture of compound 3-(6-chloropyridin-3-yl)-5-(trifluoromethyl)-1,2,4-oxadiazole (I, 1.00 g, 4.01 mmol) and tert-butyl (1S,4S)-2,5-diazabicyclo[2.2.1]heptane-2-carboxylate (1.59 g, 8.01 mmol) in NMP (25.0 mL) was added DIEPA (1.61 mL, 1.190 mg, 4.94 mmol) was added and the resulting solution is heated to 700C under nitrogen atmosphere. After heating 6 h, the reaction mixture was cool to room temperature and poured in to water (50 mL) with vigorous stirring. The desired product precipitated out. Filtered and washed the pale brown color precipitate with water and dried at room temperature in the air followed by high vacuum. The desired product was obtained as a pale brown color solid (0.350 g). The aqueous portion was washed extracted with ethyl acetate and washed with aqueous citric acid, water and brine. Removal of solvent afforded the titled compound tert-butyl (1S,4S)-5-(5-(5-(trifluoromethyl)-1,2,4-oxadiazol-3-yl)pyridin-2-yl)-2,5-diazabicyclo[2.2.1]heptane-2-carboxylate (23, 1.00 g, 61%) as a dark brown color solid. RT=5.875 min (HPLC Method-I). Q-TOF LC/MS (m/z): Calculated [M+H]⁺=411.1518; Observed [M+H]⁺=412.1745 RT=5.875 min, ¹H NMR (400 MHz, CDCl₃) δ 8.84 (dd, J=2.4, 0.7 Hz, 1H), 8.07 (dd, J=8.8, 2.3 Hz, 1H), 6.39 (d, J=8.8 Hz, 1H), 5.04 (s, 1H), 4.71 (s, 1H), 4.57 (s, 1H), 3.57 (t, J=9.7 Hz, 1H), 3.45 (d, J=12.3 Hz, 3H), 3.41-3.30 (m, 4H), 2.84 (d, J=1.0 Hz, 5H), 2.37 (t, J=8.1 Hz, 4H), 2.11-1.89 (m, 6H), 1.72 (s, 2H). ¹⁹F NMR (376 MHZ, CDCl₃) δ-65.41.

### Compound of Invention—22 (Example-31): 3-(6-((1S,4S)-2,5-diazabicyclo[2.2.1]heptan-2-yl)pyridin-3-yl)-5-(trifluoromethyl)-1,2,4-oxadiazole TFA Salt

To a stirred solution of compound tert-butyl (1S,4S)-5-(5-(5-(trifluoromethyl)-1,2,4-oxadiazol-3-yl)pyridin-2-yl)-2,5-diazabicyclo[2.2.1]heptane-2-carboxylate (23, 0.30 g, 0.729 mmol) in DCM (9 mL) was added TFA (1.2 mL, 1.66 g, 14.6 mmol) was added at room temperature under nitrogen atmosphere. After stirring 4 h at rt, the excess solvent was removed under high vacuum afforded the titled compound 3-(6-((1S,4S)-2,5-diazabicyclo[2.2.1]heptan-2-yl)pyridin-3-yl)-5-(trifluoromethyl)-1,2,4-oxadiazole TFA Salt (24, 0.300 g, 95%). HPLC Retention time: 4.58 minute, ¹H NMR (400 MHZ, CDCl₃) δ 8.69 (d, J=2.2 Hz, 1H), 8.20 (dd, J=9.1, 2.2 Hz, 1H), 6.72 (d, J=9.1 Hz, 1H), 5.10 (s, 1H), 4.52 (s, 1H), 3.87 (s, 1H), 3.78 (d, J=11.4 Hz, 1H), 3.69 (dd, J=11.5, 2.2 Hz, 1H), 3.42 (s, 3H), 3.30 (s, 95H), 2.30 (t, J=8.1 Hz, 4H), 2.17 (s, 2H), 1.96 (q, J=7.7 Hz, 4H).

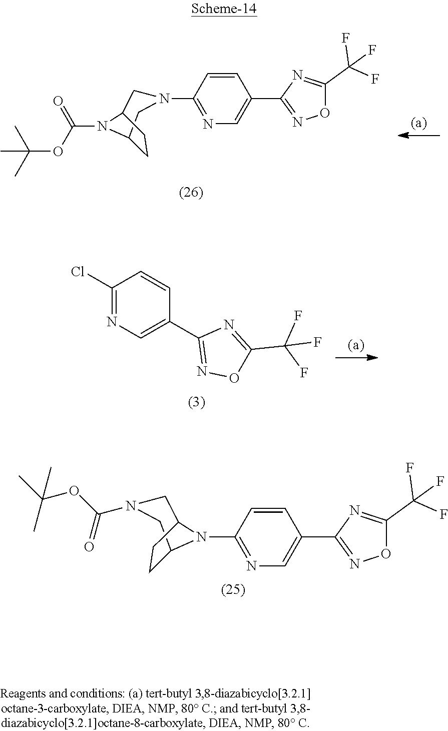 [SMILES: CC(C)(C)OC(=O)N1C2CCC1CN(c1ccc(-c3noc(C(F)(F)F)n3)cn1)C2 ; FC(F)(F)c1nc(-c2ccc(Cl)nc2)no1 ; CC(C)(C)OC(=O)N1CC2CCC(C1)N2c1ccc(-c2noc(C(F)(F)F)n2)cn1]

Intermediate—25 and 26 were prepared via the process of Scheme 14, supra, as

### Compound of Invention—23 (Example-18): tert-butyl 8-(5-(5-(trifluoromethyl)-1,2,4-oxadiazol-3-yl)pyridin-2-yl)-3,8-diazabicyclo[3.2.1]octane-3-carboxylate

To a stirred mixture of compound 3-(6-chloropyridin-3-yl)-5-(trifluoromethyl)-1,2,4-oxadiazole (I, 1.00 g, 4.01 mmol) and tert-butyl-3,8-diazabicyclo[3.2.1]octane-3-carboxylate (1.59 g, 8.01 mmol) in NMP (25.0 mL) was added DIEPA (1.61 mL, 1.190 mg, 4.94 mmol) was added and the resulting solution is heated to 70 C under nitrogen atmosphere. After heating 4 h, the reaction mixture was cool to room temperature and poured in to water (50 mL) with vigorous stirring. The desired product precipitated out. Filtered and washed the pale brown color precipitate with water and dried at room temperature in the air followed by high vacuum afforded the titled compound as a pale brown color solid (25, 0.420 g, 49%). RT=4.59 Min. (HPLC Method-I). Q-TOF LC/MS (m/z): Calculated [M+H]⁺=425.1647; Observed [M+H]⁺=426.1817; ¹H NMR (400 MHZ, CDCl₃) δ 8.87 (dd, J=2.4, 0.7 Hz, 1H), 8.09 (dd, J=8.9, 2.4 Hz, 1H), 6.66-6.60 (m, 1H), 6.59 (d, J=9.0 Hz, 1H), 4.62 (s, 2H), 4.49 (s, 2H), 3.88 (t, J=15.7 Hz, 2H), 3.80-3.65 (m, 3H), 3.21 (d, J=13.1 Hz, 2H), 3.10 (d, J=12.8 Hz, 2H), 2.08-1.97 (m, 4H), 1.94-1.77 (m, 5H), 1.73 (s, 2H), 1.45 (s, 15H), 1.43 (s, 4H).

### Compound of Invention—24 (Example-19): tert-butyl 3-(5-(5-(trifluoromethyl)-1,2,4-oxadiazol-3-yl)pyridin-2-yl)-3,8-diazabicyclo[3.2.1]octane-8-carboxylate

To a stirred mixture of compound 3-(6-chloropyridin-3-yl)-5-(trifluoromethyl)-1,2,4-oxadiazole (I, 0.50 g, 4.01 mmol) and tert-butyl 3,8-diazabicyclo[3.2.1]octane-8-carboxylate (1.59 g, 8.01 mmol) in NMP (25.0 mL) was added DIEPA (1.61 mL, 1.190 mg, 4.94 mmol) was added and the resulting solution is heated to 70 C under nitrogen atmosphere. After heating 4 h, the reaction mixture was cool to room temperature and poured in to ice cold water (50 mL) with vigorous stirring. The desired product precipitated out. Filtered and washed the pale brown color precipitate with water and dried at room temperature in the air followed by high vacuum afforded the titled compound as a pale brown color solid (26, 0.725 g, 80% yield). RT=7.07 Min. (HPLC Method-I). MS-ESI (m/z): Calculated [M+H]⁺=425.41; Observed [M+H]⁺=426.1875. ¹H NMR (400 MHZ, CDCl₃) δ 8.87 (d, J=2.3 Hz, 1H), 8.09 (ddd, J=9.1, 2.4, 0.8 Hz, 1H), 6.63 (d, J=9.0 Hz, 1H), 4.39 (s, 2H), 4.08 (s, 2H), 3.19 (s, 2H), 1.98 (dd, J=8.6, 4.3 Hz, 2H), 1.75 (d, J=7.4 Hz, 2H), 1.49 (d, J=0.7 Hz, 10H).

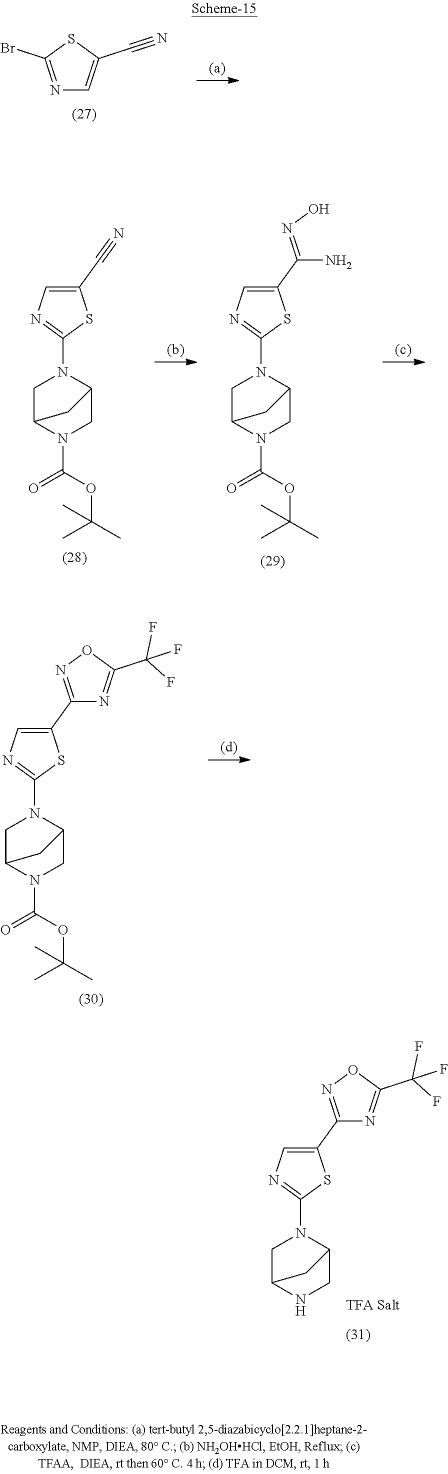 [SMILES: N#Cc1cnc(Br)s1 ; CC(C)(C)OC(=O)N1CC2CC1CN2c1ncc(C#N)s1 ; CC(C)(C)OC(=O)N1CC2CC1CN2c1ncc(/C(N)=N/O)s1 ; CC(C)(C)OC(=O)N1CC2CC1CN2c1ncc(-c2noc(C(F)(F)F)n2)s1 ; FC(F)(F)c1nc(-c2cnc(N3CC4CC3CN4)s2)no1]

Intermediate—31 was prepared via the process of Scheme 15, supra, as

### Step 1: Intermediate of Formula-28: tert-butyl 5-(5-cyanothiazol-2-yl)-2,5-diazabicyclo[2.2.1]heptane-2-Carboxylate

To a stirred mixture of compound 2-bromothiazole-5-carbonitrile (27, 1.00 g, 4.70 mmol) and tert-butyl 2,5-diazabicyclo[2.2.1]heptane-2-carboxylate (1.57 g, 7.94 mmol) in NMP (15.0 mL) was added DIEPA (2.76 mL, 2.05 g, 15.9 mmol) was added and the resulting solution is heated to 80 C under nitrogen atmosphere. After heating 4 h, the reaction mixture was cool to room temperature and continued the stirring at rt for 16 h, Then the dark brown color solution was poured dropwise into a beaker containing crushed iced cold water (50 mL) with vigorous stirring. The desired product precipitated out. Filtered and washed the tan color precipitate with water and dried at room temperature in the air overnight followed by high vacuum afforded the titled compound as a tan color solid (28, 1.48 g, 91%). RT=5.88 min, ¹H NMR (400 MHZ, CDCl₃) δ 7.26 (s, 1H), 4.70 (d, J=16.7 Hz, 2H), 4.58 (s, 1H), 3.55 (q, J=11.0, 9.7 Hz, 2H), 3.51-3.28 (m, 3H), 2.01 (d, J=9.4 Hz, 2H), 1.45 (d, J=12.7 Hz, 12H).

### Step 1: Intermediate of Formula-29: tert-butyl (Z)-5-(5-(N′-hydroxycarbamimidoyl)thiazol-2-yl)-2,5-diazabicyclo[2.2.1]heptane-2-carboxylate

To a stirred mixture of tert-butyl 5-(5-cyanothiazol-2-yl)-2,5-diazabicyclo[2.2.1]heptane-2-carboxylate (1.41 g, 4.60 mmol) and hydroxylamine hydrochloride (0.704 g, 10.1 mmol) in Ethanol (20.0 mL) was added triethylamine (1.48 mL, 1.07 g, 10.60 mmol) at room temperature. The reaction mixture becomes a clear solution within 30 minutes and the resulting mixture was allowed to reflux under nitrogen atmosphere. After refluxing 16 h, the resulting clear solution was cool to room temperature and water (20 mL) was added with vigorous stirring. The product precipitated out and the solution is concentrated to half of its volume. Filtered the white precipitate and washed it with water and dried at room temperature afforded the titled compound as a white solid (29, 1.33 g, 85%) which was used for the next step. HPLC RT=4.24 min., ¹H NMR (400 MHZ, DMSO-D₆) δ 9.43 (s, 1H), 7.51 (s, 1H), 5.79 (s, 2H), 4.49 (s, 1H), 4.42 (d, J=21.7 Hz, 1H), 3.52 (t, J=9.3 Hz, 1H), 3.36 (s, 19H), 3.20 (dd, J=27.7, 9.7 Hz, 3H), 2.48 (s, 2H), 1.91 (d, J=17.1 Hz, 2H), 1.35 (d, J=16.7 Hz, 10H).

### Compound of Invention—25 (Example-12): Step 1: Intermediate of Formula-30: tert-butyl 5-(5-(5-(trifluoromethyl)-1,2,4-oxadiazol-3-yl)thiazol-2-yl)-2,5-diazabicyclo[2.2.1]heptane-2-carboxylate

To a stirred suspension of compound tert-butyl (Z)-5-(5-(N′-hydroxycarbamimidoyl)thiazol-2-yl)-2,5-diazabicyclo[2.2.1]heptane-2-carboxylate (29,1.25 g, 3.68 mmol) in dry THF (20 mL) was added DIEA (1.92 mL, 1.43 g, 11.0 mmol) followed by addition of 2,2,2-trifluoroacetic anhydride (1.56 mL, 2.32 g, 11.0 mmol) at room temperature under nitrogen atmosphere. After stirring 15 at room temperature, the resulting solution is heated at 60° C. for 16 h. After heating 16 h at 60 C, the reaction mixture was cool to rt and quenched with water. The excess solvent was removed using rotopvap. The crude product is redissolved in DCM and washed with aqueous citric acid solution, water and brine. Purification by column Chromatography on silica gel using a gradient of 5% EA/Hex to 20% EA/Hex followed by removal of solvent afforded the titled compound tert-butyl 5-(5-(5-(trifluoromethyl)-1,2,4-oxadiazol-3-yl)thiazol-2-yl)-2,5-diazabicyclo[2.2.1]heptane-2-carboxylate (30, 1.30 g, 85%) as a pale yellow color foam. TLC Rf: 0.46 in 30% EA/Hex HPLC RT=7.04 Min. MS-ESI (m/z): Calculated [M+H]⁺=417.1471; Observed [M+H]⁺=418.1825. ¹H NMR (400 MHZ, CDCl₃) δ 7.96 (s, 1H), 4.72 (t, J=10.6 Hz, 2H), 4.58 (s, 1H), 3.60 (q, J=10.4, 9.3 Hz, 2H), 3.53-3.39 (m, 3H), 2.04 (d, J=2.7 Hz, 2H), 2.01 (d, J=4.5 Hz, 1H), 1.45 (d, J=15.6 Hz, 11H). ¹⁹F NMR (376 MHz, CDCl₃) δ-65.46.

### Compound of Invention—26 (Example-34): Step 1: Intermediate of Formula-31:3-(2-(2,5-diazabicyclo[2.2.1]heptan-2-yl)thiazol-5-yl)-5-(trifluoromethyl)-1,2,4-oxadiazole TFA Salt

To a stirred solution of compound tert-butyl 5-(5-(5-(trifluoromethyl)-1,2,4-oxadiazol-3-yl)thiazol-2-yl)-2,5-diazabicyclo[2.2.1]heptane-2-carboxylate (0.930 g, 2.23 mmol) in DCM (20 mL) was added Trifluoroacetic acid (1.72 mL, 2.54 g, 22.3 mmol) at rt under nitrogen atmosphere. After stirring at rt overnight (18 h) the solvent was removed using rotovap and the resulting gummy residue was dried under high vacuum to afford the titled product 5-(5-(5-(trifluoromethyl)-1,2,4-oxadiazol-3-yl)thiazol-2-yl)-2,5-diazabicyclo[2.2.1]heptan-2-ium TFA Salt (31, 950 mg, >99%) as a brown color solid. HPLC RT=4.77 Min.

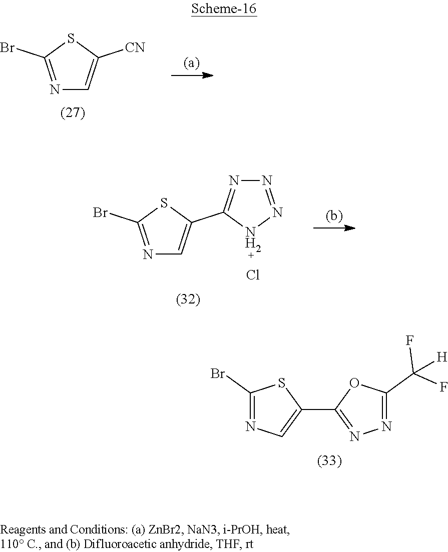 [SMILES: N#Cc1cnc(Br)s1 ; Brc1ncc(-c2nnn[nH]2)s1 ; Cl ; FC(F)c1nnc(-c2cnc(Br)s2)o1]

Intermediate—33 was prepared via the process of Scheme 16, supra, as

### Step 2: Intermediate of Formula-32:2-bromo-5-(2H-tetrazol-5-yl)thiazole

To a stirred solution of 2-bromothiazole-5-carbonitrile (27, 1.00 g, in i-PrOH (15 mL) and water 910 mL) was added sodium azide (516 mg, 7.94 mmol) followed by Zinc bromide (1.19 g, 5.29 mmol). The resulting mixture was heated to 120° C. for 5 h. After heating 5 h at 120° C., the reaction mixture was cool to room temperature and diluted with water (10 mL) and acidified using 1M HCl Solution (˜5 mL) to a pH of 3.0. The product is extracted with ethylacetate three times, combined the organic portion was washed with brine and dried over anhyd. sodium sulfate. Filter and removal of solvent afforded the titled product 2-bromo-5-(2H-tetrazol-5-yl)thiazole as a colorless solid (32, 1.39 g, 98%). HPLC RT=3.94 min. ¹H NMR (400 MHZ, CDCl₃) δ 8.13 (s, 1H).

### Step 2: Intermediate of Formula—33:2-(2-bromothiazol-5-yl)-5-(difluoromethyl)-1,3,4-oxadiazole

To a stirred cold suspension of 5-(5-bromothiophen-2-yl)-1H-tetrazole (32, 1.25 g, 4.66 mmol) in DCM (25 mL) was added 2,2-difluoroacetic anhydride (2.89 mL, 4.05 g, 23.3 mmol). The clear solution was stirred at room temperature overnight and the excess solvent was removed using rotovap. The crude product was extracted using dichloromethane and washed the organic portion with aqueous citric acid solution, water, aqueous NaHCO₃ solution, water and brine. The organic portion was dried over anhydrous sodium sulfate, filtered followed by removal of solvent afforded the desired product as a white solid (33, 1.00 g, 76%). HPLC RT=5.49 min ¹H NMR (400 MHZ, CDCl₃) δ 8.26 (s, 1H), 8.13 (s, 0H), 6.91 (t, J=51.6 Hz, 1H).

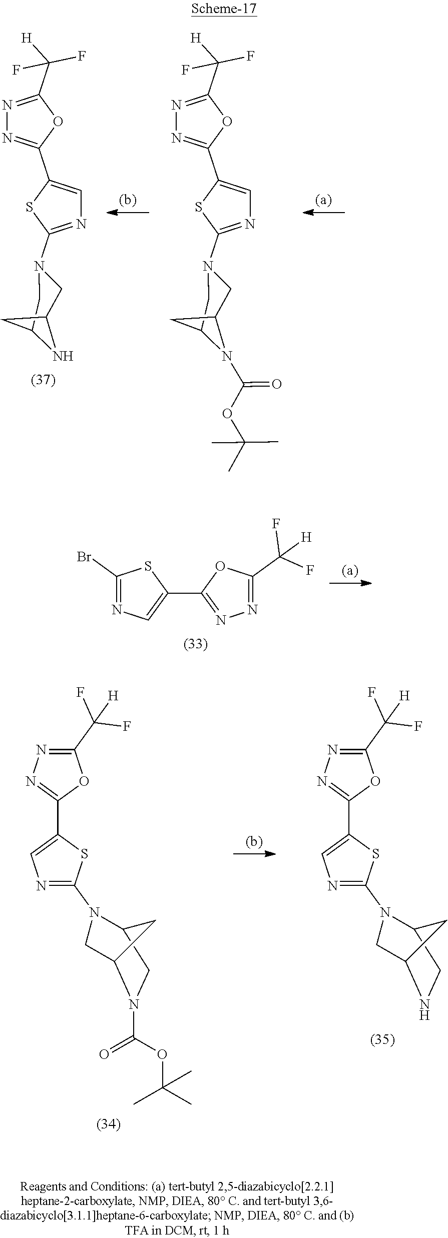 [SMILES: FC(F)c1nnc(-c2cnc(N3CC4CC(C3)N4)s2)o1 ; CC(C)(C)OC(=O)N1C2CC1CN(c1ncc(-c3nnc(C(F)F)o3)s1)C2 ; FC(F)c1nnc(-c2cnc(Br)s2)o1 ; CC(C)(C)OC(=O)N1CC2CC1CN2c1ncc(-c2nnc(C(F)F)o2)s1 ; FC(F)c1nnc(-c2cnc(N3CC4CC3CN4)s2)o1]

Intermediate—35 and 37 were prepared via the process of Scheme 16, supra, as

### Compound of Invention—27 (Example-13): Step 1: Intermediate of Formula-34: tert-butyl (1R, 4R)-5-(5-(5-(difluoromethyl)-1,3,4-oxadiazol-2-yl)thiazol-2-yl)-2,5-diazabicyclo[2.2.1]heptane-2-carboxylate

To a stirred mixture of compound 2-(2-bromothiazol-5-yl)-5-(difluoromethyl)-1,3,4-oxadiazole (33, 0.50 g, 1.77 mmol) and tert-butyl (1R,4R)-2,5-diazabicyclo[2.2.1]heptane-2-carboxylate (0.492 g, 2.48 mmol) in NMP (10.0 mL) was added DIEPA (0.926 mL, 0.687 g, 5.32 mmol) was added and the resulting solution was heated to 80° C. under nitrogen atmosphere. After heating 6 h, the reaction mixture was cool to room temperature and continued the stirring at rt for 16 h, The dark brown color solution was poured dropwise in to a beaker containing crushed iced cold water (50 mL) with vigorous stirring. The desired product precipitated out. Filtered and washed the tan color precipitate with water and dried at room temperature overnight followed by high vacuum afforded the titled product as a tan color solid (34, 0.480 g, 68%). The aqueous portion was extracted with ethyl acetate and washed with citric acid solution, water and brine. The organic portion was dried over anhydrous Na₂SO₄, filtered and removal of solvent resulted additional quantity of the desired product (34, 0.120 g, 17%). RT=6.06 min (HPLC Method-I). Q-TOF LC/MS (m/z): Calculated [M+H]⁺=399.1177; Observed [M+H]⁺=400.1268. ¹H NMR (400 MHz, CDCl₃) δ 7.92 (s, 1H), 6.85 (t, J=51.8 Hz, 1H), 4.74 (s, 2H), 3.59 (d, J=18.1 Hz, 2H), 3.47 (s, 3H), 2.04 (d, J=10.6 Hz, 2H), 1.46 (d, J=15.5 Hz, 10H H).

### Compound of Invention—28 (Example-32): Step 2: Intermediate of Formula-35:5-(5-(5-(difluoromethyl)-1,3,4-oxadiazol-2-yl)thiazol-2-yl)-2,5-diazabicyclo[2.2.1]heptan-2-ium 2,2,2-trifluoroacetate

To a stirred solution of compound tert-butyl 5-(5-(5-(difluoromethyl)-1,3,4-oxadiazol-2-yl)thiazol-2-yl)-2,5-diazabicyclo[2.2.1]heptane-2-carboxylate (34, 0.430 g, 1.08 mmol) in DCM (10 mL) was added 2,2,2-trifluoroacetic acid (0.412 mL, 5.38 mmol) at room temperature under nitrogen atmosphere. After stirring 2 h at rt, the excess solvent and volatiles were removed using rotovap and the resulting gummy material was dried under high vacuum afforded the titled product as a brown color solid (35, 255 mg) as its TFA salt. HPLC Retention time: 3.63 Min. ¹H NMR (400 MHZ, CDCl₃) δ 7.86 (s, 1H), 6.83 (t, J=51.7 Hz, 1H), 4.79 (s, 1H), 4.51 (s, 1H), 3.92 (s, 2H), 3.70-3.55 (m, 3H), 3.31 (s, 2H), 2.32-1.93 (m, 2H).

### Compound of Invention—29 (Example-14): Step 1: Intermediate of Formula-36: tert-butyl (1R,5S)-3-(5-(5-(difluoromethyl)-1,3,4-oxadiazol-2-yl)thiazol-2-yl)-3,6-diazabicyclo[3.1.1]heptane-6-carboxylate

To a stirred mixture of compound 2-(2-bromothiazol-5-yl)-5-(difluoromethyl)-1,3,4-oxadiazole (33, 0.45 g, 1.60 mmol) and tert-butyl (1R,5S)-3,6-diazabicyclo[3.1.1]heptane-6-carboxylate (0.44 g, 2.20 mmol) in NMP (15.0 mL) was added DIEPA (0.83 mL, 0.62 g, 4.80 mmol) was added and the resulting solution is heated to 80 C under nitrogen atmosphere. After heating 6 h, the reaction mixture was cool to room temperature and continued the stirring at rt for 16 h, The dark brown color solution was poured dropwise in to a beaker containing crushed iced cold water (50 mL) with vigorous stirring. Filter and washed the tan color precipitate with water and dried at room temperature in the air overnight followed by high vacuum afforded the titled product as a tan color solid (36, 0.620 g, 80%). RT=6.13 min (HPLC Method-I). Q-TOF LC/MS (m/z): Calculated [M+H]⁺=399.1177; Observed [M+H]⁺=400.1269. ¹H NMR (400 MHZ, CDCl₃) δ 7.99 (s, 1H), 6.86 (t, J=51.7 Hz, 1H), 4.30 (d, J=6.3 Hz, 3H), 4.18 (s, 2H), 2.75 (q, J=7.2 Hz, 1H), 1.60-1.50 (m, 2H), 1.38 (s, 9H).

### Compound of Invention—30 (Example-33): Step 2: Intermediate of Formula-37:3-(5-(5-(difluoromethyl)-1,3,4-oxadiazol-2-yl)thiazol-2-yl)-3,6-diazabicyclo[3.1.1]heptan-6-ium 2,2,2-trifluoroacetate

To a stirred solution of compound tert-butyl 3-(5-(5-(difluoromethyl)-1,3,4-oxadiazol-2-yl)thiazol-2-yl)-3,6-diazabicyclo[3.1.1]heptane-6-carboxylate (36, 0.470 g, 1.18 mmol) in DCM (10 mL) was added 2,2,2-trifluoroacetic acid (0.450 mL, 5.88 mmol) at room temperature under nitrogen atmosphere. After stirring 4 h, removal of excess solvent followed by drying it under I high vacuum afforded the titled product as a brown color solid (37, 430 mg) as its TFA salt. HPLC Retention time: 3.61 Min. ¹H NMR (400 MHZ, CDCl₃) δ 7.95 (s, 1H), 6.84 (t, J=51.6 Hz, 1H), 4.50 (d, J=6.6 Hz, 2H), 4.07 (d, J=12.6 Hz, 2H), 3.95 (d, J=12.7 Hz, 3H), 3.62 (s, 2H), 3.33 (s, 2H), 1.90 (d, J=10.8 Hz, 1H).

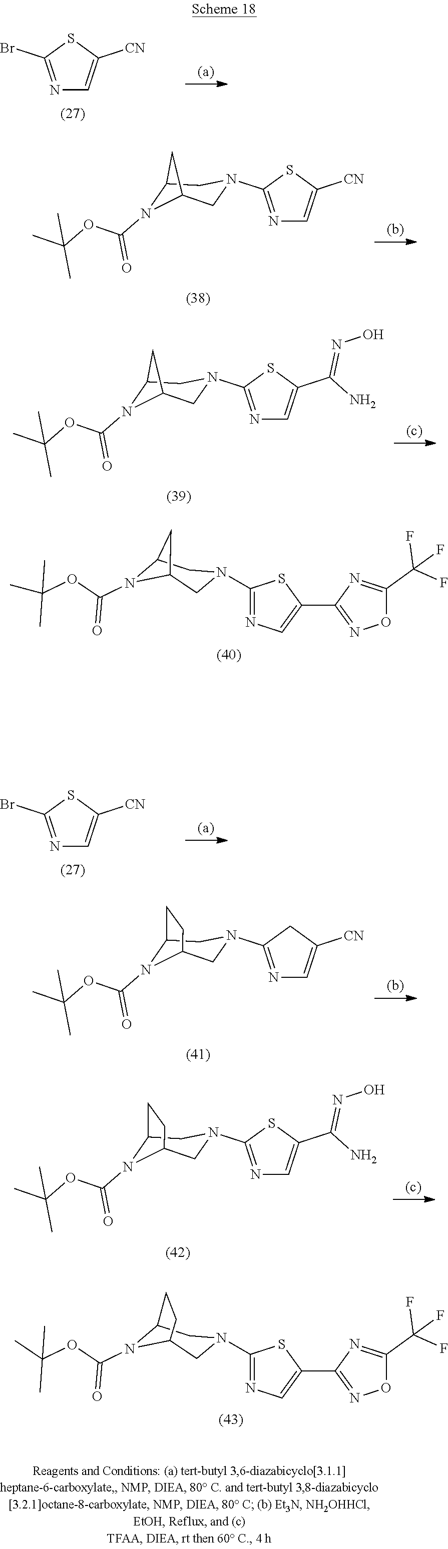

Intermediate—40 and 43 were prepared via the process of Scheme 17, supra, as

### Step 1: Intermediate of Formula-38: tert-butyl (1R,5S)-3-(5-cyanothiazol-2-yl)-3,6-diazabicyclo[3.1.1]heptane-6-Carboxylate

To a stirred mixture of compound 2-bromothiazole-5-carbonitrile (27, 1.00 g, 4.70 mmol) and tert-butyl 2,5-diazabicyclo[2.2.1]heptane-2-carboxylate (1.57 g, 7.94 mmol) in NMP (15.0 mL) was added DIEPA (2.76 mL, 2.05 g, 15.9 mmol) was added and the resulting solution was heated to 80° C. under nitrogen atmosphere. After heating 4 h, the reaction mixture was cool to room temperature and continued the stirring at rt for 16 h, The dark brown color solution was poured dropwise in to a beaker containing crushed iced cold water and (50 mL) with vigorous stirring. Filtered and washed the tan color precipitate with water and dried at room temperature in the air overnight followed by high vacuum afforded the titled product as a pale yellow color solid (38, 1.56 g, 99% yield). RT=5.88 min, ¹H NMR (400 MHZ, CDCl₃) δ 7.75 (s, 1H), 4.27 (d, J=6.0 Hz, 2H), 4.10 (s, 2H), 3.49 (s, 2H), 2.73 (q, J=7.4 Hz, 1H), 1.56-1.44 (m, 1H), 1.37 (s, 10H).

### Step 2: Intermediate of Formula-39: tert-butyl (1R,5S)-3-(5-((Z)—N′-hydroxycarbamimidoyl)thiazol-2-yl)-3,6-diazabicyclo[3.1.1]heptane-6-carboxylate

To a stirred mixture of tert-butyl (1R,5S)-3-(5-cyanothiazol-2-yl)-3,6-diazabicyclo[3.1.1]heptane-6-carboxylate (38, 1.42 g, 4.64 mmol) and hydroxylamine hydrochloride (0.709 g, 10.2 mmol) in Ethanol (20.0 mL) was added triethylamine (1.61 mL, 1.17 g, 11.60 mmol) at room temperature. The reaction mixture becomes a clear solution within 30 minutes and the resulting mixture was refluxed under nitrogen atmosphere. After refluxing 16 h, the clear solution was cool to room temperature and water (20 mL) was added with vigorous stirring. The product precipitated out and the solution is concentrated to half of its volume. Filtered the white precipitate and washed it with water followed by drying at room temperature vacuum afforded the titled product tert-butyl (1R,5S)-3-(5-((Z)—N′-hydroxycarbamimidoyl)thiazol-2-yl)-3,6-diazabicyclo[3.1.1]heptane-6-carboxylate (39, 1.280 g, 81%) as a white solid. HPLC RT=4.15 Min. ¹H NMR (400 MHZ, CDCl₃) δ 7.33 (s, 1H), 4.24-4.10 (m, 3H), 3.91 (d, J=22.5 Hz, 6H), 3.28 (s, 2H), 2.59 (d, J=9.2 Hz, 2H), 1.41 (d, J=8.8 Hz).

### Compound of Invention—31 (Example-24): Step 3: Intermediate of Formula-40: tert-butyl (1R,5S)-3-(5-(5-(trifluoromethyl)-1,2,4-oxadiazol-3-yl)thiazol-2-yl)-3,6-diazabicyclo[3.1.1]heptane-6-carboxylate

To a stirred suspension of compound tert-butyl (1R,5S)-3-(5-((Z)—N′-hydroxycarbamimidoyl)thiazol-2-yl)-3,6-diazabicyclo[3.1.1]heptane-6-carboxylate (39, 1.20 g, 3.53 mmol) in dry THF (20 mL) was added DIEA (1.85 mL, 1.37 g, 10.67 mmol) followed by addition of 2,2,2-trifluoroacetic anhydride (1.50 mL, 2.23 g, 10.61 mmol) at room temperature under nitrogen atmosphere. The resulting clear solution is heated at 60 C for 16 h. After heating 16 h, the reaction mixture was cool to rt and quenched with water and the excess solvent was removed using rotopvap. The crude product was re-dissolved in DCM and washed with aqueous citric acid solution, water and brine. Purification by column chromatography on silica gel using 30% EA/Hex followed by removal of solvent afforded the titled product tert-butyl (1R,5S)-3-(5-(5-(trifluoromethyl)-1,2,4-oxadiazol-3-yl)thiazol-2-yl)-3,6-diazabicyclo[3.1.1]heptane-6-carboxylate (40, 1.350 g, 88%) as a pale yellow color solid TLC: Rf 0.60 in 30% EA/Hex, RT=7.11 Min. (HPLC Method-I). Q-TOF LC/MS (m/z): Calculated [M+H]⁺=417.1082; Observed [M+H]⁺=418.1178. ¹H NMR (400 MHZ, CDCl₃) δ 7.97 (s, 1H), 4.24 (d, J=6.6 Hz, 2H), 4.10 (dt, J=20.2, 9.0 Hz, 2H), 3.48 (td, J=30.6, 27.8, 14.4 Hz, 2H), 2.69 (q, J=7.3 Hz, 1H), 1.31 (d, J=1.8 Hz, 9H), 1.26 (s, 1H).

### Step 1: Intermediate of Formula-41: tert-butyl (1R,5S)-3-(5-cyanothiazol-2-yl)-3,8-diazabicyclo[3.2.1]octane-8-carboxylate

To a stirred mixture of compound 2-bromothiazole-5-carbonitrile (27, 1.00 g, 4.70 mmol) and tert-butyl 2,5-diazabicyclo[2.2.1]heptane-2-carboxylate (1.57 g, 7.94 mmol) in NMP (15.0 mL) was added DIEPA (2.76 mL, 2.05 g, 15.9 mmol) was added and the resulting solution is heated to 80 C under nitrogen atmosphere. After heating 4 h, the reaction mixture was cool to room temperature and continued the stirring at rt for 16 h, Then the dark brown color solution was poured dropwise in to a beaker containing crushed iced cold water and (50 mL) with vigorous stirring. Filtered and washed the tan color precipitate with water and dried at room temperature in the air overnight followed by high vacuum afforded the titled product (41, 1.48 g, 91%) as a tan color solid. RT=6.496 Min. ¹H NMR (400 MHZ, CDCl₃) δ 7.69 (d, J=1.2 Hz, 1H), 4.37 (s, 2H), 3.65 (s, 2H), 3.39 (s, 2H), 2.09-1.88 (m, 2H), 1.75 (d, J=7.6 Hz, 2H), 1.48 (d, J=1.2 Hz, 10H).

### Step 2: Intermediate of Formula-42: tert-butyl (1R,5S)-3-(5-((Z)—N′-hydroxycarbamimidoyl)thiazol-2-yl)-3,8-diazabicyclo[3.2.1]octane-8-carboxylate

To a stirred mixture of tert-butyl (1R,5S)-3-(5-cyanothiazol-2-yl)-3,8-diazabicyclo[3.2.1]octane-8-carboxylate (41, 0.65 g, 2.03 mmol) and hydroxylamine hydrochloride (0.310 g, 4.46 mmol) in Ethanol (20.0 mL) was added triethylamine (0.71 mL, 0.513 g, 5.07 mmol) at room temperature. The reaction mixture becomes a clear solution within 30 minutes and the resulting mixture was refluxed under nitrogen atmosphere. After refluxing 16 h, the reaction was cool to room temperature and water (20 mL) was added with vigorous stirring. The product precipitated out from the solution upon concentration into half of its volume. Filtered the white precipitate and washed it with water and dried at room temperature afforded the titled product tert-butyl (1R,5S)-3-(5-((Z)—N′-hydroxycarbamimidoyl)thiazol-2-yl)-3,8-diazabicyclo[3.2.1]octane-8-carboxylate (42, 0.530 g, 74%) as a white solid. HPLC RT=4.67 Min. ¹H NMR (400 MHZ, CDCl₃) δ 7.30 (s, 1H), 4.26 (s, 2H), 3.60 (s, 5H), 3.49 (s, 4H), 3.14 (s, 2H), 1.92 (dd, J=8.5, 4.2 Hz, 2H), 1.70 (t, J=7.0 Hz, 2H), 1.39 (s, 9H).

### Compound of Invention—32 (Example-25): Step 3: Intermediate of Formula-43: tert-butyl (1R,5S)-3-(5-(5-(trifluoromethyl)-1,2,4-oxadiazol-3-yl)thiazol-2-yl)-3,8-diazabicyclo[3.2.1]octane-8-carboxylate

To a stirred suspension of compound tert-butyl (1R,5S)-3-(5-((Z)—N′-hydroxycarbamimidoyl)thiazol-2-yl)-3,8-diazabicyclo[3.2.1]octane-8-carboxylate (42, 0.450 g, 1.27 mmol) in dry THF (20 mL) was added DIEA (0.665 mL, 0.494 g, 3.82 mmol) followed by addition of 2,2,2-trifluoroacetic anhydride (0.54 mL, 0.802 g, 3.82 mmol) at room temperature under nitrogen atmosphere. After stirring 15 h at room temperature, the resulting solution is heated at 50° C. for 4 h. After heating 16 h at 60° C., the reaction mixture was cool to rt and quenched with water and the excess solvent was removed using rotopvap. The crude product is redissolved in DCM and washed with aqueous citric acid solution, water and brine. Purification by column chromatography on a silica gel column using 30% EA/Hex followed by removal of solvent afforded the titled product tert-butyl (1R,5S)-3-(5-(5-(trifluoromethyl)-1,2,4-oxadiazol-3-yl)thiazol-2-yl)-3,8-diazabicyclo[3.2.1]octane-8-carboxylate (43, 0.50 g, 88%) as a pale yellow color solid. RT=7.69 Min. (HPLC Method-I). Q-TOF LC/MS (m/z): Calculated [M+H]⁺=431.1239; Observed [M+H]⁺=432.1335. ¹H NMR (400 MHZ, CDCl₃) δ 7.93 (s, 1H), 4.35 (s, 2H), 3.68 (s, 2H), 3.38 (s, 2H), 2.00 (dd, J=8.9, 4.3 Hz, 2H), 1.77 (d, J=7.5 Hz, 2H), 1.45 (d, J=3.1 Hz, 9H), 1.30 (d, J=6.0 Hz, 2H).

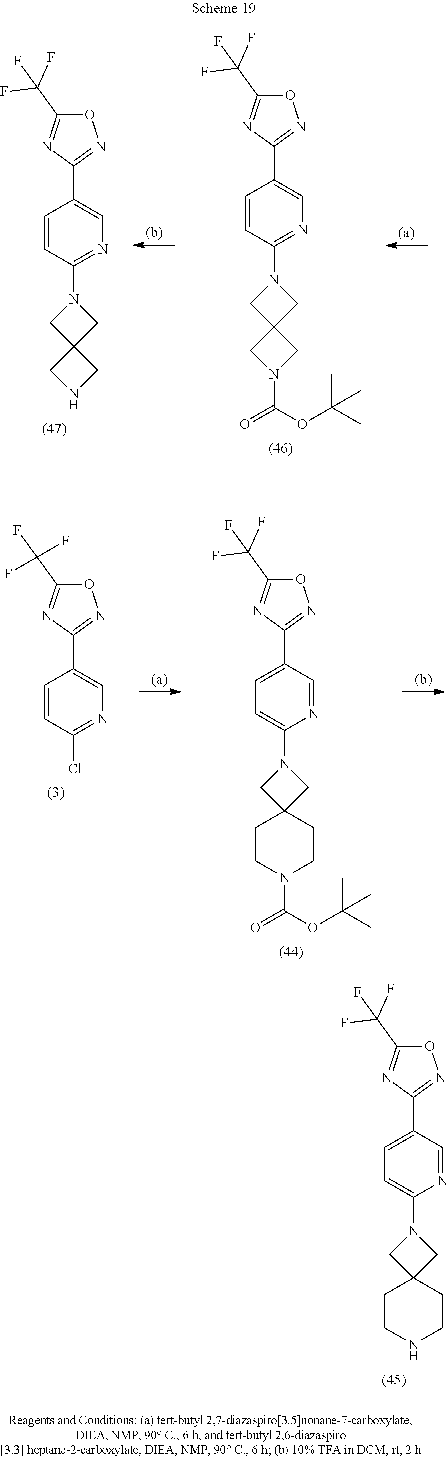 [SMILES: FC(F)(F)c1nc(-c2ccc(N3CC4(CNC4)C3)nc2)no1 ; CC(C)(C)OC(=O)N1CC2(C1)CN(c1ccc(-c3noc(C(F)(F)F)n3)cn1)C2 ; FC(F)(F)c1nc(-c2ccc(Cl)nc2)no1 ; CC(C)(C)OC(=O)N1CCC2(CC1)CN(c1ccc(-c3noc(C(F)(F)F)n3)cn1)C2 ; FC(F)(F)c1nc(-c2ccc(N3CC4(CCNCC4)C3)nc2)no1]

Intermediate—45 and 47 were prepared via the process of Scheme 18, supra, as

### Compound of Invention—33 (Example-20): Step 1: Intermediate of Formula-44: tert-butyl 2-(5-(5-(trifluoromethyl)-1,2,4-oxadiazol-3-yl)pyridin-2-yl)-2,7-diazaspiro[3.5]nonane-7-carboxylate

To a flask containing the intermediate (5) (0.75 g) and tert-butyl 2,7-diazaspiro[3.5]nonane-7-carboxylate (1.02 g, 1.5 equiv) in NMP (37.6 mL) was added DIPEA (0.78 g, 1.05 mL, 2 equiv) and heated to 90° C. under nitrogen atmosphere. After heating overnight, the reaction was cooled to room temperature. In a beaker with 50 mL of ice, the crude product was added dropwise and stirred vigorously. The precipitated product was then filtered and washed with DI water followed by drying under high vacuum afforded the titled product (tert-butyl 2-(5-(5-(trifluoromethyl)-1,2,4-oxadiazol-3-yl)pyridin-2-yl)-2,7-diazaspiro[3.5]nonane-7-carboxylate (44, 1.00 g, 75%) as a pale brown solid. HPLC Retention time 5.814 min, ¹H NMR (400 MHZ, CDCl₃) δ 8.85 (dd, J=2.3, 0.7 Hz, 1H), 8.06 (dd, J=8.8, 2.3 Hz, 1H), 6.39-6.29 (m, 1H), 3.87 (s, 4H), 3.42 (t, J=5.6 Hz, 5H), 1.86-1.72 (m, 4H), 1.56 (s, 2H), 1.47 (s, 11H).

### Compound of Invention—34 (Example-35): Step 2: Intermediate of Formula-45:3-(6-(2,7-diazaspiro[3.5]nonan-2-yl)pyridin-3-yl)-5-(trifluoromethyl)-1,2,4-oxadiazole TFA Salt

To a stirred solution of (tert-butyl 2-(5-(5-(trifluoromethyl)-1,2,4-oxadiazol-3-yl)pyridin-2-yl)-2,7-diazaspiro[3.5]nonane-7-carboxylate (44, 0.53 g) in DCM (25 mL) was added trifluoroacetic acid (3.03 g, 2.03 mL, 22.0 equiv) at room temperature under nitrogen atmosphere. After stirring overnight, the solvent and excess TFA was removed using rotovap. The resulting brown color gummy material was dissolved in DCM and the solvents were once again removed using rotovap and dried under high vacuum. The compound was dissolved with minimal DCM (˜5 mL) to which tert-butyl methyl ether was added (50 mL) and the oil was vigorously agitated. The precipitated product was then filtered, washed with hexanes and placed in high vacuum to afforded the titled product 3-(6-(2,7-diazaspiro[3.5]nonan-2-yl)pyridin-3-yl)-5-(trifluoromethyl)-1,2,4-oxadiazole TFA salt (45, 500 mg, 91%) as a grey color solid. HPLC Retention time 3.971 min, ¹H NMR (400 MHz, CDCl₃) δ 8.80 (s, 1H), 8.09 (d, J=8.8 Hz, 1H), 6.36 (d, J=8.8 Hz, 1H), 3.91 (s, 5H), 3.17 (d, J=13.6 Hz, 5H), 2.12 (d, J=17.9 Hz, 10H), 1.19 (d, J=23.1 Hz, 3H).

### Compound of Invention—35 (Example-21): Step 1: Intermediate of Formula-46: tert-butyl 6-(5-(5-(trifluoromethyl)-1,2,4-oxadiazol-3-yl)pyridin-2-yl)-2,6-diazaspiro[3.3]heptane-2-carboxylate

To a flask containing compound (3, 1.50 g) and tert-butyl 2,6-diazaspiro[3.3]heptane-2-carboxylate oxalate (2.19 g) in NMP (37.6 mL) was added DIPEA (3.11 g, 4.19 mL) and heated to 90° C. under nitrogen atmosphere. After heating overnight, the reaction was cooled to room temperature. In a beaker with 80 mL of ice, the crude product was added dropwise and stirred vigorously. The precipitated product was then filtered and washed with DI water and allowed to air dry to afford the titled product tert-butyl 6-(5-(5-(trifluoromethyl)-1,2,4-oxadiazol-3-yl)pyridin-2-yl)-2,6-diazaspiro[3.3]heptane-2-carboxylate (46, 1.92 g, 77% yield) as a white solid. HPLC Retention time 5.71 min., ¹H NMR (400 MHZ, CDCl₃) δ 8.85 (dd, J=2.3, 0.8 Hz, 1H), 8.08 (dd, J=8.8, 2.3 Hz, 1H), 6.34 (dd, J=8.8, 0.8 Hz, 1H), 4.24 (s, 2H), 4.14 (s, 4H), 1.45 (s, 10H).

### Compound of Invention—36 (Example-36): Step 2: Intermediate of Formula-47:3-(6-(2,6-diazaspiro[3.3]heptan-2-yl)pyridin-3-yl)-5-(trifluoromethyl)-1,2,4-oxadiazole TFA Salt

To a flask containing tert-butyl 6-(5-(5-(trifluoromethyl)-1,2,4-oxadiazol-3-yl)pyridin-2-yl)-2,6-diazaspiro[3.3]heptane-2-carboxylate (46, 1.00 g) in DCM (25 mL) was added TFA (3.46 g, 2.33 mL) at room temperature under nitrogen atmosphere. After stirring overnight, the solvent and excess TFA was removed using rotovap. The resulting brown color gummy material was dissolved in DCM and the solvents were once again removed using rotovap and dried under high vacuum. Upon trituration with tert-butyl methyl ether (15 mL) afforded the titled product 3-(6-(2,6-diazaspiro[3.3]heptan-2-yl)pyridin-3-yl)-5-(trifluoromethyl)-1,2,4-oxadiazole (47, 2.27 g, >99%) as a colorless solid as a TFA salt. HPLC Retention time 3.81 min, ¹H NMR (400 MHZ, CDCl₃) δ 8.68 (d, J=2.1 Hz, 1H), 8.14 (dd, J=9.0, 2.2 Hz, 1H), 6.55-6.46 (m, 1H), 4.33 (d, J=1.9 Hz, 4H), 4.27 (s, 4H), 3.54 (d, J=4.1 Hz, 5H).

### Examples of Compound of Invention 37-123 were Synthesized by Following the Method-I to Method-V

Table-I
Compounds of invention (Iaaa and Iaaa₁) and (Ibaa and Ibaa₁)-Examples 1- 36

| Example | Compound | Compound Name | Characterization |
|---|---|---|---|
| 1 | 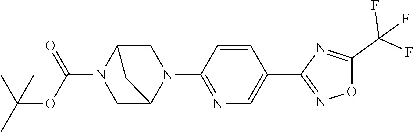 [SMILES: CC(C)(C)OC(=O)N1CC2CC1CN2c1ccc(-c2noc(C(F)(F)F)n2)cn1] | tert-butyl 5-(5-(5- (trifluoromethyl)- 1,2,4-oxadiazol-3- yl)pyridin-2-yl)-2,5- diazabicyclo[2.2.1] heptane-2- carboxylate | RT = 6.06 min (HPLC Method-I). MS-ESI (m/z): Calculated [M + H]⁺ = 412.15; Observed [M + H − 55]⁺ = 356.09. ¹H NMR (400 MHz, CDCl₃) δ 8.85 (d, J = 2.3 Hz, 1H), 8.07 (dd, J = 8.7, 2.3 Hz, 1H), 6.40 (d, J = 8.7 Hz, 1H), 5.00 (br., s, 1H), 4.645 (d, J = 64 |
| | | | Hz, 1H), 3.56 (d, J = 9.4 Hz, 1H), |
| | | | 3.51-3.33 (m, 3H), 1.98 (d, J = |
| | | | 19.8 Hz, 2H), 1.42 (s, 9H). |
| 2 | 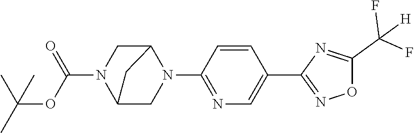 [SMILES: CC(C)(C)OC(=O)N1CC2CC1CN2c1ccc(-c2noc(C(F)F)n2)cn1] | tert-butyl 5-(5-(5- (difluoromethyl)- 1,2,4-oxadiazol-3- yl)pyridin-2-yl)-2,5- diazabicyclo[2.2.1] heptane-2- carboxylate | RT = 5.43 Min. (HPLC Method- I). ¹H NMR (400 MHz, CDCl₃) δ 8.84 (d, J = 2.3 Hz, 1H), 8.07 (dd, J = 8.8, 2.3 Hz, 1H), 6.84 (t, J = 52.3 Hz, 1H), 6.40 (d, J = 8.8 Hz, 1H), 4.64 (d, J = 56.0 Hz, 1H), 4.57 (s, 1H), 3.56 (d, J = 9.7 Hz, 1H), 3.51-3.32 (m, 3H), |
| | | | 1.97 (d, J = 19.3 Hz, 2H), 1.45 (s, |
| | | | 9H). |
| 3 | 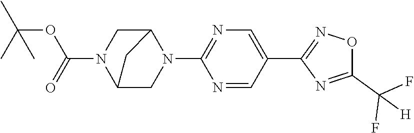 [SMILES: CC(C)(C)OC(=O)N1CC2CC1CN2c1ncc(-c2noc(C(F)F)n2)cn1] | tert-butyl 5-(5-(5- (difluoromethyl)- 1,2,4-oxadiazol-3- yl)pyrimidin-2-yl)- 2,5-diazabicyclo [2.2.1 ]heptane-2- carboxylate | RT = 4.52 Min. (HPLC Method- I). ¹H NMR (400 MHz, CDCl₃) δ 9.04 (s, 2H), 5.29 (d, J = 5.3 Hz, 1H), 4.65 (br., s, 1H), 4.19-3.89 (m, 1H), 3.80 (d, J = 20.5 Hz, 1H), 3.57 (br., s, 2H), 2.27 (s, 2H). |
| 4 | 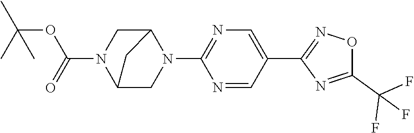 [SMILES: CC(C)(C)OC(=O)N1CC2CC1CN2c1ncc(-c2noc(C(F)(F)F)n2)cn1] | tert-butyl 5-(5-(5- (trifluoromethyl)- 1,2,4-oxadiazol-3- yl)pyrimidin-2-yl)- 2,5-diazabicyclo [2.2.1]heptane-2- carboxylate | RT = 7.25 Min. (HPLC Method- I). MS-ESI (m/z): Calculated [M + H]⁺ = 413.15; Observed [M + H − 56]⁺ = 357.09. ¹H NMR (400 MHz, CDCl₃) δ 8.92 (q, J = 3.2 Hz, 1H), 5.09 (s, 1H), 4.63 (d, J = 60 Hz, 1H), 3.69-3.59 (m, 2H), 3.45 (d, J = 8.5 Hz, 2H), |
| | | | 2.05-1.91 (m, 2H), 1.43 (d, J = |
| | | | 16.7 Hz, 9H). |
| 5 | 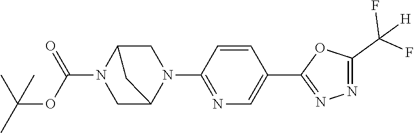 [SMILES: CC(C)(C)OC(=O)N1CC2CC1CN2c1ccc(-c2nnc(C(F)F)o2)cn1] | tert-butyl 5-(5-(5- (difluoromethyl)- 1,3,4-oxadiazol-2- yl)pyridin-2-yl)-2,5- diazabicyclo[2.2.1] heptane-2- carboxylate | RT = 5.31 Min. (HPLC Method- I). MS-ESI (m/z): Calculated [M + H]⁺ = 394.16; Observed [M + H]⁺ = 394.16. ¹H NMR (400 MHz, CDCl₃) δ 8.79 (d, J = 2.4 Hz, 1H), 8.04 (dd, J = 8.9, 2.4 Hz, 1H), 6.87 (t, J = 51.8 Hz, 1H), 6.38 (d, J = 9.0 Hz, 1H), |
| | | | 4.63 (d, J = 53.6 Hz, 1H), 3.54 (t, |
| | | | J = 9.4 Hz, 1H), 3.43 (d, J = 8.9 |
| | | | Hz, 2H), 3.38-3.31 (m, 1H), |
| | | | 2.34 (t, J = 8.1 Hz, 1H), 2.12- |
| | | | 1.86 (m, 2H), 1.44 (s, 9H). |
| 6 | 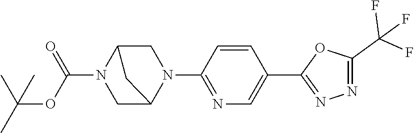 [SMILES: CC(C)(C)OC(=O)N1CC2CC1CN2c1ccc(-c2nnc(C(F)(F)F)o2)cn1] | tert-butyl 5-(5-(5- (trifluoromethyl)- 1,3,4-oxadiazol-2- yl)pyridin-2-yl)-2,5- diazabicyclo[2.2.1] heptane-2- carboxylate | RT = 6.00 Min. (HPLC Method- I). MS-ESI (m/z): Calculated [M + H]⁺ = 412.15; Observed [M + H]⁺ = 412.158. ¹H NMR (400 MHz, CDCl₃)) δ 8.76 (d, J = 2.4 Hz, 1H), 8.01 (dd, J = 8.9, 2.4 Hz, 1H), 6.37 (d, J = 8.9 Hz, 1H), 4.61 (d, J = 51.0 Hz, 1H), 3.52 (d, |
| | | | J = 9.5 Hz, 1H), 3.46-3.36 (m, |
| | | | 2H), 3.34 (dd, J = 8.7, 5.5 Hz, |
| | | | 1H), 2.97-2.56 (m, 1H), 2.07- |
| | | | 1.83 (m, 2H), 1.42 (s, 9H). |
| 7 | 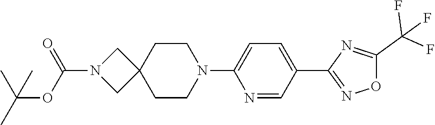 [SMILES: CC(C)(C)OC(=O)N1CC2(CCN(c3ccc(-c4noc(C(F)(F)F)n4)cn3)CC2)C1] | tert-butyl 7-(5-(5- (trifluoromethyl)- 1,2,4-oxadiazol-3- yl)pyridin-2-yl)-2,7- diazaspiro[3.5] nonane-2- carboxylate | RT = 6.68 Min. (HPLC Method- I). MS-ESI (m/z): Calculated [M + H]⁺ = 440.18; Observed [M + H]⁺ = 440.189. ¹H NMR (400 MHz, CDCl₃) δ 8.86 (d, J = 2.4 Hz, 1H), 8.07 (dd, J = 9.1, 2.4 Hz, 1H), 6.76-6.61 (m, 1H), 3.71 (s, 4H), 3.66 (t, J = 5.7 Hz, |
| | | | 4H), 1.92-1.73 (m, 4H), 1.45 (s, |
| | | | 9H). |
| 8 | 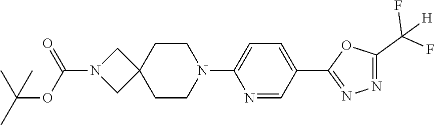 [SMILES: CC(C)(C)OC(=O)N1CC2(CCN(c3ccc(-c4nnc(C(F)F)o4)cn3)CC2)C1] | tert-butyl 7-(5-(5- (difluoromethyl)- 1,3,4-oxadiazol-2- yl)pyridin-2-yl)-2,7- diazaspiro[3.5] nonane-2- carboxylate | RT = 6.07 min, (HPLC Method- I). MS-ESI (m/z): Calculated [M + H]⁺ = 422.19; Observed [M + H]⁺ = 422.199. ¹H NMR (400 MHz, CDCl₃) δ 8.83 (d, J = 2.1 Hz, 1H), 8.07 (dt, J = 9.0, 2.1 Hz, 1H), 7.03-6.74 (m, 1H), 6.72 (d, J = 9.1 Hz, 1H), 3.72 (d, |
| | | | J = 1.6 Hz, 4H), 3.67 (dd, J = |
| | | | 8.1, 3.6 Hz, 4H), 1.84 (t, J = 5.6 |
| | | | Hz, 4H), 1.46 (s, 9H). |
| 9 | 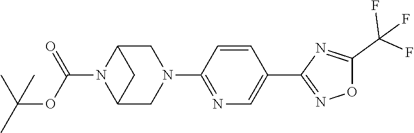 [SMILES: CC(C)(C)OC(=O)N1C2CC1CN(c1ccc(-c3noc(C(F)(F)F)n3)cn1)C2] | tert-butyl (1R,5S)-3- (5-(5- (trifluoromethyl)- 1,3,4-oxadiazol-2- yl)pyridin-2-yl)-3,6- diazabicyclo[3.1.1] heptane-6- carboxylate | RT = 6.45 Min. (HPLC Method- I). MS-ESI (m/z): Calculated [M + H]⁺ = 412.15; Observed [M + H]⁺ = 412.15 and 356.096. ¹H NMR (400 MHz, CDCl₃) 8.91 (d, J = 2.3 Hz, 1H), 8.12 (dd, J = 9.0, 2.4 Hz, 1H), 6.61 (d, J = 9.0 Hz, 1H), 4.31 (d, J = 6.2 |
| | | | Hz, 2H), 4.19 (s, 2H), 3.63-3.46 |
| | | | (m, 2H), 2.69 (h, J = 7.7, 7.1 Hz, |
| | | | 1H), 1.50 (d, J = 8.8 Hz, 1H), |
| | | | 1.35 (s, 9H). |
| 10 | 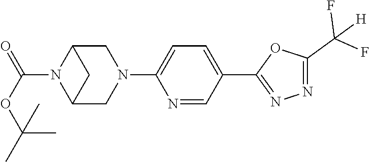 [SMILES: CC(C)(C)OC(=O)N1C2CC1CN(c1ccc(-c3nnc(C(F)F)o3)cn1)C2] | tert-butyl 3-(5-(5- (difluoromethyl)- 1,3,4-oxadiazol-2- yl)pyridin-2-yl)-3,6- diazabicyclo[3.1.1] heptane-6- carboxylate | RT = 5.76 min, (HPLC Method- I). MS-ESI (m/z): Calculated [M + H]⁺ = 394.16; Observed [M + H]⁺ = 394.16. ¹H NMR (400 MHz, CDCl₃) δ 8.90 (dd, J = 2.4, 0.7 Hz, 1H), 8.13 (dd, J = 9.0, 2.4 Hz, 1H), 6.89 (t, J = 51.8 Hz, 1H), 6.62 (dd, J = 9.0, 0.8 Hz, 1H), 4.31 (d, J = 6.2 Hz, 2H), 4.26-4.08 (m, 2H), 3.55 (s, 2H), |
| | | | 2.70 (q, J = 7.0 Hz, 1H), 1.51 (d, |
| | | | J = 8.8 Hz, 1H), 1.35 (s, 9H). |
| 11 | 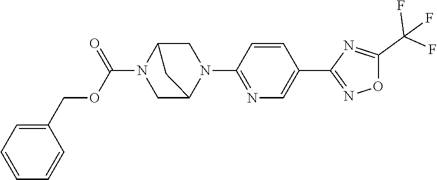 [SMILES: O=C(OCc1ccccc1)N1CC2CC1CN2c1ccc(-c2noc(C(F)(F)F)n2)cn1] | benzyl 5-(5-(5- (trifluoromethyl)- 1,2,4-oxadiazol-3- yl)pyridin-2-yl)-2,5- diazabicyclo[2.2.1] heptane-2- carboxylate | RT = 6.18 Min. (HPLC Method- I). MS-ESI (m/z): Calculated [M + H]⁺ = 446.14; Observed [M + H]⁺ = 446.14. ¹H NMR (400 MHz, CDCl₃) δ 8.84 (d, J = 2.5 Hz, 1H), 8.07 (dd, J = 8.9, 2.4 Hz, 1H), 7.37 (m, 3H), 7.32 (m, 2H), 6.39 (d, J = 8.9 Hz, 1H), 5.28-4.99 (m, 3H), 4.74 (d, J = 33.8 Hz, 1H), 3.70-3.36 (m, 4H), 2.00 (d, J = 8.2 Hz, 2H). |
| 12 | 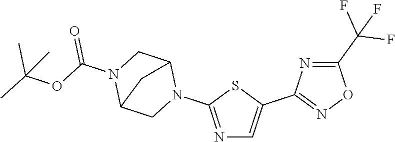 [SMILES: CC(C)(C)OC(=O)N1CC2CC1CN2c1ncc(-c2noc(C(F)(F)F)n2)s1] | tert-butyl 5-(5-(5- (trifluoromethyl)- 1,2,4-oxadiazol-3- yl)thiazol-2-yl)-2,5- diazabicyclo[2.2.1] heptane-2- carboxylate | RT = 7.04 Min. (HPLC Method- I). Q-TOF LC/MS (m/z): Calculated [M + H]⁺ = 417.1471; Observed [M + H]⁺ = 418.1825. ¹H NMR (400 MHz, CDCl₃) δ 7.96 (s, 1H), 4.72 (t, J = 10.6 Hz, 2H), 4.58 (s, 1H), 3.60 (q, J = 10.4, 9.3 Hz, 2H), 3.53-3.39 (m, 3H), 2.04 (d, J = 2.7 Hz, 2H), 2.01 (d, |
| | | | J = 4.5 Hz, 1H), 1.45 (d, J = 15.6 |
| | | | Hz, 11H). ¹⁹F NMR (376 MHz, |
| | | | CDCl₃) δ −65.46. |
| 13 | 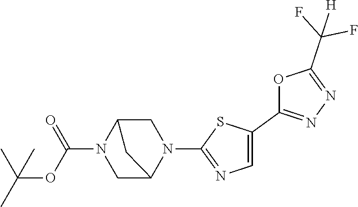 [SMILES: CC(C)(C)OC(=O)N1CC2CC1CN2c1ncc(-c2nnc(C(F)F)o2)s1] | tert-butyl 5-(5-(5- (difluoromethyl)- 1,3,4-oxadiazol-2- yl)thiazol-2-yl)-2,5- diazabicyclo[2.2.1] heptane-2- carboxylate | RT = 6.06 min (HPLC Method-I). Q-TOF LC/MS (m/z): Calculated [M + H]⁺ =399.1177; Observed [M + H]⁺ = 400.1268. ¹H NMR (400 MHz, CDCl₃) δ 7.92 (s, 1H), 6.85 (t, J = 51.8 Hz, 1H), 4.74 (s, 2H), 3.59 (d, J = 18.1 Hz, 2H), 3.47 (s, 3H), 2.04 (d, J = 10.6 Hz, 2H), 1.46 (d, J = 15.5 Hz, 10H. |
| 14 | 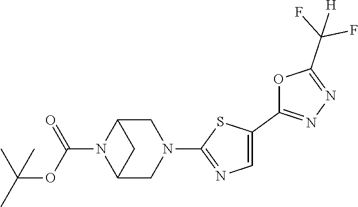 [SMILES: CC(C)(C)OC(=O)N1C2CC1CN(c1ncc(-c3nnc(C(F)F)o3)s1)C2] | tert-butyl 3-(5-(5- (difluoromethyl)- 1,3,4-oxadiazol-2- yl)thiazol-2-yl)-3,6- diazabicyclo[3.1.1] heptane-6- carboxylate | RT = 6.13 min (HPLC Method-I). Q-TOF LC/MS (m/z): Calculated [M + H]⁺ =399.1177; Observed [M + H]⁺ = 400.1269. ¹H NMR (400 MHz, CDCl₃) 8 7.99 (s, 1H), 6.86 (t, J = 51.7 Hz, 1H), 4.30 (d, J = 6.3 Hz, 3H), 4.18 (s, 2H), 2.75 (q, J = 7.2 Hz, 1H), 1.60- 1.50 (m, 2H), 1.38 (s, 9H). |
| 15 | 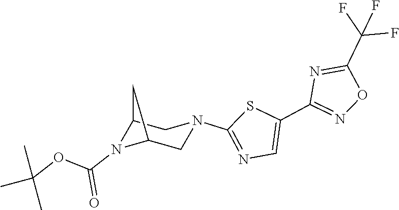 [SMILES: CC(C)(C)OC(=O)N1C2CC1CN(c1ncc(-c3noc(C(F)(F)F)n3)s1)C2] | tert-butyl (1R,5S)-3- (5-(5- (trifluoromethyl)- 1,2,4-oxadiazol-3- yl)thiazol-2-yl)-3,6- diazabicyclo[3.1.1] heptane-6- carboxylate | RT = 7.11 Min. (HPLC Method- I). Q-TOF LC/MS (m/z): Calculated [M + H]⁺ = 417.1082; Observed [M + H]⁺ = 418.1178. ¹H NMR (400 MHz, CDCl₃) δ 7.97 (s, 1H), 4.24 (d, J = 6.6 Hz, 2H), 4.10 (dt, J = 20.2, 9.0 Hz, 2H), 3.48 (td, J = 30.6, 27.8, 14.4 Hz, 2H), 2.69 (q, J = 7.3 Hz, 1H), 1.31 (d, J = 1.8 Hz, 9H), 1.26 (s, 1H). |
| 16 | 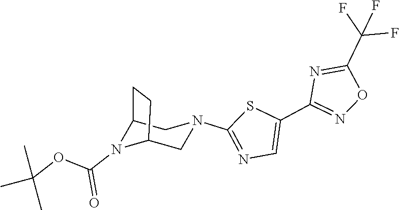 [SMILES: CC(C)(C)OC(=O)N1C2CCC1CN(c1ncc(-c3noc(C(F)(F)F)n3)s1)C2] | tert-butyl (1R,5S)-3- (5-(5- (trifluoromethyl)- 1,2,4-oxadiazol-3- yl)thiazol-2-yl)-3,8- diazabicyclo[3.2.1] octane-8- carboxylate | HPLC RT = 7.69 Min. (HPLC Method-I). Q-TOF LC/MS (m/z): Calculated [M + H]⁺ = 431.1239; Observed [M + H]⁺ = 432.1335. ¹H NMR (400 MHz, CDCl₃) δ 7.93 (s, 1H), 4.35 (s, 2H), 3.68 (s, 2H), 3.38 (s, 2H), 2.00 (dd, J = 8.9, 4.3 Hz, 2H), 1.77 (d, J = 7.5 Hz, 2H), 1.45 (d, J = 3.1 Hz, 9H), 1.30 (d, J = 6.0 Hz, 2H). |
| 17 | 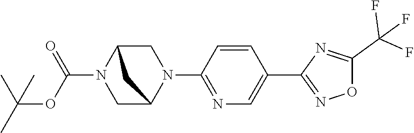 [SMILES: CC(C)(C)OC(=O)N1C[C@@H]2C[C@H]1CN2c1ccc(-c2noc(C(F)(F)F)n2)cn1] | tert-butyl (1S,4S)-5- (5-(5- (trifluoromethyl)- 1,2,4-oxadiazol-3- yl)pyridin-2-yl)-2,5- diazabicyclo[2.2.1] heptane-2- carboxylate | RT = 5.88 min (HPLC Method-I). Q-TOF LC/MS (m/z): Calculated [M + H]⁺ = 411.1518; Observed [M + H]⁺ = 412.1745 RT = 5.875 min, Purity = 96% ¹H NMR (400 MHz, CDCl³) δ 8.84 (dd, J = 2.4, 0.7 Hz, 1H), 8.07 (dd, J = 8.8, 2.3 Hz, 1H), 6.39 (d, J = 8.8 |
| | | | Hz, 1H), 5.04 (s, 1H), 4.71 (s, |
| | | | 1H), 4.57 (s, 1H), 3.57 (t, J = 9.7 |
| | | | Hz, 1H), 3.45 (d, J = 12.3 Hz, |
| | | | 3H), 3.41-3.30 (m, 4H), 2.84 (d, |
| | | | J = 1.0 Hz, 5H), 2.37 (t, J = 8.1 |
| | | | Hz, 4H), 2.11-1.89 (m, 6H), |
| | | | 1.72 (s, 2H). ¹⁹F NMR (376 MHz, |
| | | | CDCl₃) δ −65.41. |
| 18 | 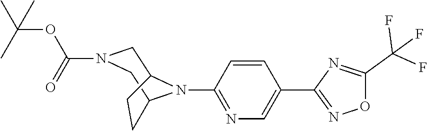 [SMILES: CC(C)(C)OC(=O)N1CC2CCC(C1)N2c1ccc(-c2noc(C(F)(F)F)n2)cn1] | tert-butyl (1R,5S)-8- (5-(5- (trifluoromethyl)- 1,2,4-oxadiazol-3- yl)pyridin-2-yl)-3,8- diazabicyclo[3.2.1] octane-3- carboxylate | RT = 4.59 Min. (HPLC Method- I). Q-TOF LC/MS (m/z): Calculated [M + H]⁺ = 425.1647; Observed [M + H]⁺ = 426.1817; ¹H NMR (400 MHz, CDCl₃) δ 8.87 (dd, J = 2.4, 0.7 Hz, 1H), 8.09 (dd, J = 8.9, 2.4 Hz, 1H), 6.66-6.60 (m, 1H), 6.59 (d, J = |
| | | | 9.0 Hz, 1H), 4.62 (s, 2H), 4.49 (s, |
| | | | 2H), 3.88 (t, J = 15.7 Hz, 2H), |
| | | | 3.80-3.65(m, 3H), 3.21 (d, J = |
| | | | 13.1 Hz, 2H), 3.10 (d, J = 12.8 |
| | | | Hz, 2H), 2.08-1.97 (m, 4H), |
| | | | 1.94-1.77 (m, 5H), 1.73 (s, 2H), |
| | | | 1.45(s, 9H), 1.43 (s, 4H). |
| 19 | 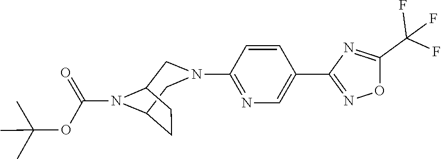 [SMILES: CC(C)(C)OC(=O)N1C2CCC1CN(c1ccc(-c3noc(C(F)(F)F)n3)cn1)C2] | tert-butyl (1R,5S)-3- (5-(5- (trifluoromethyl)- 1,2,4-oxadiazol-3- yl)pyridin-2-yl)-3,8- diazabicyclo[3.2.1] octane-8- carboxylate | RT = 7.07 Min. (HPLC Method- I). Q-TOF LC/MS (m/z): Calculated [M + H]⁺ = 425.41; Observed [M + H]⁺ = 426.1875. ¹H NMR (400 MHz, CDCl₃) δ 8.87 (d, J = 2.3 Hz, 1H), 8.09 (ddd, J = 9.1, 2.4, 0.8 Hz, 1H), 6.63 (d, J = 9.0 Hz, 1H), 4.39 (s, 2H), 4.08 (s, 2H), 3.19 (s, 2H), 1.98 (dd, J = 8.6, 4.3 Hz, 2H), |
| | | | 1.75 (d, J = 7.4 Hz, 2H), 1.49 (d, |
| | | | J = 0.7 Hz, 10H). |
| 20 | 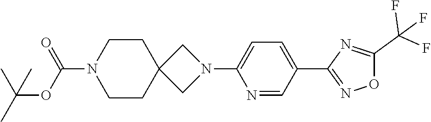 [SMILES: CC(C)(C)OC(=O)N1CCC2(CC1)CN(c1ccc(-c3noc(C(F)(F)F)n3)cn1)C2] | tert-butyl 6-(5-(5- (trifluoromethyl)- 1,2,4-oxadiazol-3- yl)pyridin-2-yl)-2,6- diazaspiro[3.3] heptane-2- carboxylate | RT = 5.81 Min. (HPLC Method- I). ¹H NMR (400 MHz, CDCl₃) δ 8.85 (dd, J = 2.3, 0.7 Hz, 1H), 8.06 (dd, J = 8.8, 2.3 Hz, 1H), 6.39-6.29 (m, 1H), 3.87 (s, 4H), 3.42 (t, J = 5.6 Hz, 5H), 1.86- 1.72 (m, 4H), 1.56 (s, 2H), 1.47 (s, 11H). |
| 21 | 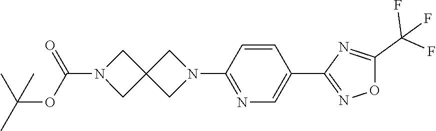 [SMILES: CC(C)(C)OC(=O)N1CC2(C1)CN(c1ccc(-c3noc(C(F)(F)F)n3)cn1)C2] | tert-butyl 2-(5-(5- (trifluoromethyl)- 1,2,4-oxadiazol-3- yl)pyridin-2-yl)-2,7- diazaspiro[3.5] nonane-7- carboxylate | RT = 5.71 min (HPLC Method-I). ¹H NMR (400 MHz, CDCl₃) δ 8.85 (dd, J = 2.3 Hz, 1H), 8.08 (dd, J = 8.8, 2.3 Hz, 1H), 6.34 (dd, J = 8.8 Hz, 1H), 4.24 (s, 2H), 4.14 (s, 4H), 1.45 (s, 10H). |
| 22 | 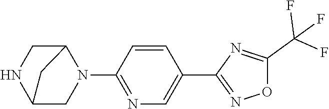 [SMILES: FC(F)(F)c1nc(-c2ccc(N3CC4CC3CN4)nc2)no1] | 3-(6-(2,5- diazabicyclo[2.2.1] heptan-2-yl) pyridin-3- yl)-5- (trifluoromethyl)- 1,2,4-oxadiazole | RT = 4. 57 Min. (HPLC Method- I). MS-ESI (m/z): Calculated [M + H]⁺ = 313.11; Observed [M + H]⁺ = 313.15. ¹H NMR (400 MHz, CDCl₃) δ 10.85 (s, 1H), 9.54 (s, 1H), 9.07 (s, 3H), 8.73 (d, J = 2.3 Hz, 1H), 8.02 (dd, J = |
| | | | 9.1, 2.3 Hz, 1H), 7.26 (s, 1H), |
| | | | 6.37 (d, J = 8.8 Hz, 1H), 5.01 (s, |
| | | | 1H), 4.42 (s, 1H), 3.60 (AB q, J = |
| | | | 52.0 and 12.0 Hz, 2H), 3.36 (s, |
| | | | 1H), 3.25 (s, 1H), 2.09 (AB q, J = |
| | | | 32.0 and 12.0 Hz, 2H). |
| 23 | 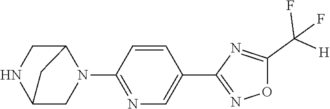 [SMILES: FC(F)c1nc(-c2ccc(N3CC4CC3CN4)nc2)no1] | 3-(6-(2,5- diazabicyclo[2.2.1] heptan-2-yl) pyridin-3-yl)-5- (difluoromethyl)- 1,2,4-oxadiazole | RT = 4.57 Min. (HPLC Method- I). MS-ESI (m/z): Calculated [M + H]⁺ = 294.11; Observed [M + H − 59]⁺ = 234.13 and 256.11. ¹H NMR (400 MHz, CDCl₃) δ 10.85 (s, 1H), 9.54 (s, 1H), 9.07 (s, 3H), 8.73 (d, J = 2.3 |
| | | | Hz, 1H), 8.02 (dd, J = 9.1, 2.3 |
| | | | Hz, 1H), 7.26 (s, 1H), 6.37 (d, J = |
| | | | 8.8 Hz, 1H), 5.01 (s, 1H), 4.42 (s, |
| | | | 1H), 3.66 (d, J = 10.9 Hz, 1H), |
| | | | 3.53 (d, J = 10.8 Hz, 1H), 3.36 (s, |
| | | | 1H), 3.25 (s, 1H), 2.18-1.98 (m, |
| | | | 2H), 1.13 (s, 5H). |
| 24 | 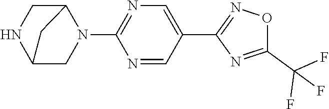 [SMILES: FC(F)(F)c1nc(-c2cnc(N3CC4CC3CN4)nc2)no1] | 3-(2-(2,5- diazabicyclo[2.2.1] heptan-2- yl)pyrimidin- 5-yl)-5- (trifluoromethyl)- 1,2,4-oxadiazole | Q-TOF LC/MS (m/z): Calculated [M + H]⁺ = 313.10; Observed [M + H]⁺ = 313.101. ¹H NMR (400 MHz, CDCl₃) δ 9.04 (s, 1H), 5.29 (d, J = 5.3 Hz, 1H), 4.19- 3.89 (m, 1H), 3.57 (s, 1H), 2.27 (s, 1H), 1.32 (dd, J = 10.7, 6.7 |
| | | | Hz, 2H). |
| 25 | 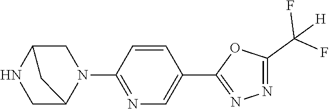 [SMILES: FC(F)c1nnc(-c2ccc(N3CC4CC3CN4)nc2)o1] | 2-(6-(2,5- diazabicyclo[2.2.1] heptan-2-yl) pyridin-3-yl)-5- (difluoromethyl)- 1,3,4-oxadiazole | RT = 0.85 Min. (HPLC Method- I). MS-ESI (m/z): Calculated [M + H]⁺ = 294.11; Observed [M + H]⁺ = 234.13. ¹H NMR (400 MHz, CD₃OD) δ 8.53-8.39 (m, 1H), 8.36-8.23 (m, 1H), 7.32 (s, 1H), 6.141 (t, J = 56.0 Hz, 1H), |
| | | | 5.23 (s, 1H), 4.65 (s, 1H), 4.06- |
| | | | 3.79 (m, 2H), 3.68-3.50 (m, |
| | | | 1H), 3.51-3.30 (m, 1H), 2.17 (d, |
| | | | J = 12.1 Hz, 1H), 2.04-1.76 (m, |
| | | | 1H). |
| 26 | 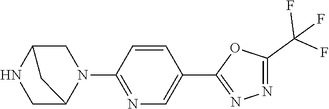 [SMILES: FC(F)(F)c1nnc(-c2ccc(N3CC4CC3CN4)nc2)o1] | 2-(6-(2,5- diazabicyclo[2.2.1] heptan-2-yl) pyridin-3-yl)-5- (trifluoromethyl)- 1,3,4-oxadiazole | RT = 0.89 Min. (HPLC Method- I). MS-ESI (m/z): Calculated [M + H]⁺ = 312.10; Observed [M + H]⁺ = 234.13. ¹H NMR (400 MHz, CD₃OD) δ 8.74-8.57 (m, 1H), 8.56-8.21 (m, 1H), 7.39 (s, 1H), 5.40 (d, J = 8.1 Hz, 1H), |
| | | | 4.85 (s, 1H), 4.08 (ddt, J = 9.1, |
| | | | 6.2, 3.1 Hz, 2H), 3.79-3.55 (m, |
| | | | 2H), 2.52 (d, J = 12.4 Hz, 1H), |
| | | | 2.37 (d, J = 9.7 Hz, 1H). |
| 27 | 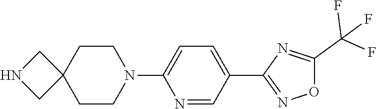 [SMILES: FC(F)(F)c1nc(-c2ccc(N3CCC4(CC3)CNC4)nc2)no1] | 3-(6-(2,7- diazaspiro[3.5]nonan- 7-yl)pyridin-3-yl)-5- (trifluoromethyl)- 1,2,4-oxadiazole | RT = 4.38 Min. (HPLC Method- I). MS-ESI (m/z): Calculated [M + H]⁺ = 340.13; Observed [M + H]⁺ = 340.137. ¹H NMR (400 MHz, CD₃OD) δ 8.71 (s, 1H), 8.28 (d, J = 9.4 Hz, 1H), 7.16 (d, J = 9.5 Hz, 1H), 3.93 (s, |
| | | | 4H), 3.71, s, 4H), 2.18-1.93 (m, |
| | | | 4H). |
| 28 | 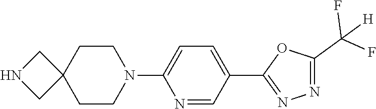 [SMILES: FC(F)c1nnc(-c2ccc(N3CCC4(CC3)CNC4)nc2)o1] | 2-(6-(2,7- diazaspiro[3.5]nonan- 7-yl)pyridin-3-yl)-5- (difluoromethyl)- 1,3,4-oxadiazole | RT = 3.03 min, (HPLC Method- I). MS-ESI (m/z): Calculated [M + H]⁺ = 322.14; Observed [M + H]⁺ = 322.146. ¹H NMR (400 MHz, CD₃OD) δ 9.43 (s, 1H), 8.71 (s, 1H), 8.15 (d, J = 9.2 Hz, 1H), 6.97 (dd, J = 30.6, 21.2 |
| | | | Hz, 2H), 3.90 (s, 4H), 3.79-3.36 |
| | | | (m, 4H), 2.49-1.75 (m, 4H). |
| 29 | 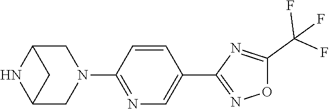 [SMILES: FC(F)(F)c1nc(-c2ccc(N3CC4CC(C3)N4)nc2)no1] | 2-(6-(3,6- diazabicyclo[3.1.1] heptan-3-yl) pyridin-3-yl)-5- trifluoromethyl)- 1,3,4-oxadiazole | RT = 4.65 Min. (HPLC Method- I). MS-ESI (m/z): Calculated [M + H]⁺ = 312.10; Observed [M + H]⁺ = 312.106. ¹H NMR (400 MHz, CDCl₃) δ 8.88 (d, J = 2.3 Hz, 1H), 8.20 (dd, J = 8.9, 2.3 Hz, 1H), 6.70 (d, J = 9.0 Hz, 1H), |
| | | | 4.53 (d, J = 6.1 Hz, 2H), 4.02 (d, |
| | | | J = 25.5 Hz, 4H), 3.17 (dd, J = |
| | | | 12.7, 6.4 Hz, 1H), 1.89 (d, J = |
| | | | 10.3 Hz, 1H). |
| 30 | 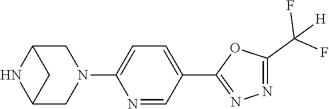 [SMILES: FC(F)c1nnc(-c2ccc(N3CC4CC(C3)N4)nc2)o1] | 2-(6-(3,6- diazabicyclo[3.1.1] heptan-3-yl) pyridin-3-yl)-5- difluoromethyl)- 1,3,4-oxadiazole | RT = 3.23 Min. (HPLC Method- I). MS-ESI (m/z): Calculated [M + H]⁺ = 294.11; Observed [M + H]⁺ = 294.116. ¹H NMR (400 MHz, CD₃OD) δ 8.83 (d, J = 2.3 Hz, 1H), 8.15 (dd, J = 9.1, 2.3 Hz, 1H), 7.05-6.75 (m, 1H), |
| | | | 6.90 (t, J = 52 Hz, 1H), 6.71 (d, |
| | | | J = 9.0 Hz, 1H), 4.52 (d, J = 6.4 |
| | | | Hz, 2H), 4.05 (s, 2H), 3.14 (dt, |
| | | | J = 10.5, 6.5 Hz, 1H), 1.88 (d, J = |
| | | | 10.5 Hz, 1H), 1.28-1.14 (m, |
| | | | 1H), 0.79 (dt, J = 12.3, 7.4 Hz, |
| | | | 1H). |
| 31 | 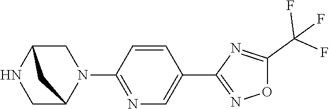 [SMILES: FC(F)(F)c1nc(-c2ccc(N3C[C@@H]4C[C@H]3CN4)nc2)no1] | 3-(6-((1S,4S)-2,5- diazabicyclo[2.2.1] heptan-2-yl) pyridin-3-yl)-5- (trifluoromethyl)- 1,2,4-oxadiazole | RT = 4.58 min, (HPLC Method- I). MS-ESI (m/z): Calculated [M + H]⁺ = 311.27; Observed [M + H]⁺ = 312.1236. ¹H NMR (400 MHz, CDCl₃) δ 8.69 (d, J = 2.2 Hz, 1H), 8.20 (dd, J = 9.1, 2.2 Hz, 1H), 6.72 (d, J = 9.1 Hz, 1H), |
| | | | 5.10 (s, 1H), 4.52 (s, 1H), 3.87 (s, |
| | | | 1H), 3.78 (d, J = 11.4 Hz, 1H), |
| | | | 3.69 (dd, J = 11.5, 2.2 Hz, 1H), |
| | | | 3.42 (s, 3H), 3.30 (s, 95H), 2.30 |
| | | | (t, J = 8.1 Hz, 4H), 2.17 (s, 2H), |
| | | | 1.96 (q, J = 7.7 Hz, 4H). |
| 32 | 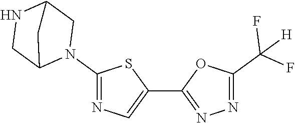 [SMILES: FC(F)c1nnc(-c2cnc(N3CC4CC3CN4)s2)o1] | 2-(2-(2,5- diazabicyclo[2.2.1] heptan-2-yl)thiazol- 5-yl)-5- (difluoromethyl)- 1,3,4-oxadiazole TFA Salt | RT = 3.63 Min. (HPLC Method- I). ¹H NMR (400 MHz, CDCl₃) δ 7.86 (s, 1H), 6.83 (t, J = 51.7 Hz, 1H), 4.79 (s, 1H), 4.51 (s, 1H), 3.92 (s, 2H), 3.70-3.55 (m, 3H), 3.31 (s, 2H), 2.32-1.93 (m, 2H). |
| 33 | 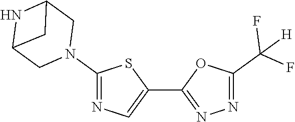 [SMILES: FC(F)c1nnc(-c2cnc(N3CC4CC(C3)N4)s2)o1] | 2-(2-(3,6- diazabicyclo[3.1.1] heptan-3-yl)thiazol- 5-yl)-5- (difluoromethyl)- 1,3,4-oxadiazole TFA Salt | RT = 3.61 Min. (HPLC Method- I). ¹H NMR (400 MHz, CDCl₃) δ 7.95 (s, 1H), 6.84 (t, J = 51.6 Hz, 1H), 4.50 (d, J = 6.6 Hz, 2H), 4.07 (d, J = 12.6 Hz, 2H), 3.95 (d, J = 12.7 Hz, 3H), 3.62 (s, 2H), 3.33 (s, 2H), 1.90 (d, J = 10.8 Hz, 1H). |
| 34 | 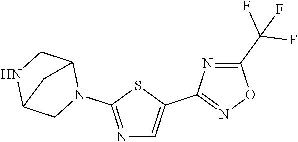 [SMILES: FC(F)(F)c1nc(-c2cnc(N3CC4CC3CN4)s2)no1] | 3-(2-(2,5- diazabicyclo[2.2.1] heptan-2-yl)thiazol- 5-yl)-5- (trifluoromethyl)- 1,2,4-oxadiazole TFA Salt | RT = 4.77Min. (HPLC Method- I). ¹H NMR (400 MHz, DMSO- d₆) δ 9.40 (s, 1H), 8.89 (s, 1H), 8.05 (s, 1H), 4.79 (s, 1H), 4.55 (s, 1H), 3.72 (dd, J = 10.9, 2.3 Hz, 1H), 3.61 (d, J = 10.9 Hz, 1H), 2.24 (dd, J = 10.9, 2.3 Hz, 1H), 1.98 (d, J = 11.2 Hz, 1H). |
| 35 | 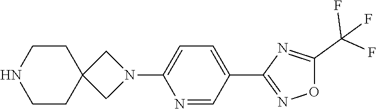 [SMILES: FC(F)(F)c1nc(-c2ccc(N3CC4(CCNCC4)C3)nc2)no1] | 3-(6-(2,7- diazaspiro[3.5]nonan- 2-yl)pyridin-3-yl)-5- (trifluoromethyl)- 1,2,4-oxadiazole | RT-3.97 Min. (HPLC Method- I). ¹H NMR (400 MHz, CDCl₃) δ 8.80 (s, 1H), 8.09 (d, J = 8.8 Hz, 1H), 6.36 (d, J = 8.8 Hz, 1H), 3.91 (s, 5H), 3.17 (d, J = 13.6 Hz, 5H), 2.12 (d, J = 17.9 Hz, 10H), 1.19 (d, J = 23.1 Hz, 3H). |
| 36 | 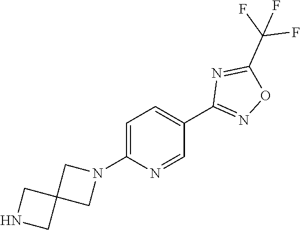 [SMILES: FC(F)(F)c1nc(-c2ccc(N3CC4(CNC4)C3)nc2)no1] | 3-(6-(2,6- diazaspiro[3.3 ] heptan-2-yl) pyridin-3-yl)-5- (trifluoromethyl)- 1,2,4-oxadiazole | RT = 3.81 Min. (HPLC Method- I). ¹H NMR (400 MHz, CDCl₃) δ 8.68 (d, J = 2.1 Hz, 1H), 8.14 (dd, J = 9.0, 2.2 Hz, 1H), 6.55- 6.46 (m, 1H), 4.33 (d, J = 1.9 Hz, 4H), 4.27 (s, 4H), 3.54 (d, J = 4.1 Hz, 5H). |

### Example of Compounds of Invention 37-123 were Synthesized by Following the Method-I to Method-VI

### General Method of Synthesis of Compounds of Invention of Formula Iaaa₂ and Ibaa₂

Method-I: To a stirred solution of the intermediate—5 TFA salt (0.10 mmol) in DCM (3 to 5 mL) was added DIPEA (0.30 mmol) followed by addition of an acid chloride (0.10 mmol) at room temperature After stirring 1 to 2 hours, the reaction was quenched with water and the product was extracted with DCM. Purification on a silica gel TLC using 30 to 70% EA/Hex afforded the compounds of invention (Iaaa₂) and (Ibaa₂).

Alternatively, the compounds of invention of formula Iaaa₂ and Ibaa₂ were synthesized using Method-II

Method-II: To a stirred solution of the intermediate—5 TFA salt (0.12 mmol), and carboxylic acid (0.12 mmol), EDC (0.24 mmol) and HOBt (0.24 mmol) in DMF (3 mL) was added at room temperature followed by addition of DIPEA (0.48 mmol). After stirring 2 to 24 hours, the reaction was quenched with water and extracted with EtOAc. The organics were combined and washed with water followed by 10% aqueous LiCl solution and brine. The organics were then dried over anhydrous Na₂SO₄, filtered, and the filtrate was concentrated under reduced pressure. Purification on a silica gel preparatory TLC glass plate using 30% to 70% EA/Hex afforded the compounds of invention (Iaaa₂) and (Ibaa₂).

Alternatively, the compounds of invention of formula Iaaa₂ and Ibaa₂ were synthesized using Method-III

Method-III: To a stirred solution of the intermediate—5 TFA salt (0.12 mmol), and carboxylic acid (0.12 mmol), HATU (121.52 mg, 1.5 equiv), and stirred at room temperature for 5 hours. The reactions flasks were then allowed to evaporate and diluted with DCM (˜2 mL) and methanol (˜0.5 mL). To the reaction flasks that are dry DCM (˜2 mL) and ethyl acetate (2-3 drops) was added. Purification on a silica gel preparatory TLC glass plate using 30% to 70% EA/Hex afforded the compounds of invention (Iaaa₂) and (Ibaa₂).

TABLE II
Compounds of invention (Iaaa₂) and (Ibaa₂) Examples 37-83

| Ex- | | | |
|---|---|---|---|
| am- | | | |
| ple | Compound | Compound Name | Characterization |
| 37 | 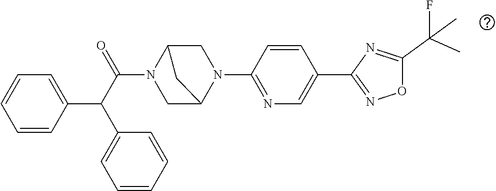 | 2,2-diphenyl-1-(5-(5- (5-(trifluoromethyl)- 1,2,4-oxadiazol-3- yl)pyridin-2-yl)-2,5- diazabicyclo[2.2.1] heptan-2-yl)ethan-1- one | RT = 6.55 Min. (HPLC Method- I). MS-ESI (m/z): Calculated [M + H]⁺= 506.17; Observed [M + H]⁺ = 506.178. ¹H NMR (400 MHz, CDCl₃) δ 8.76 (s, 1H), 8.05 (d, J = 8.9 Hz, 1H), 7.36- 7.20 (m, 8H), 7.16-7.07 (m, 2H), 6.32 (d, J = 8.9 Hz, 1H), 5.16 (s, 1H), 5.02 (br., s, 1H), 3.64-3.43 (m, 4H), 2.03-1.85 (m, 2H). |
| 38 | 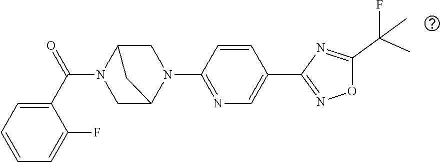 | (2-fluorophenyl)(5-(5- (5-(trifluoromethyl)- 1,2,4-oxadiazol-3- yl)pyridin-2-yl)-2,5- diazabicyclo[2.2.1] heptan-2-yl)methanone | RT = 5.60 Min. (HPLC Method- I). MS-ESI (m/z): Calculated [M + H]⁺ = 434.12; Observed [M + H]⁺ = 434.12. ¹H NMR (400 MHz, CDCl₃) δ 8.82 (d, J = 2.3 Hz, 1H), 8.13 (m, 1H), 7.46 (q, J = 8.7, 8.1 Hz, 1H), 7.38 (q, J = 7.1 Hz, 1H), 7.15 (dd, J = 10.8, 7.7 Hz, 1H), 7.05 (t, J = 9.1 Hz, 1H), 6.48 (t, J = 9.7 Hz, 1H), 5.24 |
| | | | (s, 1H), 5.16 (s, 1H), 3.94-3.48 |
| | | | (m, 4H), 2.17-1.96 (m, 2H). |
| 39 | 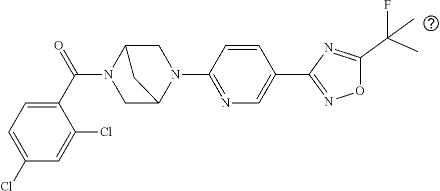 | (2,4-dichlorophenyl)(5- (5-(5-(trifluoromethyl)- 1,2,4-oxadiazol-3- yl)pyridin-2-yl)-2,5- diazabicyclo[2.2.1] heptan-2-yl)methanone | RT = 6.43 Min. (HPLC Method- I). MS-ESI (m/z): Calculated [M + H]⁺ = 484.05; Observed [M + H]⁺ = 484.05. ¹H NMR (400 MHz, CDCl₃) δ 8.81 (s, 1H), 8.10 (d, J = 8.9, Hz, 1H), 7.39-7.19 (m, 3H), 6.43 (d, J = 9.2 Hz, 1H), 5.15 (s, 1H), 5.10 (d, J = 10.9 Hz, 1H), 3.83-3.59 (m, 2H), 3.59- 3.46 (m, 1H), 3.26 (ABq, J = 9.7, 1.9 Hz, 1H), 2.19-1.94 (m, 2H). |
| 40 | 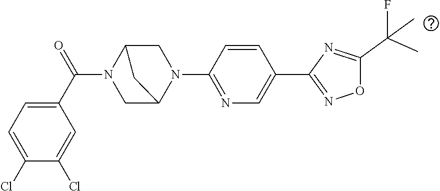 | (3,4-dichlorophenyl)(5- (5-(5-(trifluoromethyl)- 1,2,4-oxadiazol-3- yl)pyridin-2-yl)-2,5- diazabicyclo[2.2.1] heptan-2-yl)methanone | RT = 6.44 Min. (HPLC Method- I). MS-ESI (m/z): Calculated [M + H]⁺ = 484.05; Observed [M + H]⁺ = 484.05. ¹H NMR (400 MHz, CDCl₃) δ 8.80 (d, J = 2.3 Hz, 1H), 8.20-8.01 (m, 1H), 7.62 (s, 1H), 7.54 (d, J = 8.1 Hz, 1H), 7.45 (d, J = 8.3 Hz, 1H), 6.40 (d, J = 8.9 Hz, 1H), 5.15 (s, 1H), 5.10 (br., s, 1H), 3.80- 3.66 (m, 3H), 3.48 (d, J = 9.6 Hz, 1H), 2.07 (m, 2H). |
| 41 | 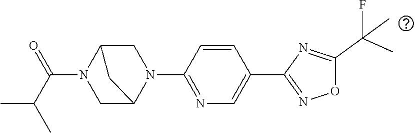 | 2-methyl-1-(5-(5-(5- (trifluoromethyl)-1,2,4- oxadiazol-3-yl)pyridin- 2-yl)-2,5-diaza- bicyclo[2.2.1]hept- an-2-yl)propan-1-one | RT = 5.10 Min. (HPLC Method- I). MS-ESI (m/z): Calculated [M + H]⁺ = 382.14 Observed [M + H]⁺ = 382.147 ¹H NMR (400 MHz, CDCl₃)) δ 8.84 (d, J = 2.4 Hz, 1H), 8.08 (dd, J = 8.8, 2.3 Hz, 1H), 6.39 (d, J = 9.0 Hz, 1H), 5.18 (s, 1H), 5.08 (s, 1H), 3.76- 3.47 (m, 3H), 3.40 (d, J = 9.4 Hz, |
| | | | 1H), 2.45 (p, J = 6.7 Hz, 1H), |
| | | | 2.02 (ABq, J = 10.3 Hz, 2H), |
| | | | 1.17 (dd, J = 13.5, 6.7 Hz, 2H), |
| | | | 1.10 (d, J = 6.7 Hz, 3H), 1.01 (d, |
| | | | J = 6.7 Hz, 3H). |
| 42 | 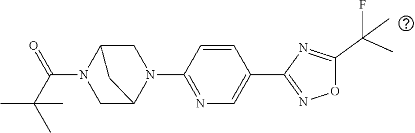 | 2,2-dimethyl-1-(5-(5- (5-(trifluoromethyl)- 1,2,4-oxadiazol-3- yl)pyridin-2-yl)-2,5- diazabicyclo[2.2.1] heptan-2-yl)propan-1- one | RT = 5.41 Min. (HPLC Method- I). MS-ESI (m/z): Calculated [M + H]⁺ = 396.16; Observed [M + H]⁺ = 396.16. ¹H NMR (400 MHz, CDCl₃) δ 8.84 (d, J = 2.3 Hz, 1H), 8.07 (dd, J = 8.9, 2.3 Hz, 1H), 6.40 (d, J = 8.9 Hz, 1H), 5.04 (s, 2H), 3.67 (s, 3H), 3.51- 3.19 (m, 1H), 2.00 (d, J = 29.7 |
| | | | Hz, 2H), 1.71 (s, 1H), 1.42-1.03 |
| | | | (m, 9H). |
| 43 | 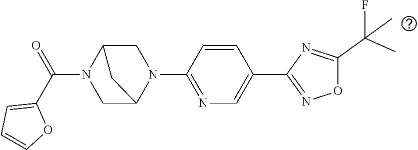 | furan-2-yl)(5-(5-(5- (trifluoromethyl)-1,2,4- oxadiazol-3-yl)pyridin- 2-yl)-2,5-diaza- bicyclo[2.2.1]hept- an-2-yl)methanone | RT = 5.32 min (HPLC Method-I). MS-ESI (m/z): Calculated [M + H]⁺ = 406.11; Observed [M + H]⁺ = 406.11. ¹H NMR (400 MHz, CDCl₃) δ 8.84 (dd, J = 8.3, 2.4 Hz, 1H), 8.07 (dt, J = 8.9, 2.6 Hz, 1H), 7.55-7.40 (m, 1H), 7.14 (dd, J = 25.0, 3.5 Hz, 1H), 6.49-6.28 (m, 2H), 5.43 (s, 1H), 5.23 (s, 1H), 4.06-3.91 (m, 1H), |
| | | | 3.86-3.41 (m, 3H), 2.16-1.84 |
| | | | (m, 2H). |
| 44 | 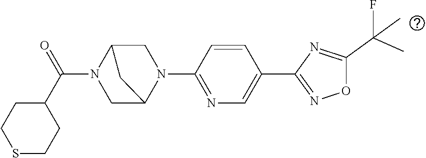 | (tetrahydro-2H- thiopyran-4-yl)(5-(5- (5-(trifluoromethyl)- 1,2,4-oxadiazol-3- yl)pyridin-2-yl)-2,5- diazABicyclo[2.2.1] heptan-2-yl)methanone | RT = 5.48 Min. (HPLC Method- I). MS-ESI (m/z): Calculated [M + H]⁺ = 440.13; Observed [M + H]⁺ = 440.135. ¹H NMR (400 MHz, CDCl₃) δ 8.84 (t, J = 2.1 Hz, 1H), 8.08 (dt, J= 8.9, 2.1 Hz, 1H), 6.41 (dd, J = 17.1, 8.8 Hz, 1H), 5.26 (br., s, 1H), 5.19 (s, 1H), 3.67-3.40 (m, 4H), 2.79- 2.49 (m, 4H), 2.27-1.62 (m, |
| | | | 7H). |
| 45 | 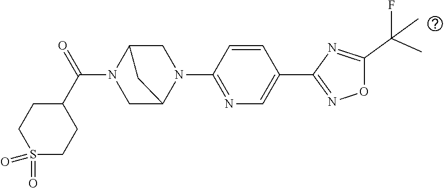 | (1,1-dioxidotetrahydro- 2H-thiopyran-4-yl)(5- (5-(5-(trifluoromethyl)- 1,2,4-oxadiazol-3- yl)pyridin-2-yl)-2,5- diazABicyclo[2.2.1] heptan-2-yl)methanone | HPLC: RT = 4.79 Min. (HPLC Method-I). MS-ESI (m/z): Calculated [M + H]⁺ = 472.12, Observed [M + H]⁺ = 472.12. ¹H NMR (400 MHz, CDCl₃)) δ 8.84 (dd, J = 5.5, 2.3 Hz, 1H), 8.10 (dd, J = 8.8, 2.3 Hz, 1H), 6.40 (d, J = 8.9 Hz, 1H), 5.21 (s, 1H), 5.10 (s, 1H), 3.68-3.28 (m, 7H), 3.13-2.78 (m, 2H), 2.49 (dq, J = 7.9, 4.0 Hz, 1H), 2.39-2.21 (m, 2H), 2.20-1.83 (m, 3H). |
| 46 | 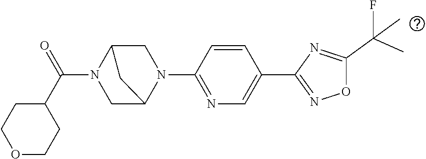 | (tetrahydro-2H-pyran- 4-yl)(5-(5-(5- (trifluoromethyl)-1,2,4- oxadiazol-3-yl)pyridin- 2-yl)-2,5- diazABicyclo[2.2.1] heptan-2-yl)methanone | RT = 4.94 Min. (HPLC Method- I). MS-ESI (m/z): Calculated [M + H]⁺ = 424.15; Observed [M + H]⁺ = 424.158. ¹H NMR (400 MHz, CDCl₃) δ 8.84 (ddd, J = 3.1, 2.3, Hz, 1H), 8.09 (dd, J = 8.8, 2.4 Hz, 1H), 6.56-6.18 (m, 1H), 5.26 (br., s, 1H), 5.19 (s, 1H), 4.16-3.92 (m, 2H), 3.69 (ddd, J = 29.6, 9.4, 1.9 Hz, 1H), |
| | | | 3.61-3.52 (m, 2H), 3.46 (tdd, J = |
| | | | 11.7, 4.0, 2.3 Hz, 1H), 3.36 |
| | | | (dtd, J = 15.8, 1.19, 2.4 Hz, 2H), |
| | | | 2.41 (tt, J = 11.4, 3.8 Hz, 1H), |
| | | | 2.15-1.76 (m, 4H), 1.74-1.53 |
| | | | (m, 1H), 1.45 (dd, J = 13.5, 4.0 |
| | | | Hz, 1H). |
| 47 | 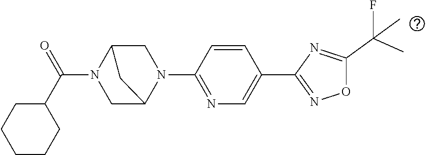 | cyclohexyl(5-(5-(5- (trifluoromethyl)-1,2,4- oxadiazol-3-yl)pyridin- 2-yl)-2,5- diazABicyclo[2.2.1] heptan-2-yl)methanone | RT = 5.87 Min. (HPLC Method- I). MS-ESI (m/z): Calculated [M + H]⁺ = 422.17; Observed [M + H]⁺ = 422.179. 1H NMR (400 MHz, CDCl3) δ 8.83 (dd, J = 2.4, 1.1 Hz, 1H), 8.07 (ddt, J = 8.8, 2.6, 1.3 Hz, 1H), 6.40 (dd, J = 15.7, 8.8 Hz, 1H), 5.24 (br., s, 1H), 5.17 (s, 1H), 3.74-3.60 (m, 1H), 3.60-3.51 (m, 2H), 3.40 (t, |
| | | | J = 17.8 Hz, 1H), 2.19-2.05 (m, |
| | | | 1H), 2.06-1.85 (m, 2H), 1.85- |
| | | | 1.61 (m, 5H), 1.60-1.39 (m, |
| | | | 2H), 1.31-1.13 (m, 3H). |
| 48 | 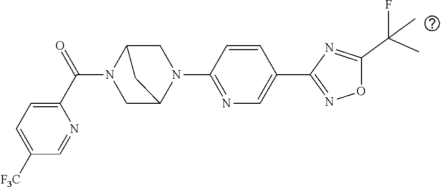 | (5-(5-(5- (trifluoromethyl)-1,2,4- oxadiazol-3-yl)pyridin- 2-yl)-2,5- diazABicyclo[2.2.1] heptan-2-yl)(5- (trifluoromethyl)pyri- din-2-yl)methanone | RT = 6.07 Min. (HPLC Method- I). MS-ESI (m/z): Calculated [M + H]⁺ = 485.11; Observed [M + H]⁺ = 485.115. ¹H NMR (400 MHz, CDCl₃) δ 8.92-8.85 (m, 1H), 8.83-8.75 (m, 1H), 8.17-7.95 (m, 3H), 6.43 (dd, J = 25.0, 8.9 Hz, 1H), 5.20 (s, 1H), 5.13 (d, J = 14.3 Hz, 1H), 4.06 (ABq, J = 96.0, 11.1, Hz, 1H), 3.86-3.66 (m, 3H), 2.26-2.07 (m, 2H). |
| 49 | 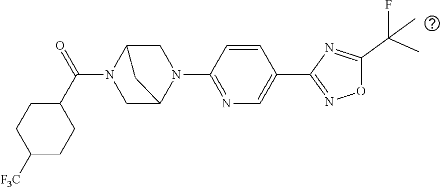 | (5-(5-(5- (trifluoromethyl)-1,2,4- oxadiazol-3-yl)pyridin- 2-yl)-2,5- diazABicyclo[2.2.1] heptan-2-yl)(4- (trifluoromethyl)cyclo- hexyl)methanone | RT = 6.20 Min. (HPLC Method- I). MS-ESI (m/z): Calculated [M + H]⁺ = 490.16; Observed [M + H]⁺ = 490.166. ¹H NMR (400 MHz, CDCl₃) δ 8.84 (d, J = 2.1 Hz, 1H), 8.09 (ddd, J = 8.8, 2.4, 1.0 Hz, 1H), 6.41 (dd, J = 16.9, 8.9 Hz, 1H), 5.19 (br., s, 1H), 5.08 (s, 1H), 3.68 (ddd, J = 35.1, 9.4, 1.9 Hz, 1H), 3.61-3.47 (m, 2H), 3.42 (dd, J = 31.9, 9.4 Hz, 1H), 2.25-1.79 (m, 6H), |
| | | | 1.69 (td, J = 13.1, 12.2, 3.3 Hz, |
| | | | 1H), 1.56 (dtd, J = 29.0, 12.8, |
| | | | 12.3, 3.4 Hz, 2H), .42-1.03 (m, |
| | | | 3H). |
| 50 | 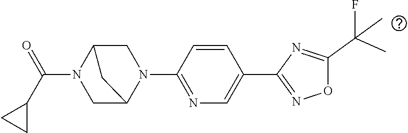 | cyclopropyl(5-(5-(5- (trifluoromethyl)-1,2,4- oxadiazol-3-yl)pyridin- 2-yl)-2,5- diazABicyclo[2.2.1] heptan-2-yl)methanone | RT = 5.07 Min. (HPLC Method- I). MS-ESI (m/z): Calculated [M + H]⁺ = 380.13; Observed [M + H]⁺ = 380.13. ¹H NMR (400 MHz, CDCl₃) δ 8.84 (dd, J = 2.4, 0.8 Hz, 1H), 8.08 (ddd, J = 8.9, 3.4, 2.3 Hz, 1H), 6.40 (d, J = 8.9 Hz, 1H), 5.20 (br., s, 1H), 5.06 (s, 1H), 3.84-3.68 (m, 2H), |
| | | | 3.67-3.51 (m, 2H), 2.07-1.92 |
| | | | (m, 2H), 1.45 (tt, J = 7.9, 4.6 Hz, |
| | | | 1H), 1.00-0.89 (m, 4H). |
| 51 | 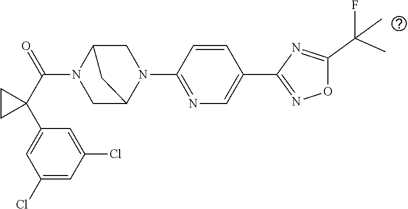 | (1-(3,5-dichloro- phenyl)cyclopro- pyl)(5-(5-(5- (trifluoromethyl)-1,2,4- oxadiazol-3-yl)pyridin- 2-yl)-2,5- diazABicyclo[2.2.1] heptan-2-yl)methanone | RT = 6.71 min (HPLC Method-I). MS-ESI (m/z): Calculated [M + H]⁺ = 524.08; Observed [M + H]⁺ = 524.086. ¹H NMR (400 MHz, CDCl₃) δ 8.79 (d, J = 2.3 Hz, 1H), 8.06 (dd, J = 8.8, 2.4 Hz, 1H), 7.30 (s, 1H), 7.23-7.10 (m, 2H), 6.33 (d, J = 8.9 Hz, 1H), 4.94 (d, J = 30.0 Hz, 2H), 3.55- 3.31 (m, 2H), 3.07 (d, J = 9.5 Hz, 1H), 2.84-2.59 (m, 1H), 1.99- 1.61 (m, 3H), 1.41-1.10 (m, 2H), 0.80 (s, 1H). |
| 52 | 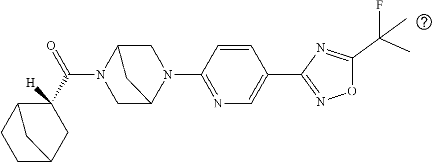 | ((2R)- bicyclo[2.2.1]heptan-2- yl)(5-(5-(5- (trifluoromethyl)-1,2,4- oxadiazol-3-yl)pyridin- 2-yl)-2,5- diazABicyclo[2.2.1] heptan-2-yl)methanone | RT = 6.04 Min. (HPLC Method- I). MS-ESI (m/z): Calculated [M + H]⁺ = 434.17; Observed [M + H]⁺ = 434.179. ¹H NMR (400 MHz, CDCl₃) δ 8.84 (dt, J = 8.0, 2.4 Hz, 1H), 8.07 (ddd, J = 8.6, 5.9, 2.3 Hz, 1H), 6.51-6.28 (m, 1H), 5.14 (s, 1H), 3.79-3.63 (m, 1H), 3.63-3.48 (m, 2H), 3.49-3.28 (m, 1H), 2.71-2.53 |
| | | | (m, 1H), 2.50-2.34 (m, 1H), |
| | | | 2.25 (dt, J = 19.8, 4.5 Hz, 2H), |
| | | | 2.16-2.00 (m, 1H), 2.01-1.85 |
| | | | (m, 2H), 1.86-1.65 (m, 1H), |
| | | | 1.64-0.96 (m, 6H). |
| 53 | 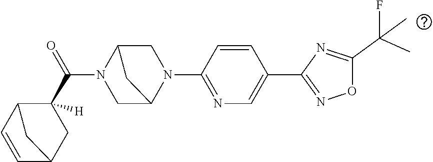 | ((2S)- bicyclo[2.2.1]hept-5- en-2-yl)(5-(5-(5- (trifluoromethyl)-1,2,4- oxadiazol-3-yl)pyridin- 2-yl)-2,5- diazABicyclo[2.2.1] heptan-2-yl)methanone | RT = 5.95 Min. (HPLC Method- I). MS-ESI (m/z): Calculated [M + H]⁺ = 432.16; Observed [M + H]⁺ = 432.16. ¹H NMR (400 MHz, CDCl₃) δ 8.84 (dt, J = 5.4, 2.2 Hz, 1H), 8.08 (ddd, J = 8.2, 5.5, 2.4 Hz, 1H), 6.41 (q, J = 9.7, 8.2 Hz, 1H), 6.23-5.95 (m, 2H), 5.17 (s, 1H), 3.72-3.50 (m, 4H), 3.42 (t, J = 8.7 Hz, 1H), |
| | | | 3.01-2.83 (m, 2H), 2.14-1.91 |
| | | | (m, 3H), 1.78-1.59 (m, 2H), |
| | | | 1.45-0.93 (m, 2H). |
| 54 | 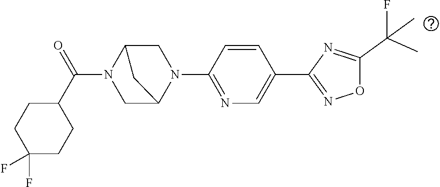 | (4,4- difluorocyclohexyl)(5- (5-(5-(trifluoromethyl)- 1,2,4-oxadiazol-3- yl)pyridin-2-yl)-2,5- diazABicyclo[2.2.1] heptan-2- yl)methanonetrifluoro- methyl)-1,2,4- oxadiazol-3-yl)pyridin- 2-yl)-2,5- diazABicyclo[2.2.1] | RT = 5.74 Min. (HPLC Method- I). MS-ESI (m/z): Calculated [M + H]⁺ = 396.16; Observed [M + H]⁺ = 396.165. ¹H NMR (400 MHz, CDCl₃) δ 8.85 (t, J = 2.7 Hz, 1H), 8.09 (dd, J = 8.8, 2.4 Hz, 1H), 6.42 (dd, J = 17.2, 8.9 Hz, 1H), 5.20 (s, 1H), 5.10 (s, 1H), 3.82-3.62 (m, 1H), 3.61- 3.52 (m, 2H), 3.39 (d, J = 9.4 Hz, 1H), 2.36-2.10 (m, 2H), 2.08- 1.62 (m, 9H). |
| | | heptan-2-yl)methanone | |
| 55 | 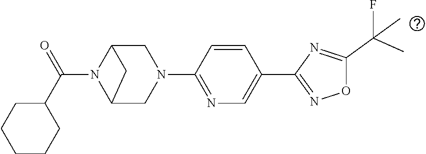 [SMILES: CC(C)(F)c1nc(-c2ccc(N3CC4CC(C3)N4C(=O)C3CCCCC3)nc2)no1] | cyclohexyl(3-(5-(5- (trifluoromethyl)-1,2,4- oxadiazol-3-yl)pyridin- 2-yl)-3,6-diaza- bicyclo[3.1.1]hept- an-6-yl)methanone | RT = 6.04 Min. (HPLC Method- I). ¹⁹F NMR (376 MHz, CDCl₃) δ −65.40. ¹H NMR (400 MHz, CDCl₃) δ 8.90 (dd, J = 2.4, 0.7 Hz, 1H), 8.13 (dd, J = 9.0, 2.4 Hz, 1H), 6.60 (d, J = 9.0 Hz, 1H), 4.59 (dd, J = 7.9, 4.2 Hz, 2H), 4.12 (d, J = 11.2 Hz, 1H), 3.91 (d, J = 10.8 Hz, 2H), 3.59 (s, 1H), 2.82-2.62 (m, 1H), 2.13 (tt, J = |
| | | | 11.5, 3.6 Hz, 1H), 1.78 (t, J = |
| | | | 11.3 Hz, 4H), 1.66 (d, J = 8.8 Hz, |
| | | | 5H), 1.56-1.31 (m, 4H), 1.24 (q, |
| | | | J = 9.8, 8.8 Hz, 4H). |
| 56 | 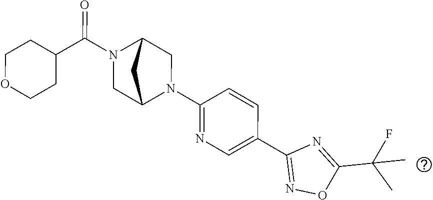 | (tetrahydro-2H-pyran- 4-yl)((1S,4S)-5-(5-(5- (trifluoromethyl)-1,2,4- oxadiazol-3-yl)pyridin- 2-yl)-2,5-diaza- bicyclo[2.2.1]hept- an-2-yl)methanone | RT = 4.83 Min. (HPLC Method- I). Q-TOF LC/MS (m/z): Calculated [M + H]+ = 423.1518; Observed [M + H]+ = 424.1631. ¹H NMR (400 MHz, CDCl₃) δ 8.83 (t, J = 2.8 Hz, 1H), 8.08 (dd, J = 8.8, 2.3 Hz, 1H), 6.41 (dd, J = 16.1, 8.9 Hz, 1H), 5.13 (d, J = 35.9 Hz, 1H), 4.23-3.82 (m, 6H), 3.78-3.65 (m, 1H), 3.60- 3.43 (m, 2H), 3.50-3.26 (m, H), 2.83 (s, 2H), 2.53 (s, 1H), 2.40 (dd, J = 10.8, 5.6 Hz, 3H), 2.07- |
| | | | 1.82 (m, 9H), 1.85 (s, 9H), 1.46- |
| | | | 1.32 (m, 2H). |
| 57 | 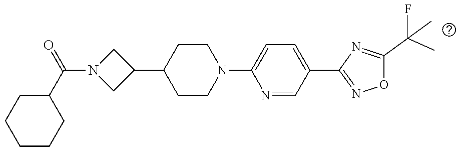 [SMILES: CC(C)(F)c1nc(-c2ccc(N3CCC(C4CN(C(=O)C5CCCCC5)C4)CC3)nc2)no1] | cyclohexyl(7-(5-(5- (trifluoromethyl)-1,2,4- oxadiazol-3-yl)pyridin- 2-yl)-2,7- diazaspiro[3.5]nonan- 2-yl)methanone | RT = 6.29 Min. (HPLC Method- I).; ¹H NMR (400 MHz, CDCl₃) δ 8.08 (dd, J = 9.0, 2.4 Hz, 1H), 6.73 (dd, J = 9.1 Hz, 1H), 3.90 (s, 1H), 3.77 (s, 1H), 3.72 (dt, J = 6.5, 3.6 Hz, 1H), 3.62 (td, J = 6.7, 6.2, 4.2 Hz, 1H), 2.17 (tt, J = 11.8, 3.5 Hz, 1H), 1.85 (dt, J = 7.3, 5.1 Hz, 2H), 1.79 (dd, J = 6.3, 3.6 Hz, 1H), 1.70 (d, J = 14.8 |
| | | | Hz, 2H), 1.57-1.42 (m, 1H), |
| | | | 1.32-1.19 (m, 2H). |
| 58 | 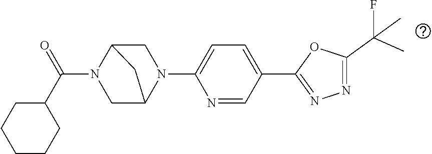 [SMILES: CC(C)(F)c1nnc(-c2ccc(N3CC4CC3CN4C(=O)C3CCCCC3)nc2)o1] | cyclohexyl(5-(5-(5- (difluoromethyl)-1,3,4- oxadiazol-2-yl)pyridin- 2-yl)-2,5-diaza- bicyclo[2.2.1]hept- an-2-yl)methanone | RT = 6.54 Min. (HPLC Method- I).; ¹H NMR (400 MHz, CDCl₃) δ 8.68 (d, J = 2.4 Hz, 1H), 8.58- 8.43 (m, 1H), 8.10 (s, 1H), 7.94 (dd, J = 8.7, 2.7 Hz, 1H), 7.86- 7.60 (m, 1H), 6.32 (d, J = 9.0 Hz, 1H), 6.23 (d, J = 9.0 Hz, 1H), 4.65 (s, 1H), 3.54-3.46 (m, 2H), 3.35 (d, J = 0.8 Hz, 2H), 3.11 (t, J = 11.4 Hz, 3H), 1.93 (d, J = 11.6 |
| | | | Hz, 2H), 1.86-1.56 (m, 3 H), |
| | | | 1.46 (t, J = 11.2 Hz, 12H), 1.37- |
| | | | 1.05 (m, 2H). |
| 59 | 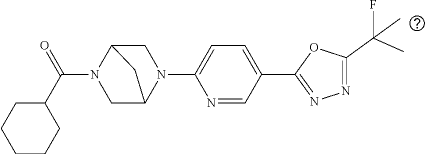 [SMILES: CC(C)(F)c1nnc(-c2ccc(N3CC4CC3CN4C(=O)C3CCCCC3)nc2)o1] | cyclohexyl(5-(5-(5- (trifluoromethyl)-1,3,4- oxadiazol-2-yl)pyridin- 2-yl)-2,5-diaza- bicyclo[2.2.1]hept- an-2-yl)methanone | RT = 6.51 Min. (HPLC Method- I). ¹H NMR (400 MHz, CDCl₃) δ 8.67 (d, J = 2.4 Hz, 1H), 8.17 (s, 1H), 7.97-7.82 (m, 1H), 6.31 (d, J = 9.0 Hz, 1H), 5.11 (s, 1H), 5.05 (s, 1H), 3.56-3.40 (m, 3H), 3.35 (d, J = 5.7 Hz, 1H), 3.11 (t, J = 11.4 Hz, 2H), 1.92 (d, J = 11.5 Hz, 6H), 1.78 (d, J = 12.1 Hz, 2H), 1.66 (d, J = 18.0 Hz, 2H), |
| | | | 1.52-1.37 (m, 2H), 1.34-1.07 |
| | | | (m, 1H). |
| 60 | 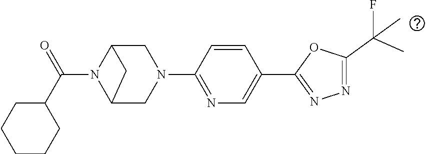 [SMILES: CC(C)(F)c1nnc(-c2ccc(N3CC4CC(C3)N4C(=O)C3CCCCC3)nc2)o1] | cyclohexyl(3-(5-(5- (difluoromethyl)-1,3,4- oxadiazol-2-yl)pyridin- 2-yl)-3,6-diaza- bicyclo[3.1.1]hept- an-6-yl)methanone | RT = 6.36 Min. (HPLC Method- I). ¹H NMR (400 MHz, CDCl₃) δ 8.55 (d, J = 2.4 Hz, 1H), 7.83- 7.71 (m, 2H), 6.44 (d, J = 9.0 Hz, 1H), 4.57 (d, J = 7.0 Hz, 3H), 4.08 (d, J = 11.2 Hz, 1H), 3.87 (s, 2H), 3.12 (t, J = 11.4 Hz, 1H), 3.04-2.91 (m, 1H), 2.75 (q, J = 7.0 Hz, 1H), 2.23 (tt, J = 11.6, 3.5 Hz, 1H), 2.12 (ddt, J = 11.7, 8.0, |
| | | | 3.6 Hz, 1H), 1.96 (d, J = 12.6 Hz, |
| | | | 3H), 1.80 (d, J = 21.7 Hz, 11H), |
| | | | 1.64 (dd, J = 16.1, 8.4 Hz, 2H), |
| | | | 1.47 (q, J = 11.7, 11.3 Hz, 2H), |
| | | | 1.23 (d, J = 10.5 Hz, 2H). |
| 61 | 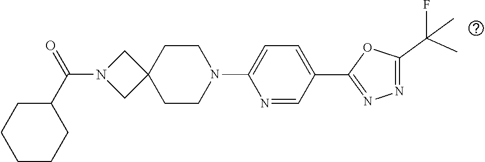 [SMILES: CC(C)(F)c1nnc(-c2ccc(N3CCC4(CC3)CN(C(=O)C3CCCCC3)C4)nc2)o1] | cyclohexyl(7-(5-(5- (difluoromethyl)-1,3,4- oxadiazol-2-yl)pyridin- 2-yl)-2,7- diazaspiro[3.5]nonan- 2-yl)methanone | RT = 6.41 Min. (HPLC Method- I). ¹H NMR (400 MHz, CDCl₃) δ 8.56-8.43 (m, 1H), 7.89 (s, 1H), 7.77 (ddd, J = 9.1, 4.9, 2.5 Hz, 1H), 6.57 (t, J = 8.7 Hz, 1H), 3.88 (d, J = 4.4 Hz, 3H), 3.80- 3.64 (m, 5H), 3.62-3.51 (m, 2H), 3.48 (s, 3H), 3.03-2.85 (m, 1H), 2.32-2.11 (m, 2H), 1.95 (d, J = 12.1 Hz, 3H), 1.87- |
| | | | 1.72 (m, 4H), 1.69 (d, J = 14.3 |
| | | | Hz, 4H), 1.47 (d, J = 11.7 Hz, |
| | | | 2H), 1.30-1.07 (m, 2H). |
| 62 | 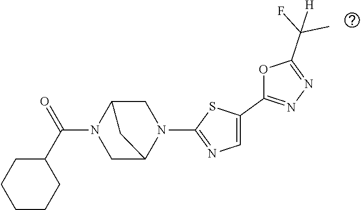 | cyclohexyl(5-(5-(5- (difluoromethyl)-1,3,4- oxadiazol-2-yl)thiazol- 2-yl)-2,5-diaza- bicyclo[2.2.1]hept- an-2-yl)methanone | RT = 5.81 Min. (HPLC Method- I).; MS-ESI (m/z): Calculated [M + H]⁺ = 409.14; Observed [M + H]⁺ = 410.1516. ¹H NMR (400 MHz, CDCl₃) δ 7.90 (d, J = 6.7 Hz, 1H), 7.78 (dd, J = 30.2, 8.0 Hz, 1H), 7.46-7.34 (m, 1H), 6.85 (t, J = 51.7 Hz, 1H), 5.08 (s, 1H), 4.89 (s, 1H), 4.72 (d, J = 14.9 Hz, 1H), 3.65-3.60 (m, 2H), 3.38 (d, J = 9.4 Hz, 1H), 2.26-2.03 (m, 2H), 1.98 (d, J = 10.3 Hz, 1H), 1.86-1.61 (m, |
| | | | 4H), 1.61-1.36 (m, 3H), 1.22 |
| | | | (dd, J = 22.3, 6.2 Hz, 4H). |
| 63 | 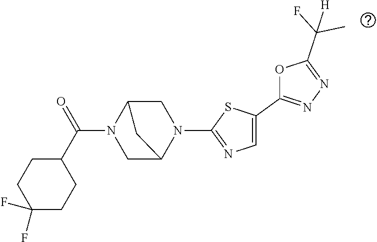 | (4,4- difluorocyclohexyl)(5- (5-(5-(difluoromethyl)- 1,3,4-oxadiazol-2- yl)thiazol-2-yl)-2,5- diazabicyclo[2.2.1] heptan-2-yl)methanone | RT = 5.62 Min. (HPLC Method- I). MS-ESI (m/z): Calculated [M + H]⁺ = 445.44; Observed [M + H]⁺ = 446.1321. ¹H NMR (400 MHz, CDCl₃) δ 7.90 (d, J = 4.6 Hz, 1H), 7.50-7.31 (m, 1H), 6.85 (t, J = 51.7 Hz, 1H), 5.09 (s, 1H), 4.92 (s, 1H), 4.72 (d, J = 27.2 Hz, 1H), 3.71-3.53 (m, 3H), 3.50-3.31 (m, 1H), 2.29- 2.04 (m, 4H), 1.95-1.56 (m, 6H). |
| 64 | 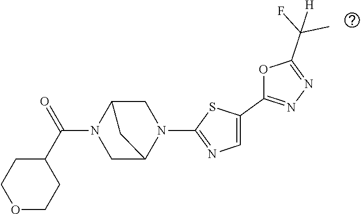 | (5-(5-(5- (difluoromethyl)-1,3,4- oxadiazol-2-yl)thiazol- 2-yl)-2,5-diaza- bicyclo[2.2.1]hept- an-2-yl)(tetrahydro-2H- pyran-4-yl)methanone | RT = 4.94 Min. (HPLC Method- I). MS-ESI (m/z): Calculated [M + H]⁺ = 413.11; Observed [M + H]⁺ = 412.1275. ¹H NMR (400 MHz, CDCl₃) δ 7.91 (d, J = 4.7 Hz, 1H), 6.86 (t, J = 51.8 Hz, 1H), 5.11 (s, 1H), 4.93 (s, 1H), 4.12-3.96 (m, 4H), 3.72-3.60 (m, 2H), 3.51-3.25 (m, 6H), 2.65-2.56 (m, 1H), 1.92-1.68 (m, 6H). |
| 65 | 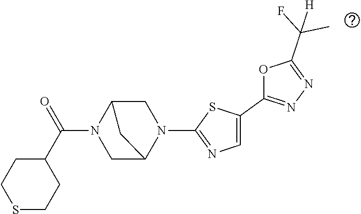 | (5-(5-(5- (difluoromethyl)-1,3,4- oxadiazol-2-yl)thiazol- 2-yl)-2,5-diaza- bicyclo[2.2.1]hept- an-2-yl)(tetrahydro-2H- thiopyran-4- yl)methanone | RT = 5.34 Min. (HPLC Method- I). MS-ESI (m/z): Calculated [M + H]⁺ = 427.09; Observed [M + H]⁺ = 428.1046. ¹H NMR (400 MHz, CDCl₃) δ 7.89 (d, J = 5.7 Hz, 1H), 6.85 (t, J = 51.7 Hz, 1H), 5.08 (s, 1H), 4.91 (s, 1H), 4.72 (d, J = 20.0 Hz, 1H), 3.66- 3.54 (m, 3H), 3.41 (dd, J = 24.6, 9.5 Hz, 1H), 2.72-2.50 (m, 4H), 2.29-2.16 (m, 1H), 2.15-2.05 (m, 1H), 2.02-1.70 (m, 5H). |
| 66 | 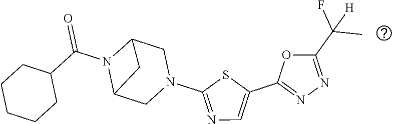 [SMILES: CC(F)c1nnc(-c2cnc(N3CC4CC(C3)N4C(=O)C3CCCCC3)s2)o1] | cyclohexyl(3-(5-(5- (difluoromethyl)-1,3,4- oxadiazol-2-yl)thiazol- 2-yl)-3,6-diaza- bicyclo[3.1.1]hept- an-6-yl)methanone | RT = 5.85 Min. (HPLC Method- I). ¹H NMR (400 MHz, CDCl₃) δ 7.97 (s, 1H), 6.85 (t, J = 51.7 Hz, 1H), 4.58 (t, J = 7.6 Hz, 2H), 4.15 (d, J = 11.0 Hz, 1H), 4.00- 3.73 (m, 2H), 3.58 (s, 1H), 2.83 (dt, J = 8.9, 6.5 Hz, 1H), 2.11 (tt, J = 11.4, 3.7 Hz, 2H), 1.86-1.61 |
| | | | (m, 7H), 1.58-1.39 (m, 4H), |
| | | | 1.30-1.14 (m, 4H). |
| 67 | 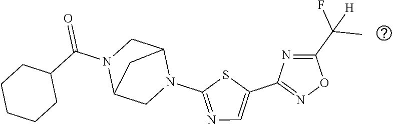 [SMILES: CC(F)c1nc(-c2cnc(N3CC4CC3CN4C(=O)C3CCCCC3)s2)no1] | cyclohexyl(5-(5-(5- (trifluoromethyl)-1,2,4- oxadiazol-3-yl)thiazol- 2-yl)-2,5-diaza- bicyclo[2.2.1]hept- an-2-yl)methanone | RT = 6.71 Min. (HPLC Method- I). ¹H NMR (400 MHz, CDCl₃) δ 7.97 (s, 1H), 5.09 (s, 1H), 4.87 (d, J = 16.3 Hz, 2H), 4.71 (d, J = 15.9 Hz, 1H), 3.75 (dd, J = 9.4, 2.0 Hz, 1H), 3.69-3.51 (m, 7H), 3.40 (d, J = 9.3 Hz, 2H), 2.41- 2.28 (m, 1H), 2.20-2.01 (m, |
| | | | 2H), 2.01-1.93 (m, 2H), 1.78 (d, |
| | | | J = 15.9 Hz, 6H), 1.69 (d, J = |
| | | | 15.6 Hz, 5H), 1.60-1.37 (m, 2), |
| | | | 1.31-1.13 (m, 4H). |
| 65 | 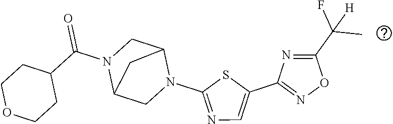 [SMILES: CC(F)c1nc(-c2cnc(N3CC4CC3CN4C(=O)C3CCOCC3)s2)no1] | (tetrahydro-2H-pyran- 4-yl)(5-(5-(5- (trifluoromethyl)-1,2,4- oxadiazol-3-yl)thiazol- 2-yl)-2,5-diaza- bicyclo[2.2.1]hept- an-2-yl)methanone | RT = 5.84 Min. (HPLC Method- I).; ¹H NMR (400 MHz, CDCl₃) δ 7.98 (d, J = 2.7 Hz, 1H), 5.10 (s, 1H), 4.91 (s, 1H), 4.72 (d, J = 15.8 Hz, 1H), 4.13-3.92 (m, 3H), 3.72-3.51 (m, 4H), 3.52- 3.26 (m, 5H), 2.80-2.56 (m, 3H), 2.42 (ddd, J = 11.3, 7.6, 3.8 Hz, 1H), 2.16-1.70 (m, 7H), 1.66-1.42 (m, 3H). |
| 69 | 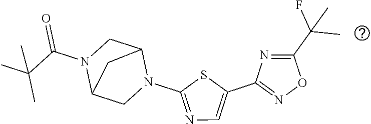 | 2,2-dimethyl-1-(5-(5- (5-(trifluoromethyl)- 1,2,4-oxadiazol-3- yl)thiazol-2-yl)-2,5- diazabicyclo[2.2.1] heptan-2-yl)propan-1- one | Q-TOF LC/MS (m/z): Calculated [M + H]⁺ = 401.1133; Observed [M + H]⁺ = 401.1425. ¹H NMR (400 MHz, CDCl₃)) δ 7.97 (s, 1H), 5.05 (s, 1H), 4.80 (s, 1H), 3.69 (s, 3H), 3.46 (d, J = 9.5 Hz, 1H), 2.13-2.04 (m, 1H), 2.02 (d, J = 8.1 Hz, 1H), 1.22 (s, 10H). |
| 70 | 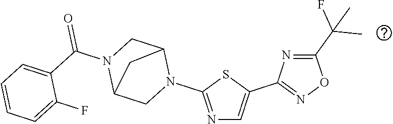 | (2-fluorophenyl)(5-(5- (5-(trifluoromethyl)- 1,2,4-oxadiazol-3- yl)thiazol-2-yl)-2,5- diazabicyclo[2.2.1] heptan-2-yl)methanone | RT = 6.45 Min. (HPLC Method- I). Q-TOF LC/MS (m/z): Calculated [M + H]⁺ = 439.0726; Observed [M + H]⁺ = 440.0945. ¹H NMR (400 MHz, CDCl₃) δ 8.00 (s, 1H), 7.51-7.44 (m, 2H), 7.43-7.35 (m, 2H), 7.29-7.22 (m, 3H), 7.19-7.12 (m, 2H), |
| | | | 7.10-7.03 (m, 1H), 5.24 (s, 1H), |
| | | | 4.85 (d, J = 11.3 Hz, 2H), 4.40 (s, |
| | | | 1H), 3.85-3.78 (m, 2H), 3.72 |
| | | | (dd, J = 9.5, 2.0 Hz, 1H), 3.64 |
| | | | (dd, J = 9.7, 1.9 Hz, 2H), 3.56 |
| | | | (dd, J = 12.1, 2.0 Hz, 2H), 3.45 |
| | | | (d, J = 10.0 Hz, 1H), 2.16 (d, J = |
| | | | 7.1 Hz, 2H), 2.11 (s, 2H). |
| 71 | 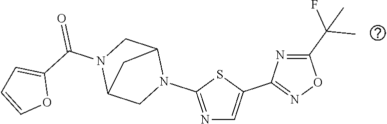 | furan-2-yl(5-(5-(5- (trifluoromethyl)-1,2,4- oxadiazol-3-yl)thiazol- 2-yl)-2,5- diazabicyclo[2.2.1] heptan-2-yl)methanone | RT = 6.17 Min. (HPLC Method- I). Q-TOF LC/MS (m/z): Calculated [M + H]⁺ = 411.0613; Observed [M + H]⁺ = 412.0771. ¹H NMR (400 MHz, CDCl₃) δ 7.97 (d, J = 4.6 Hz, 1H), 7.51 (d, J = 17.3 Hz, 1H), 7.22-7.09 (m, 1H), 6.62-6.47 (m, 1H), 5.25 (s, |
| | | | 1H), 4.93 (s, 1H), 4.82 (s, 1H), |
| | | | 4.05 (q, J = 10.5 Hz, 2H), 3.81 |
| | | | (d, J = 8.0 Hz, 1H), 3.75-3.48 |
| | | | (m, 3H), 2.31 (s, 2H), 2.11 (td, J = |
| | | | 21.2, 11.1 Hz, 3H). |
| 72 | 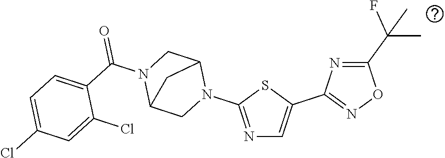 [SMILES: CC(C)(F)c1nc(-c2cnc(N3CC4CC3CN4C(=O)c3ccc(Cl)cc3Cl)s2)no1] | (2,4-dichlorophenyl)(5- (5-(5-(trifluoromethyl)- 1,2,4-oxadiazol-3- yl)thiazol-2-yl)-2,5- diazabicyclo[2.2.1] heptan-2-yl)methanone | RT = 6.99 Min. (HPLC Method- I). ¹H NMR (400 MHz, CDCl₃) δ 7.98 (d, J = 12.3 Hz, 1H), 7.48 (t, J = 1.7 Hz, 1H), 7.36 (dd, J = 8.4, 1.9 Hz, 1H), 7.32-7.27 (m, 1H), 7.22 (d, J = 8.2 Hz, 1H), 5.23 (s, 1H), 4.84 (d, J = 14.1 Hz, 1H), 4.23 (s, 1H), 3.86 (d, J = 11.8 Hz, 1H), 3.80-3.70 (m, 1H), 3.66 (d, J = 9.5 Hz, 1H), |
| | | | 3.58 (dd, J = 9.5, 2.1 Hz, 1H), |
| | | | 3.43-3.23 (m, 2H), 2.21 (d, J = |
| | | | 10.5 Hz, 1H), 2.12 (d, J = 13.7 |
| | | | Hz, 2H). |
| 73 | 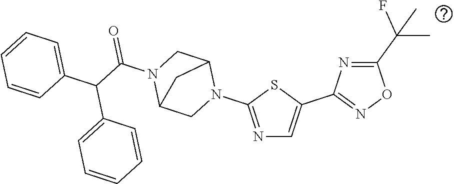 [SMILES: CC(C)(F)c1nc(-c2cnc(N3CC4CC3CN4C(=O)C(c3ccccc3)c3ccccc3)s2)no1] | 2,2-diphenyl-1-(5-(5- (5-(trifluoromethyl)- 1,2,4-oxadiazol-3- yl)thiazol-2-yl)-2,5- diazabicyclo[2.2.1] heptan-2-yl)ethan-1- one | RT = 7.14 Min. (HPLC Method- I). ¹H NMR (400 MHz, CDCl₃) δ 7.88 (s, 1H), 7.40-7.17 (m, 24H), 5.11 (d, J = 6.5 Hz, 2H), 4.85 (d, J = 6.2 Hz, 2H), 4.74 (s, 2H), 3.70-3.51 (m, 5H), 3.51- 3.35 (m, 3H), 2.15-2.01 (m, 3H), 1.98 (t, J = 9.2 Hz, 3H). |
| 74 | 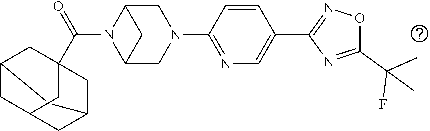 [SMILES: CC(C)(F)c1nc(-c2ccc(N3CC4CC(C3)N4C(=O)C34CC5CC(CC(C5)C3)C4)nc2)no1] | adamantan-1-yl(3-(5- (5-(trifluoromethyl)- 1,2,4-oxadiazol-3- yl)pyridin-2-yl)-3,6- diazabicyclo[3.1.1] heptan-6-yl)methanone | RT = 6.74 Min. (HPLC Method- I). ¹H NMR (400 MHz, CDCl₃) δ 8.88 (d, J = 2.3 Hz, 1H), 8.10 (dd, J = 9.0, 2.4 Hz, 1H), 6.59 (s, 1H), 4.74 (d, J = 70.9 Hz, 2H), 4.19-3.99 (m, 2H), 3.93-3.42 (m, 2), 2.77 (q, J = 7.1 Hz, 2H), 2.01-1.96 (m, 6H), 1.86 (d, J = 2.9 Hz, 2H), 1.76-1.61 (m, 8H). |
| 75 | 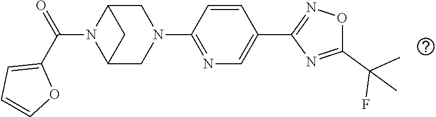 [SMILES: CC(C)(F)c1nc(-c2ccc(N3CC4CC(C3)N4C(=O)c3ccco3)nc2)no1] | furan-2-yl(3-(5-(5- (trifluoromethyl)-1,2,4- oxadiazol-3-yl)pyridin- 2-yl)-3,6-diaza- bicyclo[3.1.1]hept- an-6-yl)methanone | RT = 5.44 Min. (HPLC Method- I). ¹H NMR (400 MHz, CDCl₃) δ 8.86 (dd, J = 2.3, 0.7 Hz, 1H), 8.10 (dd, J = 9.0, 2.3 Hz, 1H), 7.48 (dd, J = 1.8, 0.9 Hz, 1H), 7.11 (dd, J = 3.5, 0.8 Hz, 1H), 6.57 (d, J = 9.0 Hz, 1H), 6.47 (dd, J = 3.5, 1.8 Hz, 1H), 5.16 (s, |
| | | | 1H), 4.77 (s, 1H), 4.24 (d, J = |
| | | | 11.4 Hz, 1H), 3.94 (s, 2H), 3.69 |
| | | | (s, 1H), 2.91 (dt, J = 8.8, 6.5 Hz, |
| | | | 1H), 1.75 (d, J = 8.8 Hz, 1H), |
| | | | 1.34-1.14 (m, 1H). |
| 76 | 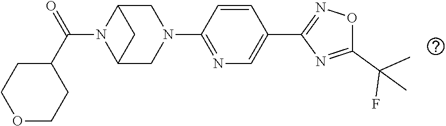 [SMILES: CC(C)(F)c1nc(-c2ccc(N3CC4CC(C3)N4C(=O)C3CCOCC3)nc2)no1] | (tetrahydro-2H-pyran- 4-yl)(3-(5-(5- (trifluoromethyl)-1,2,4- oxadiazol-3-yl)pyridin- 2-yl)-3,6-diaza- bicyclo[3.1.1]hept- an-6-yl)methanone | RT = 5.13 Min. (HPLC Method- I).; ¹H NMR (400 MHz, CDCl₃) δ 8.87 (d, J = 2.3 Hz, 1H), 8.12 (dd, J = 9.0, 2.4 Hz, 1H), 6.58 (d, J = 9.0 Hz, 1H), 4.60 (dd, J = 11.9, 6.1 Hz, 2H), 4.19-4.05 (m, 1H), 4.06-3.83 (m, 4H), 3.60 (s, 1H), 3.38 (dtd, J = 12.3, 10.7, |
| | | | 9.7, 2.5 Hz, 3H), 2.88-2.70 (m, |
| | | | 2H), 2.47-2.29 (m, 2H), 1.92- |
| | | | 1.75 (m, 2H), 1.67 (d, J = 8.8 Hz, |
| | | | 2H), 1.48-1.34 (m, 1H). |
| 77 | 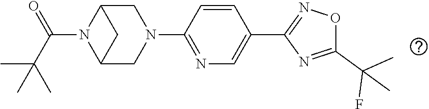 [SMILES: CC(C)(C)C(=O)N1C2CC1CN(c1ccc(-c3noc(C(C)(C)F)n3)cn1)C2] | 2,2-dimethyl-1-(3-(5- (5-(trifluoromethyl)- 1,2,4-oxadiazol-3- yl)pyridin-2-yl)-3,6- diazabicyclo[3.1.1] heptan-6-yl)propan-1- one | RT = 5.68 Min. (HPLC Method- I). ¹H NMR (400 MHz, CDCl₃) δ 8.90 (dd, J = 2.4, 0.8 Hz, 1H), 8.13 (dd, J = 9.0, 2.4 Hz, 1H), 6.60 (dd, J = 9.1, 0.8 Hz, 1H), 4.73 (s, 2H), 4.09 (d, J = 11.7 Hz, 2H), 3.80 (s, 1H), 2.80 (q, J = 7.1 |
| | | | Hz, 1H), 1.66 (s, 1H), 1.20 (s, |
| | | | 9H). |
| 78 | 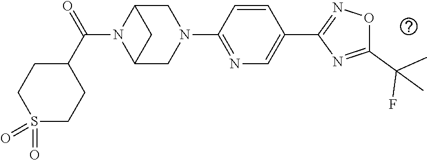 [SMILES: CC(C)(F)c1nc(-c2ccc(N3CC4CC(C3)N4C(=O)C3CCS(=O)(=O)CC3)nc2)no1] | (1,1-dioxidotetrahydro- 2H-thiopyran-4-yl)(3- (5-(5-(trifluoromethyl)- 1,2,4-oxadiazol-3- yl)pyridin-2-yl)-3,6- diazabicyclo[3.1.1] heptan-6-yl)methanone | RT = 4.95 min, (HPLC Method- I). ¹H NMR (400 MHz, CDCl₃) δ 8.82 (d, J = 2.3 Hz, 1H), 8.08 (dd, J = 9.0, 2.4 Hz, 1H), 6.77 (s, 1H), 6.61 (d, J = 9.0 Hz, 1H), 4.74 (s, 1H), 4.56 (s, 1H), 4.05 (d, J = 11.5 Hz, 1H), 3.86 (q, J = 12.5 Hz, 2H), 3.67 (dq, J = 13.4, 6.7 Hz, 2H), 3.37 (t, J = 7.1 Hz, 1H), 3.15 (dt, J = 18.3, 8.5 Hz, |
| | | | 2H), 2.76 (s, 8H), 2.39-2.08 (m, |
| | | | 2H), 2.12-1.88 (m, 2H), 1.65 (d, |
| | | | J = 8.9 Hz, 1H). |
| 79 | 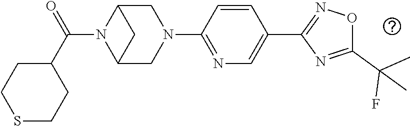 [SMILES: CC(C)(F)c1nc(-c2ccc(N3CC4CC(C3)N4C(=O)C3CCSCC3)nc2)no1] | (tetrahydro-2H- thiopyran-4-yl)(3-(5- (5-(trifluoromethyl)- 1,2,4-oxadiazol-3- yl)pyridin-2-yl)-3,6- diazabicyclo[3.1.1] heptan-6-yl)methanone | RT = 5.62 Min. (HPLC Method- I). ¹H NMR (400 MHz, CDCl₃) δ 8.86 (dd, J = 2.3, 0.7 Hz, 1H), 8.11 (dd, J = 9.0, 2.4 Hz, 1H), 6.61 (dd, J = 9.0, 0.8 Hz, 1H), 4.59 (dt, J = 17.4, 4.0 Hz, 1H), 4.15-3.97 (m, 2H), 3.88 (s, 1H), 3.76-3.53 (m, 2H), 3.18 (qd, J = |
| | | | 7.4, 4.1 Hz, 1H), 2.78 (s, 1H), |
| | | | 2.73-2.51 (m, 1H), 2.21 (tt, J = |
| | | | 10.9, 3.5 Hz, 2H), 2.11-1.99 (m, |
| | | | 2H), 1.97-1.73 (m, 1H), 1.66 (d, |
| | | | J = 8.9 Hz, 4H), 1.49-1.34 (m, |
| | | | 1H). |
| 80 | 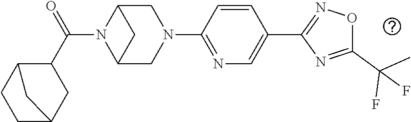 [SMILES: CC(F)(F)c1nc(-c2ccc(N3CC4CC(C3)N4C(=O)C3CC4CCC3C4)nc2)no1] | bicyclo[2.2.1]heptan-2- yl(3-(5-(5- (trifluoromethyl)-1,2,4- oxadiazol-3-yl)pyridin- 2-yl)-3,6-diaza- bicyclo[3.1.1]hept- an-6-yl)methanone | RT = 6.15 Min. (HPLC Method- I). ¹H NMR (400 MHz, CDCl₃) δ 8.89 (dd, J = 6.4, 2.4 Hz, 1H), 8.11 (dt, J = 8.9, 2.6 Hz, 1H), 6.59 (t, J = 8.2 Hz, 1H), 4.75- 4.56 (m, 2H), 4.20-4.10 (m, 1H), 4.09-3.77 (m, 2H), 2.78 (q, J = 7.1 Hz, 2H), 2.71-2.52 (m, |
| | | | 2H), 2.42 (d, J = 4.0 Hz, 1H), |
| | | | 1.67 (dt, J = 14.8, 7.3 Hz, 2H), |
| | | | 1.57-1.43 (m, 2H), 1.43-1.29 |
| | | | (m, 4H), 1.28-1.21 (m, 1H). |
| 81 | 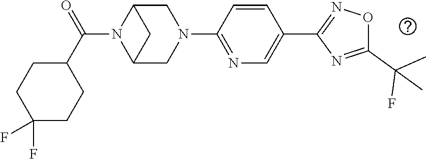 [SMILES: CC(C)(F)c1nc(-c2ccc(N3CC4CC(C3)N4C(=O)C3CCC(F)(F)CC3)nc2)no1] | (4,4- difluorocyclohexyl)(3- (5-(5-(trifluoromethyl)- 1,2,4-oxadiazol-3- yl)pyridin-2-yl)-3,6- diazabicyclo[3.1.1] heptan-6-yl)methanone | RT = 5.87 Min. (HPLC Method- I). ¹H NMR (400 MHz, CDCl₃) δ 8.83 (d, J = 2.3 Hz, 1H), 8.10 (dd, J = 9.0, 2.3 Hz, 1H), 6.61 (d, J = 9.0 Hz, 1H), 4.70-4.49 (m, 1H), 4.13-3.97 (m, 1H), 3.87 (s, 1H), 3.63 (hept, J = 6.6 Hz, 1H), 3.12 (q, J = 7.4 Hz, 2H), 2.77 (ddd, J = 13.6, 9.9, 6.8 Hz, 2H), 2.23 (ddt, J = 14.2, 10.0, 4.1 Hz, 2H), 2.17- |
| | | | 2.02 (m, 2H), 1.78 (dtd, J = 33.4, |
| | | | 11.3, 10.6, 5.4 Hz, 2H), 1.67 (s, |
| | | | 1H), 1.43-1.28 (m, 1H). |
| 82 | 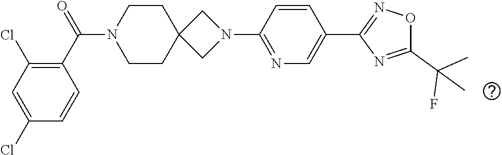 [SMILES: CC(C)(F)c1nc(-c2ccc(N3CC4(CCN(C(=O)c5ccc(Cl)cc5Cl)CC4)C3)nc2)no1] | (2,4-dichlorophenyl)(2- (5-(5-(trifluoromethyl)- 1,2,4-oxadiazol-3- yl)pyridin-2-yl)-2,7- diazaspiro[3.5]nonan- 7-yl)methanone | RT = 5.88 Min. (HPLC Method- I). ¹H NMR (400 MHz, CDCl₃) δ 8.84 (dd, J = 2.3, 0.8 Hz, 1H), 8.07 (dd, J = 8.8, 2.3 Hz, 1H), 7.31 (dd, J = 8.2, 2.0 Hz, 1H), 7.22 (d, J = 8.2 Hz, 1H), 6.33 (dd, J = 8.8, 0.9 Hz, 1H), 3.98-3.82 (m, 5H), 3.35- 3.10 (m, 2H), 2.03 (s, 1H), 1.93- 1.72 (m, 2H), 1.25 (t, J = 7.1 Hz, 1H). |
| 83 | 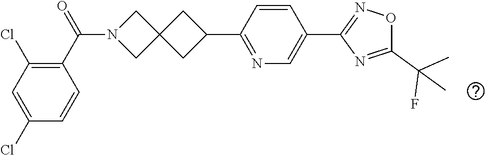 [SMILES: CC(C)(F)c1nc(-c2ccc(C3CC4(C3)CN(C(=O)c3ccc(Cl)cc3Cl)C4)nc2)no1] | (2,4-dichlorophenyl)(6- (5-(5-(trifluoromethyl)- 1,2,4-oxadiazol-3- yl)pyridin-2-yl)-2,6- diazaspiro[3.3]heptan- 2-yl)methanone | RT = 5.78 min. (HPLC Method-I). ¹H NMR (400 MHz, CDCl₃)) δ 8.85 (d, J = 2.2 Hz, 1H), 8.09 (dd, J = 8.7, 2.3 Hz, 1H), 7.44 (t, J = 1.1 Hz, 1H), 7.32 (d, J = 1.1 Hz, 2H), 6.34 (d, J = 8.8 Hz, 1H), 4.44- 4.17 (m, 8H). |

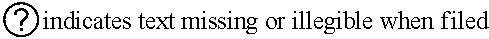

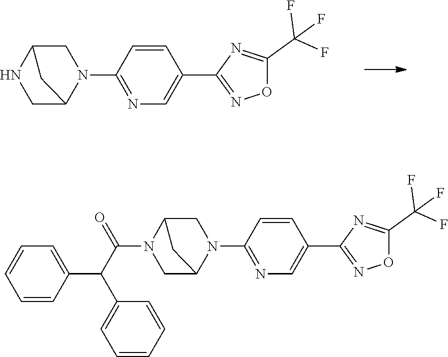 [SMILES: FC(F)(F)c1nc(-c2ccc(N3CC4CC3CN4)nc2)no1 ; O=C(C(c1ccccc1)c1ccccc1)N1CC2CC1CN2c1ccc(-c2noc(C(F)(F)F)n2)cn1]

### Compound of Invention—37, Example-37:2,2-diphenyl-1-(5-(5-(5-(trifluoromethyl)-1,2,4-oxadiazol-3-yl)pyridin-2-yl)-2,5-diazabicyclo[2.2.1]heptan-2-yl) ethan-1-one

To a stirred solution of compound 5-(5-(5-(trifluoromethyl)-1,2,4-oxadiazol-3-yl)pyridin-2-yl)-2,5-diazabicyclo[2.2.1]heptan-2-ium trifluoroacetate (5) (0.052 g, 0.096 mmol) and DIPEA (0.086 mL, 0.062 g, 0.048 mmol) in DCM (3 mL) was added 2,2-diphenylacetyl chloride (0.024 g, 0.096 mmol) at room temperature. After stirring 2 h, the reaction was quenched with water and the product was extracted using DCM. Purification on a silica gel preparatory glass TLC plate using 30% EA/Hex afforded the titled compound Example-37 as a white solid (0.031 g, 61%). RT=6.55 Min. (HPLC Method-I). MS-ESI (m/z): Calculated [M+H]⁺=506.17; Observed [M+H]⁺=506.178.

### Compound of Invention—38, Example-38: (2-fluorophenyl) (5-(5-(5-(trifluoromethyl)-1,2,4-oxadiazol-3-yl)pyridin-2-yl)-2,5-diazabicyclo[2.2.1]heptan-2-yl)methanone

Example-46 was synthesized in an analogous manner to Example-37 by following method-I using the intermediate 5. Purification on a silica gel preparatory glass TLC plate using 50% EA/Hex followed by extraction afforded the title compound Example-38 as a white solid (0.031 g, 71%). RT=5.60 Min. (HPLC Method-I). MS-ESI (m/z): Calculated [M+H]⁺=434.12; Observed [M+H]⁺=434.12.

### Compound of Invention—39, Example-39: (2,4-dichlorophenyl) (5-(5-(5-(trifluoromethyl)-1,2,4-oxadiazol-3-yl)pyridin-2-yl)-2,5-diazabicyclo[2.2.1]heptan-2-yl)methanone

Example-39 was synthesized in an analogous manner to Example-37 by following method-I using the intermediate 5. Purification on a silica gel preparatory glass TLC plate using 50% EA/Hex followed by extraction afforded the title compound Example-39 as a white solid (0.034 g, 71%). RT=6.43 Min. (HPLC Method-I). MS-ESI (m/z): Calculated [M+H]⁺=484.05; Observed [M+H]⁺=484.05.

### Compound of Invention—40, Example-40: (3,4-dichlorophenyl) (5-(5-(5-(trifluoromethyl)-1,2,4-oxadiazol-3-yl)pyridin-2-yl)-2,5-diazabicyclo[2.2.1]heptan-2-yl)methanone

Example-40 was synthesized in an analogous manner to Example-37 by following method-I using the intermediate 5. Purification on a silica gel preparatory glass TLC plate using 50% EA/Hex followed by extraction afforded the title compound Example-40 as a white solid (0.028 g, 44%). RT=6.44 Min. (HPLC Method-I). MS-ESI (m/z): Calculated [M+H]⁺=484.05; Observed [M+H]⁺=484.05.

### Compound of Invention—41, Example-41:2-methyl-1-(5-(5-(5-(trifluoromethyl)-1,2,4-oxadiazol-3-yl)pyridin-2-yl)-2,5-diazabicyclo[2.2.1]heptan-2-yl) propan-1-one

Example-41 was synthesized in an analogous manner to Example-37 by following method-I using the intermediate 5. Purification on a silica gel preparatory glass TLC plate using 50% EA/Hex followed by extraction afforded the title compound Example-41 as a white solid (20 mg, 43%). RT=5.10 Min. (HPLC Method-I). MS-ESI (m/z): Calculated [M+H]⁺=396.16; Observed [M+H]⁺=382.147

### Compound of Invention—42, Example-42:2,2-dimethyl-1-(5-(5-(5-(trifluoromethyl)-1,2,4-oxadiazol-3-yl)pyridin-2-yl)-2,5-diazabicyclo[2.2.1]heptan-2-yl) propan-1-one

Example-50 was synthesized in an analogous manner to Example-37 by following method-I using the intermediate 5. Purification on a silica gel preparatory glass TLC plate using 50% EA/Hex followed by extraction afforded the title compound Example-42 as a colorless foam (2.1 mg, 5.3%). RT=5.41 Min. (HPLC Method-I). MS-ESI (m/z): Calculated [M+H]⁺=396.16; Observed [M+H]⁺=396.164.

### Compound of Invention—43, Example-43: furan-2-yl (5-(5-(5-(trifluoromethyl)-1,2,4-oxadiazol-3-yl)pyridin-2-yl)-2,5-diazabicyclo[2.2.1]heptan-2-yl)methanone

Example-43 was synthesized in an analogous manner to Example-37 by following method-I using the intermediate 5. Purification on a silica gel preparatory glass TLC plate using 50% EA/Hex followed by extraction afforded the title compound Example-43 as a colorless foam (10.6 mg, 26%). RT=5.32 min (HPLC Method-I). MS-ESI (m/z): Calculated [M+H]⁺=406.11; Observed [M+H]⁺=406.111.

### Compound of Invention—44, Example-44: (tetrahydro-2H-thiopyran-4-yl) (5-(5-(5-(trifluoromethyl)-1,2,4-oxadiazol-3-yl)pyridin-2-yl)-2,5-diazabicyclo[2.2.1]heptan-2-yl)methanone

Example-44 was synthesized by following general method-II using the intermediate 5. Purification on a silica gel preparatory glass TLC plate using 50% EA/Hex followed by extraction afforded the title compound Example-44 as a colorless solid (9.8 mg, 17%). RT=5.48 Min. (HPLC Method-I). MS-ESI (m/z): Calculated [M+H]⁺=440.13; Observed [M+H]⁺=440.135.

### Compound of Invention—45, Example-53: (1,1-dioxidotetrahydro-2H-thiopyran-4-yl) (5-(5-(5-(trifluoromethyl)-1,2,4-oxadiazol-3-yl)pyridin-2-yl)-2,5-diazabicyclo[2.2.1]heptan-2-yl)methanone

Example-45 was synthesized by following general method-II using the intermediate 5. Purification on a silica gel preparatory glass TLC plate using 50% EA/Hex followed by extraction afforded the title compound Example-45 as a colorless solid (9.2 mg, 15%,). HPLC: RT=4.79 Min. (HPLC Method-I). MS-ESI (m/z): Calculated [M+H]⁺=472.12; Observed [M+H]⁺=472.125.

### Compound of Invention—46, Example-46: (tetrahydro-2H-pyran-4-yl) (5-(5-(5-(trifluoromethyl)-1,2,4-oxadiazol-3-yl)pyridin-2-yl)-2,5-diazabicyclo[2.2.1]heptan-2-yl)methanone

Example-46 was synthesized by following general method-II using the intermediate 5. Purification on a silica gel preparatory glass TLC plate using 50% EA/Hex followed by extraction afforded the title compound Example-46 as a colorless solid (9.1 mg, 17%). RT=4.94 Min. (HPLC Method-I). MS-ESI (m/z): Calculated [M+H]⁺=424.15; Observed [M+H]⁺=424.158.

### Compound of Invention—47, Example-47: cyclohexyl (5-(5-(5-(trifluoromethyl)-1,2,4-oxadiazol-3-yl)pyridin-2-yl)-2,5-diazabicyclo[2.2.1]heptan-2-yl)methanone

Example-47 was synthesized by following general method-II using the intermediate 5. Purification on a silica gel preparatory glass TLC plate using 50% EA/Hex followed by extraction afforded the title compound Example-47 as a colorless solid (14.8 mg, 27%). RT=5.87 Min. (HPLC Method-I). MS-ESI (m/z): Calculated [M+H]⁺=422.17; Observed [M+H]⁺=422.179.

### Compound of Invention—48, Example-48: (5-(5-(5-(trifluoromethyl)-1,2,4-oxadiazol-3-yl)pyridin-2-yl)-2,5-diazabicyclo[2.2.1]heptan-2-yl) (5-(trifluoromethyl)pyridin-2-yl)methanone

Example-48 was synthesized by following general method-II using the intermediate 5. Purification on a silica gel preparatory glass TLC plate using 50% EA/Hex followed by extraction afforded the title compound Example-48 as a colorless solid (14.7 mg, 25%). RT=6.07 Min. (HPLC Method-I). MS-ESI (m/z): Calculated [M+H]⁺=485.11; Observed [M+H]⁺=485.115.

### Compound of Invention—49, Example-49: (5-(5-(5-(trifluoromethyl)-1,2,4-oxadiazol-3-yl)pyridin-2-yl)-2,5-diazabicyclo[2.2.1]heptan-2-yl) (4-(trifluoromethyl)cyclohexyl)methanone

Example-49 was synthesized by following general method-II using the intermediate 5. Purification on a silica gel preparatory glass TLC plate using 50% EA/Hex followed by extraction afforded the title compound Example-49 as a colorless solid (8.8 mg, 13%). RT=6.20 Min. (HPLC Method-I). MS-ESI (m/z): Calculated [M+H]⁺=490.16; Observed [M+H]⁺=490.166.

### Compound of Invention 50, Example-50: cyclopropyl (5-(5-(5-(trifluoromethyl)-1,2,4-oxadiazol-3-yl)pyridin-2-yl)-2,5-diazabicyclo[2.2.1]heptan-2-yl)methanone

Example-50 was synthesized by following general method-II using the intermediate 5. Purification on a silica gel preparatory glass TLC plate using 50% EA/Hex followed by extraction afforded the title compound Example-50 as a colorless solid (6.2 mg, 17%). RT=5.07 Min. (HPLC Method-I). MS-ESI (m/z): Calculated [M+H]⁺=380.13; Observed [M+H]⁺=380.13.

### Compound of Invention—51, Example-51: (1-(3,5-dichlorophenyl)cyclopropyl) (5-(5-(5-(trifluoromethyl)-1,2,4-oxadiazol-3-yl)pyridin-2-yl)-2,5-diazabicyclo[2.2.1]heptan-2-yl)methanone

Example-51 was synthesized by following general method-II using the intermediate 5. Purification on a silica gel preparatory glass TLC plate using 50% EA/Hex followed by extraction afforded the title compound Example-51 as a colorless solid (18.8 mg, 37%). RT=6.71 min (HPLC Method-I). MS-ESI (m/z): Calculated [M+H]⁺=524.08; Observed [M+H]⁺=524.086.

### Compound of Invention—52, Example-52: (Bicyclo[2.2.1]heptan-2-yl) (5-(5-(5-(trifluoromethyl)-1,2,4-oxadiazol-3-yl)pyridin-2-yl)-2,5-diazabicyclo[2.2.1]heptan-2-yl)methanone

Example-52 was synthesized by following general method-II using the intermediate 5. Purification on a silica gel preparatory glass TLC plate using 50% EA/Hex followed by extraction afforded the title compound Example-52 as a colorless solid (8.6 mg, 20%). RT=6.04 Min. (HPLC Method-I). MS-ESI (m/z): Calculated [M+H]⁺=434.17; Observed [M+H]⁺=434.179.

### Compound of Invention—53, Example-53: ((Bicyclo[2.2.1]hept-5-en-2-yl) (5-(5-(5-(trifluoromethyl)-1,2,4-oxadiazol-3-yl)pyridin-2-yl)-2,5-diazabicyclo[2.2.1]heptan-2-yl)methanone

Example-53 was synthesized by following general method-II using the intermediate 5. Purification on a silica gel preparatory glass TLC plate using 50% EA/Hex followed by extraction afforded the title compound Example-53 as a colorless solid (9.3 mg, 22%). RT=5.95 Min. (HPLC Method-I). MS-ESI (m/z): Calculated [M+H]⁺=432.16; Observed [M+H]⁺=432.16.

### Compound of Invention—54, Example-54: (4,4-difluorocyclohexyl) (5-(5-(5-(trifluoromethyl)-1,2,4-oxadiazol-3-yl)pyridin-2-yl)-2,5-diazabicyclo[2.2.1]heptan-2-yl)methanone

Example-54 was synthesized by following general method-II using the intermediate 5. Purification on a silica gel preparatory glass TLC plate using 50% EA/Hex followed by extraction afforded the title compound Example 54 as a colorless solid (10.1 mg, 22%). RT=5.74 Min. (HPLC Method-I). MS-ESI (m/z): Calculated [M+H]⁺=396.16; Observed [M+H]⁺=396.165.

### Compound of Invention—55, Example-55: Cyclohexyl(3-(5-(5-(trifluoromethyl)-1,2,4-oxadiazol-3-yl)pyridin-2-yl)-3,6-diazabicyclo[3.1.1]heptan-6-yl)methanone

Example-55 was synthesized in an analogous manner to Example-37 by following method-I using the intermediate 16b. Purification on a silica gel preparatory glass TLC plate using 50% EA/Hex followed by extraction afforded the title compound Example-55 as a white solid (19 mg, 50%) RT=6.04 Min. (HPLC Method-I).

### Compound of Invention—56, Example-56: (Tetrahydro-2H-pyran-4-yl) ((1S,4S)-5-(5-(5-(trifluoromethyl)-1,2,4-oxadiazol-3-yl)pyridin-2-yl)-2,5-diazabicyclo[2.2.1]heptan-2-yl)methanone VMCC-MP-07-014-001

Example-56 was synthesized in an analogous manner to Example-37 by following method-I using the intermediate 24. Purification on a silica gel preparatory glass TLC plate using 70% EA/Hex followed by extraction afforded the title compound Example-56 as a white solid (77 mg, 64%), RT=4.83 Min. (HPLC Method-I). Q-TOF LC/MS (m/z): Calculated [M+H]⁺=423.1518; Observed [M+H]⁺=424.1631.

### Compound of Invention—57, Example-57: Cyclohexyl(7-(5-(5-(trifluoromethyl)-1,2,4-oxadiazol-3-yl)pyridin-2-yl)-2,7-diazaspiro[3.5]nonan-2-yl)methanone

Example-57 was synthesized in an analogous manner to Example-37 by following method-I using the intermediate 45. Purification on a silica gel preparatory glass TLC plate using 50% EA/Hex followed by extraction afforded the title compound Example-57 as a white solid (30 mg, 60%), RT=6.29 Min. (HPLC Method-I).

### Compound of Invention—58, Example-58: Cyclohexyl(5-(5-(5-(difluoromethyl)-1,3,4-oxadiazol-2-yl)pyridin-2-yl)-2,5-diazabicyclo[2.2.1]heptan-2-yl)methanone

Example-58 was synthesized in an analogous manner to Example-37 by following method-I using the intermediate 12a. Purification on a silica gel preparatory glass TLC plate using 50% EA/Hex followed by extraction afforded the title compound Example-58as a white solid (10 mg, 16%), RT=6.54 Min. (HPLC Method-I).

### Compound of Invention—59, Example-59: Cyclohexyl(5-(5-(5-(trifluoromethyl)-1,3,4-oxadiazol-2-yl)pyridin-2-yl)-2,5-diazabicyclo[2.2.1]heptan-2-yl)methanone

Example-59 was synthesized in an analogous manner to Example-37 by following method-I using the intermediate 12b. Purification on a silica gel preparatory glass TLC plate using 50% EA/Hex followed by extraction afforded the title compound Example-59 as a white solid (30 mg, 44%), RT=6.51 Min. (HPLC Method-I

### Compound of Invention—60, Example-60: Cyclohexyl(3-(5-(5-(difluoromethyl)-1,3,4-oxadiazol-2-yl)pyridin-2-yl)-3,6-diazabicyclo[3.1.1]heptan-6-yl)methanone

Example-60 was synthesized in an analogous manner to Example-37 by following method-I using the intermediate 16b. Purification on a silica gel preparatory glass TLC plate using 50% EA/Hex followed by extraction afforded the title compound Example-60 as a white solid (20 mg, 30%), RT=6.36 Min. (HPLC Method-I).

### Compound of Invention—61, Example-61: Cyclohexyl(7-(5-(5-(difluoromethyl)-1,3,4-oxadiazol-2-yl)pyridin-2-yl)-2,7-diazaspiro[3.5]nonan-2-yl)methanone

Example-61 was synthesized in an analogous manner to Example-37 by following method-I using the intermediate 14b. Purification on a silica gel preparatory glass TLC plate using 50% EA/Hex followed by extraction afforded the title compound Example-61 as a white solid (35 mg, 50%), RT=6.41 Min. (HPLC Method-I).

### Compound of Invention—62, Example-62: Cyclohexyl(5-(5-(5-(difluoromethyl)-1,3,4-oxadiazol-2-yl)thiazol-2-yl)-2,5-diazabicyclo[2.2.1]heptan-2-yl)methanone

Example-62 was synthesized by following method-II. To a stirred solution of compound 5-(5-(5-(difluoromethyl)-1,3,4-oxadiazol-2-yl)thiazol-2-yl)-2,5-diazabicyclo[2.2.1]heptan-2-ium 2,2,2-trifluoroacetate (36), (0.055 g, 0.13 mmol), cyclohexanecarboxylic acid (III, 26 mg, 0.20 mmol), HOBt (IV, 27 mg, 0.200 mmol) and EDC (31 mg, 0.200 mmol) in DMF 95 mL) was added DIEA 9 0.10 mL, 77 mg, 0.67n mmol) at room temperature. The resulting clear solution was stirred at rt for 24 h. After stirring 24 h, the reaction was quenched with a drop of water and solvent DMF was removed under high vacuum. The crude product was extracted using DCM. Purification on a silica gel preparatory glass TLC plate using 50% EA/Hex followed by extraction afforded the title compound Example-62 as a brown color solid (20 mg, 69%), RT=5.81 Min. (HPLC Method-I).

### Compound of Invention—63, Example-63: (4,4-difluorocyclohexyl) (5-(5-(5-(difluoromethyl)-1,3,4-oxadiazol-2-yl)thiazol-2-yl)-2,5-diazabicyclo[2.2.1]heptan-2-yl)methanone

Example-63 was synthesized in an analogous manner to Example-62 by following method-II using the intermediate 35. Purification by preparatory TLC plate on silica gel using 60% EA/Hex as an eluent afforded the title compound Example-63 as a brown color solid (20 mg, 24%). HPLC Retention time: 5.62 min (HPLC Method-I).

### Compound of Invention—64, Example-64:5-(5-(5-(difluoromethyl)-1,3,4-oxadiazol-2-yl)thiazol-2-yl)-2,5-diazabicyclo[2.2.1]heptan-2-yl) (tetrahydro-2HI-pyran-4-yl)methanone

Example-64 was synthesized in an analogous manner to Example-62 by following method-II using the intermediate 35. Purification by preparatory TLC plate on silica gel using 60% EA/Hex as an eluent afforded the title compound Example-64 as a brown color solid (35 mg, 51%). HPLC Retention time: 4.94 min (HPLC Method-I).

### Compound of Invention—65, Example-65:5-(5-(5-(difluoromethyl)-1,3,4-oxadiazol-2-yl)thiazol-2-yl)-2,5-diazabicyclo[2.2.1]heptan-2-yl) (tetrahydro-2H-pyran-4-yl)methanone

Example-65 was synthesized in an analogous manner to Example-62 by following method-II using the intermediate 35. Purification by preparatory TLC plate on silica gel using 60% EA/Hex as an eluent afforded the title compound Example-65 as a brown color solid (30 mg, 32%). HPLC Retention time: 5.34 min (HPLC Method-I).

### Compound of Invention—66, Example-66: Cyclohexyl(3-(5-(5-(difluoromethyl)-1,3,4-oxadiazol-2-yl)thiazol-2-yl)-3,6-diazabicyclo[3.1.1]heptan-6-yl)methanone

Example-66 was synthesized in an analogous manner to Example-62 by following method-I using the intermediate 37. Purification on a silica gel preparatory glass TLC plate using 60% EA/Hex followed by extraction afforded the title compound Example-66 as a pale brown color solid (30 mg, 40%), RT=5.85 Min. (HPLC Method-I).

### Compound of Invention—67, Example-67: Cyclohexyl(5-(5-(5-(trifluoromethyl)-1,2,4-oxadiazol-3-yl)thiazol-2-yl)-2,5-diazabicyclo[2.2.1]heptan-2-yl)methanone

Example-67 was synthesized in an analogous manner to Example 37 by following method-I using the intermediate 31. Purification on a silica gel preparatory glass TLC plate using 60% EA/Hex followed by extraction afforded the title compound Example-67 as a pale brown color solid (32 mg, 48%), RT=6.71 Min. (HPLC Method-I).

### Compound of Invention—68, Example-68: (Tetrahydro-2H-pyran-4-yl) (5-(5-(5-(trifluoromethyl)-1,2,4-oxadiazol-3-yl)thiazol-2-yl)-2,5-diazabicyclo[2.2.1]heptan-2-yl)methanone

Example-68 was synthesized in an analogous manner to Example-37 by following method-I using the intermediate 31. Purification on a silica gel preparatory glass TLC plate using 60% EA/Hex followed by extraction afforded the title compound Example-68 as a pale brown color solid (20 mg, 28%), RT=5.84 Min. (HPLC Method-I).

### Compound of Invention—69, Example-69:2,2-Dimethyl-1-(5-(5-(5-(trifluoromethyl)-1,2,4-oxadiazol-3-yl)thiazol-2-yl)-2,5-diazabicyclo[2.2.1]heptan-2-yl) propan-1-one

Example-69 was synthesized in an analogous manner to Example-37 by following method-I using the intermediate 31. Purification on a silica gel preparatory glass TLC plate using 60% EA/Hex followed by extraction afforded the title compound Example-69 as a pale brown color solid (18 mg, 26%), RT=6.43 Min. (HPLC Method-I).

### Compound of Invention—70, Example-70: (2-Fluorophenyl) (5-(5-(5-(trifluoromethyl)-1,2,4-oxadiazol-3-yl)thiazol-2-yl)-2,5-diazabicyclo[2.2.1]heptan-2-yl)methanone

Example-70 was synthesized in an analogous manner to Example-37 by following method-I using the intermediate 31. Purification on a silica gel preparatory glass TLC plate using 50% EA/Hex followed by extraction afforded the title compound Example-70 as a pale brown color solid (25 mg), RT=6.45 Min.

### Compound of Invention—71, Example-71: Furan-3-yl (5-(5-(5-(trifluoromethyl)-1,2,4-oxadiazol-3-yl)thiazol-2-yl)-2,5-diazabicyclo[2.2.1]heptan-2-yl)methanone

Example-71 was synthesized in an analogous manner to Example-37by following method-I using the intermediate 31. Purification on a silica gel preparatory glass TLC plate using 50% EA/Hex followed by extraction afforded the title compound Example-71 as a brown color solid (22 mg), RT=6.17 Min. (HPLC Method-I).

### Compound of Invention—72, Example-72: (2,4-Dichlorophenyl) (5-(5-(5-(trifluoromethyl)-1,2,4-oxadiazol-3-yl)thiazol-2-yl)-2,5-diazabicyclo[2.2.1]heptan-2-yl)methanone

Example-72 was synthesized in an analogous manner to Example-37 by following method-I using the intermediate 31. Purification on a silica gel preparatory glass TLC plate using 40% EA/Hex followed by extraction afforded the title compound Example-72 as a brown color solid (36 mg, 47%), RT=6.17 Min. (HPLC Method-I).

### Compound of Invention—73, Example-73:2,2-Diphenyl-1-(5-(5-(5-(trifluoromethyl)-1,2,4-oxadiazol-3-yl)thiazol-2-yl)-2,5-diazabicyclo[2.2.1]heptan-2-yl) ethan-1-one

Example-73 was synthesized in an analogous manner to Example-37 by following method-I using the intermediate 31. Purification on a silica gel preparatory glass TLC plate using 40% EA/Hex followed by extraction afforded the title compound Example-73 as a brown color solid (40 mg, 50%), RT=7.14 Min. (HPLC Method-I).

### Compound of Invention—74, Example-74: Adamantan-1-yl (3-(5-(5-(trifluoromethyl)-1,2,4-oxadiazol-3-yl)pyridin-2-yl)-3,6-diazabicyclo[3.1.1]heptan-6-yl)methanone

Example-74 was synthesized in an analogous manner to Example-37 by following method-I using the intermediate 16b. Purification on a silica gel preparatory glass TLC plate using 70% EA/Hex followed by extraction afforded the title compound Example-74 as a brown color solid (50 mg, 50%), RT=6.74 Min. (HPLC Method-I).

### Compound of Invention—75, Example-75: Furan-2-yl (3-(5-(5-(trifluoromethyl)-1,2,4-oxadiazol-3-yl)pyridin-2-yl)-3,6-diazabicyclo[3.1.1]heptan-6-yl)methanone

Example-75 was synthesized in an analogous manner to Example-37 by following method-I using the intermediate 16b. Purification on a silica gel preparatory glass TLC plate using 70% EA/Hex followed by extraction afforded the title compound Example-75 as a brown color solid (39.2 mg, 45%), RT=5.44 Min. (HPLC Method-I).

### Compound of Invention—76, Example-76: (Tetrahydro-2H-pyran-4-yl) (3-(5-(5-(trifluoromethyl)-1,2,4-oxadiazol-3-yl)pyridin-2-yl)-3,6-diazabicyclo[3.1.1]heptan-6-yl)methanone

Example-76 was synthesized in an analogous manner to Example-37 by following method-I using the intermediate 16b. Purification on a silica gel preparatory glass TLC plate using 70% EA/Hex followed by extraction afforded the title compound Example-76 as a brown color solid (45 mg, 50%), RT=5.13 Min. (HPLC Method-I).

### Compound of Invention—77, Example-77: (2,2-Dimethyl-1-(3-(5-(5-(trifluoromethyl)-1,2,4-oxadiazol-3-yl)pyridin-2-yl)-3,6-diazabicyclo[3.1.1]heptan-6-yl) propan-1-one

Example-77 was synthesized in an analogous manner to Example-37 by following method-I using the intermediate 16b. Purification on a silica gel preparatory glass TLC plate using 70% EA/Hex followed by extraction afforded the title compound Example-77 as a brown color solid (40 mg, 48%), RT=5.68 Min. (HPLC Method-I).

### Compound of Invention—78, Example-78: (1,1-dioxidotetrahydro-2H-thiopyran-4-yl) (3-(5-(5-(trifluoromethyl)-1,2,4-oxadiazol-3-yl)pyridin-2-yl)-3,6-diazabicyclo[3.1.1]heptan-6-yl)methanone

Example-78 was synthesized by following method-III. To a flask containing 5 mL (˜ 90.6 mg) of 3-(6-(3,6-diazabicyclo[3.1.1]heptan-3-yl)pyridin-3-yl)-5-(trifluoromethyl)-1,2,4-oxadiazole TFA salt (the intermediate 16b), (5 mL, ˜ 90.6 mg) of stock solution was added 1,1 dioxo-tetrahydrothiopyran-4-carboxylic acid (38 mg, 1.0 equiv), and HATU (122 mg, 1.5 equiv), and stirred at room temperature for 5 hours. The reactions flasks were then allowed to evaporate and diluted with DCM (˜ 2 mL) and methanol (˜ 0.5 mL). Purification on a silica gel preparatory glass TLC plate using DCM and 4% methanol followed by extraction afforded the title compound Example-78 as a brown color solid (138 mg, >99%), RT=4.95 Min. (HPLC Method-I).

### Compound of Invention—79, Example-79: (Tetrahydro-2H-thiopyran-4-yl) (3-(5-(5-(trifluoromethyl)-1,2,4-oxadiazol-3-yl)pyridin-2-yl)-3,6-diazabicyclo[3.1.1]heptan-6-yl)methanone

Example-79 was synthesized in an analogous manner to Example-78 by following method-III using the intermediate 16b. Purification on a silica gel preparatory glass TLC plate using 70% EA/Hex followed by extraction afforded the title compound Example-79 as a colorless solid (72 mg, 77%), RT=5.623 Min. (HPLC Method-I).

### Compound of Invention—80, Example-80: Bicyclo[2.2.1]heptan-2-yl (3-(5-(5-(trifluoromethyl)-1,2,4-oxadiazol-3-yl)pyridin-2-yl)-3,6-diazabicyclo[3.1.1]heptan-6-yl)methanone

Example-80 was synthesized in an analogous manner to Example-78 by following method-III using the intermediate 16b. Purification on a silica gel preparatory glass TLC plate using 70% EA/Hex followed by extraction afforded the title compound Example-80 as a colorless solid (42.4 mg, 46%), RT=6.15 Min. (HPLC Method-I).

### Compound of Invention—81, Example-81: (4,4-Difluorocyclohexyl) (3-(5-(5-(trifluoromethyl)-1,2,4-oxadiazol-3-yl)pyridin-2-yl)-3,6-diazabicyclo[3.1.1]heptan-6-yl)methanone

Example-81 was synthesized in an analogous manner to Example 78 by following method-III using the intermediate 16b. Purification on a silica gel preparatory glass TLC plate using 70% EA/Hex followed by extraction afforded the title compound Example-81 as a colorless solid (62 mg, 63%), RT=5.87 Min. (HPLC Method-I).

### Compound of Invention—82, Example-82:2,4-Dichlorophenyl) (2-(5-(5-(trifluoromethyl)-1,2,4-oxadiazol-3-yl)pyridin-2-yl)-2,7-diazaspiro[3.5]nonan-7-yl)methanone

Example-90 was synthesized in an analogous manner to Example-37 by following method-I using the intermediate 45. Purification on a silica gel preparatory glass TLC plate using 50% EA/Hex followed by extraction afforded the title compound Example-82 as a colorless solid (64 mg, 75%), RT=5.88 Min. (HPLC Method-I).

### Compound of Invention—83, Example-83: (2,4-Ddichlorophenyl) (6-(5-(5-(trifluoromethyl)-1,2,4-oxadiazol-3-yl)pyridin-2-yl)-2,6-diazaspiro[3.3]heptan-2-yl)methanone

Example-83 was synthesized in an analogous manner to Example-37 by following method-I using the intermediate 47. Purification on a silica gel preparatory glass TLC plate using 50% EA/Hex followed by extraction afforded the title compound Example-83 as a colorless solid (75 mg, 88%), RT=5.78 Min. (HPLC Method-I).

Method-IV: To a stirred solution of the intermediate—5 TFA salt (0.10 mmol) in DCM (3 to 5 mL) was added DIPEA (0.30 mmol) followed by addition of isocyante (0.12 mmol) at room temperature After stirring 1 to 2 hours, the reaction was quenched with water and the product was extracted with DCM. Purification on a silica gel preparatory TLC glass plate using 50% EA/Hex afforded the titled compound.

Method-V: To a stirred solution of the intermediate—5 TFA salt (0.10 mmol) in DCM (3 to 5 mL) was added DIPEA (0.30 mmol) followed by addition of aminocarbamoyl chloride (0.12 mmol) at room temperature After stirring 1 to 2 hours, the reaction was quenched with water and the product was extracted with DCM. Purification on a silica gel preparatory TLC glass plate using 50% EA/Hex afforded the titled compound.

TABLE III
Compounds of invention (Iaaa₃) and (Ibaa₃) Examples 84-114

| Example | Compound | Compound Name | Characterization |
|---|---|---|---|
| 84 | 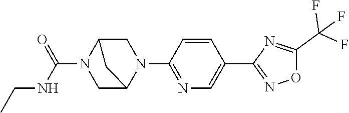 [SMILES: CCNC(=O)N1CC2CC1CN2c1ccc(-c2noc(C(F)(F)F)n2)cn1] | N-ethyl-5-(5-(5-(trifluoromethyl)- 1,2,4-oxadiazol-3-yl)pyridin-2-yl)- 2,5-diazabicyclo[2.2.1] heptane-2-carboxamide | RT = 4.69 Min. (HPLC Method- I). MS-ESI (m/z): Calculated [M + H]⁺ = 383.14; Observed [M + H]⁺ = 383.14. ¹H NMR (400 MHz, CDCl₃) δ 8.81 (dd, J = 2.4, 0.8 Hz, 1H), 8.05 (dd, J = 8.8, 2.4 Hz, 1H), 7.26 (s, 1H), |
| | | | 6.38 (d, J = 8.9 Hz, 1H), 5.10 (s, |
| | | | 1H), 4.79 (s, 1H), 4.29 (t, J = 5.6 |
| | | | Hz, 1H), 3.56 (dd, J = 9.3, 1.9 Hz, |
| | | | 1H), 3.46 (dd, J = 8.3, 2.0 Hz, |
| | | | 2H), 3.28-3.12 (m, 3H), 2.18 (s, |
| | | | 1H), 2.07-1.87 (m, 1H), 1.10 (t, |
| | | | J = 7.2 Hz, 3H). |
| 85 | 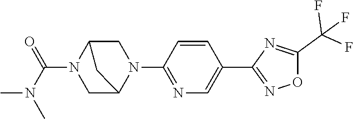 [SMILES: CN(C)C(=O)N1CC2CC1CN2c1ccc(-c2noc(C(F)(F)F)n2)cn1] | N,N-dimethyl-5-(5-(5- (trifluoromethyl)-1,2,4-oxadiazol-3- yl)pyridin-2-yl)-2,5-diazabicyclo [2.2.1]heptane-2-carboxamide | RT = 4.89 Min. (HPLC Method- I). MS-ESI (m/z): Calculated [M + H]⁺ = 383.14; Observed [M + H]⁺ = 383.14. ¹H NMR (400 MHz, CDCl₃) δ 8.82 (dd, J = 2.3, 0.8 Hz, 1H), 8.05 (dd, J = 8.9, 2.4 Hz, 1H), 7.26 (d, J = 0.4 Hz, 1H), 6.39 (d, J = 8.9 Hz, 1H), 5.07 (s, |
| | | | 1H), 4.58 (s, 1H), 3.73 (d, J = 9.5 |
| | | | Hz, 1H), 3.57 (ddd, J = 20.2, 9.3, |
| | | | 2.1 Hz, 2H), 3.31 (s, 1H), 2.82 (s, |
| | | | 6H), 1.95 (s, 2H). |
| 86 | 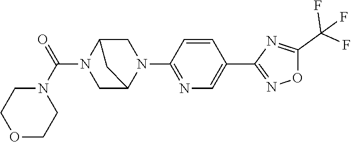 [SMILES: O=C(N1CCOCC1)N1CC2CC1CN2c1ccc(-c2noc(C(F)(F)F)n2)cn1] | morpholino (5-(5-(5- (trifluoromethyl)-1,2,4-oxadiazol-3- yl)pyridin-2-yl)-2,5-diazabicyclo [2.2.1]heptan-2-yl)methanone | RT = 4.85 Min. (HPLC Method- I). MS-ESI (m/z): Calculated [M + H]⁺ = 425.15; Observed [M + H]⁺ = 425.15. ¹H NMR (400 MHz, CDCl₃) δ 8.82 (dd, J = 2.4, 0.7 Hz, 1H), 8.06 (dd, J = 8.9, 2.3 Hz, 1H), 6.39 (d, J = 8.9 Hz, 1H), 5.08 (s, 1H), 4.58 (p, J = 1.7 Hz, 1H), 3.72-3.51 (m, 6H), 3.41-3.10 (m, 6H), 1.96 (d, J = 2.3 Hz, 2H). |
| 87 | 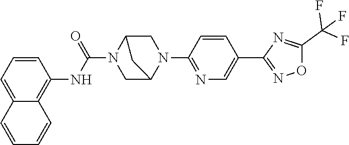 [SMILES: O=C(Nc1cccc2ccccc12)N1CC2CC1CN2c1ccc(-c2noc(C(F)(F)F)n2)cn1] | N-(naphthalen-1-yl)-5-(5-(5- (trifluoromethyl)-1,2,4-oxadiazol- 3-yl)pyridin-2-yl)-2,5- diazabicyclo[2.2.1]heptane-2- carboxamide | RT = 5.82 min (HPLC Method-I). MS-ESI (m/z): Calculated [M + H]⁺ = 480.15; Observed [M + H]⁺ = 480.15. ¹H NMR (400 MHz, CDCl₃)) δ 8.85 (dd, J = 2.3, 0.7 Hz, 1H), 8.09 (dd, J = 8.9, 2.3 Hz, 1H), 7.88-7.82 (m, 1H), 7.80-7.73 (m, 2H), 7.64 (d, J = 8.2 Hz, 1H), 7.51-7.42 (m, |
| | | | 2H), 6.51 (s, 1H), 6.40 (d, J = 8.9 |
| | | | Hz, 1H), 5.20 (s, 1H), 4.91 (s, |
| | | | 1H), 3.68 (dd, J = 8.2, 2.0 Hz, |
| | | | 1H), 3.62-3.43 (m, 3H), 2.11- |
| | | | 1.92 (m, 2H). |
| 88 | 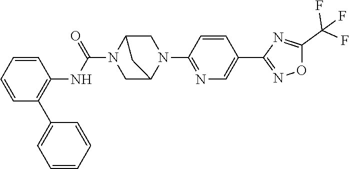 [SMILES: O=C(Nc1ccccc1-c1ccccc1)N1CC2CC1CN2c1ccc(-c2noc(C(F)(F)F)n2)cn1] | N-([1,1′-biphenyl]-2-yl)-5-(5-(5- (trifluoromethyl)-1,2,4-oxadiazol- 3-yl)pyridin-2-yl)-2,5-diazabicyclo [2.2.1]heptane-2-carboxamide | RT = 6.12 Min. (HPLC Method- I). Calculated [M + H]⁺ = 507.17; Observed [M + H]⁺ = 507.17. ¹H NMR (400 MHz, CDCl₃) δ 8.83 (dd, J = 2.4, 0.7 Hz, 1H), 8.14 (dd, J = 8.3, 1.2 Hz, 1H), 8.08 (dd, J = 8.9, 2.4 Hz, 1H), 7.38 (td, J = 5.6, 2.6 Hz, 3H), 7.31 (ddd, J = 7.4, 5.3, 1.4 Hz, 3H), 7.18 (dd, J = 7.6, 1.7 Hz, 1H), 7.08 (td, J = 7.5, 1.2 Hz, 1H), 6.38 (d, J = 8.9 |
| | | | Hz, 1H), 5.05 (s, 1H), 4.76 (s, |
| | | | 1H), 3.55 (dd, J = 9.3, 1.9 Hz, |
| | | | 1H), 3.47 (d, J = 9.6 Hz, 1H), |
| | | | 3.21 (dd, J = 8.3, 1.8 Hz, 1H), |
| | | | 3.12 (d, J = 8.1 Hz, 1H), 1.96 (s, |
| | | | 1H), 1.71 (s, 1H). |
| 89 | 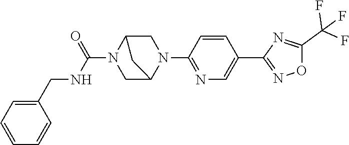 [SMILES: O=C(NCc1ccccc1)N1CC2CC1CN2c1ccc(-c2noc(C(F)(F)F)n2)cn1] | N-benzyl-5-(5-(5- (trifluoromethyl)-1,2,4-oxadiazol- 3-yl)pyridin-2-yl)-2,5-diazabicyclo [2.2.1]heptane-2-carboxamide | RT = 5.88 Min. (HPLC Method- I). ESI (m/z): Calculated [M + H]⁺ = 445.14; Observed [M + H]⁺ = 446.14. ¹H NMR (400 MHz, CDCl₃) δ 8.86 (dd, J = 3.2, 2.2 Hz, 1H), 8.22-7.90 (m, 1H), 7.96 (s, 5H), 6.43 (dd, J = 8.9, 6.3 Hz, 1H), 5.24 (s, 1H), 5.13 (d, J = 10.9 Hz, 2H), 4.91 (s, 1H), 3.77-3.59 (m, 3H), 3.49 (d, J = |
| | | | 9.8 Hz, 1H), 2.26-1.86 (m, 2H). |
| 90 | 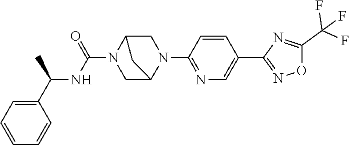 [SMILES: C[C@@H](NC(=O)N1CC2CC1CN2c1ccc(-c2noc(C(F)(F)F)n2)cn1)c1ccccc1] | N-((R)-1-phenylethyl)-5-(5-(5- (trifluoromethyl)-1,2,4-oxadiazol- 3-yl)pyridin-2-yl)-2,5-diazabicyclo [2.2.1]heptane-2-carboxamide | RT = 5.67 Min. (HPLC Method- I). MS-ESI (m/z): Calculated [M + H]⁺ = 459.17; Observed [M + H]⁺ = 459.17. ¹H NMR (400 MHz, CDCl₃) δ 8.82 (d, J = 2.3 Hz, 1H), 8.07 (dd, J = 8.9, 2.3 Hz, 1H), 7.35-7.16 (m, 5H), 7.16-7.05 (m, 1H), 6.37 (d, J = 8.8 Hz, 1H), 5.13 (br., s, 1H), |
| | | | 4.99 (p, J = 7.1 Hz, 1H), 4.33 (d, |
| | | | J = 7.6 Hz, 1H), 3.55 (d, J = 9.2 |
| | | | Hz, 1H), 3.49-3.39 (m, 2H), |
| | | | 3.34 (d, J = 8.2 Hz, 1H), 1.96 |
| | | | (br., s, 2H), 1.59-1.40 (d, J = 6.8 |
| | | | Hz, 3H). |
| 91 | 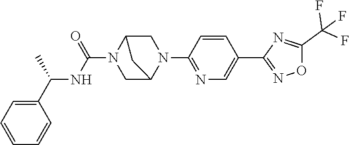 [SMILES: C[C@H](NC(=O)N1CC2CC1CN2c1ccc(-c2noc(C(F)(F)F)n2)cn1)c1ccccc1] | N-((S)-1-phenylethyl)-5-(5-(5- (trifluoromethyl)-1,2,4-oxadiazol- 3-yl)pyridin-2-yl)-2,5-diazabicyclo [2.2.1]heptane-2-carboxamide | RT = 5.65 Min. (HPLC Method- I). MS-ESI (m/z): Calculated [M + H]⁺ = 459.17; Observed [M + H]⁺ = 459.17. ¹H NMR (400 MHz, CDCl₃) δ 8.84 (dd, J = 2.3, 0.8 Hz, 1H), 8.08 (dd, J = 8.9, 2.3 Hz, 1H), 7.42-7.24 (m, 5H), 7.17-7.08 (m, 1H), 6.40 (d, J = 8.9 Hz, 1H), 5.12 (br., s, 1H), |
| | | | 4.91 (m, 1H), 4.36 (d, J = 7.5 |
| | | | Hz, 1H), ), 3.55 (d, J = 9.2 Hz, |
| | | | 1H), 3.49-3.39 (m, 2H), 3.35 |
| | | | (d, J = 8.2 Hz, 1H), 1.96 (br., s, |
| | | | 2H), 1.45 (d, J = 6.9 Hz, 3H). |
| 92 | 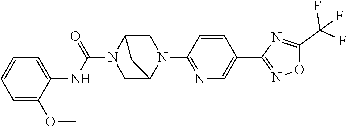 [SMILES: COc1ccccc1NC(=O)N1CC2CC1CN2c1ccc(-c2noc(C(F)(F)F)n2)cn1] | N-(2-methoxyphenyl)-5- (5-(5-(trifluoromethyl)-1,2,4- oxadiazol-3-yl)pyridin-2-yl)-2,5- diazabicyclo[2.2.1]heptane-2- carboxamide | RT = 5.68 Min. (HPLC Method- I). MS-ESI (m/z): Calculated [M + H]⁺ = 460.15; Observed [M + H]⁺ = 460.15. ¹H NMR (400 MHz, CDCl₃) δ 8.85 (dd, J = 2.3, 0.8 Hz, 1H), 8.11 (d, J = 8.10 Hz, 1H), 7.05-6.89 (m, 2H), 6.87- 6.78 (m, 2H), 6.40 (d, J = 8.9 Hz, |
| | | | 1H), 5.20 (br., s, 1H), 4.87 (sm |
| | | | 1H), 3.85 (s, 3H), 3.65 (ddd, J = |
| | | | 14.5, 8.8, 1.9 Hz, 2H), 3.56 (t, J = |
| | | | 8.8 Hz, 2H), 2.05 (d, J = 2.7 Hz, |
| | | | 2H). |
| 93 | 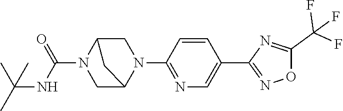 [SMILES: CC(C)(C)NC(=O)N1CC2CC1CN2c1ccc(-c2noc(C(F)(F)F)n2)cn1] | N-(tert-butyl)-5-(5- (5-(trifluoromethyl)-1,2,4- oxadiazol-3-yl)pyridin-2-yl)-2,5- diazabicyclo[2.2.1]heptane-2- carboxamide | RT = 5.32 Min. (HPLC Method- I). MS-ESI (m/z): Calculated [M + H]⁺ = 411.17; Observed [M + H]⁺ = 411.17. ¹H NMR (400 MHz, CDCl₃) δ 8.83 (dd, J = 2.3, 0.7 Hz, 1H), 8.07 (dd, J = 8.9, 2.3 Hz, 1H), 6.39 (d, J = 8.9 Hz, 1H), |
| | | | 5.12 (br., s, 1H), 4.80 (s, 1H), |
| | | | 3.56 (dd, J = 9.2, 1.9 Hz, 1H), |
| | | | 3.51-3.38 (m, 2H), 3.29 (d, J = |
| | | | 8.1 Hz, 1H), 1.96 (s, 2H), 1.31 (s, |
| | | | 9H). |
| 94 | 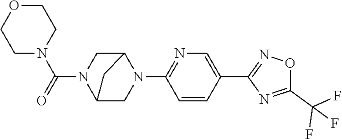 [SMILES: O=C(N1CCOCC1)N1CC2CC1CN2c1ccc(-c2noc(C(F)(F)F)n2)cn1] | morpholino (5-(5-(5- (trifluoromethyl)-1,2,4- oxadiazol-3-yl)pyrimidin-2-yl)- 2,5-diazabicyclo[2.2.1] heptan-2-yl)methanone | RT = 5.90 Min. (HPLC Method- I). MS-ESI (m/z): Calculated [M + H]⁺ = 426.15; Observed [M + H]⁺ = 426.15. ¹H NMR (400 MHz, CDCl₃) δ 9.07-8.76 (m, 2H), 5.12 (d, J = 2.3 Hz, 1H), 4.59 (d, J = 2.4 Hz, 1H), 3.88 (d, J = 11.1 Hz, 1H), 3.74-3.55 (m, 5H), 3.45-3.14 (m, 6H), 1.97 (s, |
| | | | 1H) 1.66 (s, 1H). |
| *5 | 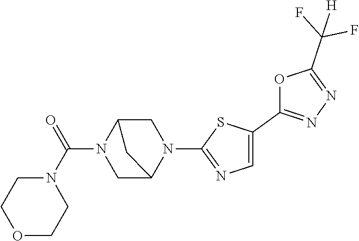 [SMILES: O=C(N1CCOCC1)N1CC2CC1CN2c1ncc(-c2nnc(C(F)F)o2)s1] | (5-(5-(5-(difluoromethyl)-1,3,4- oxadiazol-2-yl)thiazol-2-yl)-2,5- diazabicyclo[2.2.1]heptan-2- yl)(morpholino)methanone | RT = 4.89 Min. (HPLC Method- I). ¹H NMR (400 MHz, CDCl₃) δ 7.90 (s, 1H), 6.85 (t, J = 51.7 Hz, 1H), 4.80 (s, 1H), 4.60 (s, 1H), 3.72 (d, J = 9.5 Hz, 1H), 3.65 (dt, J = 5.9, 3.6 Hz, 4H), 3.59 (ddd, J = 9.6, 7.7, 2.1 Hz, 2H), 3.42 (d, J = 9.4 Hz, 1H), 3.34 (ddd, J = 13.3, 5.9, 3.8 Hz, 2H), 3.24 (ddd, J = 13.4, 6.1, 3.8 Hz, 2H), 2.12- 1.76 (m, 3H). |
| 96 | 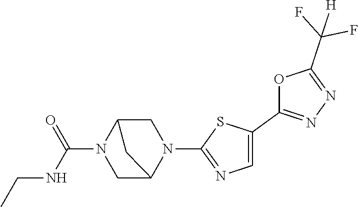 [SMILES: CCNC(=O)N1CC2CC1CN2c1ncc(-c2nnc(C(F)F)o2)s1] | 5-(5-(5-(difluoromethyl)-1,3,4- oxadiazol-2-yl)thiazol-2-yl)-N- ethyl-2,5-diazabicyclo[2.2.1] heptane-2-carboxamide | ¹H NMR (400 MHz, CDCl₃) δ 7.90 (s, 1H), 6.84 (t, J = 51.7 Hz, 1H), 4.84 (s, 2H), 4.44 (s, 1H), 4.23 (t, J = 5.5 Hz, 1H), 3.62 (dd, J = 9.3, 1.8 Hz, 1H), 3.54-3.47 (m, 1H), 3.47-3.43 (m, 2H), 3.34-3.07 (m, 4H), 2.11-1.97 (m, 2H), 1.91 (s, 1H), 1.11 (t, J = 7.2 Hz, 6H). |
| 97 | 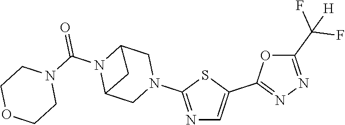 [SMILES: O=C(N1CCOCC1)N1C2CC1CN(c1ncc(-c3nnc(C(F)F)o3)s1)C2] | (3-(5-(5-(difluoromethyl)-1,3,4- oxadiazol-2-yl)thiazol-2-yl)-3,6- diazabicyclo[3.1.1]heptan-6- yl)(morpholino)methanone | RT = 4.91 Min. (HPLC Method- I). ¹H NMR (400 MHz, CDCl₃) δ 7.97 (s, 1H), 6.85 (t, J = 51.7 Hz, 1H), 4.36 (d, J = 6.3 Hz, 2H), 4.26-4.13 (m, 2H), 3.66 (dt, J = 7.2, 4.7 Hz, 10H), 3.36 (t, J = 4.8 Hz, 4H), 3.30-3.19 (m, 4H), 2.82 (dt, J = 9.1, 6.7 Hz, 1H), |
| | | | 1.65 (d, J = 9.0 Hz, 1H), 1.47 (d, |
| | | | J = 4.1 Hz, 0H), 1.43 (d, J = 7.1 |
| | | | Hz, 1H), 1.38 (d, J = 6.7 Hz, 1H), |
| | | | 1.24 (s, 1H). |
| 98 | 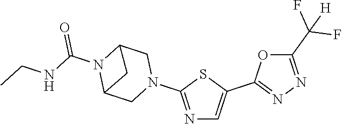 [SMILES: CCNC(=O)N1C2CC1CN(c1ncc(-c3nnc(C(F)F)o3)s1)C2] | 3-(5-(5-(difluoromethyl)-1,3,4- oxadiazol-2-yl)thiazol-2-yl)-N- ethyl-3,6-diazabicyclo[3.1.1] heptane-6-carboxamide | RT = 4.72 Min. (HPLC Method- I). ¹H NMR (400 MHz, CDCl₃) δ 7.96 (s, 1H), 6.85 (t, J = 51.7 Hz, 1H), 4.66 (t, J = 5.7 Hz, 1H), 4.54 (s, 2H), 4.34 (d, J = 6.2 Hz, 2H), 4.20 (d, J = 11.6 Hz, 2H), 3.54 (s, 2H), 3.28-3.07 (m, 8H), 2.77 (q, J = 6.8 Hz, 1H), 1.93 (s, |
| | | | 2H), 1.60 (d, J = 9.0 Hz, 1H), |
| | | | 1.11 (t, J = 7.2 Hz, 8H), 1.07 (t, |
| | | | J = 7.2 Hz, 4H). |
| 99 | 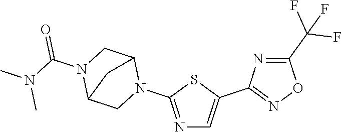 [SMILES: CN(C)C(=O)N1CC2CC1CN2c1ncc(-c2noc(C(F)(F)F)n2)s1] | N,N-dimethyl-5-(5-(5- (trifluoromethyl)-1,2,4- oxadiazol-3-yl)thiazol-2-yl)-2,5- diazabicyclo[2.2.1]heptane-2- carboxamide | RT = 6.00 Min. (HPLC Method- I). Q-TOF LC/MS (m/z): Calculated [M + H]⁺ = 388.0929; Observed [M + H]⁺ = 389.1185. ¹H NMR (400 MHz, CDCl₃) δ 7.96 (s, 1H), 4.80 (s, 1H), 4.64-4.54 (m, 1H), 3.74 (d, J = 9.4 Hz, 1H), 3.59 (ddd, J = 9.5, 6.1, 2.1 Hz, |
| | | | 2H), 3.42 (dd, J = 9.4, 1.1 Hz, |
| | | | 1H), 2.83 (s, 7H), 2.01 (dp, J = |
| | | | 2.5, 1.3 Hz, 2H). |
| 100 | 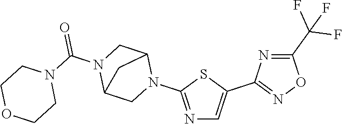 [SMILES: O=C(N1CCOCC1)N1CC2CC1CN2c1ncc(-c2noc(C(F)(F)F)n2)s1] | morpholino (5-(5-(5- (trifluoromethyl)-1,2,4-oxadiazol- 3-yl)thiazol-2-yl)-2,5- diazabicyclo[2.2.1]heptan-2-yl) methanone | RT = 5.91 Min. (HPLC Method- I). ¹H NMR (400 MHz, CDCl₃) δ 7.95 (s, 1H), 4.79 (s, 1H), 4.62- 4.55 (m, 1H), 3.69-3.64 (m, 10H), 3.62-3.54 (m, 2H), 3.43 (dd, J = 9.4, 1.4 Hz, 1H), 3.34 (ddd, J = 13.4, 5.8, 3.7 Hz, 2H), 3.26 (dd, J = 5.5, 4.1 Hz, 9H), |
| | | | 3.23 (s, 1H), 2.10-1.86 (m, 3H). |
| 101 | 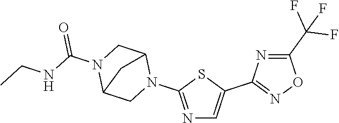 [SMILES: CCNC(=O)N1CC2CC1CN2c1ncc(-c2noc(C(F)(F)F)n2)s1] | N-ethyl-5-(5-(5- (trifluoromethyl)-1,2,4-oxadiazol- 3-yl)thiazol-2-yl)-2,5- diazabicyclo[2.2.1]heptane-2- carboxamide | RT = 5.78 Min. (HPLC Method- I). Q-TOF LC/MS (m/z): Calculated [M + H]⁺ = 388.0929; Observed [M + H]⁺ = 389.1058. ¹H NMR (400 MHz, CDCl₃) δ 7.96 (s, 1H), 4.84 (s, 2H), 4.15 (s, 1H), 3.62 (dd, J = 9.2, 1.8 Hz, 1H), 3.53-3.47 (m, 1H), 3.46-3.43 |
| | | | (m, 2H), 3.28-3.24 (m, 2H), |
| | | | 3.19 (q, J = 7.2 Hz, 3H), 2.17- |
| | | | 1.89 (m, 3H), 1.13 (td, J = 7.2, |
| | | | 5.0 Hz, 7H). |
| 102 | 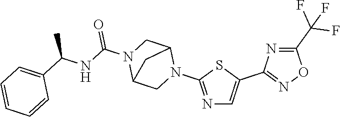 [SMILES: C[C@@H](NC(=O)N1CC2CC1CN2c1ncc(-c2noc(C(F)(F)F)n2)s1)c1ccccc1] | N-((R)-1-phenylethyl)-5-(5-(5- (trifluoromethyl)-1,2,4-oxadiazol- 3-yl)thiazol-2-yl)-2,5- diazabicyclo[2.2.1]heptane-2- carboxamide | RT = 6.49 Min. (HPLC Method- I). Q-TOF LC/MS (m/z): Calculated [M + H]⁺ = 464.1242; Observed [M + H]⁺ = 465.1370. ¹H NMR (400 MHz, CDCl₃) δ 7.95 (s, 1H), 7.38-7.17 (m, 8H), 4.99 (p, J = 7.0 Hz, 1H), 4.84 (s, 2H), 4.76 (s, |
| | | | 1H), 4.37 (d, J = 7.5 Hz, 1H), |
| | | | 3.60 (dd, J = 9.3, 1.8 Hz, 1H), |
| | | | 3.53-3.38 (m, 3H), 2.04 (td, J = |
| | | | 2.9, 1.4 Hz, 2H), 1.48 (d, J = 6.9 |
| | | | Hz, 4H), 1.39 (d, J = 6.6 Hz, 1H). |
| 103 | 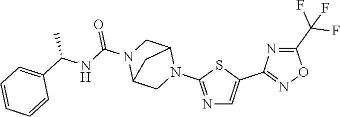 [SMILES: C[C@H](NC(=O)N1CC2CC1CN2c1ncc(-c2noc(C(F)(F)F)n2)s1)c1ccccc1] | N-((S)-1-phenylethyl)-5-(5-(5- (trifluoromethyl)-1,2,4- oxadiazol-3-yl)thiazol-2-yl)-2,5- diazabicyclo[2.2.1]heptane-2- carboxamide | RT = 6.52 Min. (HPLC Method- I). Q-TOF LC/MS (m/z): Calculated [M + H]⁺ = 464.1242; Observed [M + H]⁺ = 465.1370. ¹H NMR (400 MHz, CDCl₃) δ 7.97 (s, 1H), 7.41-7.20 (m, 11H), 4.98 (q, J = 7.0 Hz, 1H), 4.84 (s, 2H), 4.35 (d, J = 7.5 Hz, 1H), |
| | | | 3.62 (dd, J = 9.2, 1.8 Hz, 1H), |
| | | | 3.52 (d, J = 9.3 Hz, 1H), 3.47 (s, |
| | | | 3H), 2.02 (q, J = 9.9 Hz, 3H), |
| | | | 1.70 (s, 4H), 1.47 (d, J = 6.9 Hz, |
| | | | 4H), 1.40 (d, J = 6.8 Hz, 1H). |
| 104 | 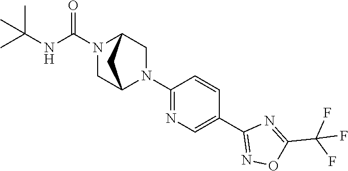 [SMILES: CC(C)(C)NC(=O)N1C[C@@H]2C[C@H]1CN2c1ccc(-c2noc(C(F)(F)F)n2)cn1] | (1S,4S)-N-(tert- butyl)-5-(5-(5-(trifluoromethyl)- 1,2,4-oxadiazol-3-yl)pyridin-2- yl)-2,5-diazabicyclo[2.2.1] heptane-2-carboxamide | RT = 5.23 Min. (HPLC Method- I). Q-TOF LC/MS (m/z): Calculated [M + H]⁺ = 4101678; Observed [M + H]⁺ = 411.1897. ¹H NMR (400 MHz, CDCl₃) δ 8.83 (dd, J = 2.3, 0.8 Hz, 1H), 8.06 (dd, J = 8.8, 2.3 Hz, 1H), 6.39 (d, J = 8.9 Hz, 1H), 5.11 (s, 1H), 4.80 (s, 1H), 3.96 (s, 1H), 3.56 (dd, J = 9.3, 1.9 Hz, 1H), 3.29 (d, J = 8.2 Hz, 1H), 2.83 (d, J = 0.8 |
| | | | Hz, 1H), 1.96 (s, 2H), 1.31 (s, |
| | | | 9H). |
| 105 | 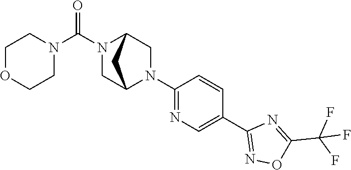 [SMILES: O=C(N1CCOCC1)N1C[C@@H]2C[C@H]1CN2c1ccc(-c2noc(C(F)(F)F)n2)cn1] | morpholino ((1S,4S)- 5-(5-(5-(trifluoromethyl)-1,2,4- oxadiazol-3-yl)pyridin-2-yl)-2,5- diazabicyclo[2.2.1]heptan-2-yl) methanone Chemical Formula: C18H19F3N6O3 Molecular Weight: 424.38 | MS-ESI (m/z): Calculated [M + H]⁺ = 4241471; Observed [M + H]⁺ = 425.1625. ¹H NMR (400 MHz, CDCl₃) δ 8.81 (d, J = 2.3 Hz, 1H), 8.05 (dd, J = 8.9, 2.3 Hz, 1H), 6.38 (d, J = 8.9 Hz, 1H), 5.07 (s, 1H), 4.63-4.42 (m, 1H), 3.75-3.52 (m, 11H), 3.42-3.28 (m, 4H), 3.28-3.12 (m, 5H), 2.83 (s, 1H), 2.36 (t, J = 8.1 Hz, 1H), 2.06-1.98 (m, 1H), 1.95 (s, 2H). |
| 106 | 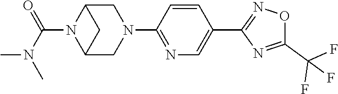 [SMILES: CN(C)C(=O)N1C2CC1CN(c1ccc(-c3noc(C(F)(F)F)n3)cn1)C2] | N,N-dimethyl-3-(5-(5- (trifluoromethyl)-1,2,4-oxadiazol- 3-yl)pyridin-2-yl)-3,6-diazabicyclo [3.1.1]heptane-6-carboxamide | RT = 5.36 Min. (HPLC Method- I). ¹H NMR (400 MHz, CDCl₃) δ 8.88 (dd, J = 2.4, 0.7 Hz, 1H), 8.10 (dd, J = 9.0, 2.4 Hz, 1H), 6.60 (dd, J = 9.0, 0.9 Hz, 1H), 4.39 (d, J = 6.2 Hz, 2H), 4.20- |
| | | | 4.03 (m, 2H), 3.81-3.40 (m, |
| | | | 2H), 2.87 (s, 6H), 2.83-2.65 (m, |
| | | | 1H), 1.57 (d, J = 8.6 Hz, 1H). |
| 107 | 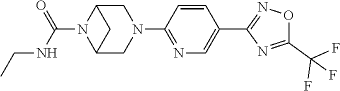 [SMILES: CCNC(=O)N1C2CC1CN(c1ccc(-c3noc(C(F)(F)F)n3)cn1)C2] | N-ethyl-3-(5-(5- (trifluoromethyl)-1,2,4-oxadiazol- 3-yl)pyridin-2-yl)-3,6-diazabicyclo [3.1.1]heptane-6-carboxamide | RT = 4.94 Min. (HPLC Method- I). ¹H NMR (400 MHz, CDCl₃) δ 8.89 (d, J = 2.3 Hz, 1H), 8.11 (dd, J = 9.0, 2.4 Hz, 1H), 6.60 (d, J = 9.0 Hz, 1H), 4.35 (dd, J = 15.8, 6.0 Hz, 3H), 4.18 (d, J = |
| | | | 11.9 Hz, 2H), 3.30-3.10 (m, |
| | | | 2H), 2.72 (q, J = 6.9 Hz, 1H), |
| | | | 1.56 (d, J = 8.7 Hz, 1H), 1.15- |
| | | | 0.97 (m, 4H) (FIG. S6a), and |
| | | | ¹⁹F NMR (376 MHz, CDCl₃) δ |
| | | | −65.42. |
| 108 | 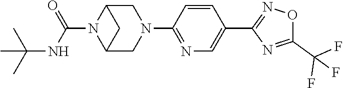 [SMILES: CC(C)(C)NC(=O)N1C2CC1CN(c1ccc(-c3noc(C(F)(F)F)n3)cn1)C2] | N-(tert-butyl)-3-(5-(5- (trifluoromethyl)-1,2,4-oxadiazol- 3-yl)pyridin-2-yl)-3,6- diazabicyclo[3.1.1]heptane-6- carboxamide | RT = 5.58 Min. (HPLC Method- I). ¹H NMR (400 MHz, CDCl₃) δ 8.89 (dd, J = 2.4, 0.8 Hz, 1H), 8.11 (dd, J = 9.1, 2.4 Hz, 1H), 6.61 (dd, J = 9.0, 0.8 Hz, 1H), 4.36-4.23 (m, 3H), 4.16 (s, 2H), |
| | | | 2.69 (t, J = 7.1 Hz, 1H), 1.52 (d, |
| | | | J = 8.7 Hz, 1H), 1.23 (s, 9H). |
| 109 | 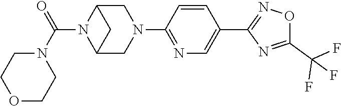 [SMILES: O=C(N1CCOCC1)N1C2CC1CN(c1ccc(-c3noc(C(F)(F)F)n3)cn1)C2] | morpholino (3-(5-(5- (trifluoromethyl)-1,2,4- oxadiazol-3-yl)pyridin-2-yl)-3,6- diazabicyclo[3.1.1]heptan-6- yl)methanone | RT = 4.81 Min. (HPLC Method- I). Q-TOF LC/MS (m/z): Calculated [M + H]⁺ = 41016780; Observed [M + H]⁺ = 411.1777. ¹H NMR (400 MHz, CDCl₃) δ 8.89 (d, J = 2.3 Hz, 1H), 8.11 (dd, J = 9.0, 2.4 Hz, 1H), 6.60 (d, J = 9.0 |
| | | | Hz, 1H), 4.38 (d, J = 6.2 Hz, 2H), |
| | | | 4.17 (d, J = 11.9 Hz, 2H), 3.82- |
| | | | 3.54 (m, 6H), 3.36 (t, J = 4.8 Hz, |
| | | | 4H), 2.77 (q, J = 6.9 Hz, 1H), |
| | | | 1.61 (d, J = 8.7 Hz, 1H), 1.24 (s, |
| | | | 1H) (FIG. S5a), and ¹⁹F NMR |
| | | | (376 MHz, _{CDCL3}) δ −65.42. |
| 110 | 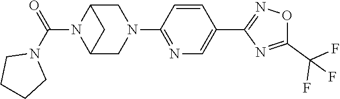 [SMILES: O=C(N1CCCC1)N1C2CC1CN(c1ccc(-c3noc(C(F)(F)F)n3)cn1)C2] | pyrrolidin-1-yl (3-(5- (5-(trifluoromethyl)-1,2,4- oxadiazol-3-yl)pyridin-2-yl)-3,6- diazabicyclo[3.1.1]heptan-6-yl) methanone | RT = 5.17 Min. (HPLC Method- I). Q-TOF LC/MS (m/z): Calculated [M + H]⁺ = 408.1522; Observed [M + H]⁺ = 409.1616. ¹H NMR (400 MHz, CDCl₃) δ 8.88 (dd, J = 2.4, 0.8 Hz, 1H), 8.10 (dd, J = 9.0, 2.4 Hz, 1H), 6.60 |
| | | | (dd, J = 9.1, 0.8 Hz, 1H), 4.40 (d, |
| | | | J = 6.2 Hz, 2H), 4.16 (s, 2H), |
| | | | 3.35 (dt, J = 11.8, 6.7 Hz, 5H), |
| | | | 2.77-2.66 (m, 1H), 1.85 (d, J = |
| | | | 6.2 Hz, 5H), 1.57 (d, J = 8.6 Hz, |
| | | | 1H). |
| 111 | 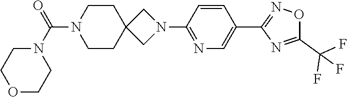 [SMILES: O=C(N1CCOCC1)N1CCC2(CC1)CN(c1ccc(-c3noc(C(F)(F)F)n3)cn1)C2] | morpholino (2-(5-(5- (trifluoromethyl)-1,2,4- oxadiazol-3-yl)pyridin-2-yl)-2,7- diazaspiro[3.5]nonan-7-yl) methanone | RT = 4.80 Min. (HPLC Method- I). ¹H NMR (400 MHz, CDCl₃) δ 8.82 (d, J = 2.2 Hz, 1H), 8.04 (dd, J = 8.8, 2.3 Hz, 1H), 6.31 (d, J = 8.8 Hz, 1H), 5.28 (s, 1H), 3.85 (s, 4H), 3.66 (t, J = 4.7 Hz, 5H), 3.24 (dt, J = 11.7, 5.2 Hz, |
| | | | 9H), 1.84 (t, J = 5.5 Hz, 4H). |
| 112 | 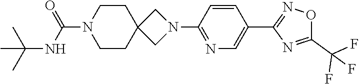 [SMILES: CC(C)(C)NC(=O)N1CCC2(CC1)CN(c1ccc(-c3noc(C(F)(F)F)n3)cn1)C2] | N-(tert-butyl)-2-(5- (5-(trifluoromethyl)-1,2,4- oxadiazol-3-yl)pyridin-2-yl)-2,7- diazaspiro[3.5]nonane-7- carboxamide | RT = 5.19 Min. (HPLC Method- I). ¹H NMR (400 MHz, CDCl₃) δ 8.83 (dd, J = 2.3, 0.8 Hz, 1H), 8.05 (dd, J = 8.8, 2.3 Hz, 1H), 6.31 (dd, J = 8.8, 0.9 Hz, 1H), 5.29 (s, 2H), 4.34 (s, 1H), 3.86 (s, |
| | | | 4H), 3.37-3.22 (m, 4H), 1.93- |
| | | | 1.75 (m, 4H), 1.34 (s, 9H). |
| 113 | 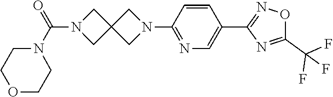 [SMILES: O=C(N1CCOCC1)N1CC2(C1)CN(c1ccc(-c3noc(C(F)(F)F)n3)cn1)C2] | morpholino (6-(5-(5- (trifluoromethyl)-1,2,4-oxadiazol- 3-yl)pyridin-2-yl)-2,6-diazaspiro [3.3]heptan-2-yl)methanone | RT = 4.61 Min. (HPLC Method- I). ¹H NMR (400 MHz, CDCl₃) δ 8.84 (d, J = 2.3 Hz, 1H), 8.07 (dd, J = 8.7, 2.3 Hz, 1H), 6.33 (d, J = 8.8 Hz, 1H), 4.21 (d, J = 21.6 Hz, 8H), 3.76-3.56 (m, 4H), 3.34 (t, J = 4.7 Hz, 4H). |
| 114 | 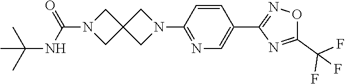 [SMILES: CC(C)(C)NC(=O)N1CC2(C1)CN(c1ccc(-c3noc(C(F)(F)F)n3)cn1)C2] | N-(tert-butyl)-6-(5-(5- (trifluoromethyl)-1,2,4-oxadiazol- 3-yl)pyridin-2-yl)-2,6-diazaspiro [3.3]heptane-2-carboxamide | RT = 4.65 Min. (HPLC Method- I). ¹H NMR (400 MHz, CDCl₃) δ 8.83 (dd, J = 2.3, 0.8 Hz, 1H), 8.06 (dd, J = 8.8, 2.3 Hz, 1H), 6.34 (d, J = 0.8 Hz, 1H), 4.23 (s, 0H), 4.09 (s, 4H), 3.94 (s, 4H), |
| | | | 1.33 (s, 9H). |

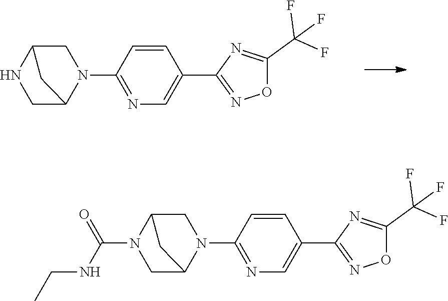 [SMILES: FC(F)(F)c1nc(-c2ccc(N3CC4CC3CN4)nc2)no1 ; CCNC(=O)N1CC2CC1CN2c1ccc(-c2noc(C(F)(F)F)n2)cn1]

### Compound of Invention—84, Example-84: N-ethyl-5-(5-(5-(trifluoromethyl)-1,2,4-oxadiazol-3-yl)pyridin-2-yl)-2,5-diazabicyclo[2.2.1]heptane-2-carboxamide (Method-IV)

To a stirred solution of compound 5-(5-(5-(trifluoromethyl)-1,2,4-oxadiazol-3-yl)pyridin-2-yl)-2,5-diazabicyclo[2.2.1]heptan-2-ium trifluoroacetate (the intermediate 5), (0.052 g, 0.096 mmol) and DIPEA (0.086 mL, 0.062 g, 0.048 mmol) in DCM (3 mL) was added isocyanatoethane (0.007 g, 0.096 mmol) at room temperature. After stirring 2 h, the reaction was quenched with water and the product was extracted using DCM. Purification on a silica gel preparatory glass TLC plate using 50% EA/Hex followed by extraction afforded the title compound Example-84 as a white solid (0.025 g, 66%). RT=4.69 Min. (HPLC Method-I). MS-ESI (m/z): Calculated [M+H]⁺=383.14; Observed [M+H]⁺=383.14.

### Compound of Invention—85, Example-85: N,N-dimethyl-5-(5-(5-(trifluoromethyl)-1,2,4-oxadiazol-3-yl)pyridin-2-yl)-2,5-diazabicyclo[2.2.1]heptane-2-carboxamide (Method-V)

To a stirred solution of compound 5-(5-(5-(trifluoromethyl)-1,2,4-oxadiazol-3-yl)pyridin-2-yl)-2,5-diazabicyclo[2.2.1]heptan-2-ium trifluoroacetate (the intermediate 5) (0.052 g, 0.096 mmol) and DIPEA (0.086 mL, 0.062 g, 0.048 mmol) in DCM (3 mL) was added dimethylcarbamic chloride (0.011 g, 0.096 mmol) at room temperature. After stirring 2 h, the reaction was quenched with water and the product was extracted using DCM. Purification on a silica gel preparatory glass TLC plate using 50% EA/Hex followed by extraction afforded the title compound Example-85 as a white solid (0.030 g, 62%). TLC: Tr=0.40 in 50% EA/Hex. RT=4.89 Min. (HPLC Method-I). MS-ESI (m/z): Calculated [M+H]⁺=383.14; Observed [M+H]⁺=383.14.

### Compound of Invention—86, Example 86: Morpholino (5-(5-(5-(trifluoromethyl)-1,2,4-oxadiazol-3-yl)pyridin-2-yl)-2,5-diazabicyclo[2.2.1]heptan-2-yl)methanone

Example-86 was synthesized in an analogous manner to Example-85 by following method-V using he intermediate 5. Purification on a silica gel preparatory glass TLC plate using 50% EA/Hex followed by extraction afforded the title compound Example-86 as a white solid (0.028 g, 68%). RT=4.85 Min. (HPLC Method-I). MS-ESI (m/z): Calculated [M+H]⁺=425.15; Observed [M+H]⁺=425.15.

### Compound of Invention—87, Example-87: N-(naphthalen-1-yl)-5-(5-(5-(trifluoromethyl)-1,2,4-oxadiazol-3-yl)pyridin-2-yl)-2,5-diazabicyclo[2.2.1]heptane-2-carboxamide

Example-87 was synthesized in an analogous manner to Example-84 by following method-IV using he intermediate 5. Purification on a silica gel preparatory glass TLC plate using 50% EA/Hex followed by extraction afforded the title compound Example-87 as a white solid (0.030 g, 60%). RT=5.82 min (HPLC Method-I). MS-ESI (m/z): Calculated [M+H]⁺=480.15; Observed [M+H]⁺=480.15. ¹H NMR (400 MHZ, CDCl₃) δ 8.85 (dd, J=2.3, 0.7 Hz, 1H), 8.09 (dd, J=8.9, 2.3 Hz, 1H), 7.88-7.82 (m, 1H), 7.80-7.73 (m, 2H), 7.64 (d, J=8.2 Hz, 1H), 7.51-7.42 (m, 3H), 6.51 (s, 1H), 6.40 (d, J=8.9 Hz, 1H), 5.20 (s, 1H), 4.91 (s, 1H), 3.68 (dd, J=8.2, 2.0 Hz, 1H), 3.62-3.43 (m, 3H), 2.11-1.92 (m, 2H).

### Compound of Invention—88, Example-88: N-([1,1′-biphenyl]-2-yl)-5-(5-(5-(trifluoromethyl)-1,2,4-oxadiazol-3-yl)pyridin-2-yl)-2,5-diazabicyclo[2.2.1]heptane-2-carboxamide

Example-88 was synthesized in an analogous manner to Example-84 by following method-IV using the intermediate 5. Purification on a silica gel preparatory glass TLC plate using 50% EA/Hex followed by extraction afforded the title compound Example-88 as a white solid (0.034 g, 63%). RT=6.12 Min. (HPLC Method-I). Calculated [M+H]⁺=507.17; Observed [M+H]⁺=507.17.

### Compound of Invention—89, Example-89 N-benzyl-5-(5-(5-(trifluoromethyl)-1,2,4-oxadiazol-3-yl)pyridin-2-yl)-2,5-diazabicyclo[2.2.1]heptane-2-carboxamide VMCC-MP-03-013-001

Example-89 was synthesized in an analogous manner to Example-84 by following method-IV using the intermediate 5. Purification on a silica gel preparatory glass TLC plate using 50% EA/Hex followed by extraction afforded the title compound Example-89 as a white solid (10 mg, 22%). RT=5.88 Min. (HPLC Method-I). ESI (m/z): Calculated [M+H]⁺=445.14; Observed [M+H]⁺=446.14.

### Compound of Invention—90, Example-90: N—((R)-1-phenylethyl)-5-(5-(5-(trifluoromethyl)-1,2,4-oxadiazol-3-yl)pyridin-2-yl)-2,5-diazabicyclo[2.2.1]heptane-2-carboxamide

Example-90 was synthesized in an analogous manner to Example-84 by following method-IV using the intermediate 5. Purification on a silica gel preparatory glass TLC plate using 50% EA/Hex followed by extraction afforded the title compound Example-90 as a white solid (10 mg, 20%). RT=5.67 Min. (HPLC Method-I). MS-ESI (m/z): Calculated [M+H]⁺=459.17; Observed [M+H]⁺=459.17.

### Compound of Invention—91, Example-91: N—((S)-1-phenylethyl)-5-(5-(5-(trifluoromethyl)-1,2,4-oxadiazol-3-yl)pyridin-2-yl)-2,5-diazabicyclo[2.2.1]heptane-2-carboxamide

Example-91 was synthesized in an analogous manner to Example-84 by following method-IV using the intermediate 5. Purification on a silica gel preparatory glass TLC plate using 50% EA/Hex followed by extraction afforded the title compound Example-91 as a white solid (30 mg, 64%) was obtained as a white solid. TLC: Tr=0.47 in 50% EA/Hex. RT=5.65 Min. (HPLC Method-I). MS-ESI (m/z): Calculated [M+H]⁺=459.17; Observed [M+H]⁺=459.17.

### Compound of Invention—92, Example-92: N-(2-methoxyphenyl)-5-(5-(5-(trifluoromethyl)-1,2,4-oxadiazol-3-yl)pyridin-2-yl)-2,5-diazabicyclo[2.2.1]heptane-2-carboxamide

Example-92 was synthesized in an analogous manner to Example-84 by following method-IV using the intermediate 5. Purification on a silica gel preparatory glass TLC plate using 50% EA/Hex followed by extraction afforded the title compound Example-92 as a white solid (20 mg, 45%). RT=5.68 Min. (HPLC Method-I). MS-ESI (m/z): Calculated [M+H]⁺=460.15; Observed [M+H]⁺=460.15.

### Compound of Invention—93, Example-93: N-(tert-butyl)-5-(5-(5-(trifluoromethyl)-1,2,4-oxadiazol-3-yl)pyridin-2-yl)-2,5-diazabicyclo[2.2.1]heptane-2-carboxamide

Example-93 was synthesized in an analogous manner to Example-84 by following method-IV using the intermediate 5. Purification on a silica gel preparatory glass TLC plate using 50% EA/Hex followed by extraction afforded the title compound Example-93 as a white solid. (30 mg, 70%). RT=5.32 Min. (HPLC Method-I). MS-ESI (m/z): Calculated [M+H]⁺=411.17; Observed [M+H]⁺=411.17.

### Compound of Invention—94, Example-94: Morpholino (5-(5-(5-(trifluoromethyl)-1,2,4-oxadiazol-3-yl)pyrimidin-2-yl)-2,5-diazabicyclo[2.2.1]heptan-2-yl)methanone

Example-94 was synthesized in an analogous manner to Example-85 by following method-V using the intermediate 21a. Purification on a silica gel preparatory glass TLC plate using 50% EA/Hex followed by extraction afforded the title compound Example-94 as a tan color solid (20 mg, 58%). RT=5.90 Min. (HPLC Method-I).

### Compound of Invention—95, Example-95: (5-(5-(5-(Difluoromethyl)-1,3,4-oxadiazol-2-yl)thiazol-2-yl)-2,5-diazabicyclo[2.2.1]heptan-2-yl) (morpholino) methanone

Example-95 was synthesized in an analogous manner to Example-85 by following method-V using the intermediate 35. Purification on a silica gel preparatory glass TLC plate using 50% EA/Hex followed by extraction afforded the title compound Example-95 as a brown color solid. (33 mg, 59%). RT=4.89 Min. (HPLC Method-I).

### Compound of Invention—96, Example-96:5-(5-(5-(Difluoromethyl)-1,3,4-oxadiazol-2-yl)thiazol-2-yl)-N-ethyl-2,5-diazabicyclo[2.2.1]heptane-2-carboxamide

Example-96 was synthesized in an analogous manner to Example-84 by following method-IV using the intermediate 35. Purification on a silica gel preparatory glass TLC plate using 60% EA/Hex followed by extraction afforded the title compound Example-96 as a brown color solid. (25 mg, 50%). RT=4.73 Min. (HPLC Method-I).

### Compound of Invention—97, Example-97: (3-(5-(5-(difluoromethyl)-1,3,4-oxadiazol-2-yl)thiazol-2-yl)-3,6-diazabicyclo[3.1.1]heptan-6-yl) (morpholino) methanone

Example-97 was synthesized in an analogous manner to Example-85 by following method-V using the intermediate 35. Purification on a silica gel preparatory glass TLC plate using 60% EA/Hex followed by extraction afforded the title compound Example-97 as a tan color solid (40 mg, 54%). RT=4.91 Min. (HPLC Method-I).

### Compound of Invention—98, Example-98:3-(5-(5-(difluoromethyl)-1,3,4-oxadiazol-2-yl)thiazol-2-yl)-N-ethyl-3,6-diazabicyclo[3.1.1]heptane-6-carboxamide

Example-98 was synthesized in an analogous manner to Example-84 by following method-IV using the intermediate 35. Purification on a silica gel preparatory glass TLC plate using 60% EA/Hex followed by extraction afforded the title compound Example-98 as a tan color solid (40 mg, 56%). RT=4.72 Min. (HPLC Method-I).

### Compound of Invention—99, Example-99: N,N-dimethyl-5-(5-(5-(trifluoromethyl)-1,2,4-oxadiazol-3-yl)thiazol-2-yl)-2,5-diazabicyclo[2.2.1]heptane-2-carboxamide

Example-99 was synthesized in an analogous manner to Example 85 by following method-V using the intermediate 31. Purification on a silica gel preparatory glass TLC plate using 60% EA/Hex followed by extraction afforded the title compound Example-99as a tan color solid (32 mg, 51%). RT=6.00 Min. (HPLC Method-I). Q-TOF LC/MS (m/z): Calculated [M+H]⁺=388.0929; Observed [M+H]⁺=389.1185.

### Compound of Invention—100, Example-100: Morpholino (5-(5-(5-(trifluoromethyl)-1,2,4-oxadiazol-3-yl)thiazol-2-yl)-2,5-diazabicyclo[2.2.1]heptan-2-yl)methanone

Example-100 was synthesized in an analogous manner to Example-85 by following method-V using the intermediate 31. Purification on a silica gel preparatory glass TLC plate using 60% EA/Hex followed by extraction afforded the title compound Example-100 as a tan color solid (48 mg, 67%). RT=5.91 Min. (HPLC Method-I).

### Compound of Invention—101, Example-101: N-Ethyl-5-(5-(5-(trifluoromethyl)-1,2,4-oxadiazol-3-yl)thiazol-2-yl)-2,5-diazabicyclo[2.2.1]heptane-2-carboxamide

Example-101 was synthesized in an analogous manner to Example-84 by following method-IV using the intermediate 31. Purification on a silica gel preparatory glass TLC plate using 60% EA/Hex followed by extraction afforded the title compound Example-101 as a brown color solid (40 mg, 57%). RT=5.78 Min. (HPLC Method-I). Q-TOF LC/MS (m/z): Calculated [M+H]⁺=388.0929; Observed [M+H]⁺=389.1058.

### Compound of Invention—102, Example-102: N—((R)-1-Phenylethyl)-5-(5-(5-(trifluoromethyl)-1,2,4-oxadiazol-3-yl)thiazol-2-yl)-2,5-diazabicyclo[2.2.1]heptane-2-carboxamide

Example-102 was synthesized in an analogous manner to Example-84 by following method-IV using the intermediate 31. Purification on a silica gel preparatory glass TLC plate using 60% EA/Hex followed by extraction afforded the title compound Example-102 as a brown color solid (32 mg, 37%). RT=6.49 Min. (HPLC Method-I). MS-ESI (m/z): Calculated [M+H]⁺=464.1242; Observed [M+H]⁺=465.1370.

### Compound of Invention—103, Example-103: N—((S)-1-Phenylethyl)-5-(5-(5-(trifluoromethyl)-1,2,4-oxadiazol-3-yl)thiazol-2-yl)-2,5-diazabicyclo[2.2.1]heptane-2-carboxamide

Example-103 was synthesized in an analogous manner to Example-84 by following method-IV using the intermediate 31. Purification on a silica gel preparatory glass TLC plate using 60% EA/Hex followed by extraction afforded the title compound Example-103 as a brown color solid (30 mg, 41%). RT=6.52 Min. (HPLC Method-I). Q-TOF LC/MS (m/z): Calculated [M+H]⁺=464.1242; Observed [M+H]⁺=465.1370.

### Compound of Invention—104, Example-104: (1S,4S)—N-(tert-butyl)-5-(5-(5-(trifluoromethyl)-1,2,4-oxadiazol-3-yl)pyridin-2-yl)-2,5-diazabicyclo[2.2.1]heptane-2-carboxamide

Example-104 was synthesized in an analogous manner to Example-84 by following method-IV using the intermediate 24. Purification on a silica gel preparatory glass TLC plate using 70% EA/Hex followed by extraction afforded the title compound Example-104as a colorless solid (42 mg, 33%). RT=5.23 Min. (HPLC Method-I). Q-TOF LC/MS (m/z): Calculated [M+H]⁺=4101678; Observed [M+H]⁺=411.1897.

### Compound of Invention—105, Example-105: Morpholino ((1S,4S)-5-(5-(5-(trifluoromethyl)-1,2,4-oxadiazol-3-yl)pyridin-2-yl)-2,5-diazabicyclo[2.2.1]heptan-2-yl)methanone

Example-105 was synthesized in an analogous manner to Example-84 by following method-V using the intermediate 24. Purification on a silica gel preparatory glass TLC plate using 70% EA/Hex followed by extraction afforded the title compound Example-105 as a colorless solid (53 mg, 46%). TLC Rf: 70% EA/Hex. RT=5.91 Min. (HPLC Method-I). Q-TOF LC/MS (m/z): Calculated [M+H]⁺=4241471; Observed [M+H]⁺=425.1625.

### Compound of Invention—106, Example-106: N,N-dimethyl-3-(5-(5-(trifluoromethyl)-1,2,4-oxadiazol-3-yl)pyridin-2-yl)-3,6-diazabicyclo[3.1.1]heptane-6-carboxamide

Example-106 was synthesized in an analogous manner to Example-85 by following method-V using the intermediate 16b. Purification on a silica gel preparatory glass TLC plate using 70% EA/Hex followed by extraction afforded the title compound Example-106 as a colorless solid (28 mg, 34%). TLC Rf: 70% EA/Hex. RT=55.36 Min. (HPLC Method-I).

### Compound of Invention 107, Example-107: N-Ethyl-3-(5-(5-(trifluoromethyl)-1,2,4-oxadiazol-3-yl)pyridin-2-yl)-3,6-diazabicyclo[3.1.1]heptane-6-carboxamide

Example-107 was synthesized in an analogous manner to Example-84 by following method-IV using the intermediate 16b. Purification on a silica gel preparatory glass TLC plate using 80% EA/Hex followed by extraction afforded the title compound Example-107 as a colorless solid (21 mg, 40%). RT=4.94 Min. (HPLC Method-I).

### Compound of Invention—108, Example-108: N-(tert-butyl)-3-(5-(5-(trifluoromethyl)-1,2,4-oxadiazol-3-yl)pyridin-2-yl)-3,6-diazabicyclo[3.1.1]heptane-6-carboxamide

Example-108 was synthesized in an analogous manner to Example-84 by following method-IV using the intermediate 16b. Purification on a silica gel preparatory glass TLC plate using 70% EA/Hex followed by extraction afforded the title compound Example-108 as a colorless solid (63 mg, 72%). RT=5.59 Min. (HPLC Method-I).

### Compound of Invention—109, Example-109: Morpholino ((1S,4S)-5-(5-(5-(trifluoromethyl)-1,2,4-oxadiazol-3-yl)pyridin-2-yl)-2,5-diazabicyclo[2.2.1]heptan-2-yl)methanone

Example-109 was synthesized in an analogous manner to Example-85 by following method-V using the intermediate 16b. Purification on a silica gel preparatory glass TLC plate using 80% EA/Hex followed by extraction afforded the title compound Example-109 as a colorless solid (18 mg, 30%). RT=4.81 Min. (HPLC Method-I). Q-TOF LC/MS (m/z): Calculated [M+H]⁺=41016780; Observed [M+H]⁺=411.1777.

### Compound of Invention—110, Example-110: Pyrrolidin-1-yl (3-(5-(5-(trifluoromethyl)-1,2,4-oxadiazol-3-yl)pyridin-2-yl)-3,6-diazabicyclo[3.1.1]heptan-6-yl)methanone

Example-110 was synthesized in an analogous manner to Example-85 by following method-V using the intermediate 16b. Purification on a silica gel preparatory glass TLC plate using DCM and 2% methanol followed by extraction afforded the title compound Example-110 as a colorless solid (50 mg, 60%). RT=5.17 Min. (HPLC Method-I). Q-TOF LC/MS (m/z): Calculated [M+H]⁺=408.1522; Observed [M+H]⁺=409.1616.

### Compound of Invention—111, Example-111: Morpholino (2-(5-(5-(trifluoromethyl)-1,2,4-oxadiazol-3-yl)pyridin-2-yl)-2,7-diazaspiro[3.5]nonan-7-yl)methanone

Example-111 was synthesized in an analogous manner to Example 85 by following method-V using the intermediate 45. Purification on a silica gel preparatory glass TLC plate using 70% EA/Hex followed by extraction afforded the title compound Example-111 as a colorless solid (42 mg, 56%). RT=4.80 Min. (HPLC Method-I).

### Compound of Invention—112, Example-120: N-(tert-butyl)-2-(5-(5-(trifluoromethyl)-1,2,4-oxadiazol-3-yl)pyridin-2-yl)-2,7-diazaspiro[3.5]nonane-7-carboxamide

Example-112 was synthesized in an analogous manner to 84 by following method-IV using the intermediate 45. Purification on a silica gel preparatory glass TLC plate using 80% EA/Hex followed by extraction afforded the title compound Example-112 as a colorless solid (46 mg. 64%). RT=5.19 Min. (HPLC Method-I).

### Compound of Invention—113, Example-113: Morpholino (6-(5-(5-(trifluoromethyl)-1,2,4-oxadiazol-3-yl)pyridin-2-yl)-2,6-diazaspiro[3.3]heptan-2-yl)methanone

Example-113 was synthesized in an analogous manner to Example-85 by following method-V using the intermediate 47. Purification on a silica gel preparatory glass TLC plate using 70% EA/Hex followed by extraction afforded the title compound Example-113 as a colorless solid (35 mg, 47%). RT=4.62 Min. (HPLC Method-I).

### Compound of Invention—114, Example-114: N-(tert-butyl)-6-(5-(5-(trifluoromethyl)-1,2,4-oxadiazol-3-yl)pyridin-2-yl)-2,6-diazaspiro[3.3]heptane-2-carboxamide

Example-114 was synthesized in an analogous manner to Example-84 by following method-IV using the intermediate 47. Purification on a silica gel preparatory glass TLC plate using 1% methanol in DCM followed by extraction afforded the title compound Example-114 as a colorless solid (46 mg. 63%). RT=4.66 Min. (HPLC Method-I).

TABLE IV
Compounds of invention (Iaaa₅) and (Ibaa₅) Examples 115-123

| Example | Compound | Compound Name | Characterization |
|---|---|---|---|
| 115 | 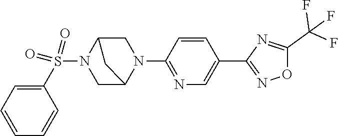 [SMILES: O=S(=O)(c1ccccc1)N1CC2CC1CN2c1ccc(-c2noc(C(F)(F)F)n2)cn1] | 3-(6-(5-(phenylsulfonyl)-2,5- diazabicyclo[2.2.1]heptan-2-yl) pyridin-3-yl)-5-(trifluoromethyl)- 1,2,4-oxadiazole | RT = 6.33 Min. (HPLC Method- I). MS-ESI (m/z): Calculated [M + H]⁺ = 452.09; Observed [M + H]⁺ = 452.09. ¹H NMR (400 MHz, CDCl₃) δ 8.80 (d, J = 2.3 Hz, 1H), 8.05 (dd, J = 8.8, 2.3 Hz, 1H), 7.96-7.80 (m, 2H), 7.70-7.54 (m, 1H), 7.54-7.45 (m, 2H), 6.30 (d, J = 8.9 Hz, 1H), |
| | | | 4.97 (s, 1H), 4.63 (s, 1H), 3.60- |
| | | | 3.44 (m, 3H), 3.34 (dd, J = 9.5, |
| | | | 2.0 Hz, 1H), 1.85 (d, J = 9.8 Hz, |
| | | | 1H), 1.45 (dd, J = 10.0, 2.4 Hz, |
| | | | 1H). |
| 116 | 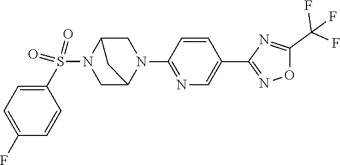 [SMILES: O=S(=O)(c1ccc(F)cc1)N1CC2CC1CN2c1ccc(-c2noc(C(F)(F)F)n2)cn1] | 3-(6-(5-((4- fluorophenyl)sulfonyl)-2,5- diazabicyclo[2.2.1]heptan-2-yl) pyridin-3-yl)-5-(trifluoromethyl)- 1,2,4-oxadiazole | RT = 6.48 Min. (HPLC Method- I). MS-ESI (m/z): Calculated [M + H]⁺ = 470.09; Observed [M + H]⁺ = 470.09. ¹H NMR (400 MHz, CDCl₃) δ 8.81 (dd, J = 2.3, 0.8 Hz, 1H), 8.07 (ddd, J = 8.9, 2.4, 0.7 Hz, 1H), 7.85 (ddd, J = 8.7, 5.1, 1.2 Hz, 2H), 7.19-7.06 (m, 2H), 6.32 (d, J = 8.9 Hz, 1H), 5.00 (s, 1H), 4.62 (s, 1H), 3.57- 3.48 (m, 3H), 3.32 (dd, J = 9.4, |
| | | | 1.9 Hz, 1H), 1.96-1.81 (m, 1H), |
| | | | 1.57-1.45 (m, 1H). |
| 117 | 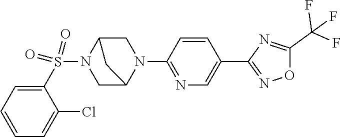 [SMILES: O=S(=O)(c1ccccc1Cl)N1CC2CC1CN2c1ccc(-c2noc(C(F)(F)F)n2)cn1] | 3-(6-(5-((2- chlorophenyl)sulfonyl)-2,5- diazabicyclo[2.2.1]heptan-2- yl)pyridin-3-yl)-5- (trifluoromethyl)-1,2,4-oxadiazole | RT = 6.66 Min. (HPLC Method- I). MS-ESI (m/z): Calculated [M + H]⁺ = 486.05; Observed [M + H]⁺ = 486.05. ¹H NMR (400 MHz, CDCl₃) δ 8.83 (dd, J = 2.3, 0.7 Hz, 1H), 8.14-7.91 (m, 2H), 7.59-7.45 (m, 2H), 7.39 (ddd, J = 7.9, 6.7, 2.0 Hz, 1H), 6.39 (d, J = 8.9 Hz, 1H), 5.10 (s, 1H), 4.81 |
| | | | (s, 1H), 3.65 (d, J = 9.6 Hz, 1H), |
| | | | 3.57 (dd, J = 9.9, 1.8 Hz, 2H), |
| | | | 3.46 (dd, J = 9.0, 2.0 Hz, 1H), |
| | | | 2.08-1.90 (m, 2H). |
| 118 | 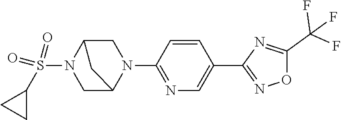 [SMILES: O=S(=O)(C1CC1)N1CC2CC1CN2c1ccc(-c2noc(C(F)(F)F)n2)cn1] | 3-(6-(5-(cyclopropylsulfonyl)- 2,5-diazabicyclo[2.2.1]heptan- 2-yl)pyridin-3-yl)-5- (trifluoromethyl)-1,2,4-oxadiazole | RT = 5.58 Min. (HPLC Method- I). MS-ESI (m/z): Calculated [M + H]⁺ = 416.09; Observed [M + H]⁺ = 416.09. ¹H NMR (400 MHz, CDCl₃) δ 8.85 (d, J = 2.3 Hz, 1H), 8.09 (dd, J = 8.8, 2.3 Hz, 1H), 6.42 (d, J = 8.9 Hz, 1H), 5.12 (s, 1H), 4.63 (s, 1H), 3.67 (d, |
| | | | J = 9.8 Hz, 1H), 3.58 (dt, J = 9.1, |
| | | | 2.1 Hz, 2H), 3.52 (d, J = 9.1 Hz, |
| | | | 1H), 2.34 (tt, J = 8.0, 4.8 Hz, 1H), |
| | | | 2.07 (q, J = 1.7 Hz, 2H), 1.35- |
| | | | 1.09 (m, 2H), 1.09-0.81 (m, 2H). |
| 119 | 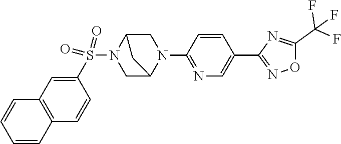 [SMILES: O=S(=O)(c1ccc2ccccc2c1)N1CC2CC1CN2c1ccc(-c2noc(C(F)(F)F)n2)cn1] | 3-(6-(5-(naphthalen-2- ylsulfonyl)-2,5-diazabicyclo [2.2.1]heptan-2-yl)pyridin-3- yl)-5-(trifluoromethyl)-1,2,4- oxadiazole | RT = 6.83 Min. (HPLC Method- I). MS-ESI (m/z): Calculated [M + H]⁺ = 502.11; Observed [M + H]⁺ = 502.11. ¹H NMR (400 MHz, CDCl₃) δ 8.70 (d, J = 2.3 Hz, 1H), 8.35 (d, J = 1.8 Hz, 1H), 7.97-7.85 (m, 4H), 7.81 (dd, J = 8.6, 1.9 Hz, 1H), 7.62 (ddd, J = 8.3, 6.8, 1.3 Hz, 1H), 7.55 (ddd, |
| | | | J = 8.1, 6.8, 1.3 Hz, 1H), 6.18 (d, |
| | | | J = 8.8 Hz, 1H), 4.91 (s, 1H), 4.70 |
| | | | (s, 1H), 3.63-3.54 (m, 1H), 3.53- |
| | | | 3.45 (m, 2H), 1.86 (d, J = 10.1 |
| | | | Hz, 1H), 1.65-1.46 (m, 1H). |
| 120 | 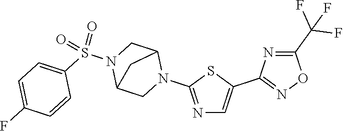 [SMILES: O=S(=O)(c1ccc(F)cc1)N1CC2CC1CN2c1ncc(-c2noc(C(F)(F)F)n2)s1] | 3-(2-(5-((4-fluorophenyl)sulfonyl)- 2,5-diazabicyclo[2.2.1]heptan-2- yl)thiazol-5-yl)-5-(trifluoromethyl)- 1,2,4-oxadiazole | RT = 6.87 Min. (HPLC Method- I). ¹H NMR (400 MHz, CDCl₃) δ 7.94 (s, 1H), 7.89-7.72 (m, 2H), 7.23-7.17 (m, 2H), 4.73 (s, 1H), 4.65 (td, J = 2.2, 1.0 Hz, 1H), 3.63-3.59 (m, 1H), 3.57 (dd, J = 9.6, 2.0 Hz, 1H), 3.51 (d, J = 10.0 Hz, 1H), 3.34 (dd, J = 9.7, 1.9 Hz, 1H), 2.03-1.89 (m, |
| | | | 1H), 1.77 (s, 1H), 1.59 (ddt, J = |
| | | | 10.4, 2.6, 1.2 Hz, 1H). |
| 121 | 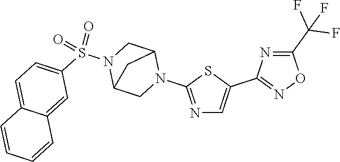 [SMILES: O=S(=O)(c1ccc2ccccc2c1)N1CC2CC1CN2c1ncc(-c2noc(C(F)(F)F)n2)s1] | 3-(2-(5-(naphthalen-2ylsulfonyl)- 2,5-diazabicyclo[2.2.1]heptan-2- yl)thiazol-5-yl)-5- (trifluoromethyl)-1,2,4-oxadiazole | RT = 7.245 Min. (HPLC Method- I). Q-TOF LC/MS (m/z): Calculated [M + H]⁺ = 507.599; Observed [M + H]⁺ = 508.0757. ¹H NMR (400 MHz, CDCl₃) δ 8.37 (d, J = 1.9 Hz, 1H), 7.98 (d, J = 8.7 Hz, 1H), 7.95-7.86 (m, 2H), 7.83 (d, J = 1.3 Hz, 1H), 7.81 (d, J = 1.9 Hz, 0H), 7.63-7.54 (m, 2H), 4.75-4.61 (m, 2H), 3.73- 3.64 (m, 1H), 3.57-3.45 (m, |
| | | | 2H), 3.43 (dd, J = 9.8, 1.9 Hz, |
| | | | 1H), 1.95 (dd, J = 10.2, 2.1 Hz, |
| | | | 1H), 1.90-1.72 (m, 1H), 1.62- |
| | | | 1.56 (m, 1H). |
| 122 | 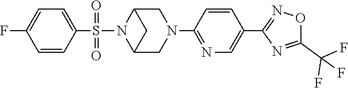 [SMILES: O=S(=O)(c1ccc(F)cc1)N1C2CC1CN(c1ccc(-c3noc(C(F)(F)F)n3)cn1)C2] | 3-(6-(6-((4-fluorophenyl) sulfonyl)-3,6-diazabicyclo [3.1.1]heptan-3-yl)pyridin-3- yl)-5-(trifluoromethyl)-1,2,4- oxadiazole | RT = 6.327 Min. (HPLC Method- I). ¹H NMR (400 MHz, CDCl₃) δ 8.89 (d, J = 2.3 Hz, 1H), 8.12 (dd, J = 9.0, 2.4 Hz, 1H), 7.99- 7.86 (m, 1H), 7.13 (t, J = 8.4 Hz, 1H), 6.52 (d, J = 9.0 Hz, 1H), |
| | | | 4.46 (d, J = 6.3 Hz, 1H), 4.04 (d, |
| | | | J = 12.1 Hz, 2H), 3.77 (s, 2H), |
| | | | 2.86 (q, J = 7.3 Hz, 1H), 1.68 (d, |
| | | | J = 8.9 Hz, 1H), 1.28 (dt, J = 12.4, 6.2 |
| | | | Hz, 1H). |
| 123 | 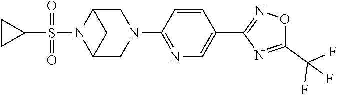 [SMILES: O=S(=O)(C1CC1)N1C2CC1CN(c1ccc(-c3noc(C(F)(F)F)n3)cn1)C2] | 3-(6-(6-(cyclopropylsulfonyl)- 3,6-diazabicyclo[3.1.1] heptan-3-yl)pyridin-3-yl)-5- (trifluoromethyl)-1,2,4-oxadiazole | RT = 5.63 Min. (HPLC Method- I). ¹H NMR (400 MHz, CDCl₃) δ 8.92 (dd, J = 2.4, 0.7 Hz, 1H), 8.14 (dd, J = 8.9, 2.4 Hz, 1H), 6.61 (dd, J = 9.0, 0.8 Hz, 1H), 4.50 (d, J = 6.3 Hz, 1H), 4.13 (d, J = 12.1 Hz, 1H), 3.81 (s, 1H), |
| | | | 3.14-3.01 (m, 1H), 2.38 (tt, J = |
| | | | 7.9, 4.8 Hz, 2H), 1.75 (d, J = 8.8 |
| | | | Hz, 2H), 1.15 (ddd, J = 6.0, 3.9, |
| | | | 2.8 Hz, 2H), 1.05-0.90 (m, 1H). |

Method-VI: To a stirred solution of the intermediate—5 TFA salt (0.10 mmol) in DCM (3 to 5 mL) was added DIPEA (0.30 mmol) followed by addition of sulfonyl chloride (0.10 mmol) at room temperature After stirring 1-2 h, the reaction was quenched with water and the product was extracted with DCM. Purification on a silica gel TLC using 50% EA/Hex afforded the titled compound.

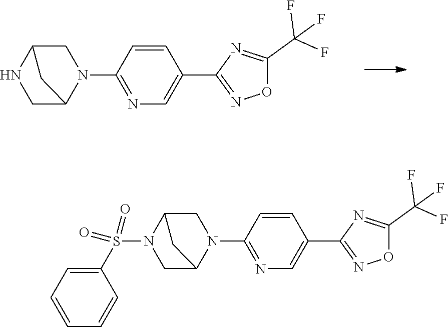 [SMILES: FC(F)(F)c1nc(-c2ccc(N3CC4CC3CN4)nc2)no1 ; O=S(=O)(c1ccccc1)N1CC2CC1CN2c1ccc(-c2noc(C(F)(F)F)n2)cn1]

### Compound of Invention—115, Example-115:3-(6-(5-(phenylsulfonyl)-2,5-diazabicyclo[2.2.1]heptan-2-yl)pyridin-3-yl)-5-(trifluoromethyl)-1,2,4-oxadiazole

To a stirred solution of compound 5-(5-(5-(trifluoromethyl)-1,2,4-oxadiazol-3-yl)pyridin-2-yl)-2,5-diazabicyclo[2.2.1]heptan-2-ium trifluoroacetate (the intermediate 5) (0.052 g, 0.096 mmol) and DIPEA (0.086 mL, 0.062 g, 0.048 mmol) in DCM (3 mL) was added benzenesulfonyl chloride (0.017 g, 0.096 mmol) at room temperature. After stirring 2 h, the reaction was quenched with water and the product was extracted using DCM. Purification on a silica gel preparatory glass TLC plate 30% EA/Hex followed by removal of solvent afforded titled compound 3-(6-(5-(phenylsulfonyl)-2,5-diazabicyclo[2.2.1]heptan-2-yl)pyridin-3-yl)-5-(trifluoromethyl)-1,2,4-oxadiazole Example-115 as a white solid (0.030 g, 66%). RT=6.33 Min. (HPLC Method-I). MS-ESI (m/z): Calculated [M+H]⁺=452.09; Observed [M+H]⁺=452.09.

### Compound of Invention—116, Example-116:3-(6-(5-((4-fluorophenyl) sulfonyl)-2,5-diazabicyclo[2.2.1]heptan-2-yl)pyridin-3-yl)-5-(trifluoromethyl)-1,2,4-oxadiazole

Example-116 was synthesized in an analogous manner to Example-115 by following method-VI using the intermediate 5. Purification on a silica gel preparatory glass TLC plate using 50% EA/Hex followed by extraction afforded the title compound Example-116 as a white solid (0.031 g, 62%). RT=6.48 Min. (HPLC Method-I). MS-ESI (m/z): Calculated [M+H]⁺=470.09; Observed [M+H]⁺=470.09.

### Compound of Invention—117, Example-117:3-(6-(5-((2-chlorophenyl) sulfonyl)-2,5-diazabicyclo[2.2.1]heptan-2-yl)pyridin-3-yl)-5-(trifluoromethyl)-1,2,4-oxadiazole

Example-117 was synthesized in an analogous manner to Example-115 by following method-VI using the intermediate 5. Purification on a silica gel preparatory glass TLC plate using 50% EA/Hex followed by extraction afforded the title compound Example-117 as a white solid. (0.028 g, 55%) TLC: Tr=0.40 in 30% EA/Hex. RT=6.66 Min. (HPLC Method-I). MS-ESI (m/z): Calculated [M+H]⁺=486.05; Observed [M+H]⁺=486.05.

### Compound of Invention—118, Example-118:3-(6-(5-(cyclopropylsulfonyl)-2,5-diazabicyclo[2.2.1]heptan-2-yl)pyridin-3-yl)-5-(trifluoromethyl)-1,2,4-oxadiazole

Example-118 was synthesized in an analogous manner to Example-115 by following method-VI using the intermediate 5. Purification on a silica gel preparatory glass TLC plate using 50% EA/Hex followed by extraction afforded the title compound Example-118as a white solid (9.6 mg, 23%). RT=5.58 Min. (HPLC Method-I). MS-ESI (m/z): Calculated [M+H]⁺=416.09; Observed [M+H]⁺=416.09.

### Compound of Invention—119, Example-119:3-(6-(5-(naphthalen-2-ylsulfonyl)-2,5-diazabicyclo[2.2.1]heptan-2-yl)pyridin-3-yl)-5-(trifluoromethyl)-1,2,4-oxadiazole

Example-119 was synthesized in an analogous manner to Example-115 by following method-VI using the intermediate 5. Purification on a silica gel preparatory glass TLC plate using 50% EA/Hex followed by extraction afforded the title compound Example-119 as a white solid (21 mg, 44%). RT=6.83 Min. (HPLC Method-I). MS-ESI (m/z): Calculated [M+H]⁺=502.11; Observed [M+H]⁺=502.11.

### Compound of Invention—120, Example-120:3-(2-(5-((4-Fluorophenyl) sulfonyl)-2,5-diazabicyclo[2.2.1]heptan-2-yl)thiazol-5-yl)-5-(trifluoromethyl)-1,2,4-oxadiazole

Example-120 was synthesized in an analogous manner to Example-115 by following method-VI using the intermediate 31. Purification on a silica gel preparatory glass TLC plate using 50% EA/Hex followed by extraction afforded the title compound Example-120 as a white solid (35 mg, 41%). RT=6.87 Min. (HPLC Method-I).

### Compound of Invention—121, Example-121: (3-(2-(5-(Naphthalen-2-ylsulfonyl)-2,5-diazabicyclo[2.2.1]heptan-2-yl)thiazol-5-yl)-5-(trifluoromethyl)-1,2,4-oxadiazole

Example-121 was synthesized in an analogous manner to Example-115 by following method-VI using the intermediate 31. Purification on a silica gel preparatory glass TLC plate using 50% EA/Hex followed by extraction afforded the title compound Example-121as a white solid (44 mg, 49%). RT=7.25 Min. (HPLC Method-I). Q-TOF LC/MS (m/z): Calculated [M+H]⁺=507.599; Observed [M+H]⁺=508.0757.

### Compound of Invention—122, Example-122:3-(6-(6-((4-Fluorophenyl) sulfonyl)-3,6-diazabicyclo[3.1.1]heptan-3-yl)pyridin-3-yl)-5-(trifluoromethyl)-1,2,4-oxadiazole

Example-122 was synthesized in an analogous manner to Example-115 by following method-VI using the intermediate 16b. Purification on a silica gel preparatory glass TLC plate using 70% EA/Hex followed by extraction afforded the title compound Example-122 as a white solid (44 mg, 44%). RT=6.87 Min. (HPLC Method-I).

### Compound of Invention—123, Example-123:3-(6-(6-(cyclopropylsulfonyl)-3,6-diazabicyclo[3.1.1]heptan-3-yl)pyridin-3-yl)-5-(trifluoromethyl)-1,2,4-oxadiazole

Example-123 was synthesized in an analogous manner to Example-115 by following method-VI using the intermediate 16b. Purification on a silica gel preparatory glass TLC plate using 70% EA/Hex followed by extraction afforded the title compound Example-123 as a white solid (47 mg, 53%). RT=5.63 Min. (HPLC Method-I).

### Example C

### Pharmacological Evaluation of Compounds of the Invention

In vitro Assays

Dose response curves of HDAC6 inhibition for the HDAC6 inhibitors CAY10603, tubastatin A HCl (TubA), and SE-7552 were obtained using the Fluorometric HDAC6 Activity Assay Kit (BioVision (Milpitas, CA, Cat #: K466) according to manufacturer's instructions. Briefly, compounds (dissolved in DMSO) or DMSO were incubated with HDAC6 diluted in the assay buffer of the kit for 10 minutes at 37° C. in a 96-well plate. The HDAC6 substrate diluted in the assay buffer was added to the wells and incubated for 30 minutes at 37° C., avoiding direct light exposure. The developer was added to the wells, incubated for 10 minutes at 37° C., and the fluorescence was measured at Ex/Em 380/490 nm in an end point mode at 37° C. Reading from the wells that were not added HDAC6 but the substrate was used as the background reading. Selected CCG analogs exhibited more than 50% inhibition at 10 μM concentration.

For western blot analysis, embryonic mouse hypothalamus cell line (mHypoE-N1) obtained from Cedarlane LABs (Cat #: CLU101) were treated with DMSO, Tubastatin A HCl (TubA), or SE-7552 for 24 hr, and then lysed in RIPA buffer (50 mM TRIS pH: 7.50, 25 mM NaF, 100 mM NaCl, SmM EDTA, 0.1% SDS, 1% TritonX-100) supplemented with protease and phosphatase inhibitors, 20 mM nicotinamide and 20 μM vorinostat. Equal amounts of total-lysates were separated on 4-15% SDS-PAGE gels (Bio-Rad), transferred to PVDF membranes (Millipore), and probed with indicated antibodies. Blots were washed with PBS/T (0.1% Tween-20 in PBS) and probed with HRP-conjugated secondary antibodies, and developed. Rabbit monoclonal anti-Acetyl-α-Tubulin (Cat #5335), Rabbit polyclonal anti-α-Tubulin (Cat #2144), were from Cell Signaling (Danvers, MA). Selected CCG analogs increased acetyl-α-Tubulin levels over vehicle (DMSO) control at 1 μM.

### In Vivo Studies

Animals were housed at a 12 hours dark/light cycle, temperature and humidity controlled rooms. Mice were purchased from the Jackson LABoratory, except the HDAC6 global knockout (KO) mice, which were provided by Dr. Timothy A. Mckinsey (University of Colorado Anschutz Medical Campus, Aurora, CO, USA.). Mice were fed either regular chow or high fat diet (60 kcal % fat, Research Diets) to induce obesity (diet-induced obesity) and had free access to food and water unless specified. Body composition was analyzed with Bruker's minispec LF50 Body Composition Analyzer. Blood glucose was measured from tail vein by Contour blood glucose monitor system (Bayer). TubA (25 mg/kg body weight) or SE-7552 (50 mg/kg body weight) were administered within 1 hr before dark cycle daily by intraperitoneal (ip.) injections. For intraperitoneal injections, drugs or vehicle was injected at 25 uL volumes per animal. Mice and their food were measured daily or weekly to track body weight and food intake. Hematoxylin and eosin (H&E) staining of liver sections of diet-induced obese mice were done after one month of vehicle or TubA injections.

### Example D

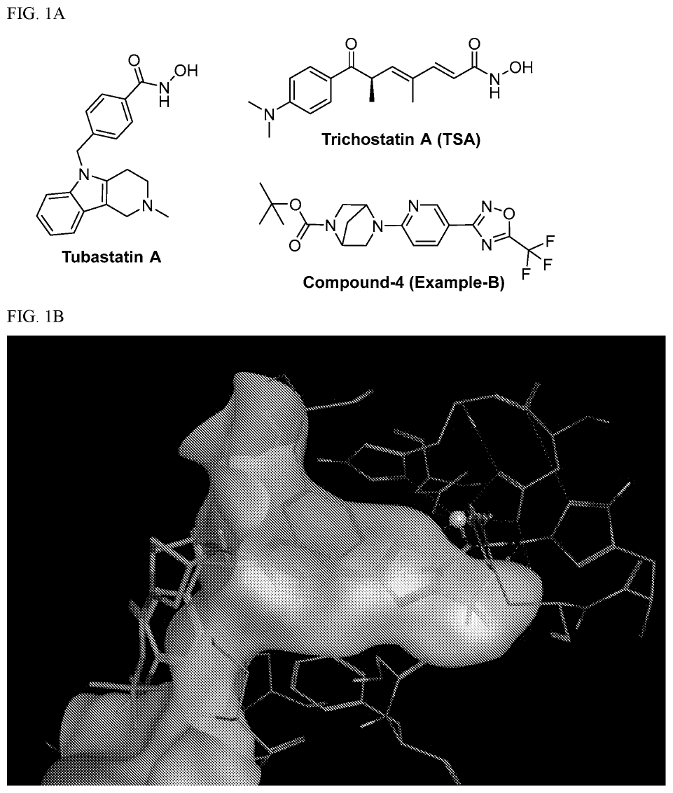

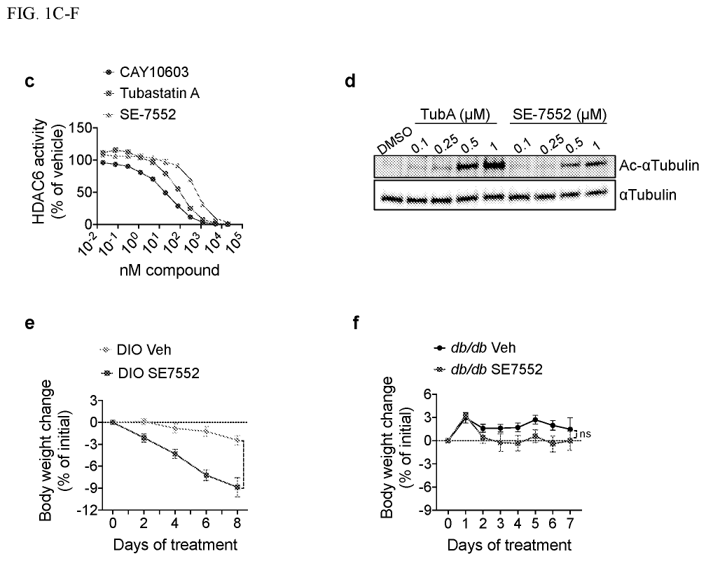

HDAC6 is a zinc dependent enzyme, and potent HDAC6-inhibitors including tubastatin contain the hydroxamic acid residue as the zinc chelating moiety (FIG. 1a). While HDAC6-specific inhibitors were shown to be safer than pan-HDAC inhibitors^{47,48}, the potential problems associated with the hydroxamates have hindered their clinical use⁴⁹. We (the inventors) identified that a non-hydroxamate HDAC6-specific inhibitor (SE7552), which has About 1000-fold selectivity versus all other known HDAC isozymes 50 and is structurally unrelated to tubastatin (FIG. 1a-d), effectively reduces obesity (FIG. 1e), serving as the proof-of-principle study for the development of non-hydroxamate HDAC6 inhibitors as safe anti-obesity therapeutics. Notably, SE7552 was also ineffective at inducing weight loss in db/db mice (FIG. 1f), further supporting our conclusion that HDAC6 inhibition-induced weight loss requires intact leptin signaling. SE-7552 was synthesized as described and dissolved in DMSO for animal studies⁷⁶.

### Example E

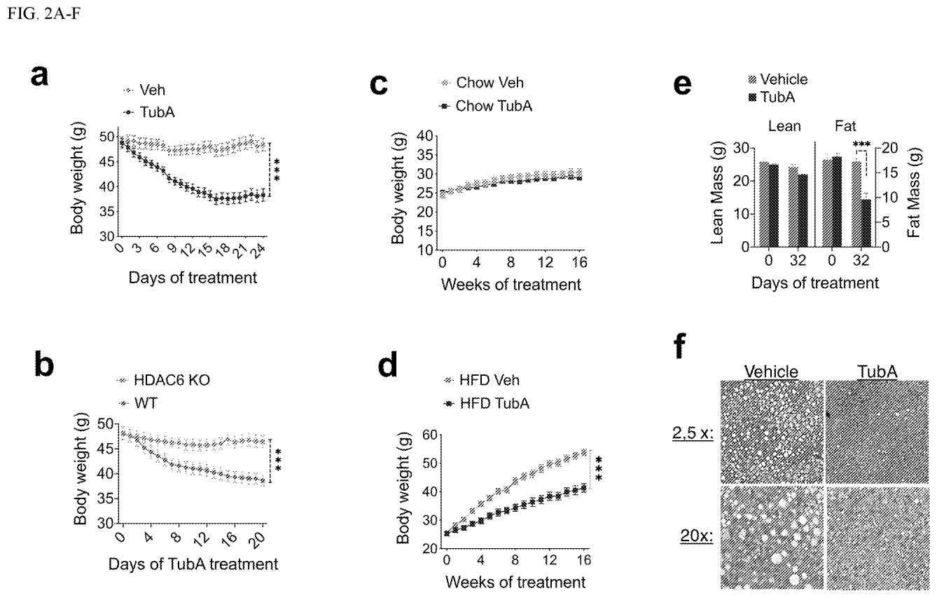

FIG. 2A-F shows that HDAC6 inhibition reverses obesity.

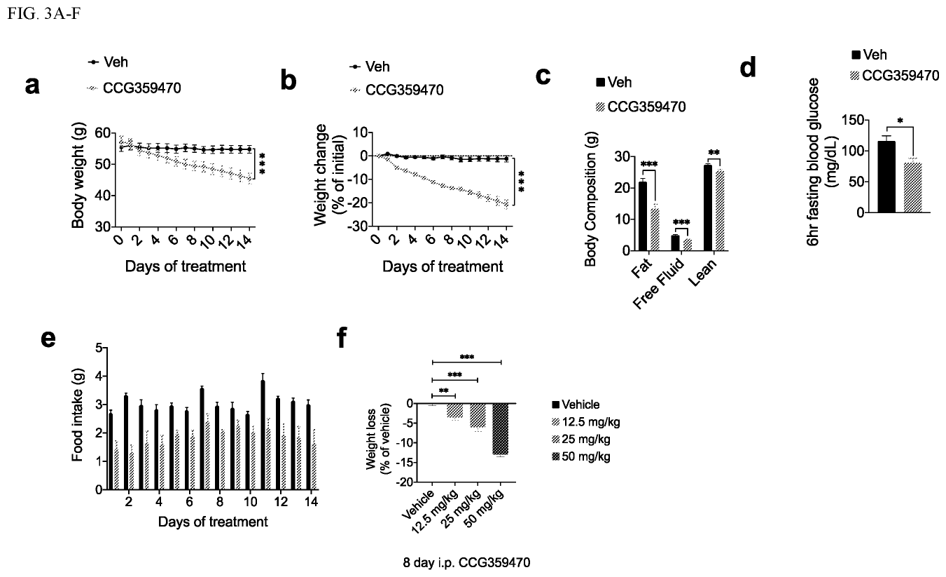

FIG. 3A-F shows that compound CCG359470 reduces body weight and fasting glucose.

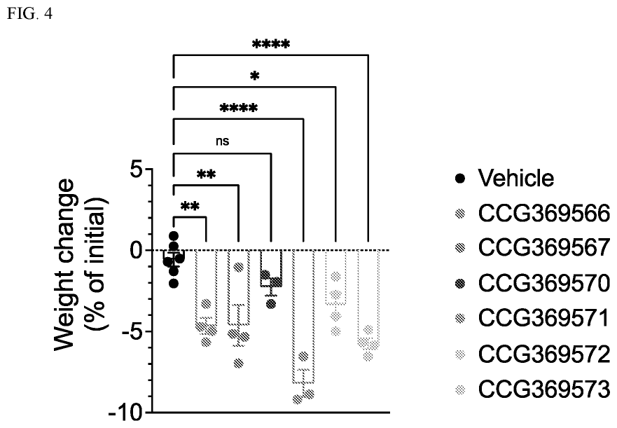

FIG. 4 shows in vivo screening of CCG compounds on diet-induced obese mice.

### INCORPORATION BY REFERENCE

The entire disclosure of each of the patent documents and scientific articles referred to herein is incorporated by reference for all purposes. The following references (numerically denoted throughout) are herein incorporated by reference in their entireties:
1. Ng, M. et al. Global, regional, and national prevalence of overweight and obesity in children and adults during 1980-2013: a systematic analysis for the Global Burden of Disease Study 2013. Lancet 384, 766-781 (2014).
2. Elmquist, J. K., Bjørbaek, C., Ahima, R. S., Flier, J. S. & Saper, C. B. Distributions of leptin receptor mRNA isoforms in the rat brain. J. Comp. Neurol. 395, 535-547 (1998).
3. Maffei, M. et al. Leptin levels in human and rodent: measurement of plasma leptin and ob RNA in obese and weight-reduced subjects. Nat. Med. 1, 1155-1161 (1995).
4. Andreoli, M. F., Donato, J., Cakir, I. & Perello, M. Leptin resensitisation: a reversion of leptin-resistant states. J. Endocrinol. 241, R81-R96 (2019).
5. Cavadas, C., Aveleira, C. A., Souza, G. F. P. & Velloso, L. A. The pathophysiology of defective proteostasis in the hypothalamus—from obesity to ageing. Nat. Rev. Endocrinol. 12, 723-733 (2016).
6. Kondo, T. et al. Heat shock response regulates insulin sensitivity and glucose homeostasis: pathophysiological impact and therapeutic potential. Curr. Diabetes Rev. 7, 264-269 (2011).
7. Quan, W., Jung, H. S. & Lee, M.-S. Role of autophagy in the progression from obesity to Diabetes and in the control of energy balance. Arch. Pharm. Res. 36, 223-229 (2013).
8. Wing, S. S. The UPS in Diabetes and obesity. BMC Biochem. 9 Suppl 1, S6 (2008).
9. Barlow, A. D. & Thomas, D. C. Autophagy in Diabetes: β-cell dysfunction, insulin resistance, and complications. DNA Cell Biol. 34, 252-260 (2015).
10. Ryter, S. W., Koo, J. K. & Choi, A. M. K. Molecular regulation of autophagy and its implications for metabolic diseases. Curr. Opin. Clin. Nutr. MetAB. Care 17, 329-337 (2014).
11. ABubaker, J. et al. DNAJB3/HSP-40 cochaperone is downregulated in obese humans and is restored by physical exercise. PLOS One 8, e69217 (2013).
12. Islam, A., Hait, S. H., Andrews-Shigaki, B., Carus, S. & Deuster, P. A. Plasma HSP70 levels correlate with health risk factors and insulin resistance in African American subjects. Exp. Clin. Endocrinol. Diabetes 122, 496-501 (2014).
13. Matz, J. M., LaVoi, K. P., Epstein, P. N. & Blake, M. J. Thermoregulatory and heat-shock protein response deficits in cold-exposed diABetic mice. Am. J. Physiol. 270, R525-32 (1996).
14. Tiss, A. et al. Immunohistochemical profiling of the heat shock response in obese non-diABetic subjects revealed impaired expression of heat shock proteins in the adipose tissue. Lipids Health Dis. 13, 106 (2014).
15. Bollinger, L. M., Powell, J. J. S., Houmard, J. A., Witczak, C. A. & Brault, J. J. Skeletal muscle myotubes in severe obesity exhibit altered ubiquitin-proteasome and autophagic/lysosomal proteolytic flux: Proteolysis of Myotubes in Obesity. Obesity 23, 1185-1193 (2015).
16. Ignacio-Souza, L. M. et al. Defective regulation of the ubiquitin/proteasome system in the hypothalamus of obese male mice. Endocrinology 155, 2831-2844 (2014).
17. Hotamisligil, G. S. Endoplasmic reticulum stress and the inflammatory basis of metabolic disease. Cell 140, 900-917 (2010).
18. Lee, M.-S. Effect of mitochondrial stress on systemic metabolism. Ann. N. Y. Acad. Sci. 1350, 61-65 (2015).
19. Zhang, M. et al. HDAC6 deacetylates and ubiquitinates MSH2 to maintain proper levels of MutSα. Mol. Cell 55, 31-46 (2014).
20. Hook, S. S., Orian, A., Cowley, S. M. & Eisenman, R. N. Histone deacetylase 6 binds polyubiquitin through its zinc finger (PAZ domain) and copurifies with deubiquitinating enzymes. Proc. Natl. Acad. Sci. U.S.A 99, 13425-13430 (2002).
21. Seigneurin-Berny, D. et al. Identification of components of the murine histone deacetylase 6 complex: link between acetylation and ubiquitination signaling pathways. Mol. Cell. Biol. 21, 8035-8044 (2001).
22. Kawaguchi, Y. et al. The deacetylase HDAC6 regulates aggresome formation and cell viABility in response to misfolded protein stress. Cell 115, 727-738 (2003).
23. Kwon, S., Zhang, Y. & Matthias, P. The deacetylase HDAC6 is a novel critical component of stress granules involved in the stress response. Genes Dev. 21, 3381-3394 (2007).
24. Lee, J.-Y. et al. HDAC6 controls autophagosome maturation essential for ubiquitin-selective quality-control autophagy. EMBO J. 29, 969-980 (2010).
25. Lee, J.-Y., Nagano, Y., Taylor, J. P., Lim, K. L. & Yao, T.-P. Disease-causing mutations in parkin impair mitochondrial ubiquitination, aggregation, and HDAC6-dependent mitophagy. J. Cell Biol. 189, 671-679 (2010).
26. Yan, J. et al. SQSTM1/p62 interacts with HDAC6 and regulates deacetylase activity. PLOS One 8, e76016 (2013).
27. Butler, K. V. et al. Rational design and simple chemistry yield a superior, neuroprotective HDAC6 inhibitor, tubastatin A. J. Am. Chem. Soc. 132, 10842-10846 (2010).
28. Jochems, J. et al. Antidepressant-like properties of novel HDAC6-selective inhibitors with improved brain bioavailABility. Neuropsychopharmacology 39, 389-400 (2014).
29. d′Ydewalle, C. et al. HDAC6 inhibitors reverse axonal loss in a mouse model of mutant HSPB1-induced Charcot-Marie-Tooth disease. Nat. Med. 17, 968 (2011).
30. Vishwakarma, S. et al. Tubastatin, a selective histone deacetylase 6 inhibitor shows anti-inflammatory and anti-rheumatic effects. Int. Immunopharmacol. 16, 72-78 (2013).
31. Xu, X., Kozikowski, A. P. & Pozzo-Miller, L. A selective histone deacetylase-6 inhibitor improves BDNF trafficking in hippocampal neurons from Mecp2 knockout mice: implications for Rett syndrome. Front. Cell. Neurosci. 8, 68 (2014).
32. Zhang, L. et al. Tubastatin A/ACY-1215 improves cognition in Alzheimer's disease transgenic mice. J. Alzheimers. Dis. 41, 1193-1205 (2014).
33. Olson, D. E. et al. Hydroxamate-based histone deacetylase inhibitors can protect neurons from oxidative stress via a histone deacetylase-independent catalase-like mechanism. Chem. Biol. 22, 439-445 (2015).
34. Kirchner, H. et al. Caloric restriction chronically impairs metabolic programming in mice. Diabetes 61, 2734-2742 (2012).
35. Demos-Davies, K. M. et al. HDAC6 contributes to pathological responses of heart and skeletal muscle to chronic angiotensin-II signaling. Am. J. Physiol. Heart Circ. Physiol. 307, H252-8 (2014).
36. Williams, K. A. et al. Extracellular signal-regulated kinase (ERK) phosphorylates histone deacetylase 6 (HDAC6) at serine 1035 to stimulate cell migration. J. Biol. Chem. 288, 33156-33170 (2013).
37. Bost, F. et al. The extracellular signal-regulated kinase isoform ERK1 is specifically required for in vitro and in vivo adipogenesis. Diabetes 54, 402-411 (2005).
38. Seeley, R. J. et al. Melanocortin receptors in leptin effects. Nature 390, 349 (1997).
39. Mazor, R. et al. Cleavage of the leptin receptor by matrix metalloproteinase-2 promotes leptin resistance and obesity in mice. Sci. Transl. Med. 10, (2018).
40. Knight, Z. A., Hannan, K. S., Greenberg, M. L. & Friedman, J. M. Hyperleptinemia is required for the development of leptin resistance. PLOS One 5, e11376 (2010).
41. Allison, M. B. et al. Defining the Transcriptional Targets of Leptin Reveals a Role For Atf3 in Leptin Action. Diabetes (2018) doi: 10.2337/db17-1395.
42. Zhang, Y. et al. Mice lacking histone deacetylase 6 have hyperacetylated tubulin but are viABle and develop normally. Mol. Cell. Biol. 28, 1688-1701 (2008).
43. Shirazi, R. et al. Glucagon-like peptide 1 receptor induced suppression of food intake, and body weight is mediated by central IL-1 and IL-6. Proc. Natl. Acad. Sci. U.S.A 110, 16199-16204 (2013).
44. Larsen, L., Le Foll, C., Dunn-Meynell, A. A. & Levin, B. E. IL-6 ameliorates defective leptin sensitivity in DIO ventromedial hypothalamic nucleus neurons. Am. J. Physiol. Regul. Integr. Comp. Physiol. 311, R764-R770 (2016).
45. Feng, X. et al. ILIR1 is required for celastrol's leptin-sensitization and antiobesity effects. Nat. Med. 25, 575-582 (2019).
46. Sadagurski, M. et al. Human IL6 enhances leptin action in mice. DiABetologia 53, 525-535 (2010).
47. Subramanian, S., Bates, S. E., Wright, J. J., Espinoza-Delgado, I. & Piekarz, R. L. Clinical Toxicities of Histone Deacetylase Inhibitors. Pharmaceuticals 3, 2751-2767 (2010).
48. Witt, O., Deubzer, H. E., Milde, T. & Oehme, I. HDAC family: What are the cancer relevant targets? Cancer Lett. 277, 8-21 (2009).
49. Shen, S. & Kozikowski, A. P. Why Hydroxamates May Not Be the Best Histone Deacetylase Inhibitors—What Some May Have Forgotten or Would Rather Forget? ChemMedChem 11, 15-21 (2016).
50. Holt, J. A. et al. SE-7552, a Highly Selective, Non-Hydroxamate Inhibitor of Histone Deacetylase-6 Blocks Multiple Myeloma Growth In Vivo. Blood 132, 3215-3215 (2018).
51. Winkler, R. et al. Histone deacetylase 6 (HDAC6) is an essential modifier of glucocorticoid-induced hepatic gluconeogenesis. Diabetes 61, 513-523 (2012).
52. Fujikawa, T. et al. Leptin engages a hypothalamic neurocircuitry to permit survival in the ABsence of insulin. Cell MetAB. 18, 431-444 (2013).
53. Ahima, R. S. et al. Role of leptin in the neuroendocrine response to fasting. Nature 382, 250-252 (1996).
54. Davie, J. R. Inhibition of histone deacetylase activity by butyrate. J. Nutr. 133, 2485S-2493S (2003).
55. Ozcan, U. et al. Chemical chaperones reduce ER stress and restore glucose homeostasis in a mouse model of type 2 Diabetes. Science 313, 1137-1140 (2006).
56. KABra, D. G. et al. Hypothalamic leptin action is mediated by histone deacetylase 5. Nat. Commun. 7, 10782 (2016).
57. Galmozzi, A. et al. Inhibition of class I histone deacetylases unveils a mitochondrial signature and enhances oxidative metabolism in skeletal muscle and adipose tissue. Diabetes 62, 732-742 (2013).
58. Ferrari, A. et al. Attenuation of diet-induced obesity and induction of white fat browning with a chemical inhibitor of histone deacetylases. Int. J. Obes. 41, 289-298 (2017).
59. Tam, J. et al. Peripheral cannABinoid-1 receptor inverse agonism reduces obesity by reversing leptin resistance. Cell MetAB. 16, 167-179 (2012).
60. Zhao, S. et al. Partial Leptin Reduction as an Insulin Sensitization and Weight Loss Strategy. Cell MetAB. 30, 706-719.e6 (2019).
61. Saito, K. et al. Celastrol Reduces Obesity in MC4R Deficiency and Stimulates Sympathetic Nerve Activity Affecting Metabolic and Cardiovascular Functions. Diabetes 68, 1210-1220 (2019).
62. Fribley, A. M. et al. Celastrol induces unfolded protein response-dependent cell death in head and neck cancer. Exp. Cell Res. 330, 412-422 (2015).
63. Cakir, I. & Nillni, E. A. Endoplasmic Reticulum Stress, the Hypothalamus, and Energy Balance. Trends Endocrinol. MetAB. 30, 163-176 (2019).
64. Qian, H. et al. HDAC6-mediated acetylation of lipid droplet-binding protein CIDEC regulates fat-induced lipid storage. J. Clin. Invest. 127, 1353-1369 (2017).
65. Forcioli-Conti, N., Estève, D., Bouloumié, A., Dani, C. & Peraldi, P. The size of the primary cilium and acetylated tubulin are modulated during adipocyte differentiation: Analysis of HDAC6 functions in these processes. Biochimie 124, 112-123 (2016).
66. Lundh, M. et al. Afadin is a scaffold protein repressing insulin action via HDAC6 in adipose tissue. EMBO Rep. e48216 (2019).
67. Harris, R. B. & Martin, R. J. Site of action of putative lipostatic factor: food intake and peripheral pentose shunt activity. Am. J. Physiol. 259, R45-52 (1990).
68. White, C. L., Purpera, M. N., Ballard, K. & Morrison, C. D. Decreased food intake following overfeeding involves leptin-dependent and leptin-independent mechanisms. Physiol. Behav. 100, 408-416 (2010).
69. Ravussin, Y., Leibel, R. L. & Ferrante, A. W., Jr. A missing link in body weight homeostasis: the catABolic signal of the overfed state. Cell MetAB. 20, 565-572 (2014).
70. Salminen, A., Kaarniranta, K. & Kauppinen, A. Regulation of longevity by FGF21: Interaction between energy metabolism and stress responses. Ageing Res. Rev. 37, 79-93 (2017).
71. Taylor, R. C., Berendzen, K. M. & Dillin, A. Systemic stress signalling: understanding the cell non-autonomous control of proteostasis. Nat. Rev. Mol. Cell Biol. 15, 211-217 (2014).
72. Xu, A. et al. Microtubule regulators act in the nervous system to modulate fat metabolism and longevity through DAF-16 in C. elegans. Aging Cell 18, e12884 (2019).
73. Ratti, F. et al. Histone deacetylase 6 is a FoxO transcription factor-dependent effector in skeletal muscle atrophy. J. Biol. Chem. 290, 4215-4224 (2015).
74. Lighton, J. R. B. Measuring Metabolic Rates: A Manual for Scientists. (Oxford University Press, 2018).
75. Weir, J. B. de V. & de V. Weir, J. B. New methods for calculating metabolic rate with special reference to protein metabolism. J. Physiol. 109, 1-9 (1949).
76. YATES, Christopher & M. METALLOENZYME INHIBITOR COMPOUNDS. World Patent (2018).
77. Molecular Operating Environment (MOE), 2019.01; Chemical Computing Group ULC, 1010 Sherbrooke St. West, Suite #910, Montreal, QC, Canada, H3A 2R7, 2019.
78. Jones, G., Willett, P., Glen, R. C., Leach, A. R. & Taylor, R. Development and validation of a genetic algorithm for flexible docking. J. Mol. Biol. 267, 727-748 (1997).

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1-25. (canceled)

26. A compound encompassed within one or more of the following forumlas: 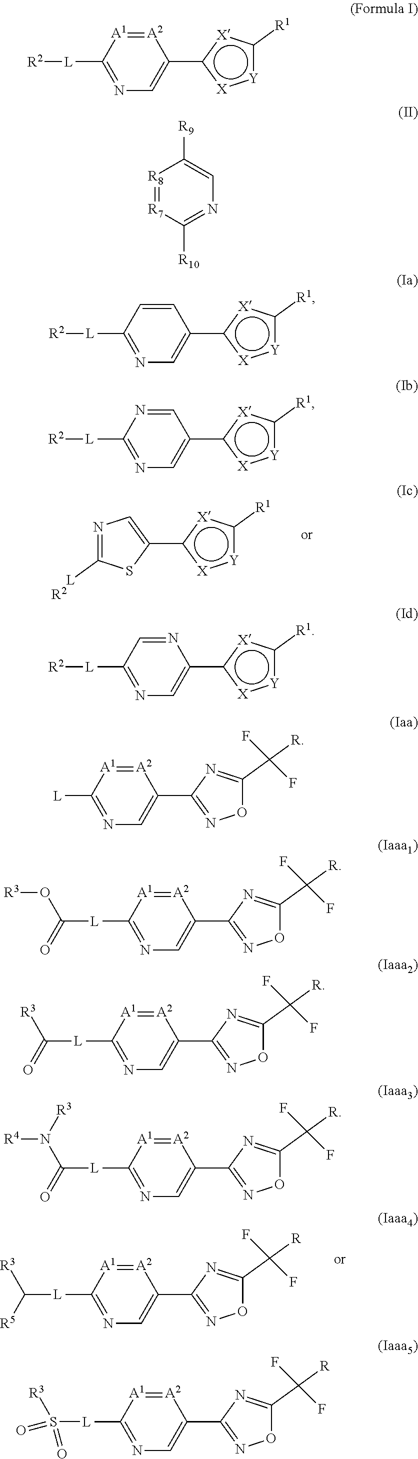 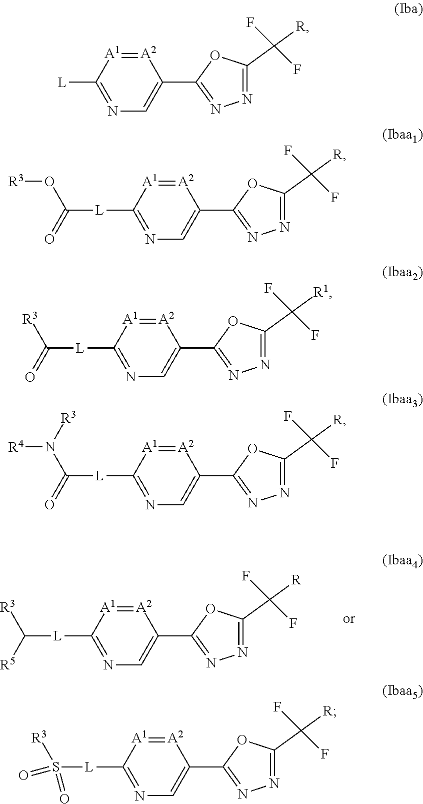
including pharmaceutically acceptable salts, solvates, and/or prodrugs thereof;
wherein X and X′ are N when Y is O, or X and Y are N when X′ is O;
wherein A¹ and A² are independently CH or N, or A¹ and A² are independently CH, or A¹ and A² taken together as S;
wherein R¹ is CF₃ or CF₂H;
wherein L is bicyclic diamines or spirocyclic diamines;
wherein R² is hydrogen, —COR³, —CO₂R³, —(CH₂),NR³R⁴, —CONR³R⁴, —(CH₂),R³R⁴, and —SO₂R³, wherein n=1 or 2 optionally substituted with a CH₃ group;
wherein R³ is hydrogen, C₁-C₆alkyl or C₁-C₆branchedalkylalkyl or C₁-C₆cycloalkyl, aryl, cycloalkylalkyl, heterocyclyl, heteroaryl, arylalkyl, arylaryl, or aryl, each of which is optionally mono-, di-, or tri-substituted independently with C₁-C₆alkyl, halogen, cycloalkyl, and aryl;
wherein R⁴ is C₁-C₆alkyl, cycloalkyl, cycloalkylalkyl, heterocyclyl, heteroaryl, arylalkyl, or aryl, each of which is optionally mono-, di-, or tri-substituted independently with C₁-C₆alkyl, halogen, cycloalkyl, and aryl; and
wherein R⁵ is hydrogen or CH₃,
wherein R is Fluorine or Hydrogen,
wherein R7, R8, R9 and R10 independently include any chemical moiety that permits the resulting compound to inhibit HDAC6 activity.

27. The compound of claim 26, wherein the linker L is a bicyclo diamino moiety selected from the group consisting of: bicyclic diamines, where the bicyclic diamines are 2,5-diazabicyclo[2.2.1]heptane, or 3,6-diazabicyclo[3.1.1]heptane, 3,6-diazabicyclo[3.1.0]hexane, 3,7-diazabicyclo[4.1.0]heptane, octahydropyrrolo[3,4-c]pyrrole, octahydro-1H-pyrrolo[3,4-c]pyridine, 3,8-diazabicyclo[3.2.1]octane, 3,6-diazabicyclo[3.2.1]octane, 3,9-diazabicyclo[3.3.1]nonane, 2,5-diazabicyclo[2.2.2]octane, or 2,5-diazabicyclo[4.2.0]octane,

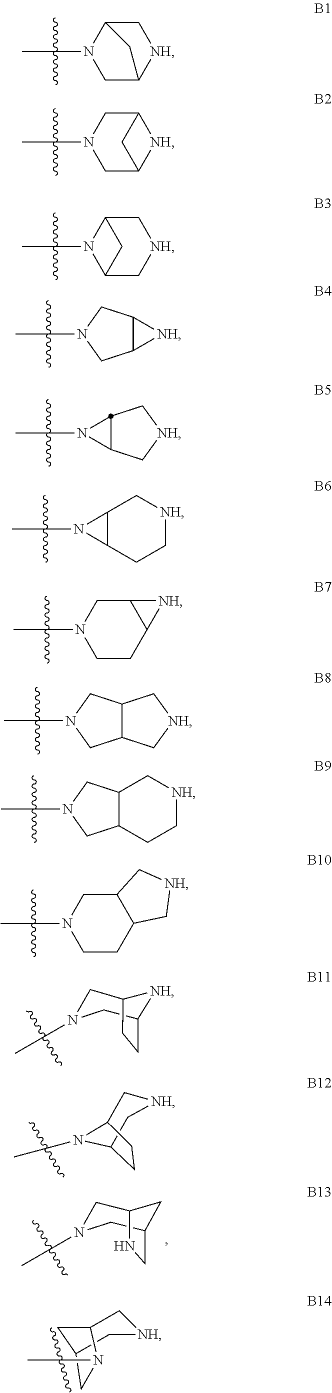

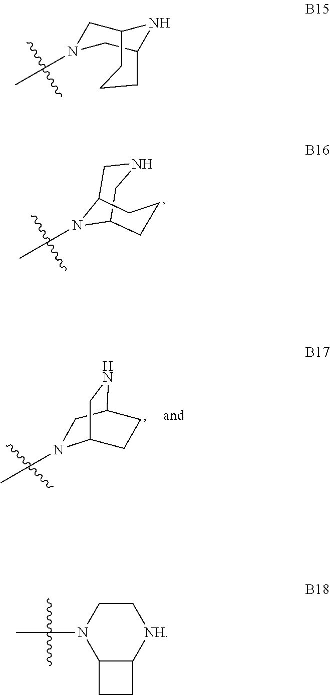

28. The compound of claim 26, wherein the linker L is a spirocyclo diamine moiety selected from the group consisting of: 2,6-diazaspiro[3.3]heptane, 2,6-diazaspiro[3.4]octane, 2,7-diazaspiro[4.4]nonane, 2,7-diazaspiro[3.5]nonane, 2,7-diazaspiro[3.5]nonane, 2,8-diazaspiro[4.5]decane, 2,9-diazaspiro[5.5]undecane, 3,9-diazaspiro[5.5]undecane and 4,7-diazaspiro[2.5]octane,

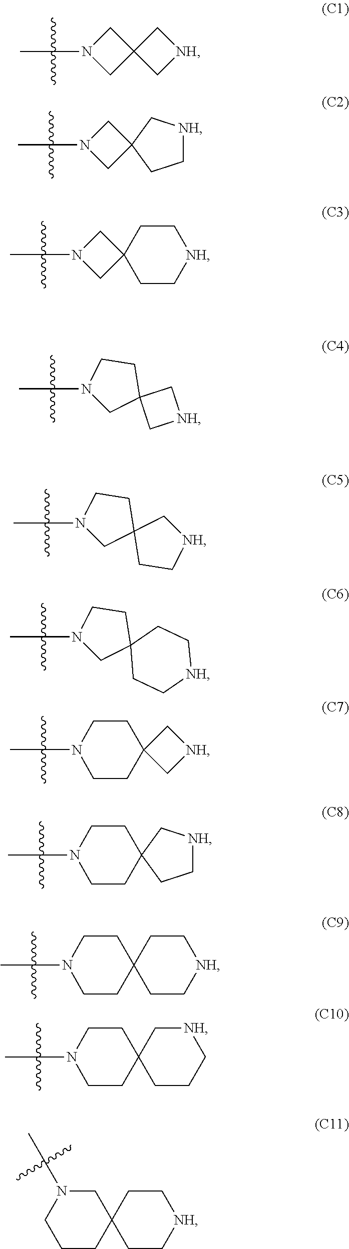

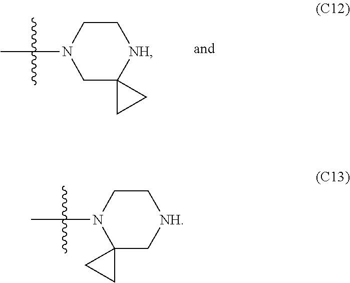 [SMILES: *N1CCNC2(CC2)C1 ; *N1CCNCC12CC2]

29. The compound of claim 26, wherein the compound is recited in Tables I, II, III, or IV.

30. The compound of claim 26, wherein R7 and R8 are each independently Carbon or Nitrogen.

31. The compound of claim 26, wherein R7 and R8 together are Sulfur such that the resulting structure is

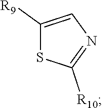

32. The compound of claim 26, wherein R8 is Carbon or Nitrogen.

33. The compound of claim 26, wherein R9 is selected from

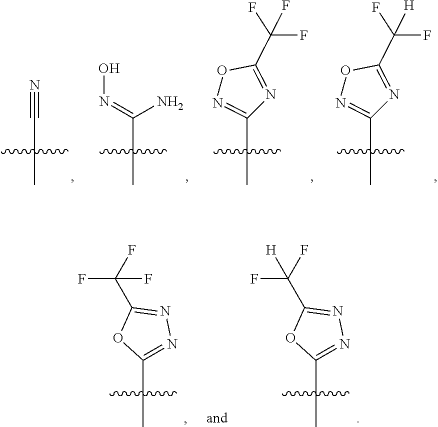

34. The compound of claim 26,
wherein R10 is selected from hydrogen, halogen (e.g., Chlorine), 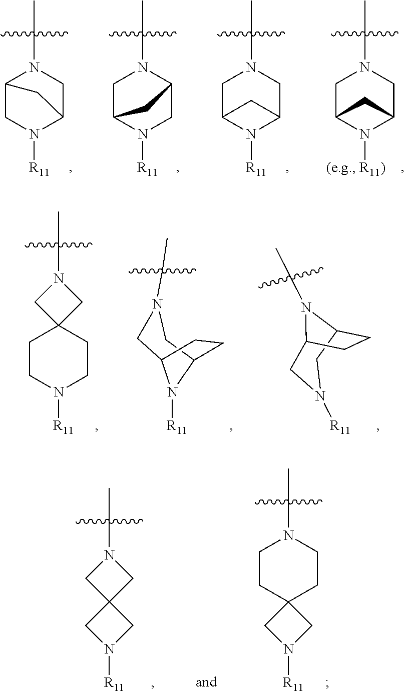
wherein R11 is selected from hydrogen,

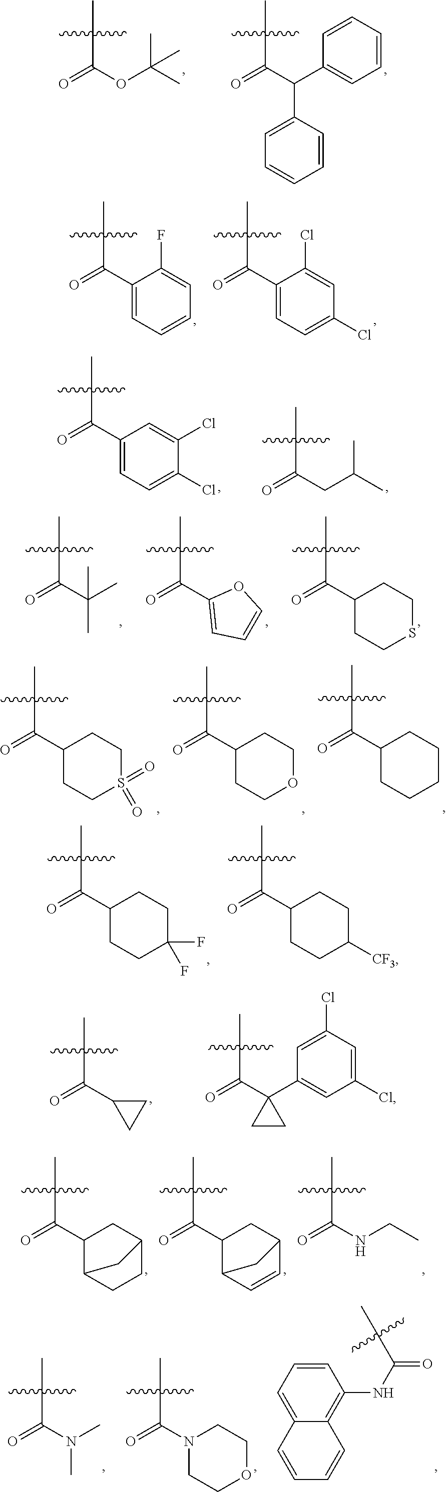

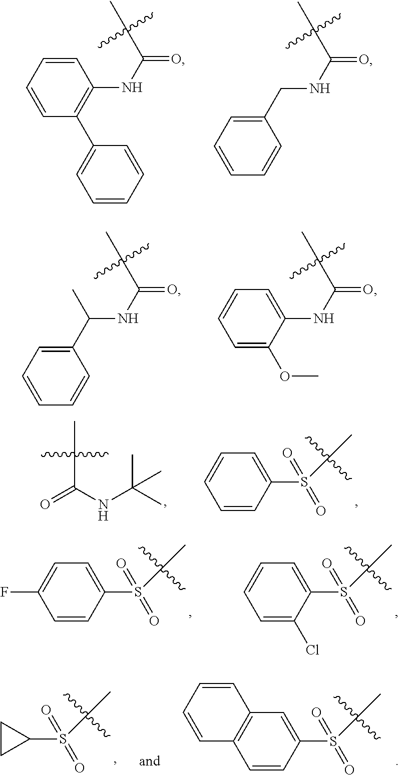

35. A method of treating, ameliorating, or preventing a disorder related to HDAC6 activity in a patient comprising administering to said patient a therapeutically effective amount of a pharmaceutical composition comprising a compound of claim 26.

36. The method of claim 35, wherein the disorder related to HDAC6 activity is one or more of a metabolic disorder (e.g., obesity, Diabetes), a neurological disorder (e.g., Alzheimer's disease, Parkinson disease, Huntington disease), cancer (e.g., multiple myeloma, biliary tract cancer, non-small cell lung cancer, chronic lymphocytic leukemia).

37. The method of claim 35, wherein the disorder related to HDAC6 activity is selected from Rett syndrome (RTT), inherited retinal disorders (IRDS), idiopathic pulmonary fibrosis (IPF), and Charcot-Marie-Tooth disease (CMT).

38. The method of claim 35, wherein said patient is a human patient.

39. The method of claim 35, further comprising administering to said patient one or more agents for treating a metabolic disorder (e.g., obesity, Diabetes), a neurological disorder (e.g., Alzheimer's disease, Parkinson disease, Huntington disease), and/or cancer (e.g., multiple myeloma, biliary tract cancer, non-small cell lung cancer, chronic lymphocytic leukemia).

40. The method of claim 35, further comprising administering to said patient one or more agents for treating RTT, IRDS, IPF, and/or CMT.

41. A method for increasing sensitivity to leptin in adipose tissue cells in a patient comprising administering to said patient a therapeutically effective amount of a pharmaceutical composition comprising a compound of claim 26.

42. The method of claim 41, wherein the patient is a human patient.

43. The method of claim 41, wherein the patient is suffering from or at risk of suffering from a disorder related to HDAC6 activity.

44. The method of claim 43, wherein the disorder related to HDAC6 activity is one or more of a metabolic disorder (e.g., obesity, Diabetes), a neurological disorder (e.g., Alzheimer's disease, Parkinson disease, Huntington disease), cancer (e.g., multiple myeloma, biliary tract cancer, non-small cell lung cancer, chronic lymphocytic leukemia).

45. The method of claim 43, wherein the disorder related to HDAC6 activity is selected from Rett syndrome (RTT), inherited retinal disorders (IRDS), idiopathic pulmonary fibrosis (IPF), and Charcot-Marie-Tooth disease (CMT).
